(19) United States
(12) Patent Application Publication
(10) Pub. No.: US 2025/0222082 A1
(43) Pub. Date: Jul. 10, 2025

(54) PROTEIN AND PEPTIDE VACCINES TARGETING METHANOGENS

(71) Applicant: Arkea Bio Corp., Charlestown, MA (US)

(72) Inventors: Matthew Dunn, Louisville, CO (US); Brigid Arciero, Boulder, CO (US); Colin South, Reno, NV (US); Kristin Adolfsen, Burlington, MA (US); Jeff Bickmeier, Arlington, MA (US); John Sullivan, Lexington, MA (US); Sara Kimler, Boston, MA (US); Lauren Fitch, Malden, MA (US); Joseph Koos, Somerville, MA (US); James Spoonamore, Somerville, MA (US); Zeljko Radulovic, Cambridge, MA (US); Caitlin Allen, Somerville, MA (US); Anna Li, Boston, MA (US); Alexander Hsu, Belmont, MA (US); Sara Tabandeh, Cambridge, MA (US); Frederick Richard Kearney, Walpole, MA (US)

(21) Appl. No.: 19/016,420

(22) Filed: Jan. 10, 2025

Related U.S. Application Data
(60) Provisional application No. 63/645,238, filed on May 10, 2024.
(60) Provisional application No. 63/619,572, filed on Jan. 10, 2024.

(51) Int. Cl. A61K 39/00; A23K 20/105; A23K 20/147; A23K 20/163; A23K 20/28; A23K 50/10; A61K 9/20; A61K 9/28; A61K 9/50; A61K 31/045; A61P 31/00; C07K 16/44

(52) U.S. Cl. CPC A61K 39/0001; A23K 20/105; A23K 20/147; A23K 20/163; A23K 20/28; A23K 50/10; A61K 9/2009; A61K 9/204; A61K 9/282; A61K 9/284; A61K 9/2866; A61K 9/2886; A61K 9/5026; A61K 9/5031; A61K 31/045; A61P 31/00; C07K 16/44; A61K 2039/542; A61K 2039/545; A61K 2039/552; A61K 2039/55555; A61K 2039/55566; A61K 2039/55572; A61K 2039/572; A61K 2039/575; A61K 2039/70

(57) ABSTRACT

The present invention relates to polypeptide and peptide vaccine compositions and methods that reduce methane and/or hydrogen production in animals. The present invention also relates to the treatment of diseases that are associated with methanogens.

### CROSS-REFERENCE TO RELATED APPLICATIONS

This application claims the benefit of U.S. Provisional Application No. 63/619,572, filed Jan. 10, 2024; and U.S. Provisional Application No. 63/645,238, filed May 10, 2024. The entire contents of each of said applications are incorporated herein in their entirety by this reference.

### INCORPORATION BY REFERENCE OF SEQUENCE LISTING

The application contains a Sequence Listing which has been submitted electronically in the form of an XML file, created Jan. 3, 2025, and named ARD-00801_SL.xml (93,100,973 bytes), the contents of which are incorporated herein by reference in their entirety.

### BACKGROUND OF THE INVENTION

Methane (CH₄) is the world's second most abundant greenhouse gas after carbon dioxide (CO₂), accounting for 16% of total greenhouse gas emissions. Livestock emissions, in particular, account for ˜32% of all anthropogenic CH₄ emissions—equating to ˜6% of all CO₂ equivalents (CO₂ₑ) of greenhouse gas emissions and ˜3 billion Tonnes/yr of CO₂ₑ. CH₄ is a powerful greenhouse gas with a potential global warming effect ˜28-fold higher than that of CO₂ over a 100 year period and ˜80-fold higher than that of CO₂ over a 20 year period. Furthermore, CH₄ has an atmospheric half-life of ˜10 years, thus reducing enteric CH₄ emissions could have an immediate and dramatic effect on limiting the rate of global warming, which would be of great significance to efforts to reduce global greenhouse gas emissions. Furthermore, CH₄ emissions also represent energy losses during ruminant production. On average, approximately 2-12% of the energy consumed in feed is lost in the form of CH₄ emissions.

For ruminants, CH₄ is predominantly formed in the ruminant fore-stomach (rumen) by methanogens, a subgroup of the Archaea. During normal rumen function, plant material is broken down by fiber-degrading microorganisms and fermented mainly to volatile fatty acids, ammonia, hydrogen (H₂) and CO₂. Ruminal methanogens principally use H₂ to reduce CO₂ to CH₄ in a series of reactions that are coupled to ATP synthesis. Additional biochemical pathways to produce CH₄ from acetate and methanol/methylamine substrates are also present, but represent a lower fraction of the total CH₄ biosynthesis in the rumen.

Attempts have been made to inhibit the action of methanogens in the rumen using a variety of interventions but most have failed, or were met with only limited success, due to low efficacy, poor selectivity, toxicity of compounds against the host, and/or build-up of resistance to anti-methanogen compounds. Further most solutions, e.g., feed additives and antibiotics, are only applicable to intensive production environments where the animal's diet can be effectively controlled to ensure precise daily dosages of the intervention. These solutions are, thus, not effective in extensive, grass-fed production environments where the animals a rarely handled and/or operations where feed cannot readily be mixed daily.

Accordingly, there is a great need in the art for effective, selective, and safe compositions and methods for inhibiting methanogens.

### SUMMARY OF THE INVENTION

The present invention is based, at least in part, on the discovery that vaccines of the present disclosure (e.g., vaccines comprising at least one polypeptide and/or at least one peptide fragment) against at least one cell surface antigen or a fragment thereof (e.g., antigenic fragment, epitope) of at least one methanogen, when administered to a subject (e.g., animal, ruminant, human), are surprisingly effective in inducing immune response and antibody production against the methanogen, and reducing the methane production in the subject.

Previous attempts to vaccinate ruminants and reduce methane production have been largely unsuccessful. Research on a vaccine targeting methanogen(s) has cost between $4 million to $5 million a year for more than 20 years.

Unfortunately, while the vaccination was able to induce immune response and production of antibodies against methanogens, it was not possible to induce production of a consistently large amount of antibodies; and to produce effective antibodies that can neutralize the growth of the methanogen and/or the production of methane. Thus, vaccination to reduce methane and/or hydrogen production in a subject (e.g., animal, ruminant, human) remained a failure.

Surprisingly, the vaccine compositions and methods of the present disclosure are surprisingly effective in reducing the CH₄ emission reductions. In addition to CH₄ emission reductions, vaccine compositions and methods of the present disclosure have shown surprising and unexpected reductions in emitted H₂ following treatment. These results are both surprising and unexpected as reductions in CH₄ following treatment with small molecule inhibitors and feed additives have contrarily shown increases in H₂ emissions. The reduction in both CH₄ and H₂ emissions suggest that the vaccine compositions and methods of the present disclosure have utility in improving the feed conversion efficiency and thereby the productivity of treated animals, e.g., increasing the production and/or ruminal concentration of one or more volatile fatty acids (e.g., propionate, butyrate, acetate) in the rumen of the animal, increasing the average daily gain of the animal, reducing the dry matter intake of the animal, reducing the feed requirements of the animal during lactation, and/or increasing the milk production of the animal. Increasing productivity of treated animals can further reduce the carbon intensity of resultant animal products (e.g., milk and meat) as treated animals are not only emit less CH₄ but also produce more product per animal. Thus, the total reduction in carbon intensity of animal-derived products from animals treated with a vaccine composition of the present disclosure may can be calculated as the composite of the CH₄ reduction of the animal following treatment in combination with the reduced carbon footprint associated with growing and maintaining those animals (e.g., less total feed, manure, urine, etc.).

The vaccine compositions and methods of the present disclosure are useful beyond reducing the methane emission in ruminants. It is well documented that methanogens are associated with various diseases, including a periodontal disease, inflammatory bowel disease (IBD), irritable bowel syndrome (ISB), IBS-C, small intestinal bacterial overgrowth (SIBO), colorectal cancer, obesity and metabolic syndrome, diverticulosis and diverticulitis, gingivitis, and bloat. Thus, the vaccine compositions and methods of the present disclosure have utility in treating these diseases in animals including humans. The vaccines of the present disclosure also provide a surprising and unexpected effect on lactic acidosis (e.g., reducing rumen lactate, increasing pH, or combination thereof). Therefore, the vaccine compositions methods of the present disclosure have utility in treating diseases associated with elevated, increased, or severe lactic acidosis, e.g., liver abscess.

### BRIEF DESCRIPTION OF FIGURES

FIG. 1A-FIG. 1B show schematic diagrams representing the relative abundance of methanogen species across geographies and herds. The diagrams indicate that methanogen populations are relatively stable across geographies and herds. For example, Methanobrevibacter gottschalkii comprises ˜30-40% of ruminal methanogens globally; thus, a cell vaccine could provide an effective global solution for reducing the amount of methane generated by ruminants.

FIG. 2 A subway diagram of our improved metatdenovo pipeline.

FIG. 3 Heatmap of the expression values of 192 genes of interest within the M. gottchalkii transcriptome. Each row represents the expression of a single gene and is labeled by its corresponding protein ID. Each column represents one M. gottschalkii monoculture harvested in exponential phase. Row annotations include any enriched GO terms (GO_group) or Pfams (Pfam_group) and the number of mass spectrometry samples in which the protein was found MS_Samples). Genes with multiple enriched GO terms or Pfams are labeled as such for simplicity.

FIG. 4 Heatmap of the normalized and transformed expression values of the metaT de novo assembly contigs that best matched to a gene of interest. Each row represents the expression of a single contig and is labeled by the corresponding gene of interest's protein ID and the contig ID, separated by an underscore. Each column represents one rumen sample harvested under different conditions.

FIG. 5 shows a schematic diagram that illustrates vaccination and post-vaccination testing of a ruminant (e.g., a cow).

FIG. 6A-FIG. 6C show schematic diagrams that illustrate exemplary tests performed for a ruminant vaccinated with a polypeptide and/or peptide vaccine of the present disclosure. FIG. 6A shows quantifying Ig antibody in serum and saliva of a vaccinated ruminant. The antibody is evaluated for methanogen binding to methanogens (e.g., affinity, specificity, etc.) and/or its effect on the fitness of methanogens (e.g., proliferation, methane production, etc.) upon binding. FIG. 6B shows measuring the amount of methane produced by a vaccinated ruminant using the GreenFeed system (C-lock Inc., Rapid City, South Dakota). FIG. 6C shows evaluation of the changes in the rumen microbiome in a vaccinated ruminant. The 16S rRNA sequencing identifies the presence and the amount of specific methanogens.

FIG. 7 shows a schematic diagram of an exemplary instrument (e.g., GreenFeed) that measures the methane produced from the rumen of a ruminant.

FIG. 8 is a schematic of a biochemical pathway and enzymes for the production of methane from acetate (i.e., the acetoclastic pathway), hydrogen and carbon dioxide (i.e., the hydrogenotrophic pathway), and methanol and derivatives thereof (i.e., the methylotrophic pathway).

FIG. 9 is a schematic describing the reduction of methyl-CoM and coenzyme B into methane by methyl-coenzyme M reductase (MCR), a key enzyme present in methane production via the acetoclastic, hydrogenotrophic, and methylotrophic pathways.

FIG. 10 is a schematic describing the application of small molecules that affect MCR activity to modulate methane production.

FIG. 11A shows a computational pipeline (left) for selection of methanogen cell surface proteins for vaccine production. The pipeline includes collation of all ORFs, filtering based on uniqueness to methanogens, cell localization, and function. A network map of key methanogen cell surface protein functions is shown to the right.

FIG. 11B shows a schematic representation of the protein fragments described in Table 21. The amino acid sequence of each folded domain is designated as a “protein fragment” in Table 21, each of which is useful as an antigen for the vaccines of the present disclosure.

FIG. 12 is a cross-sectional diagram of the application of a composition for reduction of deleterious atmospheric gases and/or precursors thereof to a flooded ecosystem.

FIG. 13 shows aerial delivery of a composition for reduction of deleterious atmospheric gases and/or precursors thereof to a flooded ecosystem.

FIG. 14 shows 3NOP concentration (mM) v. adsorbent (˜20 mM stock solution).

FIG. 15 shows 3NOP concentration (mM) v. adsorbent (˜8 mM stock solution).

FIG. 16 shows exemplary multilayer polyelectrolyte coatings of 15% activated carbon tablets.

FIG. 17 shows exemplary multilayer polyelectrolyte coatings of 25% activated carbon tablets.

FIG. 18 shows exemplary multilayer polyelectrolyte coatings of 15% activated carbon tablets with 5% sodium lignosulfonate.

FIG. 19 shows exemplary multilayer polyelectrolyte coatings of 15% activated carbon with 5% hydroxypropyl cellulose.

FIG. 20 is a graph showing the release profiled of a silica v. activated carbon adsorbent.

FIG. 21 is a table showing the composition of various polycaprolactone-based formulations according to some embodiments of the invention.

FIG. 22 is a bar graph showing 3NOP release in mM for exemplary polycaprolactone-based formulations.

FIG. 23 is a graph showing normalized 3NOP concentration v. release time in days of exemplary polybutylene succinate-based formulations.

FIG. 24 is a bar graph showing 3NOP release (%) from exemplary PEC microcapsules. Samples are prepared with a 3NOP concentration of 100 μM. Final pH solution ˜7. K, L, F, and E refer to lysine, leucine, phenylalanine, and glutamic acid, respectively. PSS refers to polystyrene sulfonate. FIG. 24 discloses (KKLF)3 as SEQ ID NO: 79312, (EELF)3 as SEQ ID NO: 79313 and (kKlF)3 as SEQ ID NO: 79314.

FIG. 25 is bar graph showing 3NOP release (%) from exemplary PEC microcapsules. Samples are prepared with a 3NOP concentration of 100 μM. Final pH solution ˜7. PLR refers to poly (L-arginine), and PLK refers to poly (L-lysine). SLS and PSS refer to sodium lignosulfonate and polystyrene sulfonate.

FIG. 26 shows average daily group ADG (kg per day) for individual animals, grouped by treatment group, as described in Example 12.

FIG. 27 shows the GreenFeed visitation for all animals, grouped by measurement period, as described in Example 12.

FIG. 28 shows the GreenFeed visitation pattern overlaid with maximum local temperature (McGregor, TX) as described in Example 12.

FIG. 29 shows visual definition of AA analysis as described in Example 12.

FIG. 30 shows ΔΔ CH4 production (g/d) for protein antigen formulations, relative to pre-prime baseline (p0), as described in Example 12.

FIG. 31 shows ΔΔ CH4 intensity (g/d) for protein antigen formulations, relative to pre-prime baseline (p0), as described in Example 12.

FIG. 32 shows RPF51698.1 ELISA IgG response, average value of treatment groups over time. Treatment groups 9G & 9H have elevated IgG response against RPF51698.1 protein relative to the control group 9A at d28 & d42 (p<0.001) and d63 (p<0.01). T-tests do not support treatment group 9I being greater than the control group at d42 (p=0.08), as described in Example 12.

FIG. 33 shows ADC46800.1 ELISA IgG response, average value of treatment groups over time. Treatment groups 9G, 9H, & 9I have elevated IgG response against ADC46800.1 protein compared to the control group 9A (p<0.001 all days except Group 9H d63 p<0.01). T-tests support a difference in Group 9C d63 (p<0.05) but do not support a difference in Group 9D d42 (p=0.2), as described in Example 12.

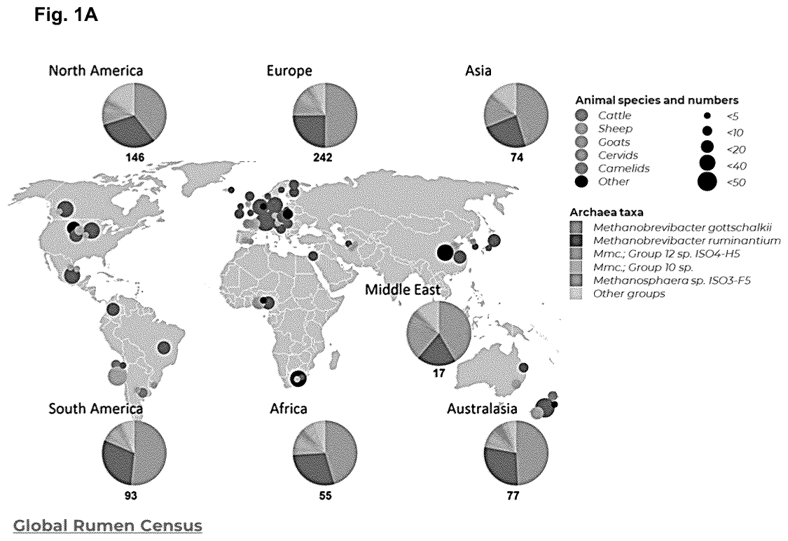

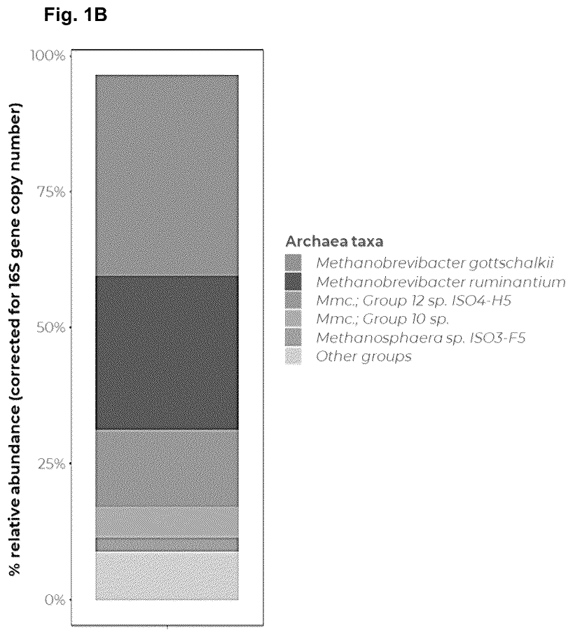

### DETAILED DESCRIPTION OF THE INVENTION

Provided herein are vaccines (e.g., comprising at least one protein and/or at least one peptide fragment) against at least one cell surface antigen or a fragment thereof (e.g., antigenic fragment, epitope) of at least one methanogen, which are effective in inducing immune response and antibody production against the methanogen antigen, and reducing the methane and/or hydrogen production in subjects. The vaccines of the present disclosure are also useful in treating diseases in subjects (e.g., animals, mammals, ruminants, humans) that are associated with methanogens (e.g., a periodontal disease, Inflammatory Bowel Disease (IBD), irritable bowel syndrome (ISB), IBS-C, small intestinal bacterial overgrowth (SIBO), colorectal cancer, obesity and metabolic syndrome, diverticulosis and diverticulitis, gingivitis, and/or bloat). The vaccines of the present disclosure are also useful in treating diseases in subjects (e.g., animals, mammals, ruminants, humans) that are associated with elevated, increased, or severe lactic acidosis, e.g., liver abscess.

### Definitions

The articles “a” and “an” are used herein to refer to one or to more than one (i.e. to at least one) of the grammatical object of the article. By way of example, “an element” means one element or more than one element.

As used herein, the term “about” when used before a numerical designation, e.g., temperature, time, amount, concentration, and such other, including a range, indicates approximations which may vary by (+) or (−) 10%, 9%, 8%, 7%, 6%, 5%, 4%, 3%, 2%, or 1%.

The term “administering” is intended to include routes of administration which allow an agent (e.g., a vaccine composition, an agent that reduces methane production in a subject) to perform its intended function. Examples of routes of administration which can be used include injection (subcutaneous, intravenous, parenteral, intraperitoneal, intrathecal, intradermal, intramuscular, etc.), oral, inhalation, and transdermal routes. The injections can be bolus injections or can be continuous infusion. Depending on the route of administration, the agent (e.g., a vaccine composition, an agent that reduces methane production in a subject) can be coated with or disposed in a selected material to protect it from natural conditions which may detrimentally affect its ability to perform its intended function. The agent may be administered alone, or in conjunction with a pharmaceutically acceptable carrier or adjuvant. The agent (e.g., a vaccine composition, an agent that reduces methane production in a subject) also may be administered as a prodrug, which is converted to its active form in vivo.

The term “conjoint” or “combination” administration, as used herein, refers to the administration of two or more agents that aid in reducing methane production in a subject. The different agents comprising the combination may be administered concomitant with, prior to, or following the administration of one or more agents.

The term “fragment,” as used herein encompasses any and all that is less than the full length. In some embodiments, a fragment of a polypeptide of the present disclosure is an antigenic fragment of the polypeptide. Such antigenic fragment may comprise at least one epitope that binds to the antibody. In preferred embodiments, a fragment of a polypeptide of the present disclosure is a fragment of the polypeptide that is effective in eliciting immune response and/or inducing antibody production when administered to a subject.

The term “methanogen,” as used herein, refers to a microorganism that produces methane as a metabolic byproduct. Methanogens belong to the domain Archaea, and include, but are not limited to those of a family Methanobacteriaceae, e.g., those of genera Methanobrevibacter, Methanosphaera, Methanomicrobium, Methanobacterium, Methanocorpusulum, Methanosaeta, Methanoculleus, Methanosarcina, and Thermoplasmatales. Specific methanogens include, but are not limited to, Methanobrevibacter ruminantium (e.g., the M1 strain or strain DSM 1093 (see e.g., World Wide Web at dsmz.de/microorganisms/html/strains/strain.dsm001093. htm) and Methanobrevibacter gottschalkii. Additional relevant species are further described below.

The term “ruminant” refers to a hoofed herbivorous grazing or browsing mammal that is able to acquire nutrients from plant-based food by fermenting it in a specialized stomach prior to digestion, principally through microbial actions. The process, which takes place in the front part of the digestive system and therefore is called foregut fermentation, typically requires the fermented ingesta (known as cud) to be regurgitated and chewed again. The roughly 200 species of ruminants include both domestic and wild species. Ruminants include, but are not limited to, cattle (e.g., large domesticated ruminant animals, e.g., cows (including dairy cattle), bulls), all domesticated and wild bovines (i.e., those belonged to the family Bovidae; e.g., cows, cattle, bulls, bisons, yaks, African buffalos, water buffalos, antelopes), goats, sheep, giraffes, deer, caribou, and gazelles. In preferred embodiments, ruminants are domesticated. As used herein, the term “ruminant” includes ruminant-like animals or pseudo-ruminant animals such as macropods, llamas, camels, and alpacas. In some embodiments, a ruminant has not been administered with an agent that reduces methane. In other embodiments, a ruminant has been administered or is being administered with an agent that reduces methane.

As used herein, the term “valency” refers to the number of antigenic components in the vaccine or polypeptide. In some embodiments, the vaccines are monovalent. In some embodiments, the vaccines are divalent. In some embodiments the vaccines are trivalent. In some embodiments the vaccines are multi-valent. Multivalent vaccines may comprise 2, 3, 4, 5, 6, 7, 8, 9, 10, 11, 12, 13, 14, 15, 16, 17, 18, 19, 20, or more antigens or antigenic moieties (e.g., antigenic peptides, etc.). The antigenic components of the vaccines may be in a single polypeptide or peptide molecule. The antigenic components of the vaccines may be in separate polypeptide or peptide molecules.

The term “subject” refers to any healthy or diseased animal, including any mammal, ruminant, canine, feline, or human.

### Methanogens

The diversity of the ruminal methanogens is much smaller, and their diversity is much lower than that of rumen bacteria, with archaeal SSU rRNA only accounting for 6.8% of rumen total SSU rRNA. Archaea in the rumen is represented by <3.3% of the total rRNA (both 16S and 18S) therein. Representative family of methanogens includes Methanobacteriaceae. Rumen methanogens typically comprises 2-3% of the total microbial biomass in the rumen.

Representative genera of methanogens include Methanobrevibacter, Methanosphaera, Methanomicrobium, Methanobacterium, Methanocorpusulum, Methanosaeta, Methanoculleus, Methanosarcina, and Thermoplasmatales.

Certain species of ruminal methanogens have been isolated into pure cultures: Methanobacterium formicicum, Methanobacterium bryantii, Methanobrevibacter ruminantium, Methanobrevibacter gottschalkii, Methanobrevibacter millerae, Methanobrevibacter olleyae, Methanomicrobium mobile, Methanoculleus olentangyi, and Methanosarcina barkeri. Additional species have been recently isolated, including Methanobrevibacter boviskoreani (isolated from the rumen of Korean native cattle), Methanobacterium beijingense (isolated from the rumen of goat), Methanoculleus marisnigri (isolated from the rumen of Indian crossbred cattle), Methanoculleus bourgensis (isolated from the rumen of Holstein cattle), and Methanosarcina mazei (isolated from the rumen of Korean Hanwoo cattle) (based on the RDP database). A Thermoplasmatales-like pyrrolysine-dependent archaeon BRNA1 was also isolated from bovine (GenBank access number: CP002916).

Collectively, 16S rRNA gene sequences from cultured methanogens only accounted for approximately 0.7% of the total archaeal sequences of rumen origin, and several taxa do not have a single cultured representative. Most of the isolates are members of the family Methanobacteriaceae. Compared to other anaerobic habitats where >100 species of methanogens of 28 genera have been isolated, the diversity and species richness of ruminal methanogens are quite low, reflecting the highly selective ruminal environment for methanogens. In addition, sequenced ruminal 16S rRNA gene clones shared >95% sequence similarity with that of Methanobrevibacter gottschalkii, Methanobrevibacter thaueri, Methanobrevibacter smithii and Methanosphaera stadtmanae, indicating that these species may be common ruminal methanogens.

Much of the ruminal methanogen diversity was characterized by 16S rRNA gene sequences. The RDP Release 11 (Update 3) contains 8,623 archaeal 16S rRNA gene sequences of rumen origin. These sequences were generated using the Sanger sequencing technology, which produces higher sequence accuracy than NGS technologies, in 96 separate studies including 48 unpublished studies. About 90% of these sequences were assigned to methanogens. These sequences were classified to 10 known genera, with Methanobrevibacter being represented by 63.2% of all the sequences followed by Methanosphaera (9.8%), Methanomicrobium (7.7%), and Methanobacterium (1.2%). The order Thermoplasmatales, which was previously referred to as the rumen cluster C (RCC) group, is represented by 7.4% of the total archaeal sequences.

### Cell Surface Proteins of Methanogens

Provided herein is at least one cell surface protein or a fragment thereof (e.g., an antigenic fragment, e.g., a fragment comprising an epitope, e.g., a fragment comprising an extracellular domain or a portion thereof) of at least one methanogen that can be used in a vaccine composition, which can elicit immune response, antibody production, and antibody-mediated neutralization of the growth of methanogens and/or production of methane. Further provided herein are nucleic acid(s) encoding the at least one cell surface protein or a fragment thereof.

In certain aspects, the at least one cell surface protein or a fragment thereof (e.g., an antigenic fragment, e.g., a fragment comprising an epitope, e.g., a fragment comprising an extracellular domain or a portion thereof) of at least one methanogen is of a family Methanobacteriaceae.

In some embodiments, the at least one methanogen is of a genus selected from: Methanobrevibacter, Methanosphaera, Methanomicrobium, Methanobacterium, Methanocorpusulum, Methanosaeta, Methanoculleus, Methanosarcina, and Thermoplasmatales.

In some embodiments, the at least one methanogen comprises Methanobacterium formicicum, Methanobacterium bryantii, Methanobrevibacter ruminantium, Methanobrevibacter millerae, Methanobrevibacter olleyae, Methanomicrobium mobile, Methanoculleus olentangyi, Methanosarcina barkeri, Methanobrevibacter boviskoreani, Methanobacterium beijingense, Methanoculleus marisnigri, Methanoculleus bourgensis, Methanosarcina mazei, Thermoplasmatales archaeon BRNA1, Methanobrevibacter gottschalkii, Methanobrevibacter thaueri, Methanobrevibacter smithii, Methanosphaera stadtmanae, Methanococcoides burtonii, Methanolobus psychrophilus R15, Methanobacterium paludism, Methanohalobium evestigatum, Methanomethylovorans hollandica, Methanothrix soehngenii, Methanocaldococcus vulcanius, Methanosalsum zhilinae, Methanocorpusculum labreanum, Methanoregula formicica, Methanoculleus marisnigri, Methanocella arvoryzae, Methanoculleus bourgensis, Methanolacinia petrolearia, Methanospirillum hungatei, Methanoplanus limicola, Methanohalophilus mahii, Methanococcus aeolicus, Methanosphaerula palustris, Methanocaldococcus fervens, Methanocaldococcus jannaschii, Methanocaldococcus sp. FS406-22, Methanoregula boonei, Methanobrevibacter sp. AbM4, Methanobrevibacter ruminantium, Methanosphaera, Methanobacterium formicicum, Methanocaldococcus villosus, Methanosarcina barkeri, Methanobacterium lacus, Methanotorris igneus, Methanotorris formicicus, Methanocaldococcus infernus, Methanofollis liminatans, Methanothermococcus okinawensis, Methanobrevibacter smithii, Methanobrevibacter, Methanocella conradii, Methanothermococcus thermolithotrophicus, Methanococcus maripaludis, Methanococcus maripaludis, Methanococcus vannielii, Methanothermus fervidus, Methanosarcina acetivorans, Methanosarcina mazei, Methanosaeta harundinacea 6Ac, Methanococcus maripaludis, Methanococcus voltae, Methanolinea tarda, Methanolobus psychrophilus, Methanosaeta harundinacea, or any combination thereof.

In some embodiments, the at least one methanogen comprises Methanobrevibacter ruminantium. In some embodiments, the at least one methanogen comprises Methanobrevibacter ruminantium M1 (DSM 1093).

In preferred embodiments, the at least one methanogen comprises Methanobrevibacter gottschalkii. In some embodiments, the at least one methanogen comprises Methanobrevibacter gottschalkii DSM11977.

In some embodiments, a vaccine composition comprises a polypeptide or a fragment thereof (e.g., an antigenic fragment, an epitope) of at least one cell surface protein. In some embodiments, the polypeptide comprises the extracellular domain or a fragment thereof of at least one cell surface protein. In some embodiments, the polypeptide or a fragment thereof does not comprise the transmembrane and/or intracellular domains or a fragment thereof of the at least one cell surface protein.

In certain aspects, provided herein are vaccine compositions comprising at least one cell surface protein or a fragment thereof (e.g., an antigenic peptide fragment, e.g., a peptide fragment comprising an epitope, e.g., a peptide fragment comprising an extracellular domain or a portion thereof, e.g., a polypeptide fragment, e.g., peptide fragment) of at least one methanogen.

In some embodiments, a vaccine composition comprises at least one polypeptide and/or at least one peptide of at least one methanogen.

In some embodiments, the at least one polypeptide or a fragment thereof does not comprise a signal peptide.

In some embodiments, the at least one polypeptide or a fragment thereof does not comprise a transmembrane domain.

In some embodiments, the at least one polypeptide or a fragment thereof comprises at least 90%, 91%, 92%, 93%, 94%, 95%, 96%, 97% 98%, 99%, or 100% sequence identity to an amino acid sequence presented herein. In some embodiments, the at least one polypeptide or a fragment thereof comprises at least 90%, 91%, 92%, 93%, 94%, 95%, 96%, 97% 98%, 99%, or 100% sequence identity to an amino acid sequence set forth in any one of Tables C-F, 1A, 1B, 2A, 2B, 3A, 3B, 4A, 4B, 5A, 5B, and 6A-6G, or a fragment thereof.

In some embodiments, the at least one polypeptide or a fragment thereof comprises at least 90%, 91%, 92%, 93%, 94%, 95%, 96%, 97% 98%, 99%, or 100% sequence identity to an amino acid sequence encoded by at least one nucleic acid presented herein. In some embodiments, the at least one polypeptide or a fragment thereof is encoded by a nucleic acid comprising the nucleotide sequence set forth in any one of Tables C-F, 1A, 1B, 2A, 2B, 3A, 3B, 4A, 4B, 5A, 5B, and 6A-6G, or a fragment thereof.

In some embodiments, the at least one polypeptide or a fragment thereof further comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 heterologous amino acid residues that are not native to the cell surface protein of a methanogen.

In some embodiments, the at least one polypeptide or a fragment thereof further comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 heterologous amino acid residues that are not native to the cell surface protein of a methanogen.

In some embodiments, the at least one polypeptide or a fragment thereof further comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 heterologous amino acid residues that are not native to the cell surface protein of a methanogen.

In some embodiments, the heterologous amino acid residues comprise a heterologous signal peptide and/or a heterologous transmembrane domain.

In some embodiments, a vaccine composition comprises at least one fragment (e.g., at least one polypeptide fragment and/or a peptide fragment) of at least one cell surface protein of at least one methanogen. In some embodiments, a vaccine composition comprises at least one fragment (e.g., polypeptide fragment and/or a peptide fragment), which is not a full-length cell surface protein of at least one methanogen.

In some embodiments, the at least one fragment of at least one cell surface protein lacks at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues from its native full-length protein sequence.

In some embodiments, the at least one fragment of at least one cell surface protein lacks about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues from its native full-length protein sequence.

In some embodiments, the at least one fragment of at least one cell surface protein lacks no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues from its native full-length protein sequence.

In some embodiments, the at least one fragment of at least one cell surface protein lacks at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues from the N-terminus of its native full-length protein sequence.

In some embodiments, the at least one fragment of at least one cell surface protein lacks at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues from the C-terminus of its native full-length protein sequence.

In preferred embodiments, the at least one fragment of at least one cell surface protein comprises one or more extracellular domain or a portion thereof. A person of ordinary skill in the art can readily predict an extracellular portion of any given sequence by using a suitable tool known in the art, e.g., TMbed or TMHMM.

In some embodiments, the at least one fragment of at least one cell surface protein lacks a native signal peptide.

In some embodiments, the at least one fragment of at least one cell surface protein lacks a native transmembrane domain.

In certain aspects, certain classes of methanogen cell surface proteins are particularly useful in generating an effective vaccine composition of the present disclosure (e.g., comprising at least one cell surface protein or a fragment thereof of at least one methanogen).

In some embodiments, the at least one cell surface protein comprises at least one of the following structures and/or functions: adhesin-like, ATP-processing, cell wall biosynthesis, cofactor biosynthesis, CRISPR (provides methanogens an immunity against viruses), energy metabolism, enzyme, fatty acid synthesis, general metabolism, membrane protein, metal-binding, methanogenesis, methanogenesis Mtr proteins, methanogenesis MtrE proteins, phage related, proteolysis, transcription regulation, ribosomal, substrate binding, transcription, transport, and a protein whose gene expression changes in response to lauric acid stress (see Table 6G below).

A person of ordinary skill in the art can determine the polypeptide sequences from the nucleic acid sequences, or determine the nucleic acid sequences from the polypeptide sequences presented herein or those known in the art.

The nucleic acid and amino acid sequence information for nucleic acid and polypeptide molecules useful in the present invention are well-known in the art and readily available on publicly available databases, such as the National Center for Biotechnology Information (NCBI).

The representative cell surface antigens of various methanogens and their nucleic acid sequences and amino acid sequences are provided in the U.S. application Ser. No. 18/350,526 (e.g., Table 2A, Table 2B, Table 3, Table 17A, Table 19, Table 20, and Table 21), U.S. Application No. 63/359,978, or U.S. Application No. 63/524,513, the entire contents of each of which are incorporated herein by reference in its entirety.

Certain representative antigens and their amino acid and nucleic acid sequences are also presented in Tables C-F, TA, 1B, 2A, 2B, 3A, 3B, 4A, 4B, 5A, 5B, and 6A-6G of the present disclosure.

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA encoding a cell surface antigen nucleic acid (or any portion thereof) can be used to derive the polypeptide amino acid sequence, using the genetic code to translate the DNA or RNA into an amino acid sequence. Likewise, for polypeptide amino acid sequences, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.
Table 1A: Representative Antigens (full-length proteins) of Methanobrevibacter gottschalkii Set 1 (amino acid sequences (SEQ ID NOS 71070-71706) and nucleic acid sequences (SEQ ID NOS 71707-72343))
Table 1B: Representative Antigens (full-length proteins) of Methanobrevibacter gottschalkii Set 1 (codon-optimized nucleic acid sequences) Left column discloses SEQ ID NOS 72344-72980 and right column discloses SEQ ID NOS 72981-73617.
Table 2A: Representative Antigens (full-length proteins) of Methanobrevibacter gottschalkii Set 2 (amino acid sequences (SEQ ID NOS 73618-73993) and nucleic acid sequences (SEQ ID NOS 73994-74369))
Table 2B: Representative Antigens (full-length proteins) of Methanobrevibacter gottschalkii Set 2 (codon-optimized nucleic acid sequences) Left column discloses SEQ ID NOS 74370-74745 and right column discloses SEQ ID NOS 74746-75121.
Table 3A: Representative Antigens (full-length proteins) of Methanobrevibacter gottschalkii Set 3 (amino acid sequences (SEQ ID NOS 75122-75220) and nucleic acid sequences (SEQ ID NOS 75221-75319)
Table 3B: Representative Antigens (full-length proteins) of Methanobrevibacter gottschalkii Set 3 (codon-optimized nucleic acid sequences) Left column discloses SEQ ID NOS 75320-75418 and right column discloses SEQ ID NOS 75419-75517.
Table 4A: Representative Antigens (fragments) of Methanobrevibacter gottschalkii Set 4 (amino acid sequences (SEQ ID NOS 75518-76049) and nucleic acid sequences (SEQ ID NOS 76050-76581))
Table 4B: Representative Antigens (fragments) of Methanobrevibacter gottschalkii Set 4 (codon-optimized nucleic acid sequences) Left column discloses SEQ ID NOS 76582-77112 and right column discloses SEQ ID NOS 77113-77643.
Table 5A: Representative Antigens (fragments) of Methanobrevibacter gottschalkii Set 5 (amino acid sequences (SEQ ID NOS 77644-77967) and nucleic acid sequences (SEQ ID NOS 77968-78291))
Table 5B: Representative Antigens (fragments) of Methanobrevibacter gottschalkii Set 5 (codon-optimized nucleic acid sequences) Left column discloses SEQ ID NOS 78292-78615 and right column discloses SEQ ID NOS 78616-78939.
Table 6A: Representative Antigens (fragments) of Methanobrevibacter gottschalkii Set 6 (amino acid sequences (SEQ ID NOS 78940-79032) and nucleic acid sequences (SEQ ID NOS 79033-79125))
Table 6B: Representative Antigens (fragments) of Methanobrevibacter gottschalkii Set 6 (codon-optimized nucleic acid sequences) Left column discloses SEQ ID NOS 79126-79218 and right column discloses SEQ ID NOS 79219-79311.
Table 6C: Representative sequences of the cell surface proteins of Methanobrevibacter ruminantium M1 (GenBank: CP001719.1)
   See SEQ ID NO. 1 to SEQ ID NO. 2217 for the native nucleic acid sequences. See SEQ ID NO. 2218 to SEQ ID NO. 4434 for the bovine codon-optimized nucleic acid sequences. See SEQ ID NO. 4435 to SEQ ID NO. 6651 for the amino acid sequences.
Table 6D: Representative sequences of the cell surface proteins of Methanobrevibacter gottschalkii
   See SEQ ID NO. 6652 to SEQ ID NO. 8451 for the native nucleic acid sequences. See SEQ ID NO. 8452 to SEQ ID NO. 10251 for the bovine codon-optimized nucleic acid sequences. See SEQ ID NO. 10252 to SEQ ID NO. 12051 for the amino acid sequences.
Table 6E: Representative mtrE sequences of methanogens
   See SEQ ID NO. 12100 to SEQ ID NO. 12147 for the nucleic acid sequences. See SEQ ID NO. 12052 to SEQ ID NO. 12099 for the amino acid sequences.

TABLE 6F
Genes and Bos taurus codon-optimized nucleic acid sequence of the genes

| | | Bos taurus optimized nucleic acid |
|---|---|---|
| Name | Species | sequence (SEQ ID NO) |
| WP_004031278.1 | Methanobacterium formicicum | SEQ ID NO: 16969 |
| WP_012956721.1 | Methanobrevibacter ruminantium | SEQ ID NO: 16970 |
| WP_019264904.1 | Methanobrevibacter smithii | SEQ ID NO: 16971 |
| WP_016359091.1 | Methanobrevibacter sp. AbM4 | SEQ ID NO: 16972 |
| WP_011033493.1 | Methanosarcina mazei | SEQ ID NO: 16973 |
| mru0143 | Methanobrevibacter ruminantium | SEQ ID NO: 16974 |
| mru0842 | Methanobrevibacter ruminantium | SEQ ID NO: 16975 |
| mru2048 | Methanobrevibacter ruminantium | SEQ ID NO: 16976 |
| mru1222 | Methanobrevibacter ruminantium | SEQ ID NO: 16977 |
| mru2047 | Methanobrevibacter ruminantium | SEQ ID NO: 16978 |

*The vaccine compositions of the present disclosure may comprise a polypeptides or a fragment thereof encoded by any one of the nucleic acids above.

TABLE 6G
Exemplary structural/functional groups of representative cell surface proteins
This table contains SEQ ID NOs of amino acid sequences of proteins; or SEQ ID NOs of nucleic acid
sequences that encode the proteins that belong to the indicated structural and/or functional groups.
(1) Adhesin-like proteins (SEQ ID NOs)

| | | | | | | | | | | | | |
|---|---|---|---|---|---|---|---|---|---|---|---|---|
| 4438 | 4449 | 4453 | 4454 | 4465 | 4472 | 4482 | 4498 | 4506 | 4510 | 4511 | 4513 | 4516 |
| | 4517 | 4518 | 4519 | 4520 | 4524 | 4577 | 4594 | 4618 | 4656 | 4679 | 4689 | 4747 |
| | 4751 | 4752 | 4760 | 4761 | 4765 | 4772 | 4850 | 4851 | 4852 | 4883 | 4884 | 4922 |
| | 4926 | 5137 | 5156 | 5160 | 5205 | 5208 | 5244 | 5257 | 5261 | 5272 | 5275 | 5276 |
| | 5329 | 5335 | 5395 | 5396 | 5403 | 5409 | 5410 | 5411 | 5412 | 5509 | 5510 | 5521 |
| | 5556 | 5563 | 5639 | 5642 | 5654 | 5679 | 5753 | 5774 | 5790 | 5808 | 5818 | 5819 |
| | 5848 | 5849 | 5856 | 5897 | 5929 | 5930 | 5931 | 5932 | 5938 | 5945 | 6036 | 6038 |
| | 6082 | 6083 | 6158 | 6230 | 6387 | 6403 | 6428 | 6475 | 6480 | 6481 | 6484 | 6485 |
| | 6486 | 6487 | 6491 | 6522 | 6555 | 6556 | 6566 | 6579 | 6610 | 6621 | 6628 | 10279 |
| | 10280 | 10281 | 10282 | 10336 | 10337 | 10341 | 10342 | 10357 | 10369 | 10514 | 10522 | 10539 |
| | 10540 | 10591 | 10613 | 10614 | 10616 | 10617 | 10618 | 10627 | 10637 | 10638 | 10639 | 10640 |
| | 10665 | 10671 | 10677 | 10697 | 10723 | 10731 | 10810 | 10935 | 11091 | 11311 | 11331 | 11528 |
| | 11594 | 11595 | 11596 | 11646 | 11902 | 11903 | 11904 | 11909 | 11961 | 11987 | 16974 | 16975 |
| | 16976 | 16977 | 16774 | 16777 | 16780 | 16784 | 16785 | 16786 | 16787 | 16789 | 16791 | 16792 |
| | 16793 | 16794 | 16795 | 16797 | 16799 | 16800 | 16801 | 16802 | 16803 | 16804 | 16805 | 16806 |
| | 16807 | 16808 | 16857 | 16861 | 16863 | 16864 | 16865 | 16868 | 16869 | 16871 | 16872 | 53218 |
| | 53219 | 53220 | 53221 | 53222 | 53223 | 53224 | 53225 | 53226 | 53227 | 53228 | 53229 | 53230 |
| | 53231 | 53232 | 53233 | 53234 | 53235 | 53236 | 53237 | 53238 | 53239 | 53240 | 53241 | 53242 |
| | 53250 | 53251 | 53252 | 53253 | 53254 | 53255 | 53256 | 53257 | 53258 | 53259 | 53260 | 53261 |
| | 53262 | 53263 | 53264 | 53265 | 53266 | 53267 | 53268 | 53269 | 53270 | 53271 | 53272 | 53273 |
| | 53274 | 53275 | 53276 | 53277 | 53278 | 53279 | 53280 | 53281 | 53282 | 53283 | 53284 | 53285 |
| | 53286 | 53287 | 53288 | 53289 | 53290 | 53291 | 53292 | 53293 | 53294 | 53295 | 53296 | 53297 |
| | 53298 | 53299 | 53300 | 53301 | 53302 | 53303 | 53304 | 53305 | 53306 | 53307 | 53308 | 53309 |
| | 53310 | 53311 | 53312 | 53313 | 53314 | 53315 | 53316 | 53317 | 53318 | 53319 | 53320 | 53321 |
| | 53322 | 53323 | 53324 | 53325 | 53326 | 53327 | 53328 | 53329 | 53330 | 53331 | 53332 | 53333 |
| | 53334 | 53335 | 53336 | 53337 | 53338 | 53339 | 53340 | 53341 | 53342 | 53343 | 53344 | 53345 |
| | 53346 | 53347 | 53348 | 53349 | 53350 | 53351 | 53352 | 53353 | 53354 | 53355 | 53356 | 53357 |
| | 53358 | 53359 | 53360 | 53361 | 53362 | 53363 | 53364 | 53365 | 53366 | 53367 | 53368 | 53370 |
| | 53371 | 53372 | 53373 | 53374 | 53375 | 53376 | 53377 | 53378 | 53379 | 53380 | 53381 | 53382 |
| | 53383 | 53384 | 53385 | 53386 | 53387 | 53388 | 53389 | 53390 | 53391 | 53392 | 53393 | 53394 |
| | 53395 | 53396 | 53397 | 53398 | 53399 | 53400 | 53401 | 53402 | 53403 | 53406 | 53407 | 53408 |
| | 53409 | 53410 | 53411 | 53412 | 53413 | 53414 | 53415 | 53416 | 53417 | 53418 | 53419 | 53420 |
| | 53421 | 53422 | 53423 | 53424 | 53425 | 53426 | 53427 | 53428 | 53429 | 53430 | 53431 | 53432 |
| | 53433 | 53434 | 53435 | 53436 | 53437 | 53438 | 53439 | 53440 | 53441 | 53442 | 53443 | 53444 |
| | 53445 | 53446 | 53447 | 53448 | 53449 | 53450 | 53451 | 53452 | 53453 | 53454 | 53455 | 53456 |
| | 53457 | 53458 | 53459 | 53460 | 53461 | 53462 | 53463 | 53464 | 53465 | 53466 | 53467 | 53468 |
| | 53477 | 53478 | 53479 | 53480 | 53481 | 53482 | 53483 | 53484 | 53485 | 53486 | 53487 | 53488 |
| | 53489 | 53490 | 53491 | 53492 | 53493 | 53494 | 53495 | 53496 | 53497 | 53498 | 53499 | 53500 |
| | 53501 | 53502 | 53503 | 53504 | 53505 | 53508 | 53509 | 53510 | 53511 | 53512 | 53522 | 53523 |
| | 53524 | 53525 | 53526 | 53527 | 53528 | 53529 | 53530 | 53531 | 53532 | 53533 | 53534 | 53535 |
| | 53536 | 53537 | 53538 | 53539 | 53540 | 53541 | 53542 | 53546 | 53547 | 53548 | 53549 | 53550 |
| | 53551 | 53552 | 53553 | 53554 | 53555 | 53556 | 53557 | 53558 | 53559 | 53560 | 53561 | 53562 |
| | 53563 | 53564 | 53571 | 53572 | 53573 | 53574 | 53575 | 53576 | 53577 | 53578 | 53579 | 53580 |
| | 53581 | 53582 | 53583 | 53584 | 53585 | 53586 | 53587 | 53594 | 53595 | 53596 | 53597 | 53598 |
| | 53599 | 53603 | 53604 | 53605 | 53606 | 53607 | 53608 | 53609 | 53610 | 53611 | 53612 | 53613 |
| | 53614 | 53615 | 53616 | 53617 | 53618 | 53619 | 53620 | 53621 | 53622 | 53623 | 53624 | 53625 |
| | 53626 | 53627 | 53628 | 53629 | 53630 | 53631 | 53632 | 53633 | 53634 | 53635 | 53636 | 53637 |
| | 53638 | 53639 | 53640 | 53641 | 53642 | 53643 | 53644 | 53645 | 53646 | 53647 | 53648 | 53649 |
| | 53657 | 53658 | 53659 | 53660 | 53661 | 53662 | 53663 | 53664 | 53665 | 53666 | 53667 | 53668 |
| | 53669 | 53670 | 53671 | 53672 | 53673 | 53674 | 53675 | 53684 | 53685 | 53694 | 53695 | 53696 |
| | 53697 | 53698 | 53699 | 53700 | 53701 | 53702 | 53703 | 53704 | 53705 | 53706 | 53707 | 53708 |
| | 53709 | 53710 | 53711 | 53712 | 53713 | 53714 | 53715 | 53716 | 53717 | 53718 | 53719 | 53720 |
| | 53721 | 53722 | 53723 | 53724 | 53725 | 53726 | 53727 | 53728 | 53729 | 53730 | 53731 | 53732 |
| | 53733 | 53734 | 53735 | 53736 | 53737 | 53738 | 53739 | 53740 | 53741 | 53742 | 53743 | 53744 |
| | 53745 | 53746 | 53747 | 53748 | 53749 | 53750 | 53751 | 53752 | 53753 | 53754 | 53755 | 53756 |
| | 53757 | 53758 | 53759 | 53760 | 53761 | 53762 | 53763 | 53764 | 53765 | 53766 | 53767 | 53768 |
| | 53769 | 53770 | 53771 | 53772 | 53773 | 53774 | 53775 | 53776 | 53777 | 53778 | 53779 | 53780 |
| | 53781 | 53782 | 53783 | 53784 | 53785 | 53786 | 53787 | 53788 | 53789 | 53790 | 53791 | 53792 |
| | 53793 | 53794 | 53796 | 53797 | 53798 | 53799 | 53800 | 53801 | 53802 | 53803 | 53804 | 53805 |
| | 53806 | 53807 | 53808 | 53809 | 53810 | 53811 | 53812 | 53813 | 53820 | 53821 | 53822 | 53823 |
| | 53824 | 53825 | 53826 | 53827 | 53828 | 53829 | 53830 | 53831 | 53832 | 53833 | 53834 | 53835 |
| | 53836 | 53837 | 53838 | 53839 | 53840 | 53841 | 53842 | 53843 | 53844 | 53845 | 53846 | 53847 |
| | 53848 | 53849 | 53850 | 53851 | 53852 | 53853 | 53854 | 53855 | 53856 | 53857 | 53858 | 53859 |
| | 53860 | 53861 | 53862 | 53866 | 53867 | 53868 | 53869 | 53870 | 53871 | 53872 | 53873 | 53874 |
| | 53875 | 53876 | 53877 | 53878 | 53879 | 53880 | 53906 | 53907 | 53908 | 53909 | 53910 | 53911 |
| | 53916 | 53917 | 53918 | 53919 | 53920 | 53921 | 53922 | 53923 | 53924 | 53925 | 53926 | 53927 |
| | 53928 | 53929 | 53930 | 53931 | 53932 | 53934 | 53935 | 53936 | 53937 | 53938 | 53939 | 53940 |
| | 53941 | 53942 | 53943 | 53944 | 53945 | 53946 | 53947 | 53948 | 53949 | 53950 | 53951 | 53952 |
| | 53953 | 53954 | 53955 | 53956 | 53957 | 53958 | 53959 | 53960 | 53961 | 53962 | 53963 | 53964 |
| | 53965 | 53966 | 53967 | 53968 | 53969 | 53970 | 53972 | 53973 | 53974 | 53976 | 53977 | 53978 |
| | 53979 | 53980 | 53981 | 53982 | 53983 | 53984 | 53986 | 53987 | 53988 | 53989 | 53990 | 54010 |
| | 54011 | 54034 | 54035 | 54036 | 54037 | 54038 | 54039 | 54040 | 54041 | 54042 | 54043 | 54044 |
| | 54045 | 54046 | 54047 | 54048 | 54049 | 54050 | 54051 | 54052 | 54053 | 54054 | 54055 | 54056 |
| | 54057 | 54058 | 54059 | 54060 | 54061 | 54062 | 54063 | 54064 | 54065 | 54066 | 54067 | 54068 |
| | 54069 | 54070 | 54071 | 54072 | 54075 | 54076 | 54077 | 54078 | 54079 | 54081 | 54082 | 54093 |
| | 54099 | 54100 | 54110 | 54111 | 54112 | 54113 | 54114 | 54115 | 54116 | 54117 | 54118 | 54122 |
| | 54132 | 54133 | 54134 | 54135 | 54136 | 54137 | 54138 | 54139 | 54140 | 54149 | 54150 | 54151 |
| | 54152 | 54155 | 54156 | 54188 | 54189 | 54190 | 54191 | 54192 | 54193 | 54194 | 54195 | 54196 |
| | 54197 | 54198 | 54199 | 54200 | 54201 | 54202 | 54203 | 54204 | 54205 | 54222 | 54223 | 54224 |
| | 54225 | 54242 | 54243 | 54247 | 54248 | 54249 | 54250 | 54251 | 54252 | 54253 | 54262 | 54263 |
| | 54264 | 54265 | 54266 | 54267 | 54268 | 54269 | 54270 | 54271 | 54272 | 54283 | 54284 | 54285 |
| | 54286 | 54287 | 54288 | 54289 | 54290 | 54291 | 54292 | 54293 | 54296 | 54297 | 54313 | 54314 |
| | 54315 | 54316 | 54318 | 54328 | 54329 | 54330 | 54331 | 54332 | 54333 | 54334 | 54335 | 54336 |
| | 54337 | 54353 | 54371 | 54372 | 54373 | 54374 | 54375 | 54376 | 54377 | 54378 | 54387 | 54392 |
| | 54393 | 54417 | 54462 | 54463 | 54464 | 54465 | 54466 | 54467 | 54468 | 54469 | 54472 | 54473 |
| | 54474 | 54475 | 54493 | 54494 | 54495 | 54496 | 54497 | 54498 | 54499 | 54500 | 54501 | 54502 |
| | 54503 | 54504 | 54505 | 54506 | 54519 | 54520 | 54521 | 54526 | 54527 | 54542 | 54543 | 54544 |
| | 54545 | 54646 | 54647 | 54648 | 54649 | 54650 | 54651 | 54652 | 54653 | 54654 | 54655 | 54656 |
| | 54657 | 54699 | 54707 | 54708 | 54709 | 54710 | 54711 | 54712 | 54713 | 54716 | 54732 | 54733 |
| | 54734 | 54735 | 54736 | 54737 | 54738 | 54739 | 54740 | 54748 | 54749 | 54750 | 54751 | 54755 |
| | 54756 | 54757 | 54758 | 54773 | 54774 | 54775 | 54826 | 54827 | 54828 | 54829 | 54830 | 54831 |
| | 54832 | 54833 | 54834 | 54835 | 54836 | 54837 | 54838 | 54854 | 54887 | 55009 | 55075 | 55076 |
| | 55077 | 55078 | 55079 | 55080 | 55081 | 55082 | 55083 | 55084 | 55088 | 55089 | 55096 | 55097 |
| | 55102 | 55103 | 55104 | 55105 | 55106 | 55114 | 55115 | 55116 | 55117 | 55118 | 55125 | 55126 |
| | 55127 | 55128 | 55129 | 55130 | 55131 | 55142 | 55161 | 55162 | 55166 | 55167 | 55168 | 55169 |
| | 55188 | 55189 | 55190 | 55191 | 55258 | 55259 | 55260 | 55261 | 55285 | 55286 | 55287 | 55288 |
| | 55290 | 55291 | 55329 | 55370 | 55375 | 55376 | 55462 | 55463 | 55464 | 55465 | 55466 | 55467 |
| | 55468 | 55469 | 55470 | 55471 | 55472 | 55473 | 55725 | 55726 | 55727 | 55732 | 55733 | 55797 |
| | 55798 | 55799 | 55800 | 55805 | 55810 | 55811 | 55812 | 55813 | 55843 | 55844 | 55894 | 55895 |
| | 55896 | 55897 | 55898 | 55899 | 55970 | 55971 | 56160 | 56161 | 56162 | 56163 | 56164 | 56165 |
| | 56166 | 56180 | 56471 | 56472 | 56473 | 56552 | 56553 | 56554 | 56555 | 56591 | 56640 | 56641 |
| | 56668 | 56669 | 56704 | 56705 | 56747 | 56748 | 56757 | 56758 | 56763 | 56764 | 56772 | 56773 |
| | 56774 | 56775 | 56776 | 56777 | 56778 | 56779 | 57110 | 57111 | 57112 | 57144 | 57145 | 57153 |
| | 57154 | 57181 | 57199 | 57200 | 57201 | 57202 | 57206 | 57221 | 57225 | 57250 | 57251 | 57252 |
| | 57265 | 57266 | 57318 | 57319 | 57343 | 57344 | 57345 | 57375 | 57376 | 57381 | 57382 | 57383 |
| | 57384 | 57407 | 57421 | 57422 | 57428 | 57429 | 57448 | 57449 | 57483 | 57484 | 57485 | 57486 |
| | 57529 | 57530 | 57546 | 57619 | 57641 | 57642 | 57643 | 57644 | 57645 | 57689 | 57696 | 57697 |
| | 57814 | 57815 | 57841 | 57842 | 57843 | 57844 | 57848 | 57922 | 58229 | 58230 | 58231 | 58232 |
| | 58266 | 58267 | 58279 | 58280 | 58281 | 58282 | 58283 | 58284 | 58311 | 58312 | 58313 | 58314 |
| | 58315 | 58351 | 58352 | 58353 | 58354 | 58355 | 58356 | 58357 | 58358 | 58578 | 58579 | 58703 |
| | 58704 | 58705 | 58706 | 58707 | 58708 | 58709 | 58740 | 58741 | 58742 | 58743 | 58746 | 58747 |
| | 58752 | 58761 | 58762 | 58768 | 58769 | 58841 | 58842 | 58940 | 58941 | 59909 | 59910 | 60356 |
| | 60357 | 60445 | 60446 | 60468 | 60569 | 60663 | 60664 | 60676 | 60677 | 60751 | 60754 | 60755 |
| | 60784 | 60785 | 60786 | 60787 | 60788 | 60789 | 60790 | 60791 | 60792 | 60814 | 60815 | 60940 |
| | 60941 | 60942 | 60943 | 60944 | 60945 | 60946 | 60947 | 60997 | 60998 | 60999 | 61000 | 61001 |
| | 61002 | 61006 | 61083 | 61093 | 61094 | 61095 | 61131 | 61136 | 61137 | 61138 | 61139 | 61140 |
| | 61141 | 61142 | 61143 | 61162 | 61378 | 61379 | 61380 | 61492 | 61493 | 61494 | 61633 | 61679 |
| | 61680 | 61681 | 61735 | 61736 | 61737 | 61738 | 61778 | 61779 | 61780 | 61911 | 61912 | 61913 |
| | 61914 | 61915 | 61959 | 62019 | 62054 | 62055 | 62060 | 62061 | 62076 | 62077 | 62078 | 62079 |
| | 62080 | 62081 | 62082 | 62083 | 62084 | 62103 | 62104 | 62107 | 62108 | 62137 | 62261 | 62262 |
| | 62263 | 62270 | 62292 | 62293 | 62294 | 62295 | 62311 | 62332 | 62345 | 62384 | 62385 | 62540 |
| | 62541 | 62542 | 62624 | 62625 | 62626 | 62627 | 62645 | 62646 | 62647 | 62648 | 62649 | 62650 |
| | 62651 | 62652 | 62916 | 62917 | 62942 | 62957 | 63054 | 63066 | 63203 | 63204 | 63205 | 63206 |
| | 63207 | 63211 | 63212 | 63213 | 63214 | 63215 | 63497 | 63551 | 63552 | 63553 | 63554 | 53555 |
| | 63556 | 63557 | 63558 | 63632 | 63703 | 63706 | 63724 | 63735 | 63899 | 64177 | 64178 | 64179 |
| | 64180 | 6418 | 64182 | 64183 | 64184 | 64200 | 64216 | 64252 | 64253 | 64254 | 64296 | 64297 |
| | 64298 | 64299 | 64314 | 64337 | 64338 | 64339 | 64340 | 64341 | 64342 | 64343 | 64390 | 64471 |
| | 64515 | 64516 | 64517 | 64528 | 64529 | 64535 | 64536 | 64537 | 64569 | 64570 | 64633 | 64634 |
| | 64635 | 64636 | 64715 | 64716 | 64723 | 64724 | 64732 | 64733 | 64734 | 64739 | 64740 | 64741 |
| | 64742 | 64755 | 64756 | 64776 | 64777 | 64778 | 64779 | 64780 | 64952 | 64953 | 64985 | 65050 |
| | 65051 | 65127 | 65170 | 65320 | 65323 | 65506 | 65507 | 65508 | 65509 | 65510 | 65523 | 65531 |
| | 65571 | 65586 | 65618 | 65642 | 65643 | 65664 | 65736 | 65810 | 65811 | 65812 | 65833 | 65948 |
| | 65949 | 65950 | 65951 | 65952 | 66006 | 66144 | 66145 | 66393 | 66394 | 66396 | 66438 | 66439 |
| | 66458 | 66538 | 66656 | 66670 | 66687 | 66716 | 66839 | 66892 | 66907 | 66908 | 66909 | 66952 |
| | 67160 | 67161 | 67221 | 67281 | 67520 | 67774 | 67775 | 67776 | 67880 | 67924 | 68140 | 68141 |
| | 68284 | 68402 | 68409 | 68421 | 68436 | 68476 | 68482 | 68571 | 68648 | 68649 | 68650 | 68690 |
| | 68695 | 68698 | 68722 | 68723 | 68741 | 68742 | 68743 | 68744 | 68911 | 68989 | 69071 | 69116 |
| | 69124 | 69160 | 69161 | 69162 | 69201 | 69202 | 69203 | 69204 | 69233 | 69278 | 69299 | 69300 |
| | 69301 | 69333 | 69343 | 69344 | 69425 | 69464 | 69508 | 69523 | 69524 | 69574 | 69577 | 69585 |
| | 69603 | 69613 | 69734 | 69839 | 69896 | 69897 | 69958 | 70062 | 70078 | 70081 | 70107 | 70114 |
| | 70142 | 70143 | 70145 | 70154 | 70162 | 70204 | 70214 | 70231 | 70241 | 70249 | 70256 | 70289 |
| | 70316 | 70320 | 70326 | 70334 | 70370 | 70402 | 70444 | 70446 | 70475 | 70500 | 70509 | 70511 |
| | 70519 | 70521 | 70524 | 70537 | 70538 | 70539 | 70543 | 70546 | 70548 | 70558 | 70559 | 70569 |
| | 70576 | 70585 | 70586 | 70587 | 70590 | 70591 | 70599 | 70601 | 70603 | 70613 | 70614 | 70616 |
| | 70617 | 70619 | 70625 | 70631 | 70646 | 70648 | 70649 | 70658 | 70659 | 70666 | 70667 | 70668 |
| | 70669 | 70677 | 70679 | 70692 | 70693 | 70696 | 70707 | 70731 | 70737 | 70741 | 70752 | 70757 |
| | 70768 | 70796 | 70807 | 70808 | 70811 | 70819 | 70820 | 70821 | 70829 | 70837 | 70841 | 70843 |
| | 70851 | 70852 | 70860 | 70865 | 70879 | 70880 | 70881 | 70882 | 70890 | 70891 | 70895 | 70896 |
| | 70905 | 70906 | 70907 | 70918 | 70920 | 70926 | 70931 | 70951 | 70993 | 71009 | 71014 | 71015 |
| | 71017 | 71020 | 71022 | 71031 | 71035 | 71038 | 71055 | 71062 | 71082 | 71083 | 71085 | 71104 |
| | 71105 | 71106 | 71107 | 71116 | 71121 | 71182 | 71186 | 71195 | 71196 | 71214 | 71217 | 71218 |
| | 71219 | 71220 | 71221 | 71226 | 71232 | 71233 | 71234 | 71235 | 71248 | 71251 | 71253 | 71255 |
| | 71262 | 71265 | 71300 | 71343 | 71383 | 71461 | 71470 | 71531 | 71560 | 71561 | 71562 | 71580 |
| | 71653 | 71654 | 71655 | 71658 | 71674 | 71682 | 75524 | 75525 | 75527 | 75544 | 75545 | 75546 |
| | 75547 | 75548 | 75549 | 75550 | 75551 | 75552 | 75553 | 75554 | 75555 | 75566 | 75571 | 75572 |
| | 75615 | 75620 | 75626 | 75627 | 75637 | 75638 | 75639 | 75640 | 75641 | 75642 | 75643 | 75644 |
| | 75645 | 75646 | 75654 | 75658 | 75659 | 75660 | 75661 | 75679 | 75681 | 75682 | 75683 | 75695 |
| | 75698 | 75725 | 75726 | 75727 | 75728 | 75729 | 75730 | 75731 | 75766 | 75798 | 75852 | 75858 |
| | 75859 | 75860 | 75861 | 75862 | 75863 | 75864 | 75865 | 75866 | 75867 | 75868 | 75869 | 75870 |
| | 75871 | 75914 | 75937 | 75938 | 75939 | 75948 | 76002 | 76003 | 76004 | 76006 | 76019 | 76020 |
| | 76021 | 76022 | 76023 | 76024 | 76025 | 76026 | 76030 | | | | | |

(2) ATP-processing proteins (SEQ ID NOs)

| | | | | | | | | | | | | |
|---|---|---|---|---|---|---|---|---|---|---|---|---|
| 5132 | 5619 | 6156 | 6234 | 6293 | 6369 | 10378 | 10839 | 10840 | 11388 | 11452 | 53881 | 53882 |
| | 53883 | 53884 | 53933 | 53991 | 53992 | 53993 | 53994 | 53995 | 54005 | 54006 | 54007 | 54008 |
| | 54009 | 54073 | 54074 | 54172 | 54173 | 54174 | 54212 | 54213 | 54238 | 54239 | 54277 | 54278 |
| | 54279 | 54280 | 54281 | 54282 | 54294 | 54295 | 54298 | 54299 | 54302 | 54303 | 54306 | 54320 |
| | 54321 | 54324 | 54325 | 54369 | 54370 | 54388 | 54389 | 54390 | 54391 | 54394 | 54395 | 54396 |
| | 54397 | 54398 | 54399 | 54400 | 54401 | 54402 | 54403 | 54404 | 54405 | 54406 | 54407 | 54408 |
| | 54409 | 54410 | 54411 | 54412 | 54413 | 54414 | 54415 | 54416 | 54418 | 54419 | 54420 | 54421 |
| | 54422 | 54423 | 54424 | 54425 | 54426 | 54427 | 54428 | 54429 | 54430 | 54431 | 54440 | 54442 |
| | 54443 | 54536 | 54537 | 54844 | 54845 | 55542 | 55559 | 55560 | 55561 | 55562 | 55567 | 55577 |
| | 55615 | 55616 | 55705 | 55724 | 55791 | 55793 | 55795 | 55796 | 55804 | 55806 | 55807 | 55809 |
| | 56212 | 56213 | 56585 | 56586 | 56725 | 56726 | 57045 | 57046 | 57196 | 57207 | 57208 | 57363 |
| | 57364 | 58862 | 58970 | 59114 | 59115 | 60701 | 61682 | 61994 | 61997 | 61998 | 61999 | 62000 |
| | 62001 | 62002 | 62003 | 62008 | 62009 | 62010 | 62014 | 62015 | 62016 | 62017 | 62018 | 62022 |
| | 62117 | 62279 | 62280 | 62281 | 62282 | 62308 | 62318 | 62333 | 62614 | 62615 | 62616 | 62617 |
| | 63506 | 66812 | 66813 | 66814 | 67056 | 67761 | 68729 | 68731 | 69049 | 69050 | 69051 | 69081 |
| | 69082 | 69168 | 69356 | 69595 | 69608 | 69614 | 69640 | 69659 | 69663 | 69665 | 69671 | 69715 |
| | 69718 | 69726 | 69732 | 69735 | 69736 | 69741 | 69745 | 69747 | 69748 | 69781 | 69794 | 69960 |
| | 70169 | 70180 | 70185 | 70350 | 70508 | 70609 | 70726 | 70761 | 70970 | 71047 | 71081 | 71095 |
| | 71097 | 71125 | 71143 | 71149 | 71150 | 71210 | 71237 | 71245 | 71286 | 71305 | 71306 | 71309 |
| | 71310 | 71386 | 71389 | 71394 | 71403 | 71409 | 71445 | 71457 | 71476 | 71493 | 71504 | 71505 |
| | 71507 | 71508 | 71583 | 71584 | 71606 | 71608 | 71684 | 71686 | 71695 | 75573 | 75732 | 75733 |
| | 75889 | | | | | | | | | | | |

(3) Cell wall biosynthesis proteins (SEQ ID NOs)

| | | | |
|---|---|---|---|
| 16876 | 69386 | 69387 | 70804 |

(4) Cofactor biosynthesis proteins (SEQ ID NOs)

| | | | | | | | | | | | | |
|---|---|---|---|---|---|---|---|---|---|---|---|---|
| 55348 | 55349 | 55350 | 56938 | 56939 | 56940 | 57423 | 57424 | 58518 | 58519 | 58520 | 58521 | 65125 |
| | 68706 | 71029 | | | | | | | | | | |

(5) CRISPR proteins (provides methanogens an immunity against viruses) (SEQ ID NOs)

| | |
|---|---|
| 70636 | 71045 |

(6) Energy metabolism proteins (SEQ ID NOs)

| | | | | |
|---|---|---|---|---|
| 58756 | 58757 | 61126 | 61127 | 61128 |

(7) Enzymes (SEQ ID NOs)

| | | | | | | | | | | | | |
|---|---|---|---|---|---|---|---|---|---|---|---|---|
| 4617 | 4774 | 4986 | 6013 | 6559 | 11013 | 11468 | 11485 | 11506 | 11773 | 11845 | 16771 | 53045 |
| | 53046 | 53047 | 53048 | 53049 | 53050 | 53051 | 53052 | 53053 | 53054 | 53055 | 53056 | 53057 |
| | 53058 | 53059 | 53060 | 53061 | 53062 | 53063 | 53064 | 53065 | 53066 | 53067 | 53068 | 53069 |
| | 53070 | 53071 | 53072 | 53073 | 53074 | 53075 | 53076 | 53077 | 53078 | 53079 | 53080 | 53081 |
| | 53082 | 53083 | 53084 | 53085 | 53086 | 53087 | 53088 | 53089 | 53090 | 53091 | 53092 | 53093 |
| | 53094 | 53095 | 53096 | 53097 | 53098 | 53099 | 53100 | 53101 | 53102 | 53103 | 53104 | 53105 |
| | 53106 | 53107 | 53108 | 53109 | 53110 | 53111 | 53112 | 53113 | 53114 | 53115 | 53116 | 53117 |
| | 53118 | 53119 | 53120 | 53121 | 53122 | 53123 | 53124 | 53125 | 53126 | 53127 | 53128 | 53129 |
| | 53130 | 53131 | 53132 | 53133 | 53134 | 53135 | 53136 | 53137 | 53138 | 53139 | 53140 | 53141 |
| | 53142 | 53143 | 53144 | 53145 | 53146 | 53147 | 53148 | 53149 | 53150 | 53151 | 53152 | 53153 |
| | 53154 | 53155 | 53156 | 53157 | 53158 | 53159 | 53160 | 53161 | 53162 | 53163 | 53164 | 53165 |
| | 53166 | 53167 | 53168 | 53169 | 53170 | 53171 | 53172 | 53173 | 53174 | 53175 | 53176 | 53177 |
| | 53178 | 53179 | 53180 | 53181 | 53182 | 53183 | 53184 | 53185 | 53186 | 53187 | 53188 | 53189 |
| | 53190 | 53191 | 53192 | 53193 | 53194 | 53195 | 53196 | 53197 | 53198 | 53199 | 53200 | 53201 |
| | 53202 | 53203 | 53204 | 53205 | 53206 | 53207 | 53208 | 53209 | 53210 | 53211 | 53212 | 53213 |
| | 53214 | 53215 | 53216 | 53217 | 53243 | 53244 | 53245 | 53246 | 53247 | 53248 | 53249 | 53404 |
| | 54379 | 54380 | 54441 | 54517 | 54518 | 54798 | 54799 | 54800 | 54801 | 54807 | 54808 | 54809 |
| | 54810 | 55017 | 55018 | 55019 | 55257 | 55434 | 55694 | 55714 | 55738 | 55767 | 55772 | 55781 |
| | 55782 | 55792 | 55794 | 55808 | 56331 | 56332 | 56333 | 56606 | 56734 | 56735 | 56744 | 56745 |
| | 56746 | 57077 | 57078 | 57120 | 57121 | 57122 | 57173 | 57174 | 57175 | 57176 | 57182 | 57183 |
| | 57184 | 57332 | 57333 | 57430 | 57431 | 57432 | 57433 | 57434 | 57439 | 57440 | 57560 | 57561 |
| | 57599 | 57600 | 57822 | 57823 | 57870 | 57871 | 58169 | 58170 | 58316 | 58436 | 58437 | 58438 |
| | 58439 | 58588 | 58712 | 58713 | 58714 | 58720 | 58721 | 58729 | 58730 | 58731 | 58855 | 58893 |
| | 58894 | 58901 | 58902 | 58909 | 58910 | 58959 | 58960 | 59122 | 59123 | 59124 | 59958 | 59959 |
| | 59960 | 60337 | 60338 | 60339 | 60361 | 60362 | 60363 | 60364 | 60507 | 60508 | 60509 | 60510 |
| | 60511 | 60512 | 60629 | 60630 | 60631 | 60632 | 60633 | 60641 | 60642 | 60643 | 60644 | 60645 |
| | 60646 | 60647 | 60648 | 60649 | 60650 | 60651 | 60652 | 60653 | 60654 | 60655 | 60656 | 60657 |
| | 60658 | 60675 | 60717 | 60718 | 60719 | 60763 | 60764 | 60765 | 60766 | 60896 | 60897 | 60898 |
| | 61029 | 61030 | 61201 | 61202 | 61203 | 61204 | 61205 | 61206 | 61207 | 61208 | 61256 | 61257 |
| | 61258 | 61259 | 61284 | 61285 | 61286 | 61287 | 61288 | 61289 | 61295 | 61383 | 61384 | 61385 |
| | 61386 | 61387 | 61388 | 61389 | 61390 | 61391 | 61392 | 61393 | 61394 | 61395 | 61396 | 61397 |
| | 61398 | 61399 | 61400 | 61401 | 61402 | 61403 | 61404 | 61409 | 61410 | 61411 | 61412 | 61413 |
| | 61414 | 61417 | 61418 | 61419 | 61420 | 61421 | 61422 | 61423 | 61424 | 61425 | 61426 | 61427 |
| | 61428 | 61429 | 61430 | 61431 | 61432 | 61433 | 61434 | 61435 | 61436 | 61437 | 61438 | 61439 |
| | 61440 | 61444 | 61445 | 61446 | 61447 | 61448 | 61449 | 61709 | 61710 | 61711 | 61752 | 61753 |
| | 61754 | 61830 | 61831 | 61832 | 61855 | 61856 | 61924 | 62056 | 62057 | 62085 | 62089 | 62099 |
| | 62110 | 62133 | 62148 | 62208 | 62209 | 62210 | 62211 | 62212 | 62228 | 62389 | 62390 | 62391 |
| | 62392 | 62393 | 62394 | 62395 | 62396 | 62405 | 62406 | 62420 | 62421 | 62422 | 62463 | 62464 |
| | 62465 | 62481 | 62482 | 62483 | 62484 | 62493 | 62494 | 62495 | 62496 | 62497 | 62498 | 62499 |
| | 62509 | 62510 | 62511 | 62512 | 62513 | 62514 | 62518 | 62519 | 62520 | 62521 | 62522 | 62523 |
| | 62524 | 62525 | 62526 | 62527 | 62535 | 62536 | 62537 | 62547 | 62548 | 62549 | 62557 | 62558 |
| | 62559 | 62565 | 62566 | 62567 | 62571 | 62572 | 62573 | 62574 | 62575 | 62576 | 62577 | 62578 |
| | 62579 | 62590 | 62591 | 62641 | 62642 | 62673 | 62674 | 62675 | 62676 | 62677 | 63385 | 63502 |
| | 63564 | 63565 | 63965 | 64067 | 64278 | 64563 | 64589 | 64604 | 64605 | 64606 | 64607 | 64608 |
| | 64626 | 64627 | 64628 | 64629 | 64637 | 64651 | 64660 | 64679 | 64682 | 64687 | 64688 | 64693 |
| | 64750 | 64784 | 64785 | 64786 | 64920 | 64921 | 65076 | 65237 | 65449 | 65479 | 65480 | 65481 |
| | 65499 | 65532 | 65537 | 65589 | 65740 | 65775 | 65776 | 65992 | 65993 | 65994 | 65995 | 65996 |
| | 65997 | 65998 | 66200 | 66201 | 66202 | 66203 | 66204 | 66205 | 66206 | 66207 | 66208 | 66229 |
| | 66230 | 66231 | 66359 | 66477 | 66523 | 66536 | 66561 | 67804 | 67884 | 67885 | 67886 | 67910 |
| | 67911 | 68392 | 68393 | 68394 | 68395 | 68396 | 68412 | 68413 | 68414 | 68661 | 68662 | 68664 |
| | 68665 | 68701 | 68702 | 68719 | 68720 | 68762 | 68763 | 68825 | 68843 | 69060 | 69061 | 69167 |
| | 69210 | 69217 | 69367 | 69368 | 69369 | 69398 | 69422 | 69423 | 69514 | 69556 | 69570 | 69600 |
| | 69616 | 69617 | 69618 | 69623 | 69638 | 69639 | 69677 | 69708 | 69723 | 69956 | 69972 | 70038 |
| | 70128 | 70171 | 70194 | 70284 | 70317 | 70318 | 70351 | 70354 | 70412 | 70413 | 70421 | 70466 |
| | 70608 | 70633 | 70650 | 70651 | 70685 | 70686 | 70714 | 70733 | 70748 | 70758 | 70765 | 70925 |
| | 71026 | 71036 | 71046 | 71058 | 71432 | 71078 | 71094 | 71117 | 71119 | 71131 | 71167 | 71177 |
| | 71187 | 71193 | 71197 | 71200 | 71205 | 71207 | 71212 | 71215 | 71229 | 71230 | 71246 | 71250 |
| | 71254 | 71258 | 71284 | 71293 | 71296 | 71331 | 71333 | 71339 | 71347 | 71350 | 71356 | 71373 |
| | 71375 | 71376 | 71378 | 71379 | 71385 | 71410 | 71417 | 71431 | 71432 | 71434 | 71435 | 71444 |
| | 71447 | 71448 | 71451 | 71458 | 71477 | 71509 | 71517 | 71526 | 71530 | 71538 | 71539 | 71556 |
| | 71566 | 71567 | 71587 | 71602 | 71603 | 71609 | 71611 | 71623 | 71624 | 71625 | 71629 | 71635 |
| | 71637 | 71639 | 71642 | 71643 | 71644 | 71656 | 71678 | 71702 | 71703 | 75778 | 75779 | 75780 |
| | 75781 | 75782 | 75783 | 75784 | 75897 | 75905 | 75906 | 75907 | 75908 | 75909 | 75910 | 75985 |
| | 75998 | | | | | | | | | | | |

(8) Fatty acid synthesis (SEQ ID NOs)

| | |
|---|---|
| 68659 | 68660 |

(9) General metabolism (SEQ ID NOs)

| | | | | | | | | | | | | |
|---|---|---|---|---|---|---|---|---|---|---|---|---|
| 5480 | 5955 | 11908 | 16866 | 16867 | 16874 | 53369 | 53405 | 54450 | 54451 | 54452 | 54453 | 54454 |
| | 54888 | 54889 | 54890 | 54891 | 54892 | 55649 | 56541 | 56542 | 56550 | 56551 | 56556 | 56557 |
| | 56558 | 56559 | 56560 | 56561 | 56562 | 56563 | 56749 | 56750 | 56759 | 56760 | 56761 | 56762 |
| | 56801 | 56802 | 56826 | 56827 | 57308 | 57309 | 57902 | 57903 | 58386 | 58387 | 58388 | 58389 |
| | 58413 | 58414 | 58415 | 58416 | 61231 | 61232 | 61233 | 62215 | 62216 | 62217 | 62223 | 62224 |
| | 62963 | 63030 | 63031 | 63582 | 63916 | 63917 | 63918 | 63919 | 63920 | 63972 | 63973 | 63974 |
| | 64450 | 64451 | 65272 | 65273 | 65539 | 65722 | 65723 | 65724 | 65766 | 65767 | 65786 | 66530 |
| | 66616 | 66655 | 67200 | 67201 | 67253 | 67934 | 67946 | 67947 | 68134 | 68135 | 68306 | 68434 |
| | 68435 | 68669 | 68670 | 68673 | 68682 | 68683 | 68709 | 68759 | 68784 | 68787 | 68840 | 68841 |
| | 69175 | 69176 | 69226 | 69586 | 69591 | 69664 | 69672 | 69690 | 70033 | 70295 | 70310 | 70381 |
| | 70485 | 70486 | 70610 | 70675 | 70921 | 71103 | 71136 | 71168 | 71192 | 71202 | 71324 | 71328 |
| | 71341 | 71374 | 71388 | 71405 | 71419 | 71422 | 71466 | 71471 | 71485 | 71573 | 71613 | 71657 |
| | 71688 | 71691 | 76005 | | | | | | | | | |

(10) Membrane proteins (SEQ ID NOs)

| | | | | | | | | | | | | |
|---|---|---|---|---|---|---|---|---|---|---|---|---|
| 4452 | 4478 | 4481 | 4483 | 4500 | 4505 | 4515 | 4529 | 4547 | 4580 | 4581 | 4584 | 4601 |
| | 4606 | 4621 | 4627 | 4630 | 4657 | 4658 | 4659 | 4664 | 4665 | 4667 | 4668 | 4671 |
| | 4682 | 4712 | 4713 | 4740 | 4749 | 4762 | 4764 | 4790 | 4791 | 4795 | 4809 | 4810 |
| | 4811 | 4812 | 4842 | 4845 | 4848 | 4857 | 4861 | 4866 | 4879 | 4893 | 4903 | 4921 |
| | 4923 | 4924 | 4928 | 4932 | 4942 | 4943 | 4945 | 4948 | 4954 | 4957 | 4975 | 4976 |
| | 4978 | 4980 | 5000 | 5014 | 5029 | 5030 | 5031 | 5035 | 5037 | 5047 | 5055 | 5058 |
| | 5064 | 5067 | 5072 | 5096 | 5103 | 5104 | 5116 | 5119 | 5126 | 5127 | 5139 | 5149 |
| | 5150 | 5151 | 5153 | 5159 | 5169 | 5178 | 5179 | 5180 | 5188 | 5192 | 5193 | 5202 |
| | 5235 | 5238 | 5264 | 5265 | 5266 | 5271 | 5273 | 5310 | 5334 | 5363 | 5373 | 5397 |
| | 5400 | 5401 | 5408 | 5422 | 5433 | 5454 | 5455 | 5460 | 5463 | 5474 | 5487 | 5492 |
| | 5513 | 5517 | 5529 | 5534 | 5549 | 5557 | 5576 | 5624 | 5626 | 5627 | 5628 | 5638 |
| | 5645 | 5651 | 5664 | 5665 | 5666 | 5721 | 5734 | 5754 | 5762 | 5763 | 5768 | 5775 |
| | 5807 | 5817 | 5855 | 5874 | 5883 | 5912 | 5925 | 5966 | 5967 | 5974 | 5982 | 5987 |
| | 6010 | 6017 | 6030 | 6037 | 6039 | 6067 | 6068 | 6073 | 6074 | 6085 | 6086 | 6089 |
| | 6125 | 6126 | 6170 | 6191 | 6217 | 6226 | 6227 | 6235 | 6264 | 6265 | 6266 | 6290 |
| | 6314 | 6316 | 6321 | 6324 | 6332 | 6378 | 6382 | 6384 | 6401 | 6422 | 6423 | 6447 |
| | 6455 | 6476 | 6482 | 6488 | 6489 | 6497 | 6538 | 6540 | 6550 | 6562 | 6568 | 6575 |
| | 6578 | 6588 | 6604 | 6607 | 6612 | 6616 | 6617 | 6634 | 10272 | 10291 | 10306 | 10322 |
| | 10325 | 10329 | 10347 | 10349 | 10350 | 10353 | 10368 | 10373 | 10383 | 10385 | 10391 | 10392 |
| | 10408 | 10432 | 10438 | 10439 | 10443 | 10460 | 10462 | 10465 | 10497 | 10501 | 10502 | 10503 |
| | 10504 | 10510 | 10512 | 10513 | 10515 | 10517 | 10538 | 10543 | 10545 | 10559 | 10564 | 10573 |
| | 10586 | 10626 | 10641 | 10653 | 10667 | 10703 | 10721 | 10727 | 10734 | 10735 | 10736 | 10743 |
| | 10744 | 10745 | 10752 | 10770 | 10792 | 10793 | 10797 | 10799 | 10848 | 10939 | 10940 | 10941 |
| | 10944 | 10945 | 10978 | 10983 | 10984 | 11021 | 11024 | 11041 | 11042 | 11056 | 11057 | 11115 |
| | 11126 | 11137 | 11138 | 11146 | 11158 | 11165 | 11177 | 11183 | 11186 | 11199 | 11217 | 11218 |
| | 11219 | 11233 | 11234 | 11238 | 11247 | 11248 | 11268 | 11272 | 11276 | 11277 | 11301 | 11305 |
| | 11312 | 11322 | 11326 | 11338 | 11351 | 11357 | 11358 | 11365 | 11385 | 11410 | 11428 | 11432 |
| | 11434 | 11457 | 11474 | 11475 | 11476 | 11482 | 11537 | 11561 | 11565 | 11597 | 11599 | 11600 |
| | 11622 | 11623 | 11631 | 11636 | 11637 | 11639 | 11643 | 11647 | 11679 | 11682 | 11683 | 11685 |
| | 11691 | 11692 | 11701 | 11703 | 11709 | 11724 | 11752 | 11753 | 11763 | 11766 | 11778 | 11779 |
| | 11784 | 11803 | 11874 | 11914 | 11916 | 11931 | 11932 | 11935 | 11937 | 11944 | 11954 | 11962 |
| | 11963 | 11967 | 11976 | 12005 | 12010 | 12020 | 12021 | 12031 | 12049 | 53513 | 53514 | 53515 |
| | 53516 | 53517 | 53518 | 53519 | 53520 | 53521 | 53601 | 53602 | 53650 | 53651 | 53652 | 53653 |
| | 53654 | 53655 | 53656 | 53676 | 53677 | 53678 | 53679 | 53680 | 53681 | 53682 | 53683 | 53686 |
| | 53687 | 53688 | 53689 | 53690 | 53691 | 53692 | 53693 | 53795 | 53912 | 53913 | 53914 | 53915 |
| | 53971 | 53975 | 54014 | 54015 | 54016 | 54017 | 54080 | 54083 | 54084 | 54085 | 54086 | 54087 |
| | 54088 | 54089 | 54090 | 54091 | 54092 | 54101 | 54102 | 54103 | 54104 | 54105 | 54106 | 54107 |
| | 54123 | 54124 | 54125 | 54126 | 54127 | 54128 | 54129 | 54130 | 54131 | 54305 | 54447 | 54448 |
| | 54449 | 54470 | 54471 | 54491 | 54492 | 54625 | 54626 | 54627 | 54660 | 54661 | 54662 | 54677 |
| | 54693 | 54694 | 54746 | 54747 | 54761 | 54762 | 54763 | 54764 | 54765 | 54766 | 54767 | 54768 |
| | 54769 | 54770 | 54771 | 54772 | 54776 | 54781 | 54782 | 54783 | 54784 | 54879 | 54880 | 54881 |
| | 55015 | 55016 | 55065 | 55074 | 55090 | 55100 | 55101 | 55110 | 55111 | 55112 | 55113 | 55119 |
| | 55120 | 55121 | 55122 | 55123 | 55124 | 55133 | 55134 | 55135 | 55136 | 55137 | 55138 | 55139 |
| | 55140 | 55141 | 55143 | 55144 | 55145 | 55146 | 55147 | 55148 | 55149 | 55150 | 55151 | 55152 |
| | 55153 | 55154 | 55155 | 55156 | 55157 | 55158 | 55159 | 55160 | 55163 | 55164 | 55165 | 55170 |
| | 55171 | 55172 | 55173 | 55175 | 55176 | 55177 | 55197 | 55198 | 55199 | 55200 | 55201 | 55202 |
| | 55205 | 55206 | 55207 | 55208 | 55209 | 55210 | 55211 | 55212 | 55213 | 55214 | 55215 | 55216 |
| | 55217 | 55218 | 55219 | 55220 | 55221 | 55222 | 55223 | 55224 | 55225 | 55226 | 55227 | 55228 |
| | 55229 | 55230 | 55231 | 55232 | 55233 | 55235 | 55236 | 55237 | 55239 | 55240 | 55241 | 55242 |
| | 55243 | 55244 | 55245 | 55246 | 55247 | 55248 | 55249 | 55250 | 55251 | 55252 | 55253 | 55254 |
| | 55255 | 55256 | 55262 | 55263 | 55264 | 55265 | 55266 | 55267 | 55268 | 55269 | 55270 | 55271 |
| | 55272 | 55273 | 55274 | 55275 | 55276 | 55277 | 55278 | 55280 | 55281 | 55282 | 55283 | 55284 |
| | 55293 | 55294 | 55295 | 55297 | 55298 | 55299 | 55300 | 55301 | 55303 | 55304 | 55305 | 55309 |
| | 55310 | 55312 | 55313 | 55314 | 55315 | 55316 | 55317 | 55318 | 55319 | 55320 | 55321 | 55322 |
| | 55323 | 55324 | 55325 | 55326 | 55327 | 55328 | 55330 | 55331 | 55332 | 55333 | 55334 | 55335 |
| | 55336 | 55337 | 55338 | 55339 | 55340 | 55341 | 55342 | 55343 | 55344 | 55345 | 55346 | 55347 |
| | 55351 | 55352 | 55353 | 55354 | 55355 | 55362 | 55363 | 55364 | 55365 | 55366 | 55367 | 55368 |
| | 55377 | 55378 | 55379 | 55380 | 55381 | 55382 | 55388 | 55391 | 55392 | 55393 | 55394 | 55395 |
| | 55396 | 55397 | 55407 | 55408 | 55409 | 55412 | 55418 | 55419 | 55420 | 55422 | 55424 | 55425 |
| | 55427 | 55428 | 55429 | 55430 | 55431 | 55432 | 55433 | 55435 | 55436 | 55437 | 55438 | 55439 |
| | 55440 | 55441 | 55442 | 55443 | 55444 | 55451 | 55452 | 55453 | 55454 | 55455 | 55456 | 55457 |
| | 55458 | 55460 | 55461 | 55474 | 55475 | 55476 | 55478 | 55479 | 55483 | 55484 | 55485 | 55492 |
| | 55493 | 55496 | 55497 | 55505 | 55506 | 55507 | 55508 | 55509 | 55510 | 55511 | 55513 | 55514 |
| | 55515 | 55516 | 55521 | 55522 | 55523 | 55524 | 55525 | 55529 | 55530 | 55531 | 55532 | 55533 |
| | 55534 | 55535 | 55536 | 55538 | 55539 | 55540 | 55541 | 55543 | 55544 | 55545 | 55548 | 55549 |
| | 55550 | 55551 | 55552 | 55553 | 55554 | 55555 | 55556 | 55557 | 55563 | 55564 | 55568 | 55569 |
| | 55570 | 55571 | 55572 | 55573 | 55574 | 55575 | 55576 | 55578 | 55579 | 55580 | 55581 | 55582 |
| | 55583 | 55584 | 55587 | 55588 | 55589 | 55593 | 55594 | 55595 | 55597 | 55598 | 55600 | 55601 |
| | 55602 | 55603 | 55604 | 55605 | 55606 | 55607 | 55608 | 55609 | 55610 | 55611 | 55612 | 55613 |
| | 55614 | 55617 | 55618 | 55619 | 55620 | 55621 | 55622 | 55623 | 55625 | 55626 | 55627 | 55628 |
| | 55635 | 55639 | 55640 | 55641 | 55642 | 55643 | 55644 | 55645 | 55646 | 55647 | 55648 | 55652 |
| | 55653 | 55656 | 55657 | 55658 | 55659 | 55660 | 55661 | 55662 | 55663 | 55664 | 55665 | 55666 |
| | 55667 | 55668 | 55669 | 55670 | 55671 | 55672 | 55673 | 55674 | 55675 | 55676 | 55677 | 55678 |
| | 55679 | 55680 | 55681 | 55682 | 55683 | 55684 | 55685 | 55686 | 55687 | 55691 | 55692 | 55693 |
| | 55701 | 55702 | 55703 | 55704 | 55706 | 55707 | 55708 | 55709 | 55710 | 55739 | 55740 | 55741 |
| | 55742 | 55743 | 55744 | 55745 | 55746 | 55747 | 55748 | 55749 | 55757 | 55758 | 55759 | 55783 |
| | 55784 | 55830 | 55831 | 55832 | 55969 | 55986 | 55987 | 56001 | 56002 | 56003 | 56004 | 56005 |
| | 56006 | 56007 | 56012 | 56013 | 56014 | 56015 | 56081 | 56082 | 56083 | 56107 | 56108 | 56109 |
| | 56110 | 56111 | 56112 | 56130 | 56131 | 56132 | 56133 | 56134 | 56135 | 56136 | 56153 | 56154 |
| | 56155 | 56156 | 56157 | 56170 | 56171 | 56172 | 56173 | 56184 | 56185 | 56186 | 56187 | 56188 |
| | 56197 | 56198 | 56199 | 56206 | 56207 | 56208 | 56209 | 56210 | 56214 | 56215 | 56216 | 56217 |
| | 56218 | 56221 | 56222 | 56223 | 56224 | 56225 | 56259 | 56260 | 56261 | 56262 | 56271 | 56272 |
| | 56273 | 56274 | 56275 | 56276 | 56277 | 56280 | 56281 | 56282 | 56283 | 56284 | 56288 | 56289 |
| | 56290 | 56295 | 56296 | 56297 | 56298 | 56299 | 56300 | 56301 | 56302 | 56303 | 56307 | 56308 |
| | 56309 | 56310 | 56311 | 56312 | 56313 | 56314 | 56315 | 56316 | 56317 | 56326 | 56327 | 56328 |
| | 56329 | 56330 | 56334 | 56335 | 56336 | 56337 | 56338 | 56339 | 56340 | 56341 | 56347 | 56348 |
| | 56349 | 56350 | 56412 | 56413 | 56414 | 56419 | 56420 | 56421 | 56449 | 56450 | 56451 | 56452 |
| | 56465 | 56466 | 56476 | 56477 | 56478 | 56486 | 56500 | 56501 | 56502 | 56504 | 56505 | 56506 |
| | 56507 | 56525 | 56526 | 56527 | 56528 | 56564 | 56565 | 56566 | 56567 | 56568 | 56569 | 56570 |
| | 56571 | 56576 | 56577 | 56587 | 56588 | 56593 | 56594 | 56595 | 56596 | 56597 | 56598 | 56601 |
| | 56654 | 56655 | 56682 | 56684 | 56685 | 56686 | 56687 | 56688 | 56689 | 56690 | 56691 | 56698 |
| | 56699 | 56710 | 56711 | 56724 | 56736 | 56737 | 56738 | 56739 | 56740 | 56751 | 56752 | 56753 |
| | 56754 | 56768 | 56769 | 56770 | 56771 | 56780 | 56781 | 56806 | 56807 | 56808 | 56830 | 56831 |
| | 56832 | 56848 | 56849 | 56850 | 56866 | 56867 | 56868 | 56903 | 56904 | 56905 | 56947 | 56948 |
| | 56960 | 56961 | 56962 | 56963 | 56996 | 56997 | 56998 | 56999 | 57016 | 57017 | 57018 | 57032 |
| | 57033 | 57034 | 57035 | 57053 | 57054 | 57055 | 57097 | 57098 | 57099 | 57100 | 57101 | 57102 |
| | 57103 | 57104 | 57105 | 57106 | 57113 | 57114 | 57115 | 57116 | 57117 | 57118 | 57119 | 57123 |
| | 57124 | 57125 | 57126 | 57127 | 57128 | 57129 | 57137 | 57138 | 57148 | 57149 | 57150 | 57151 |
| | 57165 | 57166 | 57211 | 57212 | 57294 | 57295 | 57298 | 57299 | 57300 | 57377 | 57378 | 57397 |
| | 57398 | 57402 | 57403 | 57404 | 57405 | 57406 | 57425 | 57426 | 57427 | 57441 | 57442 | 57443 |
| | 57444 | 57450 | 57451 | 57452 | 57453 | 57480 | 57481 | 57482 | 57492 | 57493 | 57497 | 57498 |
| | 57508 | 57509 | 57524 | 57525 | 57550 | 57551 | 57552 | 57553 | 57554 | 57574 | 57575 | 57656 |
| | 57657 | 57658 | 57684 | 57685 | 57698 | 57699 | 57724 | 57725 | 57726 | 57727 | 57795 | 57796 |
| | 57797 | 57798 | 57799 | 57800 | 57801 | 57802 | 57806 | 57807 | 57837 | 57893 | 57894 | 58162 |
| | 58163 | 58164 | 58168 | 58218 | 58222 | 58226 | 58239 | 58240 | 58241 | 58328 | 58329 | 58330 |
| | 58406 | 58407 | 58408 | 58429 | 58430 | 58431 | 58641 | 58642 | 58643 | 58644 | 58645 | 58646 |
| | 58661 | 58701 | 58702 | 58732 | 58733 | 58734 | 58735 | 58736 | 58737 | 58738 | 58739 | 58744 |
| | 58745 | 58758 | 58759 | 58760 | 58763 | 58764 | 58765 | 58766 | 58770 | 58771 | 58772 | 58773 |
| | 58774 | 58775 | 58776 | 58777 | 58778 | 58779 | 58780 | 58781 | 58782 | 58783 | 58784 | 58785 |
| | 58786 | 58787 | 58798 | 58799 | 58800 | 58801 | 58806 | 58807 | 58808 | 58816 | 58817 | 58825 |
| | 58826 | 58853 | 58854 | 58859 | 58860 | 58861 | 58864 | 58865 | 58866 | 58867 | 58868 | 58869 |
| | 58870 | 58871 | 58872 | 58873 | 58878 | 58879 | 58880 | 58881 | 58882 | 58883 | 58884 | 58885 |
| | 58886 | 58887 | 58888 | 58889 | 58890 | 58891 | 58892 | 58895 | 58896 | 58897 | 58898 | 58899 |
| | 58900 | 58903 | 58904 | 58905 | 58906 | 58907 | 58908 | 58918 | 58919 | 58920 | 58921 | 59018 |
| | 59169 | 59170 | 59179 | 59180 | 59184 | 59185 | 59216 | 59217 | 59236 | 59237 | 59244 | 59300 |
| | 59301 | 59333 | 59334 | 59335 | 59336 | 59344 | 59345 | 59367 | 59368 | 59387 | 59388 | 59389 |
| | 59400 | 59401 | 59436 | 59437 | 59438 | 60082 | 60083 | 60084 | 60085 | 60222 | 60268 | 60383 |
| | 60384 | 60385 | 60414 | 60415 | 60416 | 60417 | 60476 | 60477 | 60478 | 60479 | 60491 | 60543 |
| | 60599 | 60600 | 60601 | 60602 | 60662 | 60668 | 60671 | 60672 | 60673 | 60674 | 60678 | 60679 |
| | 60685 | 60694 | 60695 | 60697 | 60698 | 60727 | 60728 | 60729 | 60747 | 60748 | 60749 | 60843 |
| | 60844 | 60875 | 60876 | 60894 | 60895 | 60903 | 60904 | 60908 | 60909 | 60917 | 60918 | 60919 |
| | 60920 | 60921 | 60922 | 60923 | 60924 | 60925 | 60926 | 60927 | 60928 | 60929 | 60930 | 60931 |
| | 60932 | 60933 | 60934 | 60935 | 60936 | 60937 | 60938 | 60939 | 60952 | 60953 | 60954 | 60955 |
| | 60956 | 60957 | 60958 | 60959 | 60960 | 60966 | 60967 | 60968 | 60971 | 60972 | 60973 | 60974 |
| | 60975 | 60976 | 60977 | 60978 | 60979 | 60980 | 60981 | 60982 | 60983 | 60984 | 60985 | 60986 |
| | 60987 | 60988 | 60989 | 60990 | 60994 | 61016 | 61023 | 61027 | 61028 | 61031 | 61099 | 61106 |
| | 61112 | 61113 | 61114 | 61115 | 61116 | 61117 | 61118 | 61119 | 61144 | 61151 | 61152 | 61156 |
| | 61157 | 61158 | 61159 | 61167 | 61168 | 61169 | 61170 | 61171 | 61175 | 61176 | 61177 | 61181 |
| | 61182 | 61183 | 61184 | 61185 | 61186 | 61187 | 61188 | 61190 | 61191 | 61192 | 61193 | 61194 |
| | 61195 | 61196 | 61197 | 61209 | 61210 | 61211 | 61212 | 61213 | 61214 | 61215 | 61216 | 61217 |
| | 61221 | 61222 | 61223 | 61224 | 61225 | 61226 | 61227 | 61228 | 61229 | 61230 | 61234 | 61235 |
| | 61236 | 61237 | 61238 | 61239 | 61250 | 61251 | 61252 | 61253 | 61254 | 61255 | 61263 | 61264 |
| | 61265 | 61266 | 61267 | 61268 | 61269 | 61270 | 61276 | 61279 | 61280 | 61296 | 61315 | 61316 |
| | 61317 | 61328 | 61331 | 61374 | 61375 | 61415 | 61416 | 61456 | 61462 | 61464 | 61474 | 61475 |
| | 61476 | 61477 | 61478 | 61479 | 61480 | 61499 | 61500 | 61501 | 61528 | 61538 | 61590 | 61604 |
| | 61608 | 61630 | 61645 | 61649 | 61650 | 61656 | 61657 | 61658 | 61659 | 61663 | 61664 | 61665 |
| | 61671 | 61690 | 61695 | 61696 | 61697 | 61698 | 61699 | 61700 | 61701 | 61702 | 61716 | 61717 |
| | 61741 | 61742 | 61743 | 61746 | 61747 | 61755 | 61756 | 61757 | 61760 | 61761 | 61762 | 61763 |
| | 61764 | 61771 | 61772 | 61773 | 61781 | 61800 | 61801 | 61802 | 61811 | 61812 | 61813 | 61818 |
| | 61819 | 61820 | 61821 | 61822 | 61829 | 61836 | 61837 | 61838 | 61839 | 61840 | 61841 | 61842 |
| | 61843 | 61862 | 61863 | 61864 | 61865 | 61866 | 61882 | 61895 | 61898 | 61901 | 61902 | 61918 |
| | 61919 | 61920 | 61930 | 61931 | 61935 | 61936 | 61939 | 61940 | 61941 | 61969 | 61970 | 61971 |
| | 61989 | 62004 | 62005 | 62006 | 62007 | 62049 | 62050 | 62051 | 62052 | 62053 | 62058 | 62059 |
| | 62065 | 62066 | 62067 | 62068 | 62072 | 62073 | 62074 | 62086 | 62087 | 62088 | 62090 | 62091 |
| | 62092 | 62094 | 62095 | 62096 | 62097 | 62098 | 62100 | 62106 | 62109 | 62111 | 62112 | 62113 |
| | 62122 | 62128 | 62129 | 62134 | 62135 | 62136 | 62144 | 62145 | 62146 | 62147 | 62155 | 62156 |
| | 62157 | 62231 | 62232 | 62233 | 62234 | 62235 | 62236 | 62237 | 62240 | 62241 | 62242 | 62243 |
| | 62252 | 62265 | 62275 | 62283 | 62284 | 62285 | 62286 | 62287 | 62288 | 62289 | 62298 | 62299 |
| | 62300 | 62301 | 62302 | 62303 | 62325 | 62326 | 62327 | 62334 | 62335 | 62336 | 62337 | 62338 |
| | 62339 | 62340 | 62341 | 62342 | 62343 | 62344 | 62349 | 62350 | 62351 | 62352 | 62353 | 62354 |
| | 62355 | 62356 | 62358 | 62359 | 62360 | 62407 | 62408 | 62409 | 62410 | 62411 | 62412 | 62413 |
| | 62414 | 62415 | 62439 | 62440 | 62441 | 62442 | 62443 | 62448 | 62449 | 62450 | 62451 | 62452 |
| | 62453 | 62454 | 62467 | 62468 | 62469 | 62470 | 62471 | 62472 | 62473 | 62474 | 62475 | 62485 |
| | 62486 | 62487 | 62488 | 62489 | 62490 | 62491 | 62492 | 62500 | 62501 | 62502 | 62503 | 62528 |
| | 62529 | 62538 | 62539 | 62550 | 62551 | 62552 | 62553 | 62554 | 62555 | 62556 | 62583 | 62584 |
| | 62585 | 62586 | 62587 | 62588 | 62589 | 62607 | 62608 | 62609 | 62610 | 62611 | 62620 | 62621 |
| | 62622 | 62628 | 62630 | 62631 | 62632 | 62633 | 62643 | 62660 | 62661 | 62662 | 62663 | 62664 |
| | 62665 | 62684 | 62685 | 62686 | 62687 | 62688 | 62689 | 62690 | 62691 | 62692 | 62693 | 62694 |
| | 62695 | 62696 | 62701 | 62702 | 62703 | 62704 | 62710 | 62711 | 62714 | 62715 | 62716 | 62717 |
| | 62718 | 62719 | 62720 | 62722 | 62723 | 62724 | 62725 | 62726 | 62727 | 62728 | 62729 | 62730 |
| | 62731 | 62732 | 62733 | 62734 | 62735 | 62736 | 62737 | 62741 | 62742 | 62743 | 62744 | 62745 |
| | 62750 | 62751 | 62752 | 62753 | 62754 | 62755 | 62756 | 62757 | 62758 | 62759 | 62760 | 62761 |
| | 62763 | 62764 | 62769 | 62770 | 62775 | 62776 | 62777 | 62780 | 62781 | 62782 | 62783 | 62784 |
| | 62785 | 62786 | 62787 | 62788 | 62789 | 62790 | 62791 | 62792 | 62793 | 62796 | 62797 | 62798 |
| | 62800 | 62801 | 62802 | 62803 | 62804 | 62805 | 62806 | 62807 | 62808 | 62809 | 62810 | 62811 |
| | 62812 | 62813 | 62814 | 62815 | 62816 | 62817 | 62818 | 62819 | 62823 | 62824 | 62825 | 62826 |
| | 62830 | 62831 | 62832 | 62833 | 62834 | 62838 | 62839 | 62840 | 62841 | 62842 | 62843 | 62844 |
| | 62845 | 62846 | 62847 | 62848 | 62849 | 62850 | 62851 | 62852 | 62853 | 62854 | 62855 | 62856 |
| | 62857 | 62858 | 62859 | 62860 | 62865 | 62866 | 62867 | 62868 | 62869 | 62870 | 62871 | 62872 |
| | 62873 | 62874 | 62875 | 62876 | 62877 | 62878 | 62879 | 62883 | 62884 | 62885 | 62886 | 62887 |
| | 62888 | 62889 | 62890 | 62891 | 62892 | 62893 | 62894 | 62895 | 62896 | 62897 | 62898 | 62899 |
| | 62900 | 62901 | 62902 | 62903 | 62905 | 62906 | 62907 | 62911 | 62912 | 62918 | 62919 | 62920 |
| | 52921 | 62922 | 62923 | 62924 | 62925 | 62926 | 62927 | 62928 | 62930 | 62933 | 62934 | 62937 |
| | 62938 | 62939 | 62940 | 62941 | 62947 | 62948 | 62952 | 62956 | 62962 | 62964 | 62966 | 62970 |
| | 62973 | 62974 | 62983 | 62984 | 62985 | 62991 | 62992 | 62993 | 62994 | 62995 | 63009 | 63010 |
| | 63011 | 63015 | 63016 | 63017 | 63019 | 63020 | 63021 | 63022 | 63023 | 63024 | 63027 | 63032 |
| | 63033 | 63034 | 63037 | 63038 | 63039 | 63040 | 63041 | 63042 | 63046 | 63047 | 63048 | 63055 |
| | 63056 | 63057 | 63060 | 63061 | 63062 | 63063 | 63064 | 63065 | 63067 | 63068 | 63069 | 63080 |
| | 63081 | 63082 | 63083 | 63084 | 63087 | 63088 | 63089 | 63090 | 63091 | 63092 | 63093 | 63094 |
| | 63098 | 63099 | 63100 | 63101 | 63102 | 63103 | 63104 | 63105 | 63106 | 63107 | 63108 | 63109 |
| | 63110 | 63111 | 63112 | 63116 | 63123 | 63124 | 63125 | 63126 | 63127 | 63128 | 63134 | 63135 |
| | 63136 | 63137 | 63138 | 63139 | 63146 | 63147 | 63148 | 63149 | 63150 | 63151 | 63161 | 63162 |
| | 63163 | 63167 | 63168 | 63169 | 63170 | 63171 | 63172 | 63173 | 63174 | 63175 | 63198 | 63202 |
| | 63225 | 63226 | 63227 | 63233 | 63234 | 63235 | 63236 | 63237 | 63238 | 63239 | 63240 | 63241 |
| | 63242 | 63243 | 63244 | 63257 | 63258 | 63259 | 63266 | 63267 | 63268 | 63269 | 63270 | 63278 |
| | 63279 | 63280 | 63281 | 63282 | 63288 | 63289 | 63290 | 63291 | 63292 | 63293 | 63297 | 63298 |
| | 63299 | 63300 | 63301 | 63305 | 63306 | 63307 | 63308 | 63309 | 63311 | 63312 | 63313 | 63314 |
| | 63315 | 63316 | 63317 | 63318 | 63319 | 63320 | 63321 | 63322 | 63323 | 63333 | 63334 | 63335 |
| | 63336 | 63337 | 63338 | 63339 | 63340 | 63341 | 63342 | 63343 | 63344 | 63345 | 63346 | 63347 |
| | 63348 | 63349 | 63350 | 63351 | 63352 | 63353 | 63354 | 63355 | 63356 | 63357 | 63358 | 63359 |
| | 63363 | 63364 | 63365 | 63366 | 63367 | 63368 | 63369 | 63370 | 63371 | 63372 | 63373 | 63374 |
| | 63375 | 63376 | 63377 | 63378 | 63379 | 63380 | 63381 | 63382 | 63383 | 63384 | 63386 | 63387 |
| | 63388 | 63389 | 63390 | 63391 | 63392 | 63393 | 63394 | 63395 | 63396 | 63397 | 63398 | 63399 |
| | 63400 | 63401 | 63402 | 63403 | 63411 | 63412 | 63413 | 63414 | 63415 | 63416 | 63417 | 63418 |
| | 63420 | 63421 | 63422 | 63423 | 63424 | 63425 | 63426 | 63431 | 63432 | 63433 | 63442 | 63443 |
| | 63444 | 63445 | 63448 | 63449 | 63450 | 63451 | 63452 | 63453 | 63454 | 63455 | 63456 | 63457 |
| | 63471 | 63475 | 63476 | 63477 | 63478 | 63479 | 63480 | 63481 | 63483 | 63486 | 63487 | 63488 |
| | 63489 | 63490 | 63491 | 63492 | 63493 | 63496 | 63499 | 63503 | 63507 | 63508 | 63509 | 63510 |
| | 63511 | 63512 | 63513 | 63514 | 63515 | 63516 | 63517 | 63518 | 63519 | 63520 | 63524 | 63525 |
| | 63526 | 63527 | 63528 | 63529 | 63530 | 63531 | 63532 | 63533 | 63534 | 63539 | 63540 | 63541 |
| | 63545 | 63546 | 63547 | 63548 | 63549 | 63550 | 63561 | 63562 | 63578 | 63579 | 63580 | 63581 |
| | 63583 | 63584 | 63585 | 63586 | 63590 | 63591 | 63592 | 63593 | 63594 | 63595 | 63596 | 63597 |
| | 63598 | 63604 | 63605 | 63606 | 63607 | 63608 | 63625 | 63626 | 63627 | 63634 | 63635 | 63636 |
| | 63637 | 63638 | 63644 | 63654 | 63655 | 63656 | 63657 | 63660 | 63661 | 63662 | 63663 | 63664 |
| | 63667 | 63669 | 63670 | 63678 | 63679 | 63680 | 63681 | 63682 | 63683 | 63684 | 63687 | 63691 |
| | 63692 | 63693 | 63694 | 63698 | 63699 | 63700 | 63701 | 63702 | 63704 | 63705 | 63710 | 63711 |
| | 63712 | 63713 | 63714 | 63727 | 63728 | 63729 | 63730 | 63744 | 63745 | 63748 | 63757 | 63761 |
| | 63763 | 63775 | 63779 | 63782 | 63783 | 63784 | 63785 | 63787 | 63798 | 63800 | 63803 | 63812 |
| | 63815 | 63816 | 63817 | 63818 | 63819 | 63820 | 63821 | 63822 | 63832 | 63837 | 63838 | 63839 |
| | 63840 | 63842 | 63859 | 63860 | 63861 | 63862 | 63863 | 63864 | 63866 | 63867 | 63868 | 63873 |
| | 63874 | 63875 | 63876 | 63907 | 63908 | 63909 | 63910 | 63914 | 63915 | 63926 | 63927 | 63928 |
| | 63930 | 63931 | 63934 | 63959 | 63960 | 63961 | 63975 | 63976 | 63997 | 63998 | 63999 | 64000 |
| | 64004 | 64042 | 64047 | 64048 | 64049 | 64050 | 64070 | 64078 | 64091 | 64101 | 64125 | 64142 |
| | 64149 | 64150 | 64153 | 64154 | 64155 | 64156 | 64157 | 64158 | 64159 | 64160 | 64161 | 64162 |
| | 64163 | 64164 | 64166 | 64167 | 64168 | 64171 | 64173 | 64174 | 64175 | 64176 | 64186 | 64187 |
| | 64194 | 64195 | 64196 | 64197 | 64198 | 64201 | 64202 | 64203 | 64207 | 64208 | 64212 | 64217 |
| | 64218 | 64219 | 64220 | 64228 | 64229 | 64230 | 64231 | 64235 | 64236 | 64237 | 64238 | 64239 |
| | 64240 | 64241 | 64242 | 64243 | 64247 | 64248 | 64250 | 64251 | 64255 | 64257 | 64258 | 64259 |
| | 64261 | 64262 | 64263 | 64264 | 64267 | 64268 | 64274 | 64277 | 64279 | 64280 | 64288 | 64289 |
| | 64290 | 64293 | 64304 | 64305 | 64306 | 64307 | 64308 | 64309 | 64310 | 64313 | 64315 | 64316 |
| | 64317 | 64321 | 64322 | 64323 | 64324 | 64325 | 64326 | 64327 | 64328 | 64329 | 64330 | 64336 |
| | 64347 | 64349 | 64353 | 64354 | 64355 | 64356 | 64357 | 64358 | 64359 | 64363 | 64365 | 64367 |
| | 64368 | 64369 | 64370 | 64371 | 64372 | 64373 | 64374 | 64375 | 64376 | 64380 | 64381 | 64382 |
| | 64383 | 64385 | 64386 | 64387 | 64388 | 64389 | 64394 | 64395 | 64396 | 64397 | 64398 | 64400 |
| | 64401 | 64402 | 64406 | 64407 | 64408 | 64409 | 64410 | 64411 | 64412 | 64419 | 64420 | 64421 |
| | 64423 | 64424 | 64425 | 64435 | 64441 | 64443 | 64464 | 64465 | 64466 | 64467 | 64468 | 64469 |
| | 64480 | 64489 | 64490 | 64491 | 64495 | 64508 | 64514 | 64545 | 64558 | 64559 | 64565 | 64566 |
| | 64573 | 64575 | 64576 | 64577 | 64583 | 64590 | 64594 | 64595 | 64596 | 64597 | 64609 | 64610 |
| | 64611 | 64612 | 64613 | 64617 | 64618 | 64619 | 64630 | 64631 | 64632 | 64642 | 64655 | 64656 |
| | 64661 | 64662 | 64663 | 64664 | 64665 | 64666 | 64670 | 64671 | 64672 | 64676 | 64677 | 64678 |
| | 64683 | 64684 | 64685 | 64690 | 64691 | 64712 | 64713 | 64717 | 64718 | 64719 | 64729 | 64731 |
| | 64743 | 64744 | 64745 | 64748 | 64749 | 64751 | 64752 | 64753 | 64754 | 64762 | 64763 | 64764 |
| | 64765 | 64766 | 64768 | 64769 | 64770 | 64771 | 64772 | 64773 | 64774 | 64775 | 64783 | 64787 |
| | 64793 | 64798 | 64799 | 64800 | 64801 | 64802 | 64803 | 64804 | 64805 | 64806 | 64817 | 64822 |
| | 64823 | 64824 | 64825 | 64829 | 64831 | 64832 | 64833 | 64852 | 64853 | 64854 | 64866 | 64867 |
| | 64868 | 64884 | 64885 | 64901 | 64902 | 64903 | 64912 | 64913 | 64936 | 64937 | 64938 | 64954 |
| | 64955 | 64956 | 64969 | 64970 | 64972 | 64973 | 64974 | 64975 | 64979 | 64980 | 64981 | 64982 |
| | 64988 | 64990 | 64991 | 64992 | 64993 | 64998 | 64999 | 65000 | 65002 | 65003 | 65004 | 65008 |
| | 65009 | 65010 | 65011 | 65015 | 65016 | 65017 | 65020 | 65021 | 65022 | 65024 | 65025 | 65026 |
| | 65034 | 65035 | 65036 | 65037 | 65038 | 65039 | 65040 | 65041 | 65042 | 65043 | 65044 | 65045 |
| | 65046 | 65047 | 65048 | 65052 | 65053 | 65054 | 65055 | 65056 | 65057 | 65058 | 65059 | 65060 |
| | 65061 | 65062 | 65063 | 65068 | 65069 | 65070 | 65071 | 65072 | 65073 | 65074 | 65077 | 65078 |
| | 65079 | 65080 | 65081 | 65085 | 65086 | 65100 | 65104 | 65105 | 65106 | 65108 | 65109 | 65110 |
| | 65111 | 65112 | 65122 | 65123 | 65124 | 65131 | 65132 | 65133 | 65134 | 65135 | 65137 | 65141 |
| | 65142 | 65144 | 65145 | 65146 | 65147 | 65148 | 65149 | 65154 | 65155 | 65156 | 65157 | 65158 |
| | 65159 | 65162 | 65163 | 65164 | 65165 | 65166 | 65171 | 65172 | 65173 | 65174 | 65175 | 65176 |
| | 65177 | 65178 | 65179 | 65180 | 65181 | 65184 | 65185 | 65186 | 65187 | 65191 | 65192 | 65193 |
| | 65194 | 65195 | 65196 | 65197 | 65198 | 65199 | 65200 | 65202 | 65203 | 65204 | 65205 | 65206 |
| | 65207 | 65208 | 65209 | 65214 | 65215 | 65216 | 65219 | 65220 | 65221 | 65226 | 65227 | 65228 |
| | 65229 | 65231 | 65232 | 65233 | 65234 | 65239 | 65240 | 65241 | 65242 | 65250 | 65251 | 65252 |
| | 65253 | 65254 | 65255 | 65259 | 65260 | 65261 | 65265 | 65266 | 65267 | 65279 | 65280 | 65281 |
| | 65282 | 65284 | 65285 | 65286 | 65287 | 65288 | 65289 | 65290 | 65296 | 65297 | 65298 | 65307 |
| | 65308 | 65309 | 65313 | 65314 | 65317 | 65318 | 65325 | 65326 | 65327 | 65328 | 65330 | 65331 |
| | 65336 | 65337 | 65338 | 65339 | 65340 | 65341 | 65342 | 65343 | 65344 | 65345 | 65346 | 65347 |
| | 65348 | 65349 | 65350 | 65351 | 65352 | 65355 | 65356 | 65359 | 65360 | 65361 | 65362 | 65363 |
| | 65364 | 65365 | 65366 | 65372 | 65374 | 65377 | 65378 | 65383 | 65384 | 65393 | 65394 | 65395 |
| | 65396 | 65397 | 65398 | 65399 | 65400 | 65401 | 65402 | 65403 | 65404 | 65405 | 65410 | 65411 |
| | 65412 | 65413 | 65415 | 65416 | 65420 | 65421 | 65422 | 65423 | 65431 | 65432 | 65433 | 65434 |
| | 65435 | 65436 | 65439 | 65440 | 65442 | 65443 | 65446 | 65447 | 65448 | 65461 | 65462 | 65463 |
| | 65465 | 65466 | 65468 | 65475 | 65476 | 65482 | 65483 | 65484 | 65485 | 65487 | 65490 | 65491 |
| | 65492 | 65493 | 65498 | 65500 | 65504 | 65505 | 65514 | 65515 | 65516 | 65518 | 65524 | 65525 |
| | 65526 | 65527 | 65528 | 65529 | 65533 | 65541 | 65542 | 65543 | 65544 | 65549 | 65551 | 65556 |
| | 65557 | 65558 | 65564 | 65566 | 65567 | 65576 | 65577 | 65580 | 65585 | 65612 | 65613 | 65627 |
| | 65628 | 65629 | 65634 | 65635 | 65636 | 65637 | 65646 | 65647 | 65648 | 65649 | 65650 | 65654 |
| | 65655 | 65657 | 65658 | 65661 | 65662 | 65663 | 65680 | 65681 | 65684 | 65691 | 65692 | 65710 |
| | 65711 | 65716 | 65719 | 65720 | 65721 | 65732 | 65735 | 65742 | 65743 | 65754 | 65755 | 65759 |
| | 65760 | 65764 | 65768 | 65769 | 65770 | 65772 | 65784 | 65787 | 65791 | 65792 | 65793 | 65794 |
| | 65795 | 65796 | 65797 | 65801 | 65802 | 65807 | 65808 | 65809 | 65816 | 65817 | 65818 | 65821 |
| | 65822 | 65823 | 65827 | 65831 | 65832 | 65834 | 65835 | 65836 | 65837 | 65843 | 65844 | 65845 |
| | 65846 | 65847 | 65856 | 65857 | 65858 | 65859 | 65860 | 65861 | 65862 | 65875 | 65883 | 65884 |
| | 65885 | 65886 | 65888 | 65889 | 65890 | 65895 | 65913 | 65914 | 65915 | 65917 | 65919 | 65920 |
| | 65921 | 65922 | 65923 | 65927 | 65930 | 65931 | 65932 | 65935 | 65936 | 65938 | 65939 | 65944 |
| | 65945 | 65946 | 65953 | 65954 | 65955 | 65956 | 65957 | 65958 | 65966 | 65968 | 65970 | 65971 |
| | 65972 | 65973 | 65974 | 65975 | 65976 | 65977 | 65978 | 65979 | 65980 | 65981 | 65982 | 65985 |
| | 65986 | 65987 | 65988 | 65989 | 65990 | 65991 | 65999 | 66000 | 66003 | 66004 | 66005 | 66011 |
| | 66012 | 66017 | 66018 | 66019 | 66020 | 66021 | 66027 | 66028 | 66037 | 66043 | 66044 | 66050 |
| | 66051 | 66052 | 66055 | 66058 | 66083 | 66084 | 66086 | 66087 | 66088 | 66096 | 66103 | 66104 |
| | 66105 | 66106 | 66107 | 66108 | 66109 | 66111 | 66112 | 66113 | 66114 | 66117 | 66118 | 66119 |
| | 66122 | 66123 | 66124 | 66125 | 66126 | 66127 | 66128 | 66129 | 66130 | 66131 | 66132 | 66134 |
| | 66135 | 66136 | 66137 | 66140 | 66141 | 66148 | 66151 | 66152 | 66155 | 66156 | 66157 | 66158 |
| | 66159 | 66160 | 66162 | 66163 | 66164 | 66165 | 66169 | 66182 | 66183 | 66185 | 66188 | 66190 |
| | 66192 | 66193 | 66194 | 66211 | 66225 | 66226 | 66227 | 66232 | 66233 | 66236 | 66237 | 66238 |
| | 66243 | 66246 | 66247 | 66263 | 66267 | 66273 | 66274 | 66279 | 66280 | 66284 | 66285 | 66286 |
| | 66288 | 66293 | 66294 | 66295 | 66296 | 66297 | 66302 | 66303 | 66304 | 66305 | 66306 | 66307 |
| | 66308 | 66309 | 66311 | 66312 | 66313 | 66315 | 66316 | 66317 | 66318 | 66319 | 66320 | 66321 |
| | 66322 | 66323 | 66324 | 66325 | 66326 | 66327 | 66328 | 66329 | 66330 | 66331 | 66333 | 66334 |
| | 66335 | 66336 | 66337 | 66338 | 66339 | 66340 | 66341 | 66342 | 66343 | 66344 | 66345 | 66346 |
| | 66347 | 66348 | 66349 | 66350 | 66351 | 66352 | 66353 | 66354 | 66355 | 66356 | 66357 | 66358 |
| | 66360 | 66361 | 66362 | 66363 | 66364 | 66365 | 66366 | 66367 | 66368 | 66369 | 66372 | 66373 |
| | 66374 | 66375 | 66376 | 66377 | 66378 | 66379 | 66380 | 66381 | 66382 | 66383 | 66384 | 66385 |
| | 66388 | 66392 | 66395 | 66400 | 66403 | 66404 | 66410 | 66411 | 66412 | 66414 | 66415 | 66416 |
| | 66417 | 66418 | 66419 | 66420 | 66421 | 66422 | 66423 | 66424 | 66425 | 66427 | 66428 | 66429 |
| | 66430 | 66431 | 66432 | 66433 | 66434 | 66435 | 66436 | 66437 | 66440 | 66441 | 66442 | 66443 |
| | 66444 | 66445 | 66447 | 66451 | 66452 | 66453 | 66454 | 66455 | 66456 | 66457 | 66459 | 66460 |
| | 66461 | 66462 | 66466 | 66467 | 66468 | 66469 | 66470 | 66471 | 66472 | 66474 | 66475 | 66478 |
| | 66479 | 66481 | 66482 | 66483 | 66484 | 66485 | 66486 | 66487 | 66488 | 66489 | 66494 | 66495 |
| | 66496 | 66497 | 66498 | 66499 | 66502 | 66505 | 66506 | 66507 | 66508 | 66509 | 66513 | 66516 |
| | 66517 | 66519 | 66520 | 66521 | 66522 | 66524 | 66526 | 66527 | 66528 | 66529 | 66533 | 66534 |
| | 66535 | 66541 | 66542 | 66543 | 66544 | 66547 | 66550 | 66555 | 66556 | 66557 | 66559 | 66562 |
| | 66563 | 66564 | 66565 | 66566 | 66568 | 66569 | 66570 | 66571 | 66574 | 66575 | 66576 | 66579 |
| | 66582 | 66583 | 66584 | 66585 | 66586 | 66587 | 66588 | 66589 | 66590 | 66592 | 66593 | 66594 |
| | 66595 | 66596 | 66597 | 66598 | 66599 | 66600 | 66601 | 66602 | 66603 | 66604 | 66605 | 66606 |
| | 66607 | 66608 | 66609 | 66610 | 66612 | 66613 | 66614 | 66615 | 66617 | 66618 | 66619 | 66620 |
| | 66621 | 66624 | 66625 | 66626 | 66627 | 66631 | 66634 | 66639 | 66642 | 66652 | 66657 | 66658 |
| | 66659 | 66665 | 66671 | 66672 | 66673 | 66674 | 66675 | 66676 | 66677 | 66678 | 66679 | 66680 |
| | 66681 | 66682 | 66683 | 66686 | 66688 | 66691 | 66694 | 66695 | 66696 | 66697 | 66698 | 66699 |
| | 66702 | 66703 | 66704 | 66705 | 66706 | 66707 | 66710 | 66711 | 66712 | 66713 | 66714 | 66715 |
| | 66717 | 66720 | 66721 | 66722 | 66727 | 66728 | 66730 | 66731 | 66732 | 66733 | 66734 | 66735 |
| | 66738 | 66742 | 66743 | 66748 | 66749 | 66750 | 66751 | 66752 | 66755 | 66756 | 66757 | 66760 |
| | 66761 | 66764 | 66765 | 66771 | 66772 | 66773 | 66774 | 66775 | 66776 | 66777 | 66778 | 66779 |
| | 66780 | 66781 | 66782 | 66783 | 66785 | 66788 | 66792 | 66793 | 66799 | 66809 | 66817 | 66818 |
| | 66819 | 66820 | 66821 | 66837 | 66838 | 66840 | 66841 | 66842 | 66849 | 66850 | 66851 | 66854 |
| | 66855 | 66856 | 66859 | 66867 | 66868 | 66871 | 66872 | 66879 | 66880 | 66881 | 66882 | 66883 |
| | 66884 | 66885 | 66893 | 66894 | 66895 | 66896 | 66897 | 66898 | 66899 | 66900 | 66902 | 66903 |
| | 66919 | 66920 | 66922 | 66923 | 66924 | 66925 | 66926 | 66927 | 66930 | 66939 | 66941 | 66942 |
| | 66943 | 66946 | 66947 | 66949 | 66953 | 66954 | 66955 | 66956 | 66957 | 66958 | 66959 | 66961 |
| | 66962 | 66963 | 66964 | 66965 | 66966 | 66967 | 66968 | 66972 | 66974 | 66975 | 66976 | 66978 |
| | 66979 | 66980 | 66983 | 66984 | 66991 | 66992 | 66993 | 66994 | 66995 | 66996 | 66997 | 66998 |
| | 67002 | 67003 | 67004 | 67005 | 67006 | 67018 | 67019 | 67023 | 67024 | 67025 | 67029 | 67030 |
| | 67031 | 67032 | 67033 | 67034 | 67035 | 67036 | 67037 | 67038 | 67042 | 67043 | 67045 | 67046 |
| | 67047 | 67059 | 67062 | 67063 | 67070 | 67089 | 67090 | 67091 | 67092 | 67093 | 67098 | 67099 |
| | 67106 | 67112 | 67116 | 67117 | 67121 | 67122 | 67138 | 67142 | 67143 | 67144 | 67145 | 67146 |
| | 67147 | 67150 | 67154 | 67155 | 67156 | 67159 | 67162 | 67165 | 67166 | 67167 | 67168 | 67169 |
| | 67170 | 67172 | 67173 | 67174 | 67178 | 67179 | 67180 | 67181 | 67182 | 67183 | 67184 | 67185 |
| | 67186 | 67187 | 67188 | 67191 | 67193 | 67194 | 67195 | 67196 | 67197 | 67198 | 67199 | 67202 |
| | 67203 | 67204 | 67205 | 67206 | 67207 | 67209 | 67210 | 67211 | 67213 | 67214 | 67215 | 67216 |
| | 67217 | 67218 | 67219 | 67220 | 67222 | 67223 | 67224 | 67225 | 67226 | 67229 | 67230 | 67231 |
| | 67234 | 67235 | 67236 | 67237 | 67238 | 67239 | 67240 | 67241 | 67242 | 67243 | 67244 | 67245 |
| | 67247 | 67248 | 67249 | 67251 | 67252 | 67254 | 67257 | 67258 | 67260 | 67261 | 67262 | 67263 |
| | 67264 | 67265 | 67266 | 67267 | 67268 | 67269 | 67270 | 67271 | 67272 | 67273 | 67274 | 67282 |
| | 67283 | 67284 | 67285 | 67286 | 67290 | 67296 | 67302 | 67305 | 67306 | 67307 | 67317 | 67318 |
| | 67319 | 67320 | 67332 | 67340 | 67344 | 67359 | 67365 | 67366 | 67370 | 67371 | 67372 | 67382 |
| | 67392 | 67393 | 67397 | 67398 | 67399 | 67404 | 67405 | 67406 | 67410 | 67417 | 67421 | 67431 |
| | 67435 | 67436 | 67444 | 67445 | 67446 | 67447 | 67451 | 67455 | 67456 | 67457 | 67458 | 67459 |
| | 67460 | 67461 | 67462 | 67463 | 67464 | 67465 | 67466 | 67467 | 67468 | 67469 | 67470 | 67472 |
| | 67473 | 67474 | 67477 | 67478 | 67482 | 67483 | 67484 | 67485 | 67490 | 67491 | 67492 | 67493 |
| | 67494 | 67501 | 67502 | 67506 | 67507 | 67508 | 67513 | 67514 | 67515 | 67516 | 67517 | 67518 |
| | 67519 | 67521 | 67522 | 67523 | 67524 | 67525 | 67526 | 67527 | 67528 | 67529 | 67530 | 67533 |
| | 67534 | 67535 | 67536 | 67537 | 67538 | 67539 | 67541 | 67543 | 67544 | 67545 | 67546 | 67547 |
| | 67548 | 67550 | 67551 | 67552 | 67553 | 67554 | 67555 | 67559 | 67560 | 67562 | 67563 | 67564 |
| | 67567 | 67570 | 67574 | 67581 | 67583 | 67584 | 67585 | 67587 | 67588 | 67589 | 67590 | 67592 |
| | 67597 | 67598 | 67603 | 67604 | 67605 | 67606 | 67607 | 67608 | 67609 | 67611 | 67614 | 67615 |
| | 67616 | 67617 | 67618 | 67619 | 67620 | 67621 | 67622 | 67626 | 67627 | 67628 | 67629 | 67633 |
| | 67634 | 67635 | 67636 | 67637 | 67638 | 67639 | 67642 | 67646 | 67650 | 67651 | 67655 | 67660 |
| | 67663 | 67673 | 67674 | 67675 | 67676 | 67679 | 67680 | 67681 | 67684 | 67695 | 67708 | 67709 |
| | 67716 | 67717 | 67733 | 67734 | 67736 | 67737 | 67740 | 67741 | 67750 | 67751 | 67758 | 67764 |
| | 67771 | 67779 | 67780 | 67799 | 67813 | 67814 | 67815 | 67816 | 67817 | 67818 | 67825 | 67826 |
| | 67827 | 67830 | 67833 | 67836 | 67837 | 67838 | 67839 | 67840 | 67843 | 67844 | 67850 | 67851 |
| | 67852 | 67853 | 67854 | 67857 | 67858 | 67861 | 67862 | 67866 | 67867 | 67878 | 67879 | 67887 |
| | 67898 | 67899 | 67908 | 67915 | 67916 | 67919 | 67922 | 67923 | 67925 | 67927 | 67928 | 67929 |
| | 67943 | 67948 | 67951 | 67955 | 67964 | 67965 | 67969 | 67970 | 67973 | 67974 | 67975 | 67978 |
| | 67979 | 67980 | 67982 | 67983 | 67984 | 67990 | 67991 | 67992 | 67998 | 68000 | 68001 | 68003 |
| | 68004 | 68005 | 68006 | 68010 | 68019 | 68029 | 68030 | 68033 | 68040 | 68041 | 68042 | 68043 |
| | 68044 | 68045 | 68049 | 68060 | 68063 | 68071 | 68072 | 68078 | 68079 | 68091 | 68092 | 68095 |
| | 68096 | 68097 | 68098 | 68103 | 68104 | 68105 | 68114 | 68115 | 68116 | 68148 | 68149 | 68150 |
| | 68194 | 68195 | 68210 | 68211 | 68212 | 68213 | 68216 | 68219 | 68220 | 68221 | 68238 | 68239 |
| | 68240 | 68241 | 68260 | 68261 | 68262 | 68266 | 68279 | 68289 | 68292 | 68293 | 68298 | 68301 |
| | 68302 | 68303 | 68308 | 68309 | 68312 | 68313 | 68314 | 68315 | 68319 | 68324 | 68325 | 68334 |
| | 68335 | 68340 | 68341 | 68342 | 68346 | 68347 | 68348 | 68358 | 68360 | 68361 | 68366 | 68367 |
| | 68368 | 68376 | 68377 | 68378 | 68379 | 68380 | 68381 | 68384 | 68385 | 68386 | 68391 | 68399 |
| | 68400 | 68401 | 68403 | 68404 | 68410 | 68411 | 68415 | 68416 | 68417 | 68419 | 68422 | 68423 |
| | 68424 | 68428 | 68429 | 68430 | 68437 | 68438 | 68439 | 68440 | 68441 | 68442 | 68443 | 68444 |
| | 68445 | 68446 | 68447 | 68460 | 68465 | 68466 | 68469 | 68470 | 68471 | 68472 | 68473 | 68477 |
| | 68478 | 68480 | 68481 | 68483 | 68484 | 68485 | 68486 | 68487 | 68488 | 68489 | 68493 | 68494 |
| | 68496 | 68497 | 68498 | 68499 | 68503 | 68504 | 68505 | 68506 | 68507 | 68508 | 68509 | 68512 |
| | 68513 | 68514 | 68515 | 68516 | 68517 | 68525 | 68536 | 68537 | 68538 | 68542 | 68543 | 68547 |
| | 68548 | 68549 | 68550 | 68567 | 68572 | 68573 | 68594 | 68595 | 68603 | 68604 | 68611 | 68629 |
| | 68632 | 68633 | 68634 | 68642 | 68644 | 68645 | 68646 | 68647 | 68652 | 68653 | 68654 | 68655 |
| | 68656 | 68657 | 68663 | 68668 | 68674 | 68676 | 68677 | 68680 | 68681 | 68684 | 68685 | 68686 |
| | 68687 | 68688 | 68689 | 68691 | 68692 | 68696 | 68697 | 68699 | 68700 | 68703 | 68704 | 68705 |
| | 68707 | 68708 | 68713 | 68714 | 68715 | 68716 | 68717 | 68718 | 68726 | 68728 | 68730 | 68738 |
| | 68745 | 68746 | 68747 | 68750 | 68753 | 68754 | 68764 | 68765 | 68766 | 68767 | 68768 | 68770 |
| | 68771 | 68772 | 68775 | 68776 | 68777 | 68781 | 68782 | 68783 | 68785 | 68786 | 68788 | 68789 |
| | 68790 | 68791 | 68792 | 68793 | 68796 | 68797 | 68798 | 68799 | 68800 | 68801 | 68802 | 68804 |
| | 68805 | 68806 | 68807 | 68808 | 68809 | 68812 | 68813 | 68814 | 68815 | 68816 | 68817 | 68818 |
| | 68819 | 68820 | 68821 | 68822 | 68823 | 68824 | 68826 | 68827 | 68828 | 68832 | 68834 | 68835 |
| | 68836 | 68846 | 68847 | 68848 | 68851 | 68852 | 68853 | 68854 | 68855 | 68859 | 68860 | 68861 |
| | 68862 | 68868 | 68869 | 68873 | 68880 | 68881 | 68882 | 68883 | 68884 | 68885 | 68886 | 68887 |
| | 68890 | 68891 | 68892 | 68893 | 68894 | 68895 | 68896 | 68897 | 68900 | 68901 | 68902 | 68903 |
| | 68905 | 68906 | 68907 | 68908 | 68909 | 68910 | 68912 | 68913 | 68914 | 68915 | 68917 | 68918 |
| | 68920 | 68921 | 68922 | 68925 | 68926 | 68930 | 68931 | 68932 | 68933 | 68937 | 68940 | 68941 |
| | 68943 | 68944 | 68945 | 68946 | 68952 | 68956 | 68967 | 68968 | 68970 | 68971 | 68975 | 68976 |
| | 68979 | 68984 | 68987 | 68988 | 68992 | 68993 | 69005 | 69006 | 69012 | 69013 | 69014 | 69016 |
| | 69022 | 69023 | 69025 | 69026 | 69027 | 69028 | 69029 | 69032 | 69038 | 69039 | 69042 | 69044 |
| | 69045 | 69047 | 69052 | 69054 | 69055 | 69062 | 69063 | 69065 | 69066 | 69069 | 69070 | 69074 |
| | 69075 | 69076 | 69077 | 69083 | 69084 | 69086 | 69087 | 69088 | 69089 | 69090 | 69093 | 69094 |
| | 69096 | 69097 | 69098 | 69099 | 69105 | 69106 | 69115 | 69126 | 69133 | 69137 | 69138 | 69139 |
| | 69143 | 69144 | 69146 | 69147 | 69148 | 69149 | 69150 | 69151 | 69153 | 69154 | 69155 | 69156 |
| | 69157 | 69158 | 69159 | 69163 | 69164 | 69169 | 69170 | 69173 | 69174 | 69177 | 69178 | 69181 |
| | 69186 | 69187 | 69188 | 69191 | 69192 | 69193 | 69194 | 69195 | 69196 | 69197 | 69205 | 69207 |
| | 69209 | 69211 | 69213 | 69214 | 69216 | 69223 | 69224 | 69227 | 69228 | 69229 | 69230 | 69231 |
| | 69232 | 69234 | 69235 | 69238 | 69239 | 69240 | 69243 | 69245 | 69246 | 69247 | 69248 | 69249 |
| | 69250 | 69251 | 69252 | 69254 | 69255 | 69256 | 69257 | 69258 | 69259 | 69262 | 69263 | 69268 |
| | 69269 | 69270 | 69271 | 69272 | 69273 | 69275 | 69276 | 69277 | 69279 | 69280 | 69281 | 69282 |
| | 69284 | 69289 | 69291 | 69292 | 69293 | 69296 | 69298 | 69302 | 69306 | 69307 | 69315 | 69316 |
| | 69317 | 69318 | 69319 | 69323 | 69327 | 69330 | 69331 | 69332 | 69336 | 69337 | 69341 | 69342 |
| | 69345 | 69346 | 69347 | 69348 | 69351 | 69354 | 69355 | 69357 | 69358 | 69359 | 69363 | 69364 |
| | 69365 | 69370 | 69372 | 69373 | 69374 | 69377 | 69378 | 69382 | 69385 | 69388 | 69390 | 69391 |
| | 69393 | 69394 | 69395 | 69396 | 69397 | 69399 | 69401 | 69402 | 69403 | 69404 | 69405 | 69407 |
| | 69408 | 69409 | 69410 | 69411 | 69412 | 69413 | 69414 | 69415 | 69416 | 69417 | 69418 | 69419 |
| | 69420 | 69421 | 69426 | 69427 | 69428 | 69429 | 69431 | 69432 | 69433 | 69434 | 69435 | 69438 |
| | 69442 | 69443 | 69444 | 69445 | 69448 | 69449 | 69461 | 69462 | 69463 | 69465 | 69468 | 69473 |
| | 69474 | 69477 | 69478 | 69479 | 69480 | 69482 | 69483 | 69485 | 69492 | 69493 | 69495 | 69496 |
| | 69503 | 69504 | 69509 | 69512 | 69513 | 69517 | 69518 | 69519 | 69520 | 59521 | 69526 | 69530 |
| | 69531 | 69535 | 69536 | 69538 | 69539 | 69540 | 69543 | 69546 | 69547 | 69548 | 69549 | 69551 |
| | 69552 | 69553 | 69554 | 69555 | 69560 | 69563 | 69564 | 69566 | 69568 | 69569 | 69571 | 69572 |
| | 69573 | 69575 | 69578 | 69579 | 69580 | 69581 | 69587 | 69588 | 69589 | 69590 | 69592 | 69596 |
| | 69597 | 69598 | 69599 | 69604 | 69605 | 69606 | 69607 | 69609 | 69611 | 69619 | 69620 | 69622 |
| | 69624 | 69625 | 69626 | 69627 | 69628 | 69630 | 69631 | 69635 | 69636 | 69641 | 69643 | 69645 |
| | 69646 | 69647 | 69648 | 69649 | 69651 | 69653 | 69657 | 69660 | 69661 | 69662 | 69666 | 69670 |
| | 69673 | 69674 | 69675 | 69676 | 69680 | 69684 | 69685 | 69686 | 69687 | 69689 | 69691 | 69692 |
| | 69693 | 69694 | 69697 | 69698 | 69699 | 69700 | 69701 | 69702 | 69703 | 69704 | 69706 | 69710 |
| | 69711 | 69713 | 69714 | 69716 | 69717 | 69720 | 69721 | 69724 | 69725 | 69727 | 69728 | 69729 |
| | 69730 | 69733 | 69737 | 69738 | 69742 | 69743 | 69746 | 69749 | 69750 | 69751 | 69753 | 69754 |
| | 69755 | 69756 | 69757 | 69758 | 69759 | 69760 | 69762 | 69763 | 69764 | 69765 | 69766 | 69767 |
| | 69768 | 69770 | 69771 | 69772 | 69774 | 69775 | 69776 | 69777 | 69782 | 69783 | 69784 | 69785 |
| | 69786 | 69788 | 69789 | 69790 | 69793 | 69800 | 69801 | 69802 | 69806 | 69807 | 69808 | 69809 |
| | 69811 | 69814 | 69815 | 69821 | 69824 | 69827 | 69833 | 69835 | 69837 | 69838 | 69841 | 69842 |
| | 69844 | 69845 | 69848 | 69851 | 69852 | 69853 | 69855 | 69857 | 69858 | 69865 | 69866 | 69868 |
| | 69869 | 69870 | 69871 | 69872 | 69873 | 69875 | 69879 | 69880 | 69881 | 69884 | 69887 | 69888 |
| | 69890 | 69892 | 69894 | 69898 | 69900 | 69901 | 69904 | 69907 | 69908 | 69909 | 69911 | 69912 |
| | 69914 | 69915 | 69916 | 69917 | 69919 | 69925 | 69926 | 69929 | 69931 | 69933 | 69937 | 69945 |
| | 69946 | 69950 | 69952 | 69959 | 69985 | 69988 | 69989 | 69991 | 69992 | 69993 | 69994 | 69998 |
| | 69999 | 70002 | 70003 | 70004 | 70005 | 70006 | 70009 | 70011 | 70012 | 70013 | 70014 | 70016 |
| | 70017 | 70019 | 70023 | 70026 | 70027 | 70028 | 70029 | 70030 | 70032 | 70034 | 70036 | 70037 |
| | 70042 | 70043 | 70045 | 70046 | 70047 | 70049 | 70051 | 70052 | 70054 | 70055 | 70057 | 70059 |
| | 70061 | 70063 | 70064 | 70065 | 70072 | 70073 | 70075 | 70076 | 70079 | 70080 | 70083 | 70085 |
| | 70086 | 70089 | 70090 | 70091 | 70092 | 70093 | 70094 | 70095 | 70096 | 70098 | 70099 | 70100 |
| | 70102 | 70103 | 70105 | 70106 | 70108 | 70109 | 70110 | 70111 | 70113 | 70115 | 70116 | 70117 |
| | 70118 | 70119 | 70122 | 70127 | 70129 | 70133 | 70134 | 70135 | 70138 | 70139 | 70141 | 70144 |
| | 70147 | 70148 | 70149 | 70150 | 70151 | 70155 | 70159 | 70160 | 70161 | 70163 | 70165 | 70170 |
| | 70174 | 70176 | 70182 | 70187 | 70188 | 70191 | 70192 | 70195 | 70197 | 70198 | 70199 | 70200 |
| | 70201 | 70202 | 70203 | 70205 | 70206 | 70207 | 70208 | 70209 | 70212 | 70213 | 70215 | 70216 |
| | 70217 | 70219 | 70220 | 70221 | 70222 | 70223 | 70224 | 70225 | 70226 | 70229 | 70233 | 70234 |
| | 70235 | 70236 | 70238 | 70239 | 70240 | 70243 | 70244 | 70245 | 70247 | 70248 | 70252 | 70254 |
| | 70255 | 70257 | 70259 | 70260 | 70262 | 70264 | 70266 | 70267 | 70270 | 70271 | 70272 | 70273 |
| | 70274 | 70275 | 70276 | 70277 | 70279 | 70280 | 70281 | 70282 | 70283 | 70286 | 70287 | 70290 |
| | 70291 | 70292 | 70294 | 70296 | 70297 | 70298 | 70299 | 70300 | 70301 | 70303 | 70304 | 70307 |
| | 70308 | 70311 | 70312 | 70313 | 70314 | 70315 | 70319 | 70321 | 70322 | 70328 | 70330 | 70331 |
| | 70335 | 70337 | 70338 | 70342 | 70344 | 70345 | 70347 | 70348 | 70349 | 70352 | 70353 | 70356 |
| | 70357 | 70358 | 70359 | 70360 | 70361 | 70363 | 70364 | 70365 | 70366 | 70367 | 70368 | 70369 |
| | 70373 | 70374 | 70375 | 70376 | 70377 | 70378 | 70380 | 70382 | 70384 | 70385 | 70386 | 70387 |
| | 70390 | 70391 | 70392 | 70393 | 70394 | 70396 | 70399 | 70400 | 70401 | 70409 | 70415 | 70416 |
| | 70417 | 70422 | 70425 | 70429 | 70430 | 70436 | 70443 | 70450 | 70469 | 70473 | 70474 | 70476 |
| | 70477 | 70479 | 70481 | 70483 | 70484 | 70488 | 70489 | 70492 | 70493 | 70494 | 70495 | 70498 |
| | 70501 | 70504 | 70522 | 70526 | 70531 | 70536 | 70555 | 70562 | 70563 | 70564 | 70574 | 70577 |
| | 70579 | 70580 | 70583 | 70593 | 70594 | 70605 | 70611 | 70618 | 70624 | 70630 | 70632 | 70638 |
| | 70639 | 70640 | 70641 | 70642 | 70644 | 70647 | 70656 | 70660 | 70662 | 70663 | 70664 | 70665 |
| | 70676 | 70678 | 70691 | 70701 | 70704 | 70708 | 70709 | 70712 | 70715 | 70717 | 70718 | 70719 |
| | 70720 | 70722 | 70723 | 70727 | 70728 | 70729 | 70730 | 70732 | 70734 | 70738 | 70739 | 70740 |
| | 70743 | 70744 | 70745 | 70746 | 70747 | 70749 | 70750 | 70751 | 70756 | 70759 | 70760 | 70762 |
| | 70766 | 70767 | 70771 | 70772 | 70773 | 70774 | 70775 | 70776 | 70777 | 70779 | 70780 | 70781 |
| | 70782 | 70783 | 70785 | 70786 | 70787 | 70788 | 70789 | 70790 | 70791 | 70792 | 70793 | 70794 |
| | 70795 | 70797 | 70798 | 70799 | 70800 | 70802 | 70803 | 70805 | 70806 | 70809 | 70812 | 70813 |
| | 70814 | 70815 | 70816 | 70818 | 70822 | 70824 | 70825 | 70827 | 70828 | 70833 | 70834 | 70835 |
| | 70836 | 70838 | 70840 | 70844 | 70845 | 70846 | 70847 | 70848 | 70849 | 70853 | 70854 | 70855 |
| | 70856 | 70857 | 70858 | 70859 | 70861 | 70862 | 70863 | 70864 | 70866 | 70867 | 70868 | 70869 |
| | 70874 | 70875 | 70877 | 70878 | 70883 | 70884 | 70885 | 70886 | 70888 | 70889 | 70892 | 70897 |
| | 70898 | 70899 | 70901 | 70902 | 70903 | 70904 | 70909 | 70910 | 70911 | 70912 | 70913 | 70915 |
| | 70917 | 70919 | 70922 | 70923 | 70927 | 70930 | 70932 | 70934 | 70935 | 70937 | 70938 | 70939 |
| | 70940 | 70941 | 70942 | 70943 | 70944 | 70946 | 70947 | 70949 | 70950 | 70952 | 70953 | 70955 |
| | 70956 | 70957 | 70958 | 70960 | 70961 | 70962 | 70963 | 70964 | 70965 | 70966 | 70967 | 70968 |
| | 70969 | 70971 | 70972 | 70975 | 70976 | 70977 | 70978 | 70979 | 70981 | 70982 | 70983 | 70984 |
| | 70985 | 70986 | 70987 | 70988 | 70989 | 70990 | 70991 | 70995 | 70996 | 70998 | 70999 | 71000 |
| | 71001 | 71002 | 71003 | 71004 | 71006 | 71007 | 71008 | 71010 | 71011 | 71013 | 71016 | 71018 |
| | 71019 | 71021 | 71023 | 71024 | 71027 | 71033 | 71042 | 71044 | 71051 | 71053 | 71056 | 71057 |
| | 71061 | 71064 | 71065 | 71066 | 71679 | 71070 | 71077 | 71088 | 71093 | 71096 | 71099 | 71100 |
| | 71109 | 71110 | 71111 | 71113 | 71120 | 71123 | 71128 | 71130 | 71132 | 71133 | 71140 | 71145 |
| | 71146 | 71147 | 71148 | 71151 | 71152 | 71154 | 71156 | 71157 | 71159 | 71160 | 71161 | 71162 |
| | 71163 | 71165 | 71166 | 71170 | 71173 | 71174 | 71175 | 71176 | 71179 | 71180 | 71181 | 71183 |
| | 71185 | 71194 | 71198 | 71199 | 71203 | 71204 | 71206 | 71213 | 71225 | 71236 | 71241 | 71249 |
| | 71257 | 71261 | 71263 | 71266 | 71267 | 71268 | 71271 | 71272 | 71273 | 71276 | 71280 | 71285 |
| | 71291 | 71292 | 71294 | 71295 | 71313 | 71344 | 71345 | 71346 | 71348 | 71349 | 71352 | 71353 |
| | 71354 | 71361 | 71362 | 71367 | 71368 | 71371 | 71372 | 71391 | 71396 | 71398 | 71399 | 71404 |
| | 71406 | 71408 | 71412 | 71415 | 71418 | 71421 | 71428 | 71429 | 71430 | 71438 | 71439 | 71440 |
| | 71442 | 71443 | 71449 | 71450 | 71453 | 71454 | 71459 | 71460 | 71462 | 71464 | 71467 | 71472 |
| | 71480 | 71482 | 71483 | 71486 | 71488 | 71489 | 71492 | 71495 | 71498 | 71500 | 71502 | 71506 |
| | 71512 | 71513 | 71514 | 71516 | 71534 | 71540 | 71542 | 71543 | 71544 | 71547 | 71548 | 71551 |
| | 71563 | 71564 | 71565 | 71569 | 71570 | 71572 | 71574 | 71575 | 71577 | 71578 | 71579 | 71581 |
| | 71588 | 71591 | 71592 | 71594 | 71595 | 71596 | 71597 | 71598 | 71600 | 71604 | 71617 | 71618 |
| | 71620 | 71621 | 71626 | 71627 | 71630 | 71631 | 71632 | 71636 | 71648 | 71662 | 71664 | 71666 |
| | 71667 | 71668 | 71669 | 71670 | 71672 | 71673 | 71675 | 71676 | 71677 | 71679 | 71689 | 71692 |
| | 71699 | 71700 | 71701 | 71705 | 71706 | 75529 | 75539 | 75540 | 75542 | 75543 | 75557 | 75560 |
| | 75561 | 75562 | 75570 | 75576 | 75577 | 75578 | 75582 | 75586 | 75587 | 75588 | 75589 | 75592 |
| | 75593 | 75594 | 75595 | 75596 | 75600 | 75611 | 75612 | 75613 | 75614 | 75616 | 75619 | 75628 |
| | 75629 | 75630 | 75631 | 75653 | 75669 | 75680 | 75690 | 75694 | 75699 | 75700 | 75705 | 75706 |
| | 75743 | 75767 | 75768 | 75772 | 75773 | 75774 | 75775 | 75776 | 75777 | 75787 | 75788 | 75791 |
| | 75807 | 75808 | 75812 | 75814 | 75816 | 75818 | 75819 | 75827 | 75828 | 75829 | 75830 | 75837 |
| | 75838 | 75839 | 75840 | 75841 | 75843 | 75844 | 75848 | 75849 | 75850 | 75851 | 75853 | 75872 |
| | 75880 | 75885 | 75886 | 75888 | 75891 | 75892 | 75893 | 75901 | 75902 | 75903 | 75904 | 75917 |
| | 75923 | 75924 | 75925 | 75926 | 75928 | 75940 | 75941 | 75943 | 75945 | 75946 | 75947 | 75952 |
| | 75957 | 75958 | 75960 | 75961 | 75962 | 75963 | 75964 | 75969 | 75970 | 75978 | 75979 | 75980 |
| | 75981 | 75982 | 75983 | 75984 | 75986 | 75987 | 75992 | 76011 | 76013 | 76015 | 76016 | 76017 |
| | 76018 | 76027 | 76028 | 76029 | 76033 | 76035 | 76044 | 76045 | 76046 | 76047 | 76048 | |

(11) Metal-binding proteins (SEQ ID NOs)

| | | | | | | | | | | | | |
|---|---|---|---|---|---|---|---|---|---|---|---|---|
| 4825 | 4936 | 4973 | 5174 | 5220 | 5224 | 5382 | 6020 | 6050 | 6142 | 6317 | 6337 | 6468 |
| | 6605 | 10463 | 10856 | 10857 | 10862 | 10885 | 11032 | 11034 | 11431 | 11869 | 11994 | 12018 |
| | 16978 | 16769 | 16778 | 16779 | 16781 | 16782 | 16783 | 16858 | 16862 | 16873 | 16877 | 16878 |
| | 16879 | 16880 | 54097 | 54098 | 54143 | 54144 | 54145 | 54146 | 54147 | 54148 | 54153 | 54154 |
| | 54157 | 54158 | 54159 | 54160 | 54161 | 54162 | 54163 | 54164 | 54165 | 54166 | 54167 | 54168 |
| | 54169 | 54170 | 54171 | 54175 | 54176 | 54177 | 54178 | 54179 | 54180 | 54181 | 54182 | 54183 |
| | 54184 | 54185 | 54186 | 54187 | 54209 | 54210 | 54211 | 54214 | 54215 | 54216 | 54217 | 54218 |
| | 54219 | 54226 | 54227 | 54228 | 54229 | 54230 | 54231 | 54232 | 54233 | 54234 | 54235 | 54236 |
| | 54237 | 54240 | 54241 | 54244 | 54245 | 54246 | 54307 | 54308 | 54455 | 54456 | 54457 | 54458 |
| | 54459 | 54460 | 54511 | 54512 | 54513 | 54514 | 54665 | 54666 | 54667 | 54802 | 55005 | 55480 |
| | 55566 | 55630 | 55631 | 55632 | 55842 | 56663 | 56664 | 56700 | 56701 | 56702 | 56703 | 56731 |
| | 56732 | 56733 | 56815 | 56816 | 56828 | 56829 | 57141 | 57142 | 57143 | 57159 | 57160 | 57161 |
| | 57162 | 57213 | 57214 | 58180 | 58181 | 58262 | 58263 | 58264 | 58265 | 58268 | 58269 | 58270 |
| | 58271 | 58299 | 58300 | 58301 | 58302 | 58305 | 58306 | 58307 | 58308 | 58317 | 58318 | 58319 |
| | 58320 | 58394 | 58395 | 58396 | 58397 | 58458 | 58459 | 58460 | 58461 | 58544 | 58545 | 58546 |
| | 58547 | 59096 | 59097 | 59098 | 59099 | 59100 | 60610 | 60611 | 60612 | 60613 | 60614 | 61600 |
| | 61601 | 62043 | 62044 | 62045 | 62166 | 62167 | 62367 | 62368 | 62369 | 62370 | 62438 | 63120 |
| | 63121 | 63122 | 63179 | 63180 | 63277 | 63629 | 63630 | 63631 | 64185 | 64350 | 64351 | 64352 |
| | 65143 | 65188 | 65225 | 65324 | 65545 | 65559 | 65581 | 65582 | 65591 | 65592 | 65607 | 65828 |
| | 65829 | 65830 | 65841 | 65842 | 65848 | 65849 | 65850 | 65865 | 65918 | 65964 | 65983 | 65984 |
| | 66007 | 66008 | 66059 | 66060 | 66061 | 66062 | 66066 | 66075 | 66076 | 66077 | 66078 | 66079 |
| | 66092 | 66093 | 66094 | 66095 | 66097 | 66098 | 66177 | 66184 | 66386 | 66387 | 66532 | 66560 |
| | 66567 | 66646 | 66647 | 67110 | 67111 | 67212 | 67540 | 67586 | 67692 | 67950 | 67987 | 68345 |
| | 68510 | 68511 | 68535 | 68592 | 68593 | 68671 | 68672 | 69056 | 69057 | 69104 | 69182 | 69183 |
| | 69253 | 69265 | 69266 | 69267 | 69557 | 69558 | 69612 | 69632 | 69744 | 69797 | 69798 | 69863 |
| | 69944 | 69971 | 70020 | 70021 | 70041 | 70070 | 70104 | 70156 | 70346 | 70371 | 70427 | 70442 |
| | 70447 | 70448 | 70449 | 70451 | 70453 | 70455 | 70458 | 70459 | 70463 | 70465 | 70467 | 70468 |
| | 70470 | 70471 | 70487 | 70490 | 70491 | 70497 | 70506 | 70507 | 70510 | 70514 | 70530 | 70535 |
| | 70540 | 70554 | 70556 | 70557 | 70561 | 70626 | 70653 | 70873 | 71012 | 71054 | 71067 | 71072 |
| | 71086 | 71153 | 71320 | 71321 | 71325 | 71329 | 71332 | 71334 | 71357 | 71359 | 71364 | 71365 |
| | 71366 | 71420 | 71441 | 71456 | 71499 | 71601 | 71610 | 71622 | 71647 | 71685 | 71697 | 75590 |
| | 75753 | 75754 | 75755 | 75762 | 75785 | 75786 | 75999 | 76000 | 76001 | 76042 | | |

(12) Methanogenesis proteins (SEQ ID NOs)

| | | | | | | | | | | | | |
|---|---|---|---|---|---|---|---|---|---|---|---|---|
| 5833 | 5835 | 5836 | 5837 | 5838 | 5839 | 5840 | 5841 | 5842 | 5843 | 5844 | 6360 | 6432 |
| | 6436 | 6437 | 6438 | 6439 | 6440 | 6441 | 6442 | 6443 | 6444 | 6445 | 10474 | 10475 |
| | 10476 | 10477 | 10478 | 10479 | 10480 | 10481 | 10482 | 10483 | 10484 | 10485 | 11511 | 11549 |
| | 11554 | 11555 | 11556 | 11557 | 11558 | 11559 | 11560 | 11562 | 16859 | 54741 | 54742 | 54743 |
| | 54744 | 54745 | 54786 | 54787 | 54788 | 54789 | 54790 | 54871 | 54872 | 54873 | 54874 | 54875 |
| | 56843 | 56844 | 57209 | 57210 | 57215 | 57216 | 57217 | 57218 | 57219 | 57220 | 57230 | 57231 |
| | 57232 | 57233 | 57234 | 57235 | 57267 | 57268 | 57269 | 57270 | 57271 | 57272 | 57273 | 57274 |
| | 57275 | 57276 | 57277 | 57346 | 57347 | 57348 | 57349 | 57350 | 57351 | 57365 | 57366 | 57367 |
| | 57368 | 57416 | 57417 | 57418 | 57461 | 57462 | 57463 | 57464 | 57499 | 57500 | 57501 | 57510 |
| | 57511 | 57512 | 57706 | 57707 | 57708 | 57721 | 57722 | 57723 | 57734 | 57735 | 57736 | 57737 |
| | 57738 | 57739 | 57979 | 57980 | 57981 | 57982 | 57983 | 57984 | 57998 | 57999 | 58000 | 58001 |
| | 58002 | 58003 | 58011 | 58012 | 58013 | 58017 | 58018 | 58019 | 58023 | 58024 | 58025 | 58029 |
| | 58030 | 58031 | 58035 | 58036 | 58037 | 58047 | 58048 | 58049 | 58065 | 58066 | 58067 | 58077 |
| | 58078 | 58079 | 58080 | 58081 | 58082 | 58104 | 58105 | 58106 | 58107 | 58108 | 58109 | 58113 |
| | 58114 | 58115 | 58119 | 58120 | 58121 | 58144 | 58145 | 58146 | 58185 | 58186 | 58187 | 58188 |
| | 58189 | 58190 | 58191 | 58192 | 58193 | 58194 | 58195 | 58196 | 58200 | 58201 | 58202 | 58203 |
| | 58204 | 58205 | 58206 | 58209 | 58210 | 58211 | 58212 | 58213 | 58214 | 58242 | 58243 | 58244 |
| | 58245 | 58246 | 58843 | 58844 | 58845 | 58846 | 59842 | 59843 | 59844 | 59845 | 59846 | 59847 |
| | 60615 | 60616 | 60617 | 60618 | 60624 | 60625 | 60626 | 60627 | 60628 | 60634 | 60635 | 60636 |
| | 60637 | 60659 | 61096 | 61097 | 61098 | 61240 | 61241 | 61242 | 61243 | 61511 | 61531 | 61532 |
| | 61533 | 61534 | 61712 | 61713 | 61714 | 61715 | 61790 | 61793 | 61796 | 61806 | 61833 | 61846 |
| | 61851 | 61854 | 61857 | 61871 | 61874 | 61883 | 61888 | 61891 | 61925 | 61944 | 61945 | 61956 |
| | 61962 | 62778 | 62779 | 63283 | 63284 | 63844 | 63845 | 63846 | 63851 | 63903 | 63911 | 63924 |
| | 63925 | 63929 | 63932 | 63933 | 63935 | 63936 | 63937 | 63938 | 63939 | 63940 | 63941 | 63942 |
| | 63943 | 63944 | 63945 | 63949 | 63951 | 63955 | 63956 | 63957 | 63958 | 63962 | 63966 | 63967 |
| | 63968 | 63977 | 63981 | 63982 | 63983 | 63984 | 63985 | 63986 | 64041 | 64141 | 64165 | 65295 |
| | 65303 | 65305 | 65310 | 65315 | 65316 | 65329 | 65334 | 65467 | 65469 | 65486 | 65503 | 65511 |
| | 65517 | 65519 | 65530 | 65534 | 65538 | 65540 | 65546 | 65550 | 65656 | 65880 | 66628 | 67044 |
| | 67561 | 67566 | 67568 | 67569 | 67571 | 67572 | 67575 | 67578 | 67580 | 67582 | 67669 | 67670 |
| | 67682 | 67683 | 67694 | 67700 | 67711 | 67712 | 67718 | 67719 | 67720 | 67723 | 67724 | 67729 |
| | 67732 | 67735 | 67738 | 67739 | 67742 | 67743 | 67754 | 67755 | 67756 | 67757 | 67783 | 67784 |
| | 67789 | 67790 | 67795 | 67796 | 67802 | 67803 | 67805 | 67806 | 67809 | 67810 | 67811 | 67812 |
| | 67821 | 67822 | 67823 | 67824 | 67831 | 67832 | 67841 | 67842 | 67845 | 67846 | 67863 | 68637 |
| | 68712 | 68721 | 68732 | 68733 | 68734 | 68735 | 68736 | 68737 | 68739 | 68740 | 68748 | 68749 |
| | 68751 | 68752 | 68755 | 68757 | 68758 | 68760 | 68761 | 68769 | 68856 | 58857 | 68871 | 68874 |
| | 68878 | 68916 | 69112 | 69113 | 69114 | 69140 | 69145 | 69215 | 69244 | 69290 | 69297 | 69303 |
| | 69310 | 69329 | 69335 | 69338 | 69350 | 69360 | 69361 | 69366 | 69389 | 69392 | 69406 | 69439 |
| | 69455 | 69469 | 69475 | 69476 | 69481 | 69484 | 69489 | 69491 | 69494 | 69497 | 69498 | 69500 |
| | 69501 | 69502 | 69505 | 69506 | 69525 | 69527 | 69534 | 69582 | 69920 | 69924 | 69934 | 69939 |
| | 69940 | 69941 | 69942 | 69943 | 69948 | 69949 | 69951 | 69953 | 69954 | 69961 | 69962 | 69964 |
| | 69967 | 69968 | 69970 | 69973 | 69974 | 69975 | 69977 | 69978 | 69979 | 69980 | 69981 | 69982 |
| | 69983 | 69986 | 69987 | 69990 | 69995 | 69997 | 70022 | 70040 | 70048 | 70056 | 70068 | 70074 |
| | 70097 | 70112 | 70124 | 70146 | 70153 | 70175 | 70193 | 70261 | 70309 | 70339 | 70343 | 70355 |
| | 70389 | 70395 | 70408 | 70428 | 70452 | 70457 | 70464 | 70520 | 70523 | 70529 | 70534 | 70544 |
| | 70547 | 70550 | 70553 | 70560 | 70565 | 70566 | 70573 | 70578 | 70581 | 70584 | 70588 | 70592 |
| | 70595 | 70597 | 70598 | 70600 | 70606 | 70607 | 70670 | 70687 | 70689 | 70695 | 70697 | 70699 |
| | 70702 | 70703 | 70705 | 70706 | 70711 | 70713 | 70736 | 70769 | 70770 | 70870 | 70945 | 71358 |
| | 71336 | 71337 | 71358 | 71527 | 75913 | | | | | | | |

(13) Methanogenesis Mtr proteins (SEQ ID NOs)

| | | | | | | | | | | | | |
|---|---|---|---|---|---|---|---|---|---|---|---|---|
| 6349 | 6350 | 6351 | 6352 | 6353 | 6354 | 11495 | 11496 | 11498 | 11499 | 11500 | 65965 | 66149 |
| | 66150 | 66166 | 66173 | 66174 | 66948 | 67048 | 67049 | 67153 | 67192 | 67246 | 67443 | 67479 |
| | 67480 | 67481 | 67486 | 67489 | 67495 | 67500 | 67512 | 67542 | 67601 | 68002 | 70323 | 70324 |
| | 70332 | 70333 | 70397 | 70398 | 70671 | 70673 | 70680 | 70688 | 70690 | 70710 | 70980 | 71521 |
| | 71282 | 71282 | 71503 | 71518 | 71519 | 71520 | 71521 | 71522 | 71523 | 71524 | 71651 | 75911 |

(14) Methanogenesis MtrE proteins (SEQ ID NOs)

| | | | | | | | | | | | | |
|---|---|---|---|---|---|---|---|---|---|---|---|---|
| 6355 | 11501 | 12052 | 12053 | 12054 | 12055 | 12056 | 12057 | 12058 | 12059 | 12060 | 12061 | 12062 |
| | 12063 | 12064 | 12065 | 12066 | 12067 | 12068 | 12069 | 12070 | 12071 | 12072 | 12073 | 12074 |
| | 12075 | 12076 | 12077 | 12078 | 12079 | 12080 | 12081 | 12082 | 12083 | 12084 | 12085 | 12086 |
| | 12087 | 12088 | 12089 | 12090 | 12091 | 12092 | 12093 | 12094 | 12095 | 12096 | 12097 | 12098 |
| | 12099 | 16969 | 16970 | 16971 | 16972 | 16973 | 64574 | 65049 | 65136 | 65190 | 65201 | 65238 |
| | 65294 | 65312 | 65962 | 65963 | 66049 | 66091 | 66635 | 66636 | 66808 | 71525 | 75912 | |

(15) Phage related (SEQ ID NOs)

| | | | | | | | |
|---|---|---|---|---|---|---|---|
| 4742 | 4744 | 4745 | 4750 | 57399 | 57400 | 57401 | 69072 |

(16) Proteolysis (SEQ ID NOs)

| | | | | | | | | | | | | |
|---|---|---|---|---|---|---|---|---|---|---|---|---|
| 4466 | 4564 | 4672 | 4754 | 5032 | 5120 | 5314 | 5461 | 5560 | 5678 | 5695 | 5744 | 5936 |
| | 6069 | 6109 | 6130 | 6600 | 10411 | 10525 | 10580 | 10737 | 11111 | 11232 | 11426 | 11638 |
| | 11652 | 11798 | 16770 | 16788 | 16790 | 53469 | 53470 | 53471 | 53472 | 53473 | 53474 | 53475 |
| | 53476 | 53506 | 53507 | 53814 | 53815 | 53816 | 53817 | 53818 | 53819 | 53863 | 53864 | 53865 |
| | 53885 | 53886 | 53887 | 53888 | 53889 | 53890 | 53891 | 53892 | 53893 | 53894 | 53895 | 53896 |
| | 53897 | 53898 | 53899 | 53900 | 53901 | 53902 | 53903 | 53904 | 53905 | 53996 | 53997 | 53998 |
| | 53999 | 54000 | 54001 | 54002 | 54003 | 54004 | 54012 | 54013 | 54022 | 54023 | 54024 | 54025 |
| | 54026 | 54027 | 54028 | 54029 | 54030 | 54031 | 54032 | 54033 | 54141 | 54254 | 54300 | 54301 |
| | 54322 | 54323 | 55371 | 55372 | 55373 | 55374 | 55500 | 56158 | 56159 | 57152 | 57223 | 57224 |
| | 57359 | 57410 | 57411 | 57412 | 57885 | 57886 | 57887 | 57888 | 57889 | 58584 | 58585 | 60294 |
| | 60295 | 60296 | 60297 | 60690 | 60691 | 60692 | 60693 | 60773 | 60774 | 60775 | 60776 | 60777 |
| | 60778 | 60779 | 60845 | 60874 | 61622 | 61623 | 61909 | 61910 | 61934 | 61976 | 61979 | 61982 |
| | 61983 | 61990 | 62063 | 62064 | 62093 | 62105 | 62165 | 62175 | 62176 | 62183 | 62189 | 62190 |
| | 62191 | 62192 | 62193 | 62196 | 62197 | 62202 | 62203 | 62204 | 62213 | 62214 | 62222 | 62227 |
| | 62229 | 62230 | 62255 | 62256 | 62361 | 62362 | 62386 | 62433 | 62434 | 62436 | 62437 | 62444 |
| | 62445 | 62446 | 62447 | 62944 | 62945 | 62946 | 63152 | 63153 | 63467 | 63950 | 64060 | 64061 |
| | 64062 | 64063 | 64064 | 64065 | 64066 | 64204 | 64205 | 64206 | 64265 | 64270 | 64272 | 64273 |
| | 64294 | 64295 | 64331 | 64332 | 64333 | 64379 | 64391 | 64392 | 64393 | 64403 | 64404 | 64405 |
| | 64413 | 64414 | 64415 | 64427 | 64428 | 64429 | 64436 | 64437 | 64438 | 64452 | 64453 | 64454 |
| | 64458 | 64459 | 64460 | 64473 | 64474 | 64475 | 64482 | 64483 | 64484 | 64488 | 64506 | 64511 |
| | 64513 | 64520 | 64521 | 64522 | 64564 | 64591 | 64592 | 64593 | 64598 | 64599 | 64600 | 64620 |
| | 64621 | 64622 | 64643 | 64644 | 64645 | 64646 | 64647 | 64648 | 64652 | 64653 | 64654 | 64667 |
| | 64668 | 64669 | 64699 | 64700 | 64701 | 64720 | 64929 | 64930 | 64957 | 64958 | 64959 | 65012 |
| | 65013 | 65014 | 65120 | 65121 | 65138 | 65139 | 65140 | 65277 | 65278 | 65283 | 65301 | 65302 |
| | 65357 | 65358 | 65367 | 65368 | 65379 | 65380 | 65381 | 65382 | 65385 | 65386 | 65387 | 65388 |
| | 65389 | 65390 | 65391 | 65406 | 65407 | 65408 | 65409 | 65418 | 65419 | 65424 | 65425 | 65426 |
| | 65427 | 65429 | 65430 | 65437 | 65438 | 65444 | 65445 | 65450 | 65451 | 65453 | 65454 | 65455 |
| | 65456 | 65457 | 65458 | 65459 | 65460 | 65470 | 65565 | 65788 | 65789 | 65790 | 65798 | 65799 |
| | 65800 | 65813 | 65814 | 65815 | 65874 | 65891 | 65892 | 66033 | 66034 | 66038 | 66133 | 66161 |
| | 66171 | 66172 | 66191 | 66212 | 66228 | 66239 | 66240 | 66264 | 66700 | 66886 | 66887 | 66888 |
| | 67060 | 67061 | 67325 | 67326 | 67327 | 67531 | 67532 | 67666 | 67667 | 67705 | 67706 | 67707 |
| | 67807 | 67808 | 67855 | 67856 | 67859 | 67860 | 67936 | 67995 | 68017 | 68018 | 68022 | 68023 |
| | 68076 | 68077 | 68110 | 68111 | 68112 | 68113 | 68131 | 68132 | 68142 | 68143 | 68146 | 68147 |
| | 68166 | 68167 | 68168 | 68169 | 68170 | 68171 | 68176 | 68177 | 68182 | 68183 | 68186 | 68187 |
| | 68196 | 68197 | 68202 | 68203 | 68204 | 68205 | 68214 | 68215 | 68222 | 68223 | 68226 | 68227 |
| | 68228 | 68229 | 68230 | 68231 | 68242 | 68243 | 68244 | 68245 | 68250 | 68251 | 68254 | 68255 |
| | 68269 | 68270 | 68271 | 68272 | 68273 | 68274 | 68326 | 68327 | 68328 | 68329 | 68362 | 68373 |
| | 68374 | 68418 | 68602 | 68628 | 68635 | 68636 | 68643 | 68675 | 69127 | 69128 | 69129 | 69179 |
| | 69180 | 69199 | 69200 | 69236 | 69237 | 69328 | 69446 | 69447 | 69593 | 69787 | 69862 | 69882 |
| | 69883 | 70035 | 70157 | 70184 | 70288 | 70340 | 70379 | 70383 | 70411 | 70461 | 70462 | 70533 |
| | 70654 | 70655 | 70894 | 71084 | 71135 | 71142 | 71188 | 71211 | 71269 | 71390 | 71411 | 71437 |
| | 71497 | 71576 | 71582 | 71634 | 75526 | 75584 | 75585 | 75621 | 75622 | 75623 | 75636 | 75701 |
| | 75835 | 75836 | 75890 | 75990 | 75991 | | | | | | | |

(17) Transcription regulation (SEQ ID NOs)

| | | | | |
|---|---|---|---|---|
| 54777 | 54778 | 54779 | 54780 | 70620 |

(18) Ribosomal (SEQ ID NOs)

| | | | | | | | | | | | |
|---|---|---|---|---|---|---|---|---|---|---|---|
| 61896 | 61897 | 62721 | 66723 | 66724 | 66725 | 66726 | 66729 | 67400 | 71216 | 71355 | 71652 |

(19) Substrate binding (SEQ ID NOs)

16860

(20) Transcription (SEQ ID NOs)

| | | | | | | | | | | | | |
|---|---|---|---|---|---|---|---|---|---|---|---|---|
| 57620 | 57621 | 59813 | 65150 | 65151 | 65152 | 68305 | 68658 | 69018 | 69019 | 69020 | 70177 | 70179 |
| | 70285 | 70621 | 70622 | 71134 | 71231 | 71360 | 71427 | 71607 | 71646 | | | |

(21) Transport (SEQ ID NOs)

| | | | | | | | | | | | | |
|---|---|---|---|---|---|---|---|---|---|---|---|---|
| 4437 | 4442 | 4450 | 4451 | 4480 | 4503 | 4527 | 4550 | 4573 | 4574 | 4575 | 4632 | 4634 |
| | 4635 | 4640 | 4641 | 4649 | 4650 | 4673 | 4685 | 4686 | 4699 | 4717 | 4763 | 4766 |
| | 4786 | 4792 | 4800 | 4813 | 4839 | 4840 | 4841 | 4900 | 4915 | 4929 | 4930 | 4967 |
| | 4970 | 4972 | 4974 | 4991 | 4992 | 5003 | 5004 | 5040 | 5042 | 5049 | 5069 | 5092 |
| | 5093 | 5129 | 5130 | 5138 | 5157 | 5222 | 5241 | 5254 | 5260 | 5308 | 5316 | 5317 |
| | 5318 | 5383 | 5419 | 5426 | 5435 | 5503 | 5514 | 5515 | 5516 | 5623 | 5633 | 5634 |
| | 5653 | 5717 | 5757 | 5764 | 5765 | 5772 | 5802 | 5805 | 5815 | 5860 | 5869 | 5871 |
| | 5888 | 5942 | 5954 | 5956 | 6029 | 6044 | 6048 | 6049 | 6059 | 6060 | 6090 | 6134 |
| | 6140 | 6141 | 6145 | 6153 | 6154 | 6167 | 6183 | 6197 | 6208 | 6209 | 6218 | 6221 |
| | 6272 | 6273 | 6312 | 6377 | 6385 | 6400 | 6416 | 6452 | 6456 | 6477 | 6478 | 6479 |
| | 6483 | 6608 | 6609 | 6629 | 6638 | 6640 | 6650 | 10256 | 10271 | 10275 | 10276 | 10284 |
| | 10295 | 10296 | 10302 | 10303 | 10343 | 10352 | 10354 | 10355 | 10365 | 10380 | 10381 | 10405 |
| | 10406 | 10407 | 10409 | 10425 | 10496 | 10499 | 10516 | 10527 | 10528 | 10578 | 10623 | 10624 |
| | 10625 | 10629 | 10630 | 10648 | 10651 | 10652 | 10655 | 10656 | 10657 | 10663 | 10701 | 10718 |
| | 10728 | 10738 | 10753 | 10841 | 10842 | 10845 | 10847 | 10850 | 10851 | 10854 | 10855 | 10863 |
| | 10864 | 10870 | 10905 | 10932 | 10976 | 11054 | 11073 | 11083 | 11095 | 11121 | 11124 | 11134 |
| | 11140 | 11141 | 11142 | 11159 | 11205 | 11214 | 11215 | 11227 | 11274 | 11329 | 11341 | 11342 |
| | 11343 | 11355 | 11371 | 11421 | 11433 | 11469 | 11470 | 11513 | 11514 | 11530 | 11544 | 11564 |
| | 11572 | 11574 | 11575 | 11583 | 11626 | 11671 | 11677 | 11680 | 11681 | 11684 | 11705 | 11725 |
| | 11742 | 11751 | 11787 | 11813 | 11817 | 11839 | 11912 | 11980 | 11991 | 12007 | 12013 | 12017 |
| | 12019 | 16772 | 16773 | 16775 | 16776 | 16796 | 16798 | 16870 | 16875 | 53543 | 53544 | 53545 |
| | 53565 | 53566 | 53567 | 53568 | 53569 | 53570 | 53588 | 53589 | 53590 | 53591 | 53592 | 53593 |
| | 53600 | 53985 | 54018 | 54019 | 54020 | 54021 | 54094 | 54095 | 54096 | 54119 | 54120 | 54121 |
| | 54142 | 54206 | 54207 | 54208 | 54220 | 54221 | 54255 | 54256 | 54257 | 54258 | 54259 | 54260 |
| | 54261 | 54273 | 54274 | 54275 | 54276 | 54304 | 54309 | 54310 | 54311 | 54312 | 54317 | 54319 |
| | 54326 | 54327 | 54338 | 54339 | 54340 | 54341 | 54342 | 54343 | 54344 | 54345 | 54346 | 54347 |
| | 54348 | 54349 | 54350 | 54351 | 54352 | 54354 | 54355 | 54356 | 54357 | 54358 | 54359 | 54360 |
| | 54361 | 54362 | 54363 | 54364 | 54365 | 54366 | 54367 | 54368 | 54381 | 54382 | 54383 | 54384 |
| | 54385 | 54386 | 54432 | 54433 | 54434 | 54435 | 54436 | 54437 | 54438 | 54439 | 54444 | 54445 |
| | 54446 | 54461 | 54476 | 54477 | 54507 | 54508 | 54509 | 54510 | 54515 | 54516 | 54522 | 54523 |
| | 54524 | 54525 | 54528 | 54529 | 54530 | 54531 | 54532 | 54533 | 54534 | 54535 | 54538 | 54539 |
| | 54540 | 54541 | 54546 | 54547 | 54548 | 54549 | 54550 | 54551 | 54552 | 54553 | 54554 | 54555 |
| | 54556 | 54557 | 54558 | 54559 | 54560 | 54561 | 54562 | 54563 | 54564 | 54565 | 54566 | 54567 |
| | 54568 | 54569 | 54570 | 54571 | 54572 | 54573 | 54574 | 54575 | 54576 | 54577 | 54578 | 54579 |
| | 54580 | 54581 | 54582 | 54583 | 54584 | 54585 | 54586 | 54587 | 54588 | 54589 | 54590 | 54591 |
| | 54592 | 54593 | 54594 | 54595 | 54596 | 54597 | 54598 | 54599 | 54600 | 54601 | 54602 | 54603 |
| | 54604 | 54605 | 54606 | 54607 | 54608 | 54609 | 54610 | 54611 | 54612 | 54613 | 54614 | 54615 |
| | 54616 | 54617 | 54618 | 54619 | 54620 | 54621 | 54622 | 54623 | 54624 | 54628 | 54629 | 54630 |
| | 54631 | 54632 | 54633 | 54634 | 54635 | 54636 | 54637 | 54638 | 54639 | 54640 | 54641 | 54642 |
| | 54643 | 54644 | 54645 | 54658 | 54659 | 54663 | 54664 | 54668 | 54669 | 54670 | 54671 | 54672 |
| | 54673 | 54674 | 54675 | 54676 | 54678 | 54679 | 54680 | 54681 | 54682 | 54683 | 54684 | 54685 |
| | 54686 | 54687 | 54688 | 54689 | 54690 | 54691 | 54692 | 54695 | 54696 | 5469 | 54698 | 54700 |
| | 54701 | 54702 | 54703 | 54704 | 54705 | 54714 | 54715 | 54717 | 54718 | 54719 | 54720 | 54721 |
| | 54722 | 54723 | 54724 | 54725 | 54726 | 54727 | 54728 | 54729 | 54730 | 54731 | 54752 | 54753 |
| | 54754 | 54759 | 54760 | 54785 | 54795 | 54796 | 54797 | 54803 | 54804 | 54805 | 54806 | 54815 |
| | 54816 | 54817 | 54841 | 54842 | 54843 | 54846 | 54847 | 54848 | 54849 | 54850 | 54851 | 54852 |
| | 54853 | 54855 | 54856 | 54857 | 54858 | 54859 | 54860 | 54861 | 54862 | 54863 | 54864 | 54865 |
| | 54866 | 54867 | 54868 | 54869 | 54870 | 54876 | 54877 | 54878 | 54882 | 54883 | 54884 | 54885 |
| | 54886 | 54893 | 54894 | 54895 | 54896 | 54897 | 54898 | 54899 | 54900 | 54901 | 54902 | 54903 |
| | 54904 | 54905 | 54906 | 54907 | 54908 | 54909 | 54910 | 54911 | 54912 | 54913 | 54914 | 54915 |
| | 54916 | 54917 | 54918 | 54919 | 54920 | 54921 | 54922 | 54923 | 54924 | 54925 | 54926 | 54927 |
| | 54928 | 54929 | 54930 | 54931 | 54932 | 54933 | 54934 | 54935 | 54936 | 54937 | 54938 | 54939 |
| | 54940 | 54941 | 54942 | 54943 | 54944 | 54945 | 54946 | 54947 | 54948 | 54949 | 54950 | 54951 |
| | 54952 | 54953 | 54954 | 54955 | 54956 | 54957 | 54958 | 54959 | 54960 | 54961 | 54962 | 54963 |
| | 54964 | 54965 | 54966 | 54967 | 54968 | 54969 | 54970 | 54971 | 54972 | 54973 | 54974 | 54975 |
| | 54976 | 54977 | 54978 | 54979 | 54980 | 54981 | 54982 | 54983 | 54984 | 54985 | 54986 | 54987 |
| | 54988 | 54989 | 54990 | 54991 | 54992 | 54993 | 54994 | 54995 | 54996 | 54997 | 54998 | 54999 |
| | 55000 | 55001 | 55002 | 55003 | 55004 | 55006 | 55007 | 55008 | 55010 | 55011 | 55012 | 55013 |
| | 55014 | 55020 | 55021 | 55022 | 55023 | 55024 | 55025 | 55026 | 55027 | 55028 | 55029 | 55030 |
| | 55031 | 55032 | 55033 | 55034 | 55035 | 55036 | 55037 | 55038 | 55039 | 55040 | 55041 | 55042 |
| | 55043 | 55044 | 55045 | 55046 | 55047 | 55048 | 55049 | 55050 | 55051 | 55052 | 55053 | 55054 |
| | 55055 | 55056 | 55057 | 55058 | 55059 | 55060 | 55061 | 55062 | 55063 | 55064 | 55066 | 55067 |
| | 55068 | 55069 | 55070 | 55071 | 55072 | 55073 | 55085 | 55086 | 55087 | 55091 | 55092 | 55093 |
| | 55094 | 55095 | 55098 | 55099 | 55107 | 55108 | 55109 | 55132 | 55174 | 55178 | 55179 | 55180 |
| | 55181 | 55182 | 55192 | 55193 | 55194 | 55195 | 55196 | 55203 | 55204 | 55234 | 55238 | 55279 |
| | 55289 | 55292 | 55296 | 55302 | 55306 | 55307 | 55308 | 55311 | 55356 | 55357 | 55358 | 55359 |
| | 55360 | 55361 | 55369 | 55389 | 55390 | 55398 | 55399 | 55400 | 55401 | 55402 | 55403 | 55404 |
| | 55405 | 55406 | 55410 | 55411 | 55413 | 55414 | 55415 | 55416 | 55417 | 55421 | 55423 | 55426 |
| | 55445 | 55446 | 55447 | 55448 | 55449 | 55450 | 55459 | 55477 | 55481 | 55482 | 55486 | 55487 |
| | 55488 | 55489 | 55490 | 55491 | 55494 | 55495 | 55498 | 55499 | 55501 | 55502 | 55503 | 55504 |
| | 55512 | 55517 | 55518 | 55519 | 55520 | 55526 | 55527 | 55528 | 55537 | 55546 | 55547 | 55558 |
| | 55565 | 55585 | 55586 | 55590 | 55591 | 55592 | 55596 | 55599 | 55624 | 55629 | 55633 | 55634 |
| | 55636 | 55637 | 55638 | 55650 | 55651 | 55654 | 55655 | 55688 | 55689 | 55690 | 55695 | 55696 |
| | 55697 | 55698 | 55699 | 55700 | 55711 | 55712 | 55713 | 55715 | 55716 | 55717 | 55718 | 55719 |
| | 55720 | 55721 | 55722 | 55723 | 55728 | 55729 | 55730 | 55731 | 55734 | 55735 | 55736 | 55737 |
| | 55750 | 55751 | 55752 | 55753 | 55754 | 55755 | 55756 | 55760 | 55761 | 55762 | 55763 | 55764 |
| | 55765 | 55766 | 55768 | 55769 | 55770 | 55771 | 55773 | 55774 | 55775 | 55776 | 55777 | 55778 |
| | 55779 | 55780 | 55785 | 55786 | 55787 | 55788 | 55789 | 55790 | 55801 | 55802 | 55803 | 55814 |
| | 55815 | 55816 | 55817 | 55818 | 55819 | 55820 | 55821 | 55822 | 55823 | 55824 | 55825 | 55826 |
| | 55827 | 55828 | 55829 | 55833 | 55834 | 55835 | 55836 | 55837 | 55838 | 55839 | 55840 | 55841 |
| | 55845 | 55846 | 55847 | 55848 | 55849 | 55850 | 55851 | 55852 | 55853 | 55854 | 55855 | 55856 |
| | 55857 | 55858 | 55859 | 55860 | 55861 | 55862 | 55863 | 55864 | 55865 | 55866 | 55867 | 55868 |
| | 55869 | 55870 | 55871 | 55872 | 55873 | 55874 | 55875 | 55876 | 55877 | 55878 | 55879 | 55880 |
| | 55881 | 55882 | 55883 | 55884 | 55885 | 55886 | 55887 | 55888 | 55889 | 55890 | 55891 | 55892 |
| | 55893 | 55900 | 55901 | 55902 | 55903 | 55904 | 55905 | 55906 | 55907 | 55908 | 55909 | 55910 |
| | 55911 | 55912 | 55913 | 55914 | 55915 | 55916 | 55917 | 55918 | 55919 | 55920 | 55921 | 55922 |
| | 55923 | 55924 | 55925 | 55926 | 55927 | 55928 | 55929 | 55930 | 55931 | 55932 | 55938 | 55939 |
| | 55940 | 55941 | 55942 | 55943 | 55944 | 55945 | 55946 | 55947 | 55948 | 55949 | 55950 | 55951 |
| | 55952 | 55953 | 55954 | 55955 | 55956 | 55957 | 55958 | 55959 | 55960 | 55961 | 55962 | 55963 |
| | 55964 | 55965 | 55966 | 55967 | 55968 | 55972 | 55973 | 55974 | 55975 | 55976 | 55977 | 55978 |
| | 55979 | 55980 | 55981 | 55982 | 55983 | 55984 | 55985 | 55988 | 55989 | 55990 | 55991 | 55992 |
| | 55993 | 55994 | 55995 | 55996 | 55997 | 55998 | 55999 | 56000 | 56008 | 56009 | 56010 | 56011 |
| | 56016 | 56017 | 56018 | 56019 | 56020 | 56021 | 56022 | 56023 | 56024 | 56025 | 56026 | 56027 |
| | 56028 | 56029 | 56030 | 56031 | 56032 | 56033 | 56034 | 56035 | 56036 | 56037 | 56038 | 56039 |
| | 56040 | 56041 | 56042 | 56043 | 56044 | 56045 | 56046 | 56047 | 56048 | 56049 | 56050 | 56051 |
| | 56052 | 56053 | 56054 | 56055 | 56056 | 56057 | 56058 | 56059 | 56060 | 56061 | 56062 | 56063 |
| | 56064 | 56065 | 56066 | 56067 | 56068 | 56069 | 56070 | 56071 | 56072 | 56073 | 56074 | 56075 |
| | 56076 | 56077 | 56078 | 56079 | 56080 | 56084 | 56085 | 56086 | 56087 | 56088 | 56089 | 56090 |
| | 56091 | 56092 | 56093 | 56094 | 56095 | 56096 | 56097 | 56098 | 56099 | 56100 | 56101 | 56102 |
| | 56103 | 56104 | 56105 | 56106 | 56113 | 56114 | 56115 | 56116 | 56117 | 56118 | 56119 | 56120 |
| | 56121 | 56122 | 56123 | 56124 | 56125 | 56126 | 56127 | 56128 | 56129 | 56137 | 56138 | 56139 |
| | 56140 | 56141 | 56142 | 56143 | 56144 | 56145 | 56146 | 56147 | 56148 | 56149 | 56150 | 56151 |
| | 56152 | 56167 | 56168 | 56169 | 56174 | 56175 | 56176 | 56177 | 56178 | 56179 | 56181 | 56182 |
| | 56183 | 56189 | 56190 | 56191 | 56192 | 56193 | 56194 | 56195 | 56196 | 56200 | 56201 | 56202 |
| | 56203 | 56204 | 56205 | 56211 | 56219 | 56220 | 56226 | 56227 | 56228 | 56229 | 56230 | 56231 |
| | 56232 | 56233 | 56234 | 56235 | 56236 | 56237 | 56238 | 56239 | 56240 | 56241 | 56242 | 56243 |
| | 56244 | 56245 | 56246 | 56247 | 56248 | 56249 | 56250 | 56251 | 56252 | 56253 | 56254 | 56255 |
| | 56256 | 56257 | 56258 | 56263 | 56264 | 56265 | 56266 | 56267 | 56268 | 56269 | 56270 | 56278 |
| | 56279 | 56285 | 56286 | 56287 | 56291 | 56292 | 56293 | 56294 | 56304 | 56305 | 56306 | 56318 |
| | 56319 | 56320 | 56321 | 56322 | 56323 | 56324 | 56325 | 56342 | 56343 | 56344 | 56351 | 56352 |
| | 56353 | 56354 | 56355 | 56356 | 56357 | 56358 | 56359 | 56360 | 56361 | 56362 | 56363 | 56364 |
| | 56365 | 56366 | 56367 | 56368 | 56369 | 56370 | 56371 | 56372 | 56373 | 56374 | 56375 | 56376 |
| | 56377 | 56378 | 56379 | 56380 | 56381 | 56382 | 56383 | 56384 | 56385 | 56386 | 56387 | 56388 |
| | 56389 | 56390 | 56391 | 56392 | 56393 | 56394 | 56395 | 56396 | 56397 | 56398 | 56399 | 56400 |
| | 56401 | 56402 | 56403 | 56404 | 56405 | 56406 | 56407 | 56408 | 56409 | 56410 | 56411 | 56415 |
| | 56416 | 56417 | 56418 | 56422 | 56423 | 56424 | 56425 | 56426 | 56427 | 56428 | 56429 | 56430 |
| | 56431 | 56432 | 56433 | 56434 | 56435 | 56436 | 56437 | 56440 | 56441 | 56442 | 56443 | 56444 |
| | 56445 | 56446 | 56447 | 56448 | 56455 | 56456 | 56457 | 56458 | 56459 | 56462 | 56463 | 56464 |
| | 56467 | 56468 | 56469 | 56470 | 56474 | 56475 | 56479 | 56480 | 56481 | 56482 | 56483 | 56484 |
| | 56485 | 56487 | 56488 | 56489 | 56490 | 56491 | 56492 | 56493 | 56494 | 56495 | 56496 | 56497 |
| | 56498 | 56499 | 56503 | 56508 | 56509 | 56510 | 56511 | 56512 | 56513 | 56514 | 56515 | 56516 |
| | 56517 | 56518 | 56519 | 56520 | 56521 | 56522 | 56523 | 56524 | 56529 | 56530 | 56531 | 56532 |
| | 56533 | 56534 | 56535 | 56536 | 56537 | 56538 | 56539 | 56540 | 56543 | 56544 | 56545 | 56546 |
| | 56547 | 56548 | 56549 | 56572 | 56573 | 56574 | 56575 | 56578 | 56579 | 56580 | 56581 | 56582 |
| | 56583 | 56584 | 56589 | 56590 | 56592 | 56602 | 56603 | 56604 | 56605 | 56607 | 56608 | 56609 |
| | 56610 | 56611 | 56612 | 56613 | 56614 | 56615 | 56616 | 56617 | 56618 | 56619 | 56620 | 56621 |
| | 56622 | 56623 | 56624 | 56625 | 56626 | 56627 | 56628 | 56629 | 56630 | 56631 | 56632 | 56633 |
| | 56634 | 56635 | 56636 | 56637 | 56638 | 56639 | 56642 | 56643 | 56644 | 56645 | 56646 | 56647 |
| | 56648 | 56649 | 56650 | 56651 | 56652 | 56653 | 56658 | 56659 | 56660 | 56661 | 56662 | 56665 |
| | 56666 | 56667 | 56670 | 56671 | 56672 | 56673 | 56674 | 56675 | 56676 | 56677 | 56678 | 56679 |
| | 56683 | 56692 | 56693 | 56694 | 56695 | 56696 | 56697 | 56706 | 56707 | 56708 | 56709 | 56712 |
| | 56713 | 56714 | 56715 | 56716 | 56717 | 56727 | 56728 | 56729 | 56730 | 56741 | 56742 | 56743 |
| | 56765 | 56766 | 56767 | 56782 | 56783 | 56784 | 56785 | 56786 | 56787 | 56788 | 56798 | 56799 |
| | 56800 | 56803 | 56804 | 56805 | 56809 | 56810 | 56811 | 56812 | 56813 | 56814 | 56817 | 56818 |
| | 56819 | 56820 | 56821 | 56822 | 56823 | 56824 | 56825 | 56833 | 56834 | 56835 | 56836 | 56837 |
| | 56838 | 56839 | 56840 | 56841 | 56842 | 56845 | 56846 | 56847 | 56851 | 56852 | 56853 | 56854 |
| | 56855 | 56856 | 56857 | 56858 | 56859 | 56860 | 56861 | 56862 | 56863 | 56864 | 56865 | 56869 |
| | 56870 | 56871 | 56872 | 56873 | 56874 | 56875 | 56876 | 56877 | 56878 | 56879 | 56880 | 56881 |
| | 56882 | 56883 | 56884 | 56885 | 56886 | 56887 | 56888 | 56889 | 56890 | 56891 | 56892 | 56893 |
| | 56894 | 56895 | 56896 | 56897 | 56898 | 56899 | 56900 | 56901 | 56902 | 56906 | 56907 | 56908 |
| | 56909 | 56910 | 56911 | 56912 | 56913 | 56914 | 56915 | 56916 | 56917 | 56918 | 56919 | 56920 |
| | 56921 | 56922 | 56923 | 56924 | 56925 | 56926 | 56927 | 56928 | 56929 | 56930 | 56931 | 56932 |
| | 56933 | 56934 | 56935 | 56936 | 56937 | 56941 | 56942 | 56943 | 56944 | 56945 | 56946 | 56949 |
| | 56950 | 56951 | 56952 | 56953 | 56954 | 56955 | 56956 | 56957 | 56958 | 56959 | 56964 | 56965 |
| | 56966 | 56967 | 56968 | 56969 | 56970 | 56971 | 56972 | 56973 | 56974 | 56975 | 56976 | 56977 |
| | 56978 | 56979 | 56980 | 56981 | 56982 | 56983 | 56984 | 56985 | 56986 | 56987 | 56988 | 56993 |
| | 56994 | 56995 | 57000 | 57001 | 57002 | 57003 | 57004 | 57005 | 57006 | 57007 | 57008 | 57009 |
| | 57010 | 57011 | 57012 | 57013 | 57014 | 57015 | 57019 | 57020 | 57021 | 57022 | 57023 | 57024 |
| | 57025 | 57026 | 57027 | 57028 | 57029 | 57030 | 57031 | 57036 | 57037 | 57038 | 57039 | 57040 |
| | 57041 | 57042 | 57043 | 57044 | 57047 | 57048 | 57049 | 57050 | 57051 | 57052 | 57056 | 57057 |
| | 57058 | 57059 | 57060 | 57061 | 57062 | 57063 | 57064 | 57065 | 57066 | 57067 | 57068 | 57069 |
| | 57070 | 57071 | 57072 | 57073 | 57074 | 57075 | 57076 | 57079 | 57080 | 57081 | 57082 | 57083 |
| | 57084 | 57085 | 57086 | 57087 | 57088 | 57089 | 57090 | 57091 | 57092 | 57093 | 57094 | 57095 |
| | 57096 | 57107 | 57108 | 57109 | 57130 | 57131 | 57132 | 57133 | 57134 | 57135 | 57136 | 57139 |
| | 57140 | 57146 | 57147 | 57155 | 57156 | 57157 | 57158 | 57163 | 57164 | 57167 | 57168 | 57169 |
| | 57170 | 57171 | 57172 | 57177 | 57178 | 57179 | 57180 | 57185 | 57186 | 57187 | 57188 | 57189 |
| | 57190 | 57191 | 57192 | 57193 | 57194 | 57195 | 57197 | 57198 | 57203 | 57204 | 57205 | 57222 |
| | 57226 | 57227 | 57228 | 57229 | 57236 | 57237 | 57238 | 57239 | 57240 | 57241 | 57242 | 57243 |
| | 57244 | 57245 | 57246 | 57247 | 57248 | 57249 | 57253 | 57254 | 57255 | 57256 | 57257 | 57258 |
| | 57259 | 57260 | 57261 | 57262 | 57263 | 57264 | 57278 | 57279 | 57280 | 57281 | 57286 | 57287 |
| | 57288 | 57289 | 57290 | 57291 | 57292 | 57293 | 57296 | 57297 | 57301 | 57302 | 57303 | 57304 |
| | 57305 | 57306 | 57307 | 57310 | 57311 | 57312 | 57313 | 57314 | 57315 | 57316 | 57317 | 57322 |
| | 57323 | 57324 | 57325 | 57326 | 57327 | 57328 | 57329 | 57330 | 57331 | 57334 | 57335 | 57336 |
| | 57337 | 57338 | 57339 | 57340 | 57341 | 57342 | 57352 | 57353 | 57354 | 57355 | 57356 | 57357 |
| | 57358 | 57369 | 57370 | 57371 | 57372 | 57373 | 57374 | 57408 | 57409 | 57413 | 57414 | 57415 |
| | 57435 | 57436 | 57437 | 57438 | 57445 | 57446 | 57447 | 57454 | 57455 | 57456 | 57457 | 57458 |
| | 57459 | 57460 | 57465 | 57466 | 57467 | 57468 | 57469 | 57470 | 57471 | 57472 | 57473 | 57474 |
| | 57475 | 57476 | 57477 | 57478 | 57479 | 57487 | 57488 | 57489 | 57490 | 57491 | 57494 | 57495 |
| | 57496 | 57505 | 57506 | 57507 | 57513 | 57514 | 57515 | 57516 | 57517 | 57518 | 57519 | 57520 |
| | 57521 | 57522 | 57523 | 57526 | 57527 | 57528 | 57531 | 57532 | 57533 | 57534 | 57535 | 57536 |
| | 57537 | 57538 | 57539 | 57540 | 57541 | 57542 | 57543 | 57544 | 57545 | 57547 | 57548 | 57549 |
| | 57555 | 57556 | 57557 | 57558 | 57559 | 57562 | 57563 | 57564 | 57565 | 57566 | 57567 | 57568 |
| | 57569 | 57570 | 57571 | 57572 | 57573 | 57576 | 57577 | 57578 | 57579 | 57580 | 57581 | 57582 |
| | 57583 | 57584 | 57585 | 57586 | 57587 | 57588 | 57589 | 57590 | 57591 | 57592 | 57593 | 57594 |
| | 57595 | 57596 | 57597 | 57598 | 57601 | 57602 | 57603 | 57604 | 57605 | 57606 | 57607 | 57608 |
| | 57609 | 57610 | 57611 | 57612 | 57613 | 57614 | 57615 | 57616 | 57617 | 57618 | 57622 | 57623 |
| | 57624 | 57625 | 57626 | 57627 | 57628 | 57629 | 57630 | 57631 | 57632 | 57633 | 57634 | 57635 |
| | 57636 | 57637 | 57638 | 57639 | 57640 | 57646 | 57647 | 57648 | 57649 | 57650 | 57653 | 57654 |
| | 57655 | 57659 | 57660 | 57661 | 57662 | 57663 | 57664 | 57665 | 57666 | 57667 | 57668 | 57669 |
| | 57670 | 57671 | 57672 | 57673 | 57674 | 57675 | 57676 | 57677 | 57678 | 57679 | 57680 | 57681 |
| | 57682 | 57683 | 57686 | 57687 | 57688 | 57690 | 57691 | 57692 | 57693 | 57694 | 57695 | 57700 |
| | 57701 | 57702 | 57703 | 57704 | 57705 | 57709 | 57710 | 57711 | 57712 | 57713 | 57714 | 57715 |
| | 57716 | 57717 | 57718 | 57719 | 57720 | 57728 | 57729 | 57730 | 57731 | 57740 | 57741 | 57742 |
| | 57743 | 57744 | 57745 | 57746 | 57747 | 57748 | 57749 | 57750 | 57751 | 57752 | 57753 | 57754 |
| | 57755 | 57756 | 57757 | 57758 | 57759 | 57760 | 57763 | 57764 | 57765 | 57766 | 57767 | 57768 |
| | 57769 | 57770 | 57771 | 57772 | 57773 | 57774 | 57775 | 57776 | 57777 | 57778 | 57779 | 57780 |
| | 57781 | 57782 | 57783 | 57784 | 57785 | 57786 | 57787 | 57788 | 57789 | 57790 | 57791 | 57792 |
| | 57793 | 57794 | 57803 | 57804 | 57805 | 57808 | 57809 | 57810 | 57811 | 57812 | 57813 | 57816 |
| | 57817 | 57818 | 57819 | 57820 | 57821 | 57824 | 57825 | 57826 | 57827 | 57828 | 57829 | 57830 |
| | 57834 | 57835 | 57836 | 57838 | 57839 | 57840 | 57845 | 57846 | 57847 | 57849 | 57850 | 57851 |
| | 57852 | 57853 | 57854 | 57855 | 57856 | 57857 | 57858 | 57859 | 57860 | 57861 | 57862 | 57863 |
| | 57864 | 57865 | 57866 | 57867 | 57868 | 57869 | 57872 | 57873 | 57874 | 57875 | 57876 | 57877 |
| | 57878 | 57879 | 57880 | 57881 | 57882 | 57883 | 57884 | 57890 | 57891 | 57892 | 57895 | 57896 |
| | 57897 | 57898 | 57899 | 57900 | 57901 | 57904 | 57905 | 57906 | 57907 | 57908 | 57909 | 57910 |
| | 57911 | 57912 | 57913 | 57914 | 57915 | 57916 | 57917 | 57918 | 57919 | 57920 | 57921 | 57923 |
| | 57924 | 57925 | 57926 | 57927 | 57928 | 57929 | 57930 | 57931 | 57932 | 57933 | 57934 | 57935 |
| | 57936 | 57937 | 57938 | 57939 | 57940 | 57941 | 57942 | 57943 | 57944 | 57945 | 57946 | 57947 |
| | 57948 | 57949 | 57950 | 57951 | 57952 | 57953 | 57954 | 57955 | 57956 | 57957 | 57958 | 57959 |
| | 57960 | 57961 | 57962 | 57963 | 57964 | 57965 | 57966 | 57967 | 57968 | 57969 | 57970 | 57971 |
| | 57972 | 57973 | 57974 | 57975 | 57976 | 57977 | 57978 | 57985 | 57986 | 57987 | 57988 | 57989 |
| | 57990 | 57991 | 57992 | 57993 | 57994 | 57995 | 57996 | 57997 | 58004 | 58005 | 58006 | 58007 |
| | 58008 | 58009 | 58010 | 58014 | 58015 | 58016 | 58020 | 58021 | 58022 | 58026 | 58027 | 58028 |
| | 58032 | 58033 | 58034 | 58038 | 58039 | 58040 | 58041 | 58042 | 58043 | 58044 | 58045 | 58046 |
| | 58050 | 58051 | 58052 | 58053 | 58054 | 58055 | 58056 | 58057 | 58058 | 58059 | 58060 | 58061 |
| | 58062 | 58063 | 58064 | 58068 | 58069 | 58070 | 58071 | 58072 | 58073 | 58074 | 58075 | 58076 |
| | 58083 | 58084 | 58085 | 58086 | 58087 | 58088 | 58089 | 58090 | 58091 | 58092 | 58093 | 58094 |
| | 58095 | 58096 | 58097 | 58098 | 58099 | 58100 | 58101 | 58102 | 58103 | 58110 | 58111 | 58112 |
| | 58116 | 58117 | 58118 | 58122 | 58123 | 58124 | 58125 | 58126 | 58127 | 58128 | 58129 | 58130 |
| | 58131 | 58132 | 58133 | 58134 | 58135 | 58136 | 58137 | 58138 | 58139 | 58140 | 58141 | 58142 |
| | 58143 | 58147 | 58148 | 58149 | 58150 | 58151 | 58152 | 58153 | 58154 | 58155 | 58156 | 58157 |
| | 58158 | 58159 | 58160 | 58161 | 58165 | 58166 | 58167 | 58171 | 58172 | 58173 | 58174 | 58175 |
| | 58176 | 58177 | 58178 | 58179 | 58182 | 58183 | 58184 | 58197 | 58198 | 58199 | 58215 | 58216 |
| | 58217 | 58219 | 58220 | 58221 | 58223 | 58224 | 58225 | 58227 | 58228 | 58233 | 58234 | 58235 |
| | 58236 | 58237 | 58238 | 58247 | 58248 | 58249 | 58250 | 58251 | 58252 | 58253 | 58254 | 58255 |
| | 58256 | 58257 | 58258 | 58259 | 58260 | 58261 | 58272 | 58273 | 58274 | 58275 | 58276 | 58277 |
| | 58278 | 58285 | 58286 | 58287 | 58288 | 58289 | 58290 | 58291 | 58292 | 58293 | 58294 | 58295 |
| | 58296 | 58297 | 58298 | 58309 | 58310 | 58321 | 58322 | 58323 | 58324 | 58325 | 58326 | 58327 |
| | 58331 | 58332 | 58333 | 58334 | 58335 | 58336 | 58337 | 58338 | 58339 | 58340 | 58341 | 58342 |
| | 58343 | 58344 | 58345 | 58346 | 58347 | 58348 | 58349 | 58350 | 58359 | 58360 | 58361 | 58362 |
| | 58363 | 58364 | 58365 | 58366 | 58367 | 58368 | 58369 | 58370 | 58371 | 58372 | 58373 | 58374 |
| | 58375 | 58376 | 58377 | 58378 | 58379 | 58380 | 58381 | 58382 | 58383 | 58384 | 58385 | 58390 |
| | 58391 | 58392 | 58393 | 58398 | 58399 | 58400 | 58401 | 58402 | 58403 | 58404 | 58405 | 58409 |
| | 58410 | 58411 | 58412 | 58417 | 58418 | 58419 | 58420 | 58421 | 58422 | 58423 | 58424 | 58425 |
| | 58426 | 58427 | 58428 | 58432 | 58433 | 58434 | 58435 | 58440 | 58441 | 58442 | 58443 | 58444 |
| | 58445 | 58446 | 58447 | 58448 | 58449 | 58450 | 58451 | 58452 | 58453 | 58454 | 58455 | 58456 |
| | 58457 | 58462 | 58463 | 58464 | 58465 | 58466 | 58467 | 58468 | 58469 | 58470 | 58471 | 58472 |
| | 58473 | 58474 | 58475 | 58476 | 58477 | 58478 | 58479 | 58480 | 58481 | 58482 | 58483 | 58484 |
| | 58485 | 58486 | 58487 | 58488 | 58489 | 58490 | 58491 | 58492 | 58493 | 58494 | 58495 | 58496 |
| | 58497 | 58498 | 58499 | 58500 | 58501 | 58502 | 58503 | 58504 | 58505 | 58506 | 58507 | 58508 |
| | 58509 | 58510 | 58511 | 58512 | 58513 | 58514 | 58515 | 58516 | 58517 | 58522 | 58523 | 58524 |
| | 58525 | 58526 | 58527 | 58528 | 58529 | 58530 | 58531 | 58532 | 58533 | 58534 | 58535 | 58536 |
| | 58537 | 58538 | 58539 | 58540 | 58541 | 58542 | 58543 | 58548 | 58549 | 58550 | 58551 | 58552 |
| | 58553 | 58554 | 58555 | 58556 | 58557 | 58558 | 58559 | 58560 | 58561 | 58562 | 58563 | 58564 |
| | 58565 | 58566 | 58567 | 58568 | 58569 | 58570 | 58571 | 58572 | 58573 | 58574 | 58575 | 58576 |
| | 58577 | 58580 | 58581 | 58582 | 58583 | 58586 | 58587 | 58589 | 58590 | 58591 | 58592 | 58593 |
| | 58594 | 58595 | 58596 | 58597 | 58598 | 58599 | 58600 | 58601 | 58602 | 58603 | 58604 | 58605 |
| | 58606 | 58607 | 58608 | 58609 | 58610 | 58611 | 58612 | 58613 | 58614 | 58615 | 58616 | 58617 |
| | 58618 | 58619 | 58620 | 58621 | 58622 | 58623 | 58624 | 58625 | 58626 | 58627 | 58628 | 58629 |
| | 58630 | 58631 | 58632 | 58633 | 58634 | 58635 | 58636 | 58637 | 58638 | 58639 | 58640 | 58647 |
| | 58648 | 58649 | 58650 | 58651 | 58652 | 58653 | 58654 | 58655 | 58656 | 58657 | 58658 | 58659 |
| | 58660 | 58662 | 58663 | 58664 | 58665 | 58666 | 58667 | 58668 | 58669 | 58670 | 58671 | 58672 |
| | 58673 | 58674 | 58675 | 58676 | 58677 | 58678 | 58679 | 58680 | 58681 | 58682 | 58683 | 58684 |
| | 58685 | 58686 | 58687 | 58688 | 58689 | 58690 | 58691 | 58692 | 58693 | 58694 | 58695 | 58696 |
| | 58697 | 58698 | 58699 | 58700 | 58710 | 58711 | 58715 | 58716 | 58717 | 58718 | 58719 | 58722 |
| | 58723 | 58724 | 58725 | 58726 | 58727 | 58728 | 58748 | 58749 | 58750 | 58751 | 58753 | 58754 |
| | 58755 | 58767 | 58788 | 58789 | 58790 | 58791 | 58792 | 58793 | 58794 | 58795 | 58796 | 58797 |
| | 58802 | 58803 | 58804 | 58805 | 58809 | 58810 | 58811 | 58812 | 58813 | 58814 | 58815 | 58818 |
| | 58819 | 58820 | 58821 | 58822 | 58823 | 58824 | 58827 | 58828 | 58829 | 58830 | 58831 | 58832 |
| | 58833 | 58834 | 58835 | 58836 | 58837 | 58838 | 58839 | 58840 | 58847 | 58848 | 58849 | 58850 |
| | 58851 | 58852 | 58856 | 58857 | 58858 | 58863 | 58874 | 58875 | 58876 | 58877 | 58911 | 58912 |
| | 58913 | 58914 | 58915 | 58916 | 58917 | 58922 | 58923 | 58924 | 58925 | 58926 | 58927 | 58928 |
| | 58929 | 58930 | 58931 | 58932 | 58933 | 58934 | 58935 | 58936 | 58937 | 58938 | 58939 | 58942 |
| | 58943 | 58944 | 58945 | 58946 | 58947 | 58948 | 58949 | 58950 | 58951 | 58952 | 58953 | 58954 |
| | 58955 | 58956 | 58957 | 58958 | 58961 | 58962 | 58963 | 58964 | 58965 | 58966 | 58967 | 58968 |
| | 58969 | 58971 | 58972 | 58973 | 58974 | 58975 | 58976 | 58977 | 58978 | 58979 | 58980 | 58981 |
| | 58982 | 58983 | 58984 | 58985 | 58986 | 58987 | 58988 | 58989 | 58990 | 58991 | 58992 | 58993 |
| | 58994 | 58995 | 58996 | 58997 | 58998 | 58999 | 59000 | 59001 | 59002 | 59003 | 59004 | 59005 |
| | 59006 | 59007 | 59011 | 59012 | 59013 | 59014 | 59015 | 59016 | 59017 | 59019 | 59020 | 59021 |
| | 59022 | 59023 | 59024 | 59025 | 59026 | 59027 | 59028 | 59029 | 59030 | 59031 | 59032 | 59033 |
| | 59034 | 59035 | 59036 | 59037 | 59038 | 59039 | 59040 | 59041 | 59042 | 59043 | 59044 | 59045 |
| | 59046 | 59047 | 59048 | 59049 | 59050 | 59051 | 59052 | 59053 | 59054 | 59055 | 59056 | 59057 |
| | 59058 | 59059 | 59060 | 59061 | 59062 | 59063 | 59064 | 59065 | 59066 | 59067 | 59068 | 59069 |
| | 59070 | 59071 | 59072 | 59073 | 59074 | 59075 | 59076 | 59077 | 59078 | 59079 | 59080 | 59081 |
| | 59082 | 59083 | 59084 | 59085 | 59086 | 59087 | 59088 | 59089 | 59090 | 59091 | 59092 | 59093 |
| | 59094 | 59095 | 59101 | 59102 | 59103 | 59104 | 59105 | 59106 | 59107 | 59108 | 59109 | 59110 |
| | 59111 | 59112 | 59113 | 59116 | 59117 | 59118 | 59119 | 59120 | 59121 | 59125 | 59126 | 59127 |
| | 59128 | 59129 | 59130 | 59131 | 59132 | 59133 | 59134 | 59135 | 59136 | 59137 | 59138 | 59139 |
| | 59140 | 59141 | 59142 | 59143 | 59144 | 59145 | 59146 | 59147 | 59148 | 59149 | 59150 | 59151 |
| | 59152 | 59153 | 59154 | 59155 | 59156 | 59157 | 59158 | 59159 | 59160 | 59161 | 59162 | 59163 |
| | 59164 | 59165 | 59166 | 59167 | 59168 | 59171 | 59172 | 59173 | 59174 | 59175 | 59176 | 59177 |
| | 59178 | 59181 | 59182 | 59183 | 59186 | 59187 | 59188 | 59189 | 59190 | 59191 | 59192 | 59193 |
| | 59194 | 59195 | 59196 | 59197 | 59198 | 59199 | 59200 | 59201 | 59202 | 59203 | 59204 | 59205 |
| | 59206 | 59207 | 59208 | 59209 | 59210 | 59211 | 59212 | 59213 | 59214 | 59215 | 59218 | 59219 |
| | 59220 | 59221 | 59222 | 59223 | 59224 | 59225 | 59226 | 59227 | 59228 | 59229 | 59230 | 59231 |
| | 59232 | 59233 | 59234 | 59235 | 59238 | 59239 | 59240 | 59241 | 59242 | 59243 | 59245 | 59246 |
| | 59247 | 59248 | 59249 | 59250 | 59251 | 59252 | 59253 | 59254 | 59255 | 59256 | 59257 | 59258 |
| | 59259 | 59260 | 59261 | 59262 | 59263 | 59264 | 59265 | 59266 | 59267 | 59268 | 59269 | 59270 |
| | 59271 | 59272 | 59273 | 59274 | 59275 | 59276 | 59277 | 59278 | 59279 | 59280 | 59281 | 59282 |
| | 59283 | 59284 | 59285 | 59286 | 59287 | 59288 | 59289 | 59290 | 59291 | 59292 | 59293 | 59294 |
| | 59295 | 59296 | 59297 | 59298 | 59299 | 59302 | 59303 | 59304 | 59305 | 59306 | 59307 | 59308 |
| | 59309 | 59310 | 59311 | 59312 | 59313 | 59314 | 59315 | 59316 | 59317 | 59318 | 59319 | 59320 |
| | 59321 | 59322 | 59323 | 59324 | 59325 | 59326 | 59327 | 59328 | 59329 | 59330 | 59331 | 59332 |
| | 59337 | 59338 | 59339 | 59340 | 59341 | 59342 | 59343 | 59346 | 59347 | 59348 | 59349 | 59350 |
| | 59351 | 59352 | 59353 | 59354 | 59355 | 59356 | 59357 | 59358 | 59359 | 59360 | 59361 | 59362 |
| | 59363 | 59364 | 59365 | 59366 | 59369 | 59370 | 59371 | 59372 | 59373 | 59374 | 59375 | 59376 |
| | 59377 | 59378 | 59379 | 59380 | 59381 | 59382 | 59383 | 59384 | 59385 | 59386 | 59393 | 59394 |
| | 59395 | 59396 | 59397 | 59398 | 59399 | 59402 | 59403 | 59404 | 59405 | 59406 | 59407 | 59408 |
| | 59409 | 59410 | 59411 | 59412 | 59413 | 59414 | 59415 | 59416 | 59417 | 59418 | 59419 | 59420 |
| | 59421 | 59422 | 59423 | 59424 | 59425 | 59426 | 59427 | 59428 | 59429 | 59430 | 59431 | 59432 |
| | 59433 | 59434 | 59435 | 59439 | 59440 | 59441 | 59442 | 59443 | 59444 | 59445 | 59446 | 59447 |
| | 59448 | 59449 | 59450 | 59451 | 59452 | 59453 | 59454 | 59455 | 59456 | 59457 | 59458 | 59459 |
| | 59460 | 59461 | 59462 | 59463 | 59464 | 59465 | 59466 | 59467 | 59468 | 59469 | 59470 | 59471 |
| | 59472 | 59473 | 59474 | 59475 | 59476 | 59477 | 59478 | 59479 | 59480 | 59481 | 59482 | 59483 |
| | 59484 | 59485 | 59486 | 59487 | 59488 | 59489 | 59490 | 59491 | 59492 | 59493 | 59494 | 59495 |
| | 59496 | 59497 | 59498 | 59499 | 59500 | 59501 | 59502 | 59503 | 59504 | 59505 | 59506 | 59507 |
| | 59508 | 59509 | 59510 | 59511 | 59512 | 59513 | 59514 | 59515 | 59516 | 59517 | 59518 | 59519 |
| | 59520 | 59521 | 59522 | 59523 | 59524 | 59525 | 59526 | 59527 | 59528 | 59529 | 59530 | 59531 |
| | 59532 | 59533 | 59534 | 59535 | 59536 | 59537 | 59538 | 59539 | 59540 | 59541 | 59542 | 59543 |
| | 59544 | 59545 | 59546 | 59547 | 59548 | 59549 | 59550 | 59551 | 59552 | 59553 | 59554 | 59555 |
| | 59556 | 59557 | 59558 | 59559 | 59560 | 59561 | 59562 | 59563 | 59564 | 59565 | 59566 | 59567 |
| | 59568 | 59569 | 59570 | 59571 | 59572 | 59573 | 59574 | 59575 | 59576 | 59577 | 59578 | 59579 |
| | 59580 | 59581 | 59582 | 59583 | 59584 | 59585 | 59586 | 59587 | 59588 | 59589 | 59590 | 59591 |
| | 59592 | 59593 | 59594 | 59595 | 59596 | 59597 | 59598 | 59599 | 59600 | 59601 | 59602 | 59603 |
| | 59604 | 59605 | 59606 | 59607 | 59608 | 59609 | 59610 | 59611 | 59612 | 59613 | 59614 | 59615 |
| | 59616 | 59617 | 59618 | 59619 | 59620 | 59621 | 59622 | 59623 | 59624 | 59625 | 59626 | 59627 |
| | 59628 | 59629 | 59630 | 59631 | 59632 | 59633 | 59634 | 59635 | 59636 | 59637 | 59638 | 59639 |
| | 59640 | 59641 | 59642 | 59643 | 59644 | 59645 | 59646 | 59647 | 59648 | 59649 | 59650 | 59651 |
| | 59652 | 59653 | 59654 | 59655 | 59656 | 59657 | 59658 | 59659 | 59660 | 59661 | 59662 | 59663 |
| | 59664 | 59665 | 59666 | 59667 | 59668 | 59669 | 59670 | 59671 | 59672 | 59673 | 59674 | 59675 |
| | 59676 | 59677 | 59678 | 59679 | 59680 | 59681 | 59682 | 59683 | 59684 | 59685 | 59686 | 59687 |
| | 59688 | 59689 | 59690 | 59691 | 59692 | 59693 | 59694 | 59695 | 59696 | 59697 | 59698 | 59699 |
| | 59700 | 59701 | 59702 | 59703 | 59704 | 59705 | 59706 | 59707 | 59708 | 59709 | 59710 | 59711 |
| | 59712 | 59713 | 59714 | 59715 | 59716 | 59717 | 59718 | 59719 | 59720 | 59721 | 59722 | 59723 |
| | 59724 | 59725 | 59726 | 59727 | 59728 | 59729 | 59730 | 59731 | 59732 | 59733 | 59734 | 59735 |
| | 59736 | 59737 | 59738 | 59739 | 59740 | 59741 | 59742 | 59743 | 59744 | 59745 | 59746 | 59747 |
| | 59748 | 59749 | 59750 | 59751 | 59752 | 59753 | 59754 | 59755 | 59756 | 59757 | 59758 | 59759 |
| | 59760 | 59761 | 59762 | 59763 | 59764 | 59765 | 59766 | 59767 | 59768 | 59769 | 59770 | 59771 |
| | 59772 | 59773 | 59774 | 59775 | 59776 | 59777 | 59778 | 59779 | 59780 | 59781 | 59782 | 59783 |
| | 59784 | 59785 | 59786 | 59787 | 59788 | 59789 | 59790 | 59791 | 59792 | 59793 | 59794 | 59795 |
| | 59796 | 59797 | 59798 | 59799 | 59800 | 59801 | 59802 | 59803 | 59804 | 59805 | 59806 | 59807 |
| | 59808 | 59809 | 59810 | 59811 | 59812 | 59814 | 59815 | 59816 | 59817 | 59818 | 59819 | 59820 |
| | 59821 | 59822 | 59823 | 59824 | 59825 | 59826 | 59827 | 59828 | 59829 | 59830 | 59831 | 59832 |
| | 59833 | 59834 | 59835 | 59836 | 59837 | 59838 | 59839 | 59840 | 59841 | 59848 | 59849 | 59850 |
| | 59851 | 59852 | 59853 | 59854 | 59855 | 59856 | 59857 | 59858 | 59859 | 59860 | 59861 | 59862 |
| | 59863 | 59864 | 59865 | 59866 | 59867 | 59868 | 59869 | 59870 | 59871 | 59872 | 59873 | 59874 |
| | 59875 | 59876 | 59877 | 59878 | 59879 | 59880 | 59881 | 59882 | 59883 | 59884 | 59885 | 59886 |
| | 59887 | 59888 | 59889 | 59890 | 59891 | 59892 | 59893 | 59894 | 59895 | 59896 | 59897 | 59898 |
| | 59899 | 59900 | 59901 | 59902 | 59903 | 59904 | 59905 | 59906 | 59907 | 59908 | 59911 | 59912 |
| | 59913 | 59914 | 59915 | 59916 | 59917 | 59918 | 59919 | 59920 | 59921 | 59922 | 59923 | 59924 |
| | 59925 | 59926 | 59927 | 59928 | 59929 | 59930 | 59931 | 59932 | 59933 | 59934 | 59935 | 59936 |
| | 59937 | 59938 | 59939 | 59940 | 59941 | 59942 | 59943 | 59944 | 59945 | 59946 | 59947 | 59948 |
| | 59949 | 59950 | 59951 | 59952 | 59953 | 59954 | 59955 | 59956 | 59957 | 59961 | 59962 | 59963 |
| | 59964 | 59965 | 59966 | 59967 | 59968 | 59969 | 59970 | 59971 | 59972 | 59973 | 59974 | 59975 |
| | 59976 | 59977 | 59978 | 59979 | 59980 | 59981 | 59982 | 59983 | 59984 | 59985 | 59986 | 59987 |
| | 59988 | 59989 | 59990 | 59991 | 59992 | 59993 | 59994 | 59995 | 59996 | 59997 | 59998 | 59999 |
| | 60000 | 60001 | 60002 | 60003 | 60004 | 60005 | 60006 | 60007 | 60008 | 60009 | 60010 | 60011 |
| | 60012 | 60013 | 60014 | 60015 | 60016 | 60017 | 60018 | 60019 | 60020 | 60021 | 60022 | 60023 |
| | 60024 | 60025 | 60026 | 60027 | 60028 | 60029 | 60030 | 60031 | 60032 | 60033 | 60034 | 60035 |
| | 60036 | 60037 | 60038 | 60039 | 60040 | 60041 | 60042 | 60043 | 60044 | 60045 | 60046 | 60047 |
| | 60048 | 60049 | 60050 | 60051 | 60052 | 60053 | 60054 | 60055 | 60056 | 60057 | 60058 | 60059 |
| | 60060 | 60061 | 60062 | 60063 | 60064 | 60065 | 60066 | 60067 | 60068 | 60069 | 60070 | 60071 |
| | 60072 | 60073 | 60074 | 60075 | 60076 | 60077 | 60078 | 60079 | 60080 | 60081 | 60086 | 60087 |
| | 60088 | 60089 | 60090 | 60091 | 60092 | 60093 | 60094 | 60095 | 60096 | 60097 | 60098 | 60099 |
| | 60100 | 60101 | 60102 | 60103 | 60104 | 60105 | 60106 | 60107 | 60108 | 60109 | 60110 | 60111 |
| | 60112 | 60113 | 60114 | 60115 | 60116 | 60117 | 60118 | 60119 | 60120 | 60121 | 60122 | 60123 |
| | 60124 | 60125 | 60126 | 60127 | 60128 | 60129 | 60130 | 60131 | 60132 | 60133 | 60134 | 60135 |
| | 60136 | 60137 | 60138 | 60139 | 60140 | 60141 | 60142 | 60143 | 60144 | 60145 | 60146 | 60147 |
| | 60148 | 60149 | 60150 | 60151 | 60152 | 60153 | 60154 | 60155 | 60156 | 60157 | 60158 | 60159 |
| | 60160 | 60161 | 60162 | 60163 | 60164 | 60165 | 60166 | 60167 | 60168 | 60169 | 60170 | 60171 |
| | 60172 | 60173 | 60174 | 60175 | 60176 | 60177 | 60178 | 60179 | 60180 | 60181 | 60182 | 60183 |
| | 60184 | 60185 | 60186 | 60187 | 60188 | 60189 | 60190 | 60191 | 60192 | 60193 | 60194 | 60195 |
| | 60196 | 60197 | 60198 | 60199 | 60200 | 60201 | 60202 | 60203 | 60204 | 60205 | 60206 | 60207 |
| | 60208 | 60209 | 60210 | 60211 | 60212 | 60213 | 60214 | 60215 | 60216 | 60217 | 60218 | 60219 |
| | 60220 | 60221 | 60223 | 60224 | 60225 | 60226 | 60227 | 60228 | 60229 | 60230 | 60231 | 60232 |
| | 60233 | 60234 | 60235 | 60236 | 60237 | 60238 | 60239 | 60240 | 60241 | 60242 | 60243 | 60244 |
| | 60245 | 60246 | 60247 | 60248 | 60249 | 60250 | 60251 | 60252 | 60253 | 60254 | 60255 | 60256 |
| | 60257 | 60258 | 60259 | 60260 | 60261 | 60262 | 60263 | 60264 | 60265 | 60266 | 60267 | 60269 |
| | 60270 | 60271 | 60272 | 60273 | 60274 | 60275 | 60276 | 60277 | 60278 | 60279 | 60280 | 60281 |
| | 60282 | 60283 | 60284 | 60285 | 60286 | 60287 | 60288 | 60289 | 60290 | 60291 | 60292 | 60293 |
| | 60298 | 60299 | 60300 | 60301 | 60302 | 60303 | 60304 | 60305 | 60306 | 60307 | 60308 | 60309 |
| | 60310 | 60311 | 60312 | 60313 | 60314 | 60315 | 60316 | 60317 | 60318 | 60319 | 60320 | 60321 |
| | 60322 | 60323 | 60324 | 60325 | 60326 | 60327 | 60328 | 60329 | 60330 | 60333 | 60334 | 60335 |
| | 60336 | 60340 | 60341 | 60342 | 60343 | 60344 | 60345 | 60346 | 60347 | 60348 | 60349 | 60350 |
| | 60351 | 60352 | 60353 | 60354 | 60355 | 60358 | 60359 | 60360 | 60365 | 60366 | 60367 | 60368 |
| | 60369 | 60370 | 60371 | 60372 | 60373 | 60374 | 60375 | 60376 | 60377 | 60378 | 60379 | 60380 |
| | 60381 | 60382 | 60386 | 60387 | 60388 | 60392 | 60393 | 60394 | 60395 | 60396 | 60397 | 60398 |
| | 60399 | 60400 | 60401 | 60402 | 60403 | 60404 | 60405 | 60406 | 60407 | 60408 | 60409 | 60410 |
| | 60411 | 60412 | 60413 | 60418 | 60419 | 60420 | 60421 | 60422 | 60423 | 60424 | 60425 | 60426 |
| | 60427 | 60428 | 60429 | 60430 | 60431 | 60432 | 60433 | 60434 | 60435 | 60436 | 60437 | 60438 |
| | 60439 | 60440 | 60441 | 60442 | 60443 | 60444 | 60447 | 60448 | 60449 | 60450 | 60451 | 60452 |
| | 60453 | 60454 | 60455 | 60456 | 60457 | 60458 | 60459 | 60460 | 60469 | 60470 | 60471 | 60472 |
| | 60473 | 60474 | 60475 | 60480 | 60481 | 60482 | 60483 | 60484 | 60485 | 60486 | 60487 | 60488 |
| | 60489 | 60490 | 60492 | 60493 | 60494 | 60495 | 60496 | 60497 | 60498 | 60499 | 60500 | 60501 |
| | 60502 | 60503 | 60504 | 60505 | 60506 | 60513 | 60514 | 60515 | 60516 | 60517 | 60518 | 60519 |
| | 60520 | 60521 | 60522 | 60523 | 60524 | 60525 | 60526 | 60527 | 60528 | 60529 | 60530 | 60531 |
| | 60532 | 60533 | 60534 | 60535 | 60536 | 60537 | 60538 | 60539 | 60540 | 60541 | 60542 | 60544 |
| | 60545 | 60546 | 60547 | 60548 | 60549 | 60550 | 60551 | 60552 | 60553 | 60554 | 60555 | 60556 |
| | 60557 | 60558 | 60559 | 60560 | 60561 | 60562 | 60563 | 60564 | 60565 | 60566 | 60567 | 60568 |
| | 60570 | 60571 | 60572 | 60573 | 60574 | 60575 | 60576 | 60577 | 60578 | 60579 | 60580 | 60581 |
| | 60582 | 60583 | 60584 | 60585 | 60586 | 60587 | 60588 | 60589 | 60590 | 60591 | 60592 | 60593 |
| | 60594 | 60595 | 60596 | 60597 | 60598 | 60603 | 60604 | 60605 | 60606 | 60607 | 60608 | 60609 |
| | 60619 | 60620 | 60621 | 60622 | 60623 | 60638 | 60639 | 60640 | 60660 | 60661 | 60665 | 60666 |
| | 60667 | 60669 | 60670 | 60680 | 60681 | 60682 | 60683 | 60684 | 60686 | 60687 | 60688 | 60689 |
| | 60696 | 60699 | 60700 | 60702 | 60703 | 60704 | 60705 | 60706 | 60707 | 60708 | 60709 | 60710 |
| | 60711 | 60712 | 60713 | 60714 | 60715 | 60716 | 60720 | 60721 | 60722 | 60723 | 60724 | 60725 |
| | 60726 | 60730 | 60731 | 60732 | 60733 | 60734 | 60735 | 60736 | 60737 | 60738 | 60739 | 60740 |
| | 60741 | 60742 | 60743 | 60744 | 60745 | 60746 | 60750 | 60752 | 60753 | 60756 | 60757 | 60758 |
| | 60759 | 60760 | 60761 | 60762 | 60767 | 60768 | 60769 | 60770 | 60771 | 60772 | 60780 | 60781 |
| | 60782 | 60783 | 60793 | 60794 | 60795 | 60796 | 60797 | 60798 | 60799 | 60800 | 60801 | 60802 |
| | 60803 | 60804 | 60805 | 60806 | 60807 | 60808 | 60809 | 60810 | 60811 | 60812 | 60813 | 60816 |
| | 60817 | 60818 | 60819 | 60820 | 60821 | 60822 | 60823 | 60824 | 60825 | 60826 | 60827 | 60828 |
| | 60829 | 60830 | 60831 | 60832 | 60833 | 60834 | 60835 | 60836 | 60837 | 60838 | 60839 | 60840 |
| | 60841 | 60842 | 60846 | 60847 | 60848 | 60849 | 60850 | 60851 | 60852 | 60853 | 60854 | 60855 |
| | 60856 | 60857 | 60858 | 60859 | 60860 | 60861 | 60862 | 60863 | 60864 | 60865 | 60866 | 60867 |
| | 60868 | 60869 | 60870 | 60871 | 60872 | 60873 | 60877 | 60878 | 60879 | 60880 | 60881 | 60882 |
| | 60883 | 60884 | 60885 | 60886 | 60887 | 60888 | 60889 | 60890 | 60891 | 60892 | 60893 | 60899 |
| | 60900 | 60901 | 60902 | 60905 | 60906 | 60907 | 60910 | 60911 | 60912 | 60913 | 60914 | 60915 |
| | 60916 | 60948 | 60949 | 60950 | 60951 | 60961 | 60962 | 60963 | 60964 | 60965 | 60969 | 60970 |
| | 60991 | 60992 | 60993 | 60995 | 60996 | 61003 | 61004 | 61005 | 61007 | 61008 | 61009 | 61010 |
| | 61011 | 61012 | 61013 | 61014 | 61015 | 61017 | 61018 | 61019 | 61020 | 61021 | 61022 | 61024 |
| | 61025 | 61026 | 61032 | 61033 | 61034 | 61035 | 61036 | 61037 | 61038 | 61039 | 61040 | 61041 |
| | 61042 | 61043 | 61044 | 61045 | 61046 | 61047 | 61048 | 61049 | 61050 | 61051 | 61052 | 61053 |
| | 61054 | 61055 | 61056 | 61057 | 61058 | 61059 | 61060 | 61061 | 61062 | 61063 | 61064 | 61065 |
| | 61066 | 61067 | 61068 | 61069 | 61070 | 61071 | 61072 | 61073 | 61074 | 61075 | 61076 | 61077 |
| | 61078 | 61079 | 61080 | 61081 | 61082 | 61084 | 61085 | 61086 | 61087 | 61088 | 61089 | 61090 |
| | 61091 | 61092 | 61100 | 61101 | 61102 | 61103 | 61104 | 61105 | 61107 | 61108 | 61109 | 61110 |
| | 61111 | 61122 | 61123 | 61124 | 61125 | 61132 | 61133 | 61134 | 61135 | 61145 | 61146 | 61147 |
| | 61148 | 61149 | 61150 | 61153 | 61154 | 61155 | 61160 | 61161 | 61163 | 61164 | 61165 | 61166 |
| | 61172 | 61173 | 61174 | 61178 | 61179 | 61180 | 61189 | 61198 | 61199 | 61200 | 61218 | 61219 |
| | 61220 | 61244 | 61245 | 61246 | 61247 | 61248 | 61249 | 61260 | 61261 | 61262 | 61271 | 61272 |
| | 61273 | 61274 | 61275 | 61277 | 61278 | 61281 | 61282 | 61283 | 61290 | 61291 | 61292 | 61293 |
| | 61294 | 61297 | 61298 | 61299 | 61300 | 61301 | 61302 | 61303 | 61304 | 61305 | 61306 | 61307 |
| | 61308 | 61309 | 61310 | 61311 | 61312 | 61313 | 61314 | 61318 | 61319 | 61320 | 61321 | 61322 |
| | 61323 | 61324 | 61325 | 61326 | 61327 | 61329 | 61330 | 61332 | 61333 | 61334 | 61335 | 61336 |
| | 61337 | 61338 | 61339 | 61340 | 61341 | 61342 | 61343 | 61344 | 61345 | 61346 | 61347 | 61348 |
| | 61349 | 61350 | 61351 | 61352 | 61353 | 61354 | 61355 | 61356 | 61357 | 61358 | 61359 | 61360 |
| | 61361 | 61362 | 61363 | 61364 | 61365 | 61366 | 61367 | 61368 | 61369 | 61370 | 61371 | 61372 |
| | 61373 | 61376 | 61377 | 61381 | 61382 | 61405 | 61406 | 61407 | 61408 | 61441 | 61442 | 61443 |
| | 61450 | 61451 | 61452 | 61453 | 61454 | 61455 | 61457 | 61458 | 61459 | 61460 | 61461 | 61463 |
| | 61465 | 61466 | 61467 | 61468 | 61469 | 61470 | 61471 | 61472 | 61473 | 61481 | 61482 | 61483 |
| | 61484 | 61485 | 61488 | 61489 | 61490 | 61491 | 61495 | 61496 | 61497 | 61498 | 61502 | 61503 |
| | 61504 | 61505 | 61506 | 61507 | 61508 | 61509 | 61510 | 61512 | 61513 | 61514 | 61515 | 61516 |
| | 61517 | 61518 | 61519 | 61520 | 61521 | 61522 | 61523 | 61524 | 61525 | 61526 | 61527 | 61529 |
| | 61530 | 61535 | 61536 | 61537 | 61539 | 61540 | 61541 | 61542 | 61543 | 61544 | 61545 | 61546 |
| | 61547 | 61548 | 61549 | 61550 | 61551 | 61552 | 61553 | 61554 | 61555 | 61556 | 61557 | 61558 |
| | 61559 | 61560 | 61561 | 61562 | 61563 | 61564 | 61565 | 61566 | 61567 | 61568 | 61569 | 61570 |
| | 61571 | 61572 | 61573 | 61574 | 61575 | 61576 | 61577 | 61578 | 61579 | 61580 | 61581 | 61582 |
| | 61583 | 61584 | 61585 | 61586 | 61587 | 61588 | 61589 | 61591 | 61592 | 61593 | 61594 | 61595 |
| | 61596 | 61597 | 61598 | 61599 | 61602 | 61603 | 61605 | 61606 | 61607 | 61609 | 61610 | 61611 |
| | 61612 | 61613 | 61614 | 61615 | 61616 | 61617 | 61618 | 61619 | 61620 | 61621 | 61624 | 61625 |
| | 61626 | 61627 | 61628 | 61629 | 61631 | 61632 | 61634 | 61635 | 61636 | 61637 | 61638 | 61639 |
| | 61640 | 61641 | 61642 | 61643 | 61644 | 61646 | 61647 | 61648 | 61651 | 61652 | 61653 | 61654 |
| | 61655 | 61660 | 61661 | 61662 | 61666 | 61667 | 61668 | 61669 | 61670 | 61672 | 61673 | 61674 |
| | 61675 | 61676 | 61677 | 61678 | 61683 | 61684 | 61685 | 61686 | 61687 | 61688 | 61689 | 61691 |
| | 61692 | 61693 | 61694 | 61703 | 61704 | 61705 | 61706 | 61707 | 61708 | 61718 | 61719 | 61720 |
| | 61721 | 61722 | 61723 | 61724 | 61725 | 61726 | 61727 | 61728 | 61729 | 61730 | 61731 | 61732 |
| | 61733 | 61734 | 61739 | 61740 | 61744 | 61745 | 61748 | 61749 | 61758 | 61759 | 61765 | 61766 |
| | 61767 | 61768 | 61769 | 61770 | 61774 | 61775 | 61782 | 61783 | 61784 | 61785 | 61786 | 61787 |
| | 61788 | 61789 | 61791 | 61792 | 61794 | 61795 | 61797 | 61798 | 61799 | 61803 | 61804 | 61805 |
| | 61807 | 61808 | 61809 | 61810 | 61814 | 61815 | 61816 | 61817 | 61823 | 61824 | 61825 | 61826 |
| | 61827 | 61828 | 61834 | 61835 | 61844 | 61845 | 61847 | 61848 | 61849 | 61850 | 61852 | 61853 |
| | 61858 | 61859 | 61860 | 61861 | 61867 | 61868 | 61869 | 61870 | 61872 | 61873 | 61875 | 61876 |
| | 61877 | 61878 | 61879 | 61880 | 61881 | 61884 | 61885 | 61886 | 61887 | 61889 | 61890 | 61892 |
| | 61893 | 61894 | 61899 | 61900 | 61903 | 61904 | 61905 | 61906 | 61907 | 61908 | 61916 | 61917 |
| | 61921 | 61922 | 61923 | 61932 | 61933 | 61937 | 61938 | 61942 | 61943 | 61946 | 61947 | 61948 |
| | 61949 | 61950 | 61951 | 61952 | 61953 | 61954 | 61955 | 61957 | 61958 | 61960 | 61961 | 61963 |
| | 61964 | 61965 | 61966 | 61967 | 61968 | 61972 | 61973 | 61974 | 61975 | 61977 | 61978 | 61980 |
| | 61981 | 61984 | 61985 | 61986 | 61987 | 1988 | 61991 | 61992 | 61993 | 61995 | 61996 | 52011 |
| | 62012 | 62013 | 62020 | 62021 | 62023 | 62024 | 62025 | 62026 | 62027 | 62028 | 62029 | 62030 |
| | 62031 | 62032 | 62033 | 62034 | 62035 | 62036 | 62037 | 62038 | 62039 | 62040 | 62041 | 62042 |
| | 62046 | 62047 | 62048 | 62062 | 62069 | 62070 | 62071 | 62075 | 62101 | 62102 | 62114 | 62115 |
| | 62116 | 62118 | 62119 | 62120 | 62121 | 62123 | 62124 | 62125 | 62126 | 62127 | 62130 | 62131 |
| | 62132 | 62138 | 62139 | 62140 | 62141 | 62142 | 62143 | 62149 | 62150 | 62151 | 62152 | 62153 |
| | 62154 | 62158 | 62159 | 62160 | 62161 | 62162 | 62163 | 62164 | 62170 | 62171 | 62172 | 62173 |
| | 62174 | 62177 | 62178 | 62179 | 62180 | 62181 | 62182 | 62184 | 62185 | 62186 | 62187 | 62188 |
| | 62194 | 62195 | 62198 | 62199 | 62200 | 62201 | 62205 | 62206 | 62207 | 62225 | 62226 | 62238 |
| | 62239 | 62244 | 62245 | 62246 | 62247 | 62248 | 62249 | 62250 | 62251 | 62253 | 62254 | 62264 |
| | 62266 | 62267 | 62268 | 62269 | 62271 | 62272 | 62273 | 62274 | 62276 | 62277 | 62278 | 62290 |
| | 62291 | 62296 | 62297 | 62304 | 62305 | 62306 | 62307 | 62309 | 62310 | 62312 | 62313 | 62314 |
| | 62315 | 62316 | 62317 | 62319 | 62320 | 62321 | 62322 | 62323 | 62324 | 62328 | 62329 | 62330 |
| | 62331 | 62346 | 62347 | 62348 | 62357 | 62363 | 62364 | 62365 | 62366 | 62371 | 62372 | 62373 |
| | 62374 | 62375 | 62376 | 62377 | 62378 | 62379 | 62380 | 62381 | 62382 | 62383 | 62387 | 62388 |
| | 62397 | 62398 | 62399 | 62400 | 62401 | 62402 | 62403 | 62404 | 62416 | 62417 | 62418 | 62419 |
| | 62423 | 62424 | 62425 | 62426 | 62427 | 62428 | 62429 | 62430 | 62431 | 62432 | 62435 | 62455 |
| | 62456 | 62457 | 62458 | 62459 | 62460 | 62461 | 62462 | 62466 | 62476 | 62477 | 62478 | 62479 |
| | 62480 | 62504 | 62505 | 62506 | 62507 | 62508 | 62515 | 62516 | 62517 | 62530 | 62531 | 62532 |
| | 62533 | 62534 | 62543 | 62544 | 62545 | 62546 | 62560 | 62561 | 62562 | 62563 | 62564 | 62568 |
| | 62569 | 62570 | 62580 | 62581 | 62582 | 62592 | 62593 | 62594 | 62595 | 62596 | 62597 | 62598 |
| | 62599 | 62600 | 62601 | 62602 | 62603 | 62604 | 62605 | 62606 | 62612 | 62613 | 62618 | 62619 |
| | 62623 | 62629 | 62634 | 62635 | 62636 | 62637 | 62638 | 62639 | 62640 | 62644 | 62653 | 62654 |
| | 62655 | 62656 | 62657 | 62658 | 62659 | 62666 | 62667 | 62668 | 62669 | 62670 | 62671 | 62672 |
| | 62678 | 62679 | 62680 | 62681 | 62682 | 62683 | 62697 | 62698 | 62699 | 62700 | 62705 | 62706 |
| | 62707 | 62708 | 62709 | 62712 | 62713 | 62738 | 62739 | 62740 | 62746 | 62747 | 62748 | 62749 |
| | 62762 | 62765 | 62766 | 62767 | 62768 | 62771 | 62772 | 62773 | 62774 | 62794 | 62795 | 62799 |
| | 62820 | 62821 | 62822 | 62827 | 62828 | 62829 | 62835 | 62836 | 62837 | 62861 | 62862 | 62863 |
| | 62864 | 62880 | 62881 | 62882 | 62904 | 62908 | 62909 | 62910 | 62913 | 62914 | 62915 | 62929 |
| | 62931 | 62932 | 62935 | 62936 | 62943 | 62949 | 62950 | 62951 | 62953 | 62954 | 62955 | 62958 |
| | 62959 | 62960 | 62961 | 62965 | 62967 | 62968 | 62969 | 62971 | 62972 | 62975 | 62976 | 62977 |
| | 62978 | 62979 | 62980 | 62981 | 62982 | 62986 | 62987 | 62988 | 62989 | 62990 | 62996 | 62997 |
| | 62998 | 62999 | 63000 | 63001 | 63002 | 63003 | 63004 | 63005 | 63006 | 63007 | 63008 | 63018 |
| | 63025 | 63026 | 63028 | 63029 | 63035 | 63036 | 63043 | 63044 | 63045 | 63049 | 63050 | 63051 |
| | 63052 | 63053 | 63058 | 63059 | 63070 | 63071 | 63072 | 63073 | 63085 | 63086 | 63095 | 63096 |
| | 63097 | 63113 | 63114 | 63115 | 63117 | 63118 | 63119 | 63129 | 63130 | 63131 | 63132 | 63133 |
| | 63140 | 63141 | 63142 | 63143 | 63144 | 63145 | 63154 | 63155 | 63156 | 63157 | 63158 | 63159 |
| | 63160 | 63164 | 63165 | 63166 | 63176 | 63177 | 63178 | 63181 | 63182 | 63183 | 63184 | 63185 |
| | 63186 | 63187 | 63188 | 63189 | 63190 | 63191 | 63192 | 63193 | 63194 | 63195 | 63196 | 63197 |
| | 63199 | 63200 | 63201 | 63208 | 63209 | 63210 | 63216 | 63217 | 63218 | 63219 | 63220 | 63221 |
| | 63222 | 63223 | 63224 | 63228 | 63229 | 63230 | 63231 | 63232 | 63245 | 63246 | 63247 | 63248 |
| | 63249 | 63250 | 63251 | 63252 | 63253 | 63254 | 63255 | 63256 | 63260 | 63261 | 63262 | 63263 |
| | 63264 | 63265 | 63271 | 63272 | 63273 | 63274 | 63275 | 63276 | 63285 | 63286 | 63287 | 63294 |
| | 63295 | 63296 | 63302 | 63303 | 63304 | 63310 | 63324 | 63325 | 63326 | 63327 | 63328 | 63329 |
| | 63330 | 63331 | 63332 | 63360 | 63361 | 63362 | 63404 | 63405 | 63406 | 63407 | 63408 | 63409 |
| | 63410 | 63419 | 63427 | 63428 | 63429 | 63430 | 63434 | 63435 | 63436 | 63437 | 63438 | 63439 |
| | 63440 | 63441 | 63446 | 63447 | 63458 | 63459 | 63460 | 63461 | 63462 | 63463 | 63464 | 63465 |
| | 63466 | 63468 | 63469 | 63470 | 63472 | 63473 | 63474 | 63482 | 63484 | 63485 | 63494 | 63495 |
| | 63498 | 63500 | 63501 | 63504 | 63505 | 63521 | 63522 | 63523 | 63535 | 63536 | 63537 | 63538 |
| | 63542 | 63543 | 63544 | 63559 | 63560 | 63563 | 63566 | 63567 | 63568 | 63569 | 63570 | 63571 |
| | 63572 | 63573 | 63574 | 63575 | 63576 | 63577 | 63587 | 63588 | 63589 | 63599 | 63600 | 63601 |
| | 63602 | 63603 | 63609 | 63610 | 63611 | 63612 | 63613 | 63614 | 63615 | 63616 | 63617 | 63618 |
| | 63619 | 63620 | 63621 | 63622 | 63623 | 63624 | 63628 | 63633 | 63639 | 63640 | 63641 | 63642 |
| | 63643 | 63645 | 63646 | 63647 | 63648 | 63649 | 63650 | 63651 | 63652 | 63653 | 63658 | 63659 |
| | 63665 | 63666 | 63668 | 63671 | 63672 | 63673 | 63674 | 63675 | 63676 | 63677 | 63685 | 63686 |
| | 63688 | 63689 | 63690 | 63695 | 63696 | 63697 | 63707 | 63708 | 63709 | 63715 | 63716 | 63717 |
| | 63718 | 63719 | 63720 | 63721 | 63722 | 63723 | 63725 | 63726 | 63731 | 63732 | 63733 | 63734 |
| | 63736 | 63737 | 63738 | 63739 | 63740 | 63741 | 63742 | 63743 | 63746 | 63747 | 63749 | 63750 |
| | 63751 | 63752 | 63753 | 63754 | 63755 | 63756 | 63758 | 63759 | 63760 | 63762 | 63764 | 63765 |
| | 63766 | 63767 | 63768 | 63769 | 63770 | 63771 | 63772 | 63773 | 63774 | 63776 | 63777 | 63778 |
| | 63780 | 63781 | 63786 | 63788 | 63789 | 63790 | 63791 | 63792 | 63793 | 63794 | 63795 | 63796 |
| | 63797 | 63799 | 63801 | 63802 | 63804 | 63805 | 63806 | 63807 | 63808 | 63809 | 63810 | 63811 |
| | 63813 | 63814 | 63823 | 63824 | 63825 | 63826 | 63827 | 63828 | 63829 | 63830 | 63831 | 63833 |
| | 63834 | 63835 | 63836 | 63841 | 63843 | 63847 | 63848 | 63849 | 63850 | 63852 | 63853 | 63854 |
| | 63855 | 63856 | 63857 | 63858 | 63865 | 63869 | 63870 | 63871 | 63872 | 63877 | 63878 | 63879 |
| | 63880 | 63881 | 63882 | 63883 | 63884 | 63885 | 63886 | 63887 | 63888 | 63889 | 63890 | 63891 |
| | 63892 | 63893 | 63894 | 63895 | 63896 | 63897 | 63898 | 63900 | 63901 | 63902 | 63904 | 63905 |
| | 63906 | 63912 | 63913 | 63921 | 63922 | 63923 | 63946 | 63947 | 63948 | 63952 | 63953 | 53954 |
| | 63963 | 63964 | 63969 | 63970 | 63971 | 63978 | 63979 | 63980 | 63987 | 63988 | 63989 | 63990 |
| | 63991 | 63992 | 63993 | 63994 | 63995 | 63996 | 64001 | 64002 | 64003 | 64005 | 64006 | 64007 |
| | 64008 | 64009 | 64010 | 64011 | 64012 | 64013 | 64014 | 64015 | 64016 | 64017 | 64018 | 64019 |
| | 64020 | 64021 | 64022 | 64023 | 64024 | 64025 | 64026 | 64027 | 64028 | 64029 | 64030 | 64031 |
| | 64032 | 64033 | 64034 | 64035 | 64036 | 64037 | 64038 | 64039 | 64040 | 64043 | 64044 | 64045 |
| | 64046 | 64051 | 64052 | 64053 | 64054 | 64055 | 64056 | 64057 | 64058 | 64059 | 64068 | 64069 |
| | 64071 | 64072 | 64073 | 64074 | 64075 | 64076 | 64077 | 64079 | 64080 | 64081 | 64082 | 64083 |
| | 64084 | 64085 | 64086 | 64087 | 64088 | 64089 | 64090 | 64092 | 64093 | 64094 | 64095 | 64096 |
| | 64097 | 64098 | 64099 | 64100 | 64102 | 64103 | 64104 | 64105 | 64106 | 64107 | 64108 | 64109 |
| | 64110 | 64111 | 64112 | 64113 | 64114 | 64115 | 64116 | 64117 | 64118 | 64119 | 64120 | 64121 |
| | 64122 | 64123 | 64124 | 64126 | 64127 | 64128 | 64129 | 64130 | 64131 | 64132 | 64133 | 64134 |
| | 64135 | 64136 | 64137 | 64138 | 64139 | 64140 | 64143 | 64144 | 64145 | 64146 | 64147 | 64148 |
| | 64151 | 64152 | 64169 | 64170 | 64172 | 64188 | 64189 | 64190 | 64191 | 64192 | 64193 | 64199 |
| | 64209 | 64210 | 64211 | 64213 | 64214 | 64215 | 64221 | 64222 | 64223 | 64224 | 64225 | 64226 |
| | 64227 | 64232 | 64244 | 64245 | 64246 | 64249 | 64256 | 64260 | 64266 | 64269 | 64271 | 64275 |
| | 64276 | 64281 | 64282 | 64283 | 64284 | 64285 | 64286 | 64287 | 64291 | 64292 | 64300 | 64301 |
| | 64302 | 64303 | 64311 | 64312 | 64318 | 64319 | 64320 | 64334 | 64335 | 64344 | 64345 | 64346 |
| | 64348 | 64360 | 64361 | 64362 | 64364 | 64366 | 64377 | 64378 | 64384 | 64399 | 64416 | 64417 |
| | 64418 | 64422 | 64426 | 64430 | 64431 | 64432 | 64433 | 64434 | 64439 | 64440 | 64442 | 64444 |
| | 64445 | 64446 | 64447 | 64448 | 64449 | 64455 | 64456 | 64457 | 64461 | 64462 | 64463 | 64470 |
| | 64472 | 64476 | 64477 | 64478 | 64479 | 64481 | 64485 | 64486 | 64487 | 64492 | 64493 | 64494 |
| | 64496 | 64497 | 64498 | 64499 | 64500 | 64501 | 64502 | 64503 | 64504 | 64505 | 64507 | 64509 |
| | 64510 | 64512 | 64518 | 64519 | 64523 | 64524 | 64525 | 64526 | 64527 | 64530 | 64531 | 64532 |
| | 64533 | 64534 | 64538 | 64539 | 64540 | 64541 | 64542 | 64543 | 64544 | 64546 | 64547 | 64548 |
| | 64549 | 64550 | 64551 | 64552 | 64553 | 64554 | 64555 | 64556 | 64557 | 64560 | 64561 | 64562 |
| | 64567 | 64568 | 64571 | 64572 | 64578 | 64579 | 64580 | 64581 | 64582 | 64584 | 64585 | 64586 |
| | 64587 | 64588 | 64601 | 64602 | 64603 | 64614 | 64615 | 64616 | 64623 | 64624 | 64625 | 64638 |
| | 64639 | 64640 | 64641 | 64649 | 64650 | 64657 | 64658 | 64659 | 64673 | 64674 | 64675 | 64680 |
| | 64681 | 64686 | 64689 | 64692 | 64694 | 64695 | 64696 | 64697 | 64698 | 64702 | 64703 | 64704 |
| | 64705 | 64706 | 64707 | 64708 | 64709 | 64710 | 64711 | 64714 | 64721 | 64722 | 64725 | 64726 |
| | 64727 | 64728 | 64730 | 64735 | 64746 | 64747 | 64757 | 64758 | 64759 | 64761 | 64767 | 64781 |
| | 64782 | 64788 | 64789 | 64790 | 64791 | 64792 | 64794 | 64795 | 64796 | 64797 | 64807 | 64808 |
| | 64809 | 64810 | 64811 | 64812 | 64813 | 64814 | 64815 | 64816 | 64818 | 64819 | 64820 | 64821 |
| | 64826 | 64827 | 64828 | 64830 | 64834 | 64835 | 64836 | 64837 | 64838 | 64839 | 64840 | 64841 |
| | 64842 | 64843 | 64844 | 64845 | 64846 | 64847 | 64848 | 64849 | 64850 | 64851 | 64855 | 64856 |
| | 64857 | 64858 | 64859 | 64860 | 64861 | 64862 | 64863 | 64864 | 64865 | 64869 | 64870 | 64871 |
| | 64872 | 64873 | 64874 | 64875 | 64876 | 64877 | 64878 | 64879 | 64880 | 64881 | 64882 | 54883 |
| | 64886 | 64887 | 64888 | 64889 | 64890 | 64891 | 64892 | 64893 | 64894 | 64895 | 64896 | 64897 |
| | 64898 | 64899 | 64900 | 64904 | 64905 | 64906 | 64907 | 64908 | 64909 | 64910 | 64911 | 64914 |
| | 64915 | 64916 | 64917 | 64918 | 64919 | 64922 | 64923 | 64924 | 64925 | 64926 | 64927 | 64928 |
| | 64931 | 64932 | 64933 | 64934 | 64935 | 64939 | 64940 | 64941 | 64942 | 64943 | 64944 | 64945 |
| | 64946 | 64947 | 64948 | 64949 | 64950 | 64951 | 64960 | 64961 | 64962 | 64963 | 64964 | 64965 |
| | 64966 | 64967 | 64968 | 64971 | 64976 | 64977 | 64978 | 64983 | 64984 | 64986 | 64987 | 64989 |
| | 64994 | 64995 | 64996 | 64997 | 65001 | 65005 | 65006 | 65007 | 65018 | 65019 | 65023 | 65027 |
| | 65028 | 65029 | 65030 | 65031 | 65032 | 65033 | 65064 | 65065 | 65066 | 65067 | 65075 | 65082 |
| | 65083 | 65084 | 65087 | 65088 | 65089 | 65090 | 65091 | 65092 | 65093 | 65094 | 65095 | 65096 |
| | 65097 | 65098 | 65099 | 65101 | 65102 | 65103 | 65107 | 65113 | 65114 | 65115 | 65116 | 65117 |
| | 65118 | 65119 | 65126 | 65128 | 65129 | 65130 | 65153 | 65160 | 65161 | 65167 | 65168 | 65169 |
| | 65182 | 65183 | 65189 | 65210 | 65211 | 65212 | 65213 | 65222 | 65223 | 65224 | 65230 | 65235 |
| | 65236 | 65243 | 65244 | 65245 | 65246 | 65247 | 65248 | 65249 | 65256 | 65257 | 65258 | 65262 |
| | 65263 | 65264 | 65268 | 65269 | 65270 | 65271 | 65274 | 65275 | 65276 | 65291 | 65292 | 65293 |
| | 65299 | 65300 | 65304 | 65306 | 65311 | 65319 | 65321 | 65322 | 65332 | 65333 | 65335 | 65369 |
| | 65370 | 65371 | 65373 | 65375 | 65376 | 65392 | 65414 | 65417 | 65428 | 65441 | 65452 | 65464 |
| | 65473 | 65474 | 65477 | 65478 | 65488 | 65489 | 65494 | 65495 | 65496 | 65497 | 65501 | 65502 |
| | 65512 | 65513 | 65520 | 65521 | 65522 | 65535 | 65536 | 65547 | 65548 | 65552 | 65553 | 65554 |
| | 65555 | 65560 | 65561 | 65562 | 65563 | 65568 | 65569 | 65570 | 65572 | 65573 | 65574 | 65575 |
| | 65578 | 65579 | 65583 | 65584 | 65587 | 65588 | 65590 | 65593 | 65594 | 65595 | 65596 | 65597 |
| | 65598 | 65599 | 65600 | 65601 | 65602 | 65603 | 65604 | 65605 | 65606 | 65608 | 65609 | 65610 |
| | 65611 | 65614 | 65615 | 65616 | 65617 | 65619 | 65620 | 65621 | 65622 | 65623 | 65624 | 65625 |
| | 65626 | 65630 | 65631 | 65632 | 65633 | 65640 | 65641 | 65644 | 65645 | 65659 | 65660 | 65665 |
| | 65666 | 65667 | 65668 | 65669 | 65670 | 65671 | 65672 | 65673 | 65674 | 65675 | 65676 | 65677 |
| | 65678 | 65679 | 65682 | 65683 | 65685 | 65686 | 65687 | 65688 | 65689 | 65690 | 65693 | 65694 |
| | 65695 | 65696 | 65699 | 65700 | 65701 | 65702 | 65703 | 65704 | 65705 | 65706 | 65707 | 65708 |
| | 65709 | 65712 | 65713 | 65714 | 65715 | 65717 | 65718 | 65725 | 65726 | 65727 | 65728 | 65729 |
| | 65730 | 65731 | 65733 | 65734 | 65737 | 65739 | 65741 | 65744 | 65745 | 65746 | 65747 | 65748 |
| | 65749 | 65750 | 65751 | 65752 | 65753 | 65756 | 65757 | 65758 | 65761 | 65762 | 65763 | 65765 |
| | 65771 | 65773 | 65774 | 65777 | 65778 | 65779 | 65780 | 65781 | 65782 | 65783 | 65785 | 65803 |
| | 65804 | 65805 | 65806 | 65819 | 65820 | 65824 | 65825 | 65826 | 65838 | 65839 | 65840 | 65851 |
| | 65852 | 65853 | 65854 | 65855 | 65863 | 65864 | 65866 | 65867 | 65868 | 65869 | 65870 | 65871 |
| | 65872 | 65873 | 65876 | 65877 | 65878 | 65879 | 65881 | 65882 | 65887 | 65893 | 65894 | 65896 |
| | 65897 | 65898 | 65899 | 65900 | 65901 | 65902 | 65903 | 65904 | 65905 | 65906 | 65907 | 65908 |
| | 65909 | 65910 | 65911 | 65912 | 65916 | 65924 | 65925 | 65926 | 65928 | 65929 | 65933 | 65934 |
| | 65937 | 65940 | 65941 | 65942 | 65943 | 65947 | 65959 | 55960 | 65961 | 65967 | 65969 | 66001 |
| | 66002 | 66013 | 66014 | 66015 | 66016 | 66022 | 66023 | 66024 | 66025 | 66026 | 66029 | 66030 |
| | 66031 | 66032 | 66035 | 66036 | 66039 | 66040 | 66041 | 66042 | 66045 | 66046 | 66047 | 66048 |
| | 66053 | 66054 | 66063 | 66064 | 66065 | 66067 | 66068 | 66069 | 66070 | 66071 | 66072 | 66073 |
| | 66074 | 66080 | 66081 | 66082 | 66085 | 66089 | 66090 | 66099 | 66100 | 66101 | 66102 | 66115 |
| | 66116 | 66120 | 66121 | 66138 | 66139 | 66142 | 66143 | 66146 | 66147 | 66153 | 66154 | 66167 |
| | 66168 | 66175 | 66176 | 66178 | 66179 | 66180 | 66181 | 66186 | 66187 | 66189 | 66195 | 66196 |
| | 66197 | 66198 | 66199 | 66209 | 66210 | 66213 | 66214 | 66215 | 66216 | 66217 | 66218 | 66219 |
| | 66220 | 66221 | 66222 | 66223 | 66224 | 66234 | 66235 | 66241 | 66242 | 66244 | 66245 | 66248 |
| | 66249 | 66250 | 66251 | 66252 | 66253 | 66254 | 66255 | 66256 | 66257 | 66258 | 66259 | 66265 |
| | 66266 | 66268 | 66269 | 66270 | 66271 | 66272 | 66275 | 66276 | 66277 | 66278 | 66281 | 66282 |
| | 66283 | 66287 | 66289 | 66290 | 66298 | 66299 | 66310 | 66314 | 66332 | 66370 | 66371 | 66389 |
| | 66390 | 66391 | 66397 | 66398 | 66399 | 66401 | 66402 | 66405 | 66406 | 66407 | 66408 | 66409 |
| | 66413 | 66426 | 66446 | 66448 | 66449 | 66450 | 66463 | 66464 | 66465 | 66473 | 66476 | 66480 |
| | 66490 | 66491 | 66492 | 66493 | 66500 | 66501 | 66503 | 66504 | 66510 | 66511 | 66512 | 66514 |
| | 66515 | 66518 | 66525 | 66531 | 66537 | 66539 | 66540 | 66545 | 66546 | 66548 | 66549 | 66551 |
| | 66552 | 66553 | 66554 | 66558 | 66572 | 66573 | 66577 | 66578 | 66580 | 66581 | 66591 | 66611 |
| | 66622 | 66623 | 66629 | 66630 | 66632 | 66633 | 66637 | 66638 | 66640 | 66641 | 66643 | 66644 |
| | 66645 | 66648 | 66649 | 66650 | 66651 | 66653 | 66654 | 66660 | 66661 | 66662 | 66663 | 66664 |
| | 66666 | 66667 | 66668 | 66669 | 66684 | 66685 | 66689 | 66690 | 66692 | 66693 | 66701 | 66708 |
| | 66709 | 66718 | 66719 | 66736 | 66737 | 66744 | 66745 | 66746 | 66747 | 66753 | 66754 | 66758 |
| | 66759 | 66762 | 66763 | 66766 | 66767 | 66768 | 66769 | 66770 | 66786 | 66787 | 66789 | 66790 |
| | 66791 | 66794 | 66795 | 66796 | 66797 | 66798 | 66800 | 66801 | 66802 | 66803 | 66804 | 66805 |
| | 66806 | 66807 | 66810 | 66811 | 66815 | 66816 | 66822 | 66823 | 66824 | 66825 | 66826 | 66827 |
| | 66828 | 66829 | 66830 | 66831 | 66832 | 66833 | 66834 | 66835 | 66836 | 66843 | 66844 | 66845 |
| | 66846 | 66847 | 66848 | 66852 | 66853 | 66857 | 66858 | 66860 | 66861 | 66862 | 66863 | 66864 |
| | 66865 | 66866 | 66869 | 66870 | 66873 | 66874 | 66875 | 66876 | 66877 | 66878 | 66889 | 66890 |
| | 66891 | 66901 | 66904 | 66905 | 66906 | 66910 | 66911 | 66914 | 66915 | 66916 | 66917 | 66918 |
| | 66921 | 66928 | 66929 | 66931 | 66932 | 66933 | 66937 | 66938 | 66940 | 66944 | 66945 | 66950 |
| | 66951 | 66960 | 66969 | 66970 | 66971 | 66973 | 66977 | 66981 | 66982 | 66985 | 66986 | 66987 |
| | 66988 | 66989 | 66990 | 66999 | 67000 | 67001 | 67007 | 67008 | 67009 | 67010 | 67011 | 67012 |
| | 67013 | 67014 | 67015 | 67016 | 67017 | 67020 | 67021 | 67022 | 67026 | 67027 | 67028 | 67039 |
| | 67040 | 67041 | 67050 | 67051 | 67052 | 67053 | 67054 | 67055 | 67057 | 67058 | 67064 | 67065 |
| | 67066 | 67067 | 67068 | 67069 | 67071 | 67072 | 67073 | 67074 | 67075 | 67076 | 67077 | 67078 |
| | 67079 | 67080 | 67081 | 67082 | 67083 | 67084 | 67085 | 67086 | 67087 | 67088 | 67094 | 67095 |
| | 67096 | 67097 | 67100 | 6710 | 67102 | 67103 | 67104 | 67105 | 67107 | 67108 | 67109 | 67113 |
| | 67114 | 67115 | 67118 | 67119 | 67120 | 67123 | 67124 | 67125 | 67126 | 67127 | 67128 | 67129 |
| | 67130 | 67131 | 67132 | 67133 | 67134 | 67135 | 67136 | 67137 | 67139 | 67140 | 67141 | 67148 |
| | 67149 | 67151 | 67152 | 67157 | 67158 | 67163 | 67164 | 67171 | 67175 | 67176 | 67177 | 67189 |
| | 67190 | 67208 | 67227 | 67228 | 67232 | 67233 | 67250 | 67255 | 67256 | 67259 | 67275 | 67276 |
| | 67277 | 67278 | 67279 | 67280 | 67287 | 67288 | 67289 | 67291 | 67292 | 67293 | 67294 | 67295 |
| | 67297 | 67298 | 67299 | 67300 | 67301 | 67303 | 67304 | 67308 | 67309 | 67310 | 67311 | 67312 |
| | 67313 | 67314 | 67315 | 67316 | 67321 | 67322 | 67323 | 67324 | 67328 | 67329 | 67330 | 67331 |
| | 67333 | 67334 | 67335 | 67336 | 67337 | 67338 | 67339 | 67341 | 67342 | 67343 | 67345 | 67346 |
| | 67347 | 67348 | 67349 | 67350 | 67351 | 67352 | 67353 | 67354 | 67355 | 67356 | 67357 | 67358 |
| | 67360 | 67361 | 67362 | 67363 | 67364 | 67367 | 67368 | 67369 | 67373 | 67374 | 67375 | 67376 |
| | 67377 | 67378 | 67379 | 67380 | 67381 | 67383 | 67384 | 67385 | 67386 | 67387 | 67388 | 67389 |
| | 67390 | 67391 | 67394 | 67395 | 67396 | 67401 | 67402 | 67403 | 67407 | 67408 | 67409 | 67411 |
| | 67412 | 67413 | 67414 | 67415 | 67416 | 67418 | 67419 | 67420 | 67422 | 67423 | 67424 | 67425 |
| | 67426 | 67427 | 67428 | 67429 | 67430 | 67432 | 67433 | 67434 | 67437 | 67438 | 67439 | 67440 |
| | 67441 | 67442 | 67448 | 67449 | 67450 | 67452 | 67453 | 67454 | 57471 | 67475 | 67476 | 67487 |
| | 67488 | 67496 | 67497 | 67498 | 67499 | 67503 | 67504 | 67505 | 67509 | 67510 | 67511 | 67549 |
| | 67556 | 67557 | 67558 | 67573 | 67576 | 67577 | 67579 | 67591 | 67593 | 67594 | 67595 | 67596 |
| | 67599 | 67600 | 67602 | 67610 | 67612 | 67613 | 67623 | 67624 | 67625 | 67630 | 67631 | 67632 |
| | 67640 | 67641 | 67643 | 67644 | 67645 | 67647 | 67648 | 67649 | 67652 | 67653 | 67654 | 67656 |
| | 67657 | 67658 | 67659 | 67661 | 67662 | 67664 | 67665 | 67668 | 67671 | 67672 | 67677 | 67678 |
| | 67685 | 67686 | 67687 | 67688 | 67689 | 67690 | 67691 | 67693 | 67696 | 67697 | 67698 | 67699 |
| | 67701 | 67702 | 67703 | 67704 | 67710 | 67713 | 67714 | 67715 | 67721 | 67722 | 67725 | 67726 |
| | 67727 | 67728 | 67730 | 67731 | 67744 | 67745 | 67746 | 67747 | 67748 | 67749 | 67752 | 67753 |
| | 67759 | 67760 | 67762 | 67763 | 67765 | 67766 | 67767 | 67768 | 67769 | 67770 | 67772 | 67773 |
| | 67777 | 67778 | 67781 | 67782 | 67785 | 67786 | 67787 | 67788 | 67791 | 67792 | 67793 | 67794 |
| | 67797 | 67798 | 67800 | 67801 | 67819 | 67820 | 67828 | 67829 | 67834 | 67835 | 67847 | 67848 |
| | 67849 | 67864 | 67865 | 67868 | 67869 | 67870 | 67871 | 67872 | 67873 | 67874 | 67875 | 67876 |
| | 67877 | 67881 | 67882 | 67883 | 67888 | 67889 | 67890 | 67891 | 67892 | 67893 | 67894 | 67895 |
| | 67896 | 67897 | 67900 | 67901 | 67902 | 67903 | 67904 | 67905 | 67906 | 67907 | 67909 | 67912 |
| | 67913 | 67914 | 67917 | 67918 | 67920 | 67921 | 67926 | 67930 | 67931 | 67932 | 67933 | 67935 |
| | 67937 | 67938 | 67939 | 67940 | 67941 | 67942 | 67944 | 67945 | 67953 | 67954 | 67956 | 67957 |
| | 67958 | 67959 | 67960 | 67961 | 67962 | 67963 | 67967 | 67968 | 67971 | 67972 | 67976 | 67977 |
| | 67981 | 67985 | 67986 | 67988 | 67989 | 67993 | 67994 | 67996 | 67997 | 67999 | 68007 | 68008 |
| | 68009 | 68011 | 68012 | 68013 | 68014 | 68015 | 68016 | 68020 | 68021 | 68024 | 68025 | 68026 |
| | 68027 | 68028 | 68031 | 68032 | 68034 | 68035 | 68036 | 68037 | 68038 | 68039 | 68046 | 68047 |
| | 68048 | 68050 | 68051 | 68052 | 68053 | 68054 | 68055 | 68056 | 68057 | 68058 | 68059 | 68061 |
| | 68062 | 68064 | 68065 | 68066 | 68067 | 68068 | 68069 | 68070 | 68073 | 68074 | 68075 | 68080 |
| | 68081 | 68082 | 68083 | 68084 | 68085 | 68086 | 68087 | 68088 | 68089 | 68090 | 68093 | 68094 |
| | 68099 | 68100 | 68101 | 68102 | 68106 | 68107 | 68108 | 68109 | 68117 | 68118 | 68119 | 68120 |
| | 68121 | 68122 | 68123 | 68124 | 68125 | 68126 | 68127 | 68128 | 68129 | 68130 | 68133 | 68136 |
| | 68137 | 68138 | 68139 | 68144 | 68145 | 68151 | 68152 | 68153 | 68154 | 68155 | 68156 | 68157 |
| | 68158 | 68159 | 68160 | 68161 | 68162 | 68163 | 68164 | 68165 | 68172 | 68173 | 68174 | 68175 |
| | 68178 | 68179 | 68180 | 68181 | 68184 | 68185 | 68188 | 68189 | 68190 | 68191 | 68192 | 68193 |
| | 68198 | 68199 | 68200 | 68201 | 68206 | 68207 | 68208 | 68209 | 68217 | 68218 | 68224 | 68225 |
| | 68232 | 68233 | 68234 | 68235 | 68236 | 68237 | 68246 | 68247 | 68248 | 68249 | 68252 | 68253 |
| | 68256 | 68257 | 68258 | 68259 | 68263 | 68264 | 68265 | 68267 | 68268 | 68275 | 68276 | 68277 |
| | 68278 | 68280 | 68281 | 68282 | 68283 | 68285 | 68286 | 68287 | 68288 | 68290 | 68291 | 68294 |
| | 68295 | 68296 | 68297 | 68299 | 68300 | 68304 | 68307 | 68310 | 68311 | 68316 | 68317 | 68318 |
| | 68320 | 68321 | 68322 | 68323 | 68330 | 68331 | 68332 | 68333 | 68336 | 68337 | 68338 | 68339 |
| | 68343 | 68344 | 68349 | 68350 | 68351 | 68352 | 68353 | 68354 | 68355 | 68356 | 68357 | 68359 |
| | 68363 | 68364 | 68365 | 68369 | 68370 | 68371 | 68372 | 68375 | 68382 | 68383 | 68387 | 68388 |
| | 68389 | 68390 | 68397 | 68398 | 68405 | 68406 | 68407 | 68408 | 68420 | 68425 | 68426 | 68427 |
| | 68431 | 68432 | 68433 | 68448 | 68449 | 68450 | 68451 | 68452 | 68453 | 68454 | 68455 | 68456 |
| | 68457 | 68458 | 68459 | 68461 | 68462 | 68463 | 68464 | 68467 | 68468 | 68474 | 68475 | 68479 |
| | 68490 | 68491 | 68492 | 68495 | 68500 | 68501 | 68502 | 68518 | 68519 | 68520 | 68521 | 68522 |
| | 68523 | 68524 | 68526 | 68527 | 68528 | 68529 | 68530 | 68531 | 68532 | 68533 | 68534 | 68539 |
| | 68540 | 68541 | 68544 | 68545 | 68546 | 68551 | 68552 | 68553 | 68554 | 68555 | 68556 | 68557 |
| | 68559 | 68560 | 68561 | 68562 | 68563 | 68564 | 68565 | 68566 | 68568 | 68569 | 68570 | 68574 |
| | 68575 | 68576 | 68577 | 68578 | 68579 | 68580 | 68581 | 68582 | 68583 | 68584 | 68585 | 68586 |
| | 68587 | 68588 | 68589 | 68590 | 68591 | 68596 | 68597 | 68598 | 68599 | 68600 | 68601 | 68605 |
| | 68606 | 68607 | 68608 | 68609 | 68610 | 68612 | 68613 | 68614 | 68615 | 68616 | 68617 | 68618 |
| | 68619 | 68620 | 68621 | 68622 | 68623 | 68624 | 68625 | 68626 | 68627 | 68630 | 68631 | 68638 |
| | 68639 | 68640 | 68641 | 68651 | 68666 | 68667 | 68678 | 68679 | 68693 | 68694 | 68710 | 68711 |
| | 68724 | 68725 | 68727 | 68756 | 68773 | 68774 | 68778 | 68779 | 68780 | 68794 | 68795 | 68803 |
| | 68810 | 68811 | 68829 | 68830 | 68831 | 68837 | 68838 | 68839 | 68842 | 68844 | 68845 | 68849 |
| | 68850 | 68858 | 68863 | 68864 | 68865 | 68866 | 68867 | 68870 | 68872 | 68875 | 68876 | 68877 |
| | 68879 | 68888 | 68889 | 68898 | 68899 | 68904 | 68919 | 68923 | 68924 | 68927 | 68928 | 68929 |
| | 68936 | 68938 | 68939 | 68942 | 68947 | 68948 | 68949 | 68950 | 68951 | 68953 | 68954 | 68955 |
| | 68957 | 68958 | 68959 | 68960 | 68961 | 68962 | 68963 | 68964 | 68965 | 68966 | 68969 | 68972 |
| | 68973 | 68974 | 68977 | 68978 | 68980 | 68981 | 68982 | 68983 | 68985 | 68986 | 68990 | 68991 |
| | 68994 | 68995 | 68996 | 68997 | 68998 | 68999 | 69000 | 69001 | 69002 | 69003 | 69004 | 69007 |
| | 69008 | 69009 | 69010 | 69011 | 69015 | 69017 | 69021 | 69030 | 69031 | 69033 | 69034 | 69035 |
| | 69036 | 69037 | 69040 | 69041 | 69043 | 69046 | 69048 | 69053 | 69058 | 69064 | 69073 | 69078 |
| | 69079 | 69080 | 69085 | 69091 | 69092 | 69095 | 69100 | 69101 | 69102 | 69103 | 69107 | 69108 |
| | 69109 | 69110 | 69111 | 69117 | 69120 | 69121 | 69125 | 69130 | 69131 | 69132 | 69134 | 69135 |
| | 69136 | 69141 | 69142 | 69152 | 69184 | 69185 | 69189 | 69190 | 69198 | 69206 | 69208 | 69218 |
| | 69219 | 69220 | 69221 | 69222 | 69225 | 69260 | 69261 | 69274 | 69283 | 69285 | 69286 | 69287 |
| | 69288 | 69294 | 69295 | 69304 | 69305 | 69308 | 69309 | 69311 | 69312 | 69313 | 69314 | 69320 |
| | 69321 | 69322 | 69325 | 69326 | 69334 | 69349 | 69352 | 69353 | 69362 | 69371 | 69375 | 69376 |
| | 69379 | 69380 | 69381 | 69383 | 69384 | 69400 | 69424 | 69430 | 69436 | 69437 | 69440 | 69441 |
| | 69450 | 69451 | 69452 | 69453 | 69454 | 69456 | 69457 | 69458 | 69459 | 69460 | 69466 | 69467 |
| | 69470 | 69471 | 69472 | 69486 | 69487 | 69488 | 69490 | 69499 | 69507 | 69510 | 69511 | 69515 |
| | 69516 | 69522 | 69528 | 69529 | 69532 | 69533 | 69537 | 69541 | 69542 | 69544 | 69545 | 69550 |
| | 69559 | 69561 | 69562 | 69565 | 69567 | 69583 | 69584 | 69594 | 69601 | 69610 | 69615 | 69621 |
| | 69629 | 69633 | 69634 | 69637 | 69642 | 69644 | 69650 | 69652 | 69654 | 69655 | 69656 | 69658 |
| | 69667 | 69668 | 69678 | 69679 | 69681 | 69682 | 69683 | 69688 | 69695 | 69696 | 69705 | 69707 |
| | 69709 | 69712 | 69719 | 69722 | 69731 | 69739 | 69740 | 69761 | 69769 | 69773 | 69778 | 69779 |
| | 69780 | 69791 | 69792 | 69795 | 69796 | 69799 | 69803 | 69804 | 69805 | 69810 | 69812 | 69813 |
| | 69816 | 69817 | 69818 | 69819 | 69820 | 69822 | 69823 | 69825 | 69826 | 69828 | 69829 | 69830 |
| | 69831 | 69832 | 69834 | 69836 | 69840 | 69843 | 69846 | 69847 | 69849 | 69850 | 69854 | 69856 |
| | 69859 | 69860 | 69861 | 69864 | 69867 | 69874 | 69876 | 69877 | 69878 | 69885 | 69886 | 69889 |
| | 69891 | 69893 | 69895 | 69899 | 69903 | 69905 | 69906 | 69910 | 69913 | 69918 | 69921 | 69922 |
| | 69923 | 69927 | 69928 | 69930 | 69932 | 69935 | 69936 | 69938 | 69947 | 69957 | 69965 | 69966 |
| | 69969 | 69976 | 69996 | 70000 | 70001 | 70007 | 70008 | 70010 | 70015 | 70024 | 70025 | 70031 |
| | 70039 | 70044 | 70050 | 70053 | 70058 | 70060 | 70069 | 70082 | 70084 | 70087 | 70088 | 70101 |
| | 70120 | 70121 | 70123 | 70125 | 70126 | 70130 | 70131 | 70136 | 70137 | 70152 | 70164 | 70166 |
| | 70167 | 70168 | 70172 | 70183 | 70186 | 70189 | 70196 | 70210 | 70211 | 70218 | 70227 | 70228 |
| | 70230 | 70232 | 70237 | 70242 | 70246 | 70250 | 70251 | 70253 | 70258 | 70263 | 70265 | 70268 |
| | 70269 | 70278 | 70302 | 70305 | 70306 | 70325 | 70327 | 70329 | 70336 | 70341 | 70362 | 70372 |
| | 70388 | 70403 | 70404 | 70405 | 70406 | 70407 | 70410 | 70418 | 70419 | 70420 | 70423 | 70424 |
| | 70426 | 70431 | 70432 | 70433 | 70434 | 70435 | 70437 | 70438 | 70439 | 70440 | 70441 | 70445 |
| | 70456 | 70460 | 70472 | 70478 | 70480 | 70482 | 70499 | 70502 | 70503 | 70505 | 70512 | 70513 |
| | 70515 | 70516 | 70517 | 70518 | 70525 | 70527 | 70528 | 70532 | 70541 | 70542 | 70545 | 70549 |
| | 70551 | 70552 | 70567 | 70568 | 70570 | 70571 | 70572 | 70575 | 70582 | 70589 | 70596 | 70602 |
| | 70604 | 70612 | 70615 | 70623 | 70627 | 70628 | 70629 | 70634 | 70635 | 70637 | 70643 | 70645 |
| | 70652 | 70661 | 70672 | 70674 | 70681 | 70682 | 70683 | 70694 | 70700 | 70721 | 70724 | 70725 |
| | 70735 | 70742 | 70753 | 70754 | 70755 | 70763 | 70764 | 70778 | 70784 | 70801 | 70810 | 70817 |
| | 70823 | 70826 | 70831 | 70832 | 70842 | 70850 | 70871 | 70872 | 70887 | 70893 | 70900 | 70908 |
| | 70914 | 70916 | 70924 | 70928 | 70929 | 70933 | 70936 | 70948 | 70954 | 70959 | 70973 | 70974 |
| | 70992 | 70994 | 70997 | 71005 | 71028 | 71030 | 71032 | 71034 | 71037 | 71039 | 71040 | 71041 |
| | 71043 | 71048 | 71049 | 71050 | 71052 | 71059 | 71060 | 71063 | 71469 | 71124 | 71694 | 71189 |
| | 71071 | 71076 | 71079 | 71080 | 71087 | 71089 | 71090 | 71091 | 71092 | 71108 | 71112 | 71114 |
| | 71115 | 71118 | 71124 | 71126 | 71127 | 71137 | 71138 | 71139 | 71141 | 71144 | 71155 | 71158 |
| | 71164 | 71169 | 71172 | 71184 | 71189 | 71190 | 71191 | 71209 | 71222 | 71223 | 71224 | 71227 |
| | 71228 | 71238 | 71239 | 71240 | 71242 | 71243 | 71244 | 71247 | 71256 | 71260 | 71264 | 71270 |
| | 71277 | 71307 | 71308 | 71311 | 71312 | 71314 | 71315 | 71316 | 71317 | 71318 | 71319 | 71322 |
| | 71323 | 71326 | 71327 | 71330 | 71335 | 71342 | 71351 | 71370 | 71377 | 71381 | 71384 | 71387 |
| | 71393 | 71395 | 71397 | 71400 | 71401 | 71402 | 71407 | 71423 | 71425 | 71426 | 71433 | 71452 |
| | 71469 | 71473 | 71474 | 71475 | 71479 | 71481 | 71490 | 71491 | 71496 | 71501 | 71510 | 71511 |
| | 71528 | 71529 | 71532 | 71536 | 71537 | 71541 | 71545 | 71546 | 71549 | 71550 | 71552 | 71553 |
| | 71554 | 71555 | 71557 | 71558 | 71571 | 71585 | 71586 | 71589 | 71590 | 71593 | 71599 | 71605 |
| | 71612 | 71615 | 71616 | 71628 | 71633 | 71638 | 71640 | 71641 | 71660 | 71681 | 71683 | 71690 |
| | 71694 | 71696 | 71698 | 75518 | 75519 | 75520 | 75521 | 75522 | 75523 | 75528 | 75530 | 75531 |
| | 75532 | 75533 | 75534 | 75535 | 75536 | 75537 | 75538 | 75556 | 75558 | 75559 | 75563 | 75564 |
| | 75565 | 75567 | 75568 | 75569 | 75574 | 75575 | 75579 | 75580 | 75581 | 75583 | 75591 | 75597 |
| | 75598 | 75599 | 75607 | 75608 | 75609 | 75610 | 75617 | 75618 | 75624 | 75625 | 75633 | 75634 |
| | 75635 | 75647 | 75648 | 75649 | 75650 | 75651 | 75652 | 75655 | 75656 | 75657 | 75662 | 75663 |
| | 75664 | 75665 | 75666 | 75667 | 75668 | 75670 | 75671 | 75672 | 75673 | 75674 | 75675 | 75676 |
| | 75677 | 75678 | 75684 | 75685 | 75686 | 75687 | 75688 | 75689 | 75691 | 75692 | 75693 | 75696 |
| | 75697 | 75702 | 75703 | 75704 | 75734 | 75735 | 75736 | 75737 | 75738 | 75739 | 75740 | 75741 |
| | 75742 | 75744 | 75745 | 75746 | 75747 | 75748 | 75749 | 75750 | 75751 | 75752 | 75756 | 75757 |
| | 75758 | 75759 | 75760 | 75761 | 75763 | 75765 | 75769 | 75770 | 75771 | 75789 | 75790 | 75792 |
| | 75793 | 75794 | 75795 | 75796 | 75799 | 75800 | 75802 | 75803 | 75804 | 75805 | 75806 | 75809 |
| | 75810 | 75811 | 75813 | 75820 | 75821 | 75822 | 75831 | 75832 | 75833 | 75834 | 75845 | 75846 |
| | 75847 | 75857 | 75873 | 75874 | 75875 | 75876 | 75877 | 75878 | 75879 | 75881 | 75882 | 75883 |
| | 75884 | 75887 | 75894 | 75895 | 75896 | 75898 | 75899 | 75900 | 75915 | 75918 | 75919 | 75920 |
| | 75921 | 75922 | 75927 | 75929 | 75930 | 75931 | 75932 | 75933 | 75934 | 75935 | 75944 | 75949 |
| | 75950 | 75951 | 75953 | 75954 | 75955 | 75956 | 75959 | 75965 | 75966 | 75967 | 75968 | 75971 |
| | 75972 | 75973 | 75975 | 75976 | 75977 | 75988 | 75989 | 75993 | 75994 | 75995 | 75996 | 75997 |
| | 76008 | 76009 | 76010 | 76031 | 76032 | 76034 | 76037 | 76038 | 76039 | 76040 | 76041 | 76043 |

(22) Protein whose gene expression changes in response to lauric acid stress (SEQ ID NOs)

| | | | | | | | | | | | | |
|---|---|---|---|---|---|---|---|---|---|---|---|---|
| 4449 | 4453 | 4454 | 4465 | 4503 | 4518 | 4594 | 4617 | 4618 | 4634 | 4635 | 4665 | 4679 |
| | 4685 | 4686 | 4742 | 4744 | 4745 | 4747 | 4749 | 4750 | 4751 | 4760 | 4761 | 4763 |
| | 4765 | 4766 | 4772 | 4786 | 4850 | 4972 | 4973 | 5120 | 5396 | 5556 | 5695 | 5815 |
| | 5840 | 5842 | 5849 | 5897 | 5932 | 5938 | 5942 | 6013 | 6049 | 6082 | 6140 | 6141 |
| | 6142 | 6158 | 6208 | 6218 | 6293 | 6350 | 6468 | 6475 | 6481 | 6522 | 6579 | 10271 |
| | 10275 | 10276 | 10280 | 10281 | 10295 | 10296 | 10357 | 10369 | 10476 | 10478 | 10498 | 10514 |
| | 10516 | 10539 | 10540 | 10580 | 10614 | 10618 | 10638 | 10640 | 10656 | 10657 | 10677 | 10723 |
| | 10731 | 10745 | 10752 | 10855 | 10862 | 10863 | 10864 | 10905 | 11057 | 11091 | 11238 | 11311 |
| | 11343 | 11452 | 11496 | 11594 | 11622 | 11623 | 11682 | 11684 | 11839 | 11904 | 11987 | 12018 |
| | 12019 | 16769 | 16770 | 16771 | 16772 | 16773 | 16774 | 16775 | 16776 | 16777 | 16780 | 16782 |
| | 16783 | 16784 | 16785 | 16786 | 16787 | 16788 | 16789 | 16790 | 16791 | 16792 | 16793 | 16794 |
| | 16795 | 16799 | 16800 | 16801 | 16802 | 16803 | 16804 | 16805 | 16806 | 16807 | 16808 | 71282 |
| | 71076 | 71079 | 71080 | 71081 | 71083 | 71084 | 71089 | 71090 | 71116 | 71121 | 71157 | 71159 |
| | 71171 | 71177 | 71182 | 71184 | 71195 | 71196 | 71211 | 71218 | 71221 | 71233 | 71235 | 71243 |
| | 71244 | 71245 | 71253 | 71262 | 71265 | 71273 | 71276 | 71282 | 71287 | 71293 | 71317 | 71319 |
| | 71323 | 71325 | 71326 | 71327 | 71335 | 71360 | 71372 | 71383 | 71413 | 71440 | 71445 | 71461 |
| | 71463 | 71475 | 71476 | 71494 | 71505 | 71508 | 71520 | 71560 | 71569 | 71570 | 71591 | 71593 |
| | 71602 | 71628 | 71641 | 71650 | 71655 | 71682 | 71697 | 71698 | 75518 | 75519 | 75520 | 75521 |
| | 75522 | 75523 | 75525 | 75526 | 75530 | 75531 | 75532 | 75566 | 75571 | 75572 | 75601 | 75602 |
| | 75603 | 75604 | 75605 | 75606 | 75615 | 75617 | 75618 | 75626 | 75627 | 75636 | 75639 | 75646 |
| | 75659 | 75661 | 75671 | 75672 | 75673 | 75674 | 75675 | 75682 | 75695 | 75698 | 75706 | 75750 |
| | 75751 | 75752 | 75755 | 75756 | 75757 | 75758 | 75759 | 75760 | 75763 | 75791 | 75798 | 75839 |
| | 75852 | 75877 | 75878 | 75879 | 75937 | 75943 | 75957 | 75959 | 75996 | 75997 | 76004 | 76030 |
| | 76042 | 76043 | | | | | | | | | | |

Included in all nucleic acid sequences disclosed herein are DNA nucleic acid molecules, RNA nucleic acid molecules (e.g., thymidine replaced with uridine), nucleic acid molecules encoding orthologs of the encoded proteins, as well as DNA or RNA nucleic acid sequences or any variant thereof (a structural variant or a chemical variant (e.g., chemically modified nucleotide)) comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO presented herein, or a portion thereof. Such nucleic acid molecules can have a function of the full-length nucleic acid (e.g., for the intended function of inducing an immune response) as described further herein.

Included in all amino acid sequences disclosed herein are amino acid sequences or any variant thereof (a structural variant or a chemical variant) comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the amino acid sequence of any SEQ ID NO listed in presented herein, or a portion thereof. Such polypeptides can have a function of the full-length polypeptide (e.g., for the intended function of inducing an immune response) as described further herein.

### Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Homology, as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 81%, 82%, 83%, 84%, 85%, 86%, 87%, 88%, 89%, 90%, 91%, 92%, 93%, 94%, 95%, 96%, or more of the nucleotides, and more preferably at least about 97%, 98%, 99% or more of the nucleotides. Alternatively, substantial homology exists when the segments will hybridize under selective hybridization conditions, to the complement of the strand.

The percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25(17):3389 3402. When utilizing BLAST and Gapped BLAST programs, the default parameters of the respective programs (e.g., XBLAST and NBLAST) can be used (available on the world wide web at the NCBI website).

### Codon Optimization

In preferred embodiments, the nucleic acid (e.g., DNA or RNA) vaccines of the present disclosure comprise those that are codon-optimized for expression in a host cell or a subject.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage. Codon usage tables are readily available, for example, at the “Codon Usage Database,” available World Wide Web at kazusa.or.jp/codon/, and these tables can be adapted in a number of ways. See Nakamura, Y., et al. “Codon usage tabulated from the international DNA sequence databases: status for the year 2000” Nucl. Acids Res. 28:292 (2000). In preferred embodiments, codon tables from the following website are used: World Wide Web at kazusa.or.jp/codon/.

Accordingly, the term “codon-optimized” encompasses any modification of the nucleic acid sequence to comprise at least one codon that is more frequently used in a given host cell or subject. The term “codon-optimized” is not intended to mean that all codons in the nucleic acid are optimized for expression in a given host cell or subject.

Polypeptide and/or Peptide Vaccines

In certain aspects, provided herein are vaccines that utilize peptides and/or polypeptides that comprise the sequence of at least one cell surface protein of at least one methanogen, or any portion thereof. Such peptides and/or polypeptides can be chemically synthesized, or produced using an expression vector (e.g., bacteria, yeast, insect cells, mammalian cells) or in vitro translated, e.g., via methods described herein and/or those known in the art.

The polypeptide and/or peptide vaccines of the present disclosure may comprise a single peptide or a single polypeptide. Alternatively, the polypeptide and/or peptide vaccines of the present disclosure may comprise a mixture of various peptides and/or polypeptides that target at least 1, 2, 3, 4, 5, 6, 7, 8, 9, 10, 11, 12, 13, 14, 15, 16, 17, 18, 19, or 20 cell surface protein of at least 1, 2, 3, 4, 5, 6, 7, 8, 9, 10, 11, 12, 13, 14, 15, 16, 17, 18, 19, or 20 methanogens.

In some embodiments, the polypeptide and/or peptide vaccine of the present disclosure comprises at least two different fragments of the same protein. In some embodiments, the polypeptide and/or peptide vaccine comprises at least two different fragments and/or two different polypeptides of different proteins. In some embodiments, the polypeptide and/or peptide vaccine comprises multiple fragments and/or polypeptides of different proteins of different methanogens. In some embodiments, the polypeptide and/or peptide vaccine comprises multiple fragments and/or polypeptides of different proteins of the same methanogen.

In some embodiments, the polypeptide(s)/peptide(s) concentration in a vaccine composition is from about 0.001 mg to about 500 mg per mL. In some embodiments, the concentration of the polypeptide(s)/peptide(s) in a vaccine composition is from about 0.01 mg to about 50 mg per mL. In some embodiments, the concentration range may be between about 0.1 mg and about 5 mg per mL.

In some embodiments, the polypeptide(s)/peptide(s) concentration in a vaccine composition is at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 mg per mL.

The polypeptide and/or peptide vaccines of the present disclosure may be administered in a pharmaceutical composition described herein. The protein vaccines of the present disclosure may be administered with an adjuvant and/or other agents that enhance immune response. Multiple dosings of the protein vaccine is contemplated herein as described.

Production of Polypeptides and/or Peptides

Methanogen proteins can be produced by recombinant DNA techniques. For example, a nucleic acid molecule encoding the protein is cloned into an expression vector, the expression vector is introduced into a host cell (as described above), and the methanogen protein is expressed in the host cell. The methanogen protein can then be isolated from the cells by an appropriate purification scheme using standard protein purification techniques. Alternative to recombinant expression, a methanogen protein, polypeptide, or peptide can be synthesized chemically using standard peptide synthesis techniques. Accordingly, the amino acid sequences disclosed herein will enable those of skill in the art to produce polypeptides corresponding peptide sequences and sequence variants thereof. Such polypeptides can be produced in host cells by expression of polynucleotides encoding the peptide sequence, frequently as part of a larger polypeptide. A host cell for expression can be any prokaryotic or eukaryotic cell. For example, protein can be expressed in bacterial cells such as E. coli, insect cells (e.g., SF9, SF21, etc.), yeast (e.g., Saccharomyces cerevisiae, Pichia pastoris, etc.) or mammalian cells (such as Fao hepatoma cells, primary hepatocytes, Chinese hamster ovary cells (CHO), COS cells, etc.). Other suitable host cells are known to those skilled in the art. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

### Pharmaceutical Composition

Vaccines, antibodies, milk, animal feed, agents (e.g., an agent that reduces methane production in a subject, a probiotic bacterial strain, etc.), or other compositions of the present disclosure may be in a pharmaceutical composition, and thus further comprise at least one excipient and/or carrier described herein or those known in the art.

The vaccine, antibody, milk, animal feed, or agent of the present disclosure (e.g., polypeptide and/or peptide vaccines) may comprise at least one excipient that (1) increases stability; (2) permits the sustained or delayed release (e.g., from a depot formulation); and/or (3) alters the biodistribution (e.g., target to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure include, without limitation, lipidoids, liposomes, lipid nanoparticles, polymers, lipoplexes, core-shell nanoparticles, peptides, proteins, hyaluronidase, nanoparticle mimics, and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient (e.g., the vaccine). The amount of the active ingredient may be generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.01% and 99% (w/w) of the active ingredient. By way of example, the composition may comprise between 0.01% and 100%, e.g., between 0.05 and 50%, between 0.1-30%, between 5-80%, at least 80% (w/w) active ingredient.

Pharmaceutical compositions may comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Controlled/Sustained Release

In some embodiments, the compositions or agents of the present invention can be formulated for controlled release and/or targeted delivery. As used herein, “controlled release” refers to a pharmaceutical composition or compound release profile that conforms to a particular pattern of release to effect a specific outcome.

In some embodiments, the compositions or agents may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

### Excipients

Vaccines, antibodies, milk, animal feed, agents (e.g., an agent that reduces methane production in a subject, a probiotic bacterial strain, etc.), or other compositions of the present disclosure may comprise at least one excipient and/or carrier described herein or those known in the art (e.g., a pharmaceutically acceptable excipient and/or carrier).

A pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired.

In some embodiments, one or more excipients or accessory ingredients may make up greater than 50% of the total mass or volume of a pharmaceutical composition. In some embodiments, the one or more excipients or accessory ingredients may make up at least about 50%, 60%, 70%, 80%, 90%, or more of a pharmaceutical convention.

Various excipients for formulating pharmaceutical compositions and techniques for preparing the composition are known in the art (see Remington: The Science and Practice of Pharmacy, 21^{st} Edition, A. R. Gennaro (Lippincott, Williams & Wilkins, Baltimore, MD, 2006; incorporated herein by reference in its entirety).

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRU®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and/or combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for many compounds. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®, NEOLONE™, KATHON™, and/or EUXYL®.

In some embodiments, the pH of the vaccine solutions are maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and/or combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

### Cryoprotectants

In some embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

In some embodiments, vaccine formulations may comprise cryoprotectants. As used herein, there term “cryoprotectant” refers to one or more agent that when combined with a given substance, helps to reduce or eliminate damage to that substance that occurs upon freezing. In some embodiments, cryoprotectants are combined with vaccines in order to stabilize them during freezing. Frozen storage of vaccines between −20° C. and −80° C. may be advantageous for long term (e.g., 36 months) storage. In some embodiments, cryoprotectants are included in vaccine formulations through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

### Inactive Ingredients

In some embodiments, vaccine formulations may comprise at least one excipient which is an inactive ingredient. As used herein, the term “inactive ingredient” refers to one or more inactive agents included in formulations. Exemplary non-exhaustive lists of inactive ingredients and the routes of administration the inactive ingredients may be formulated in are described in Tables 7-8.

TABLE 7
Exemplary inactive ingredients

| Inactive Ingredient | Route of Administration |
|---|---|
| Alpha-Terpineol | Topical |
| Alpha-Tocopherol | Intravenous; Topical |
| Alpha-Tocopherol Acetate, DI- | Topical |
| Alpha-Tocopherol, DI- | Intravenous; Topical |
| 1,2,6-Hexanetriol | Topical |
| 1,2-Dimyristoyl-Sn-Glycero-3-(Phospho-S- | Intravenous; Infusion (IV) |
| (1-Glycerol)) | |
| 1,2-Dimyristoyl-Sn-Glycero-3- | Intravenous; Infusion (IV) |
| Phosphocholine | |
| 1,2-Dioleoyl-Sn-Glycero-3-Phosphocholine | Epidural |
| 1,2-Dipalmitoyl-Sn-Glycero-3-(Phospho- | Epidural |
| Rac-(1-Glycerol)) | |
| 1,2-Distearoyl-Sn-Glycero-3-(Phospho-Rac- | Intravenous |
| (1-Glycerol)) | |
| 1,2-Distearoyl-Sn-Glycero-3-Phosphocholine | Intravenous |
| 1-O-Tolylbiguanide | Topical |

TABLE 8
Exemplary inactive ingredients

| Route of Administration | Inactive Ingredient |
|---|---|
| Intrathecal (AN, CNBLK) | Acetone Sodium Bisulfite; Citric Acid; Hydrochloric Acid; Sodium |
| | Chloride; Sodium Hydroxide; Sodium Metabisulfite |
| Infiltration (AN) | Acetic Acid; Acetone Sodium Bisulfite; Ascorbic Acid; Benzyl |
| | Alcohol; Calcium Chloride; Carbon Dioxide; Chlorobutanol; Citric |
| | Acid; Citric Acid Monohydrate; Edetate Calcium Disodium; Edetate |
| | Disodium; Hydrochloric Acid; Hydrochloric Acid, Diluted; Lactic Acid; |
| | Methylparaben; Monothioglycerol; Nitrogen; Potassium Chloride; |
| | Potassium Metabisulfite; Potassium Phosphate, Monobasic; |
| | Propylparaben; Sodium Bisulfite; Sodium Carbonate; Sodium Chlorate; |
| | Sodium Chloride; Sodium Citrate; Sodium Hydroxide; Sodium Lactate; |
| | Sodium Metabisulfite; Sodium Phosphate, Dibasic, Heptahydrate |
| Sympathetic NBLK (AN) | Hydrochloric Acid; Sodium Chloride; Sodium Hydroxide |
| Auricular (Otic) | Acetic Acid; Aluminum Acetate; Aluminum Sulfate Anhydrous; |
| | Benzalkonium Chloride; Benzethonium Chloride; Benzyl Alcohol; |
| | Boric Acid; Calcium Carbonate; Cetyl Alcohol; Chlorobutanol; |
| | Chloroxylenol; Citric Acid; Creatinine; Cupric Sulfate; Cupric Sulfate |
| | Anhydrous; Edetate Disodium; Edetic Acid; Glycerin; Glyceryl |
| | Stearate; Hydrochloric Acid; Hydrocortisone; Hydroxyethyl Cellulose; |
| | Isopropyl Myristate; Lactic Acid; Lecithin, Hydrogenated; |
| | Methylparaben; Mineral Oil; Petrolatum; Petrolatum, White; |
| | Phenylethyl Alcohol; Polyoxyl 40 Stearate; Polyoxyl Stearate; |
| | Polysorbate 20; Polysorbate 80; Polyvinyl Alcohol; Potassium |
| | Metabisulfite; Potassium Phosphate, Monobasic; Povidone K901; |
| | Povidones; Propylene Glycol; Propylene Glycol Diacetate; |
| | Propylparaben; Sodium Acetate; Sodium Bisulfite; Sodium Borate; |
| | Sodium Chloride; Sodium Citrate; Sodium Hydroxide; Sodium |
| | Phosphate, Dibasic, Anhydrous; Sodium Phosphate, Dibasic, |
| | I Teptahydrate; Sodium Phosphate, Monobasic, Anhydrous; Sodium |

In Table 8, “AN” means anesthetic, “CNBLK” means cervical nerve block, “NBLK” means nerve block, and “IV” means intravenous.

### Naked Delivery

The compositions or agents of the present invention may be delivered to a subject naked or in saline. The naked compositions or agents may be administered to a subject using routes of administration known in the art and described herein.

### Administration

Vaccines, antibodies, milk, animal feed, agents (e.g., an agent that reduces methane production in a subject, a probiotic bacterial strain, a small molecule inhibitor, etc.), or other compositions of the present disclosure (e.g., those reducing methane production in a subject) may be administered to a subject by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the auras media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique, ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

Non-limiting routes of administration for the compositions or agents of the present disclosure are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration may comprise at least one inactive ingredient. A non-exhaustive list of inactive ingredients for use in pharmaceutical compositions for parenteral administration includes hydrochloric acid, mannitol, nitrogen, sodium acetate, sodium chloride and sodium hydroxide. Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation may also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations may be for direct injection into a region of a tissue, organ and/or subject. As a non-limiting example, a tissue, organ and/or subject may be directly injected a formulation by intramyocardial injection into the ischemic region. (See e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal (e.g., transvaginal) administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

As a non-limiting example, the formulations for rectal and/or vaginal administration may be prepared by mixing the drug with a suitable non-irritating excipient that is solid at ordinary temperatures but liquid at the rectal temperature and will therefore melt in the rectum and/or vagina to release the drug. Such materials include cocoa butter and polyethylene glycols.

A pharmaceutical composition for rectal administration may comprise at least one inactive ingredient. A non-exhaustive list of inactive ingredients for use in pharmaceutical compositions for rectal administration includes alcohol, alcohol, dehydrated, aluminum subacetate, anhydrous citric acid, aniseed oil, ascorbic acid, ascorbyl palmitate, balsam peru, benzoic acid, benzyl alcohol, bismuth subgallate, butylated hydroxyanisole, butylated hydroxytoluene, butylparaben, caramel, carbomer 934, carbomer 934p, carboxypolymethylene, cerasynt-se, cetyl alcohol, cocoa butter, coconut oil, hydrogenated, coconut oil/palm kernel oil glycerides, hydrogenated, cola nitida seed extract, d&c yellow no. 10, dichlorodifluoromethane, dichlorotetrafluoroethane, dimethyldioctadecylammonium bentonite, edetate calcium disodium, edetate disodium, edetic acid, epilactose, ethylenediamine, fat, edible, fat, hard, fd&c blue no. 1, fd&c green no. 3, fd&c yellow no. 6, flavor fig 827118, flavor raspberry pfc-8407, fructose, galactose, glycerin, glyceryl palmitate, glyceryl stearate, glyceryl stearate/peg stearate, glyceryl stearate/peg-40 stearate, glycine, hydrocarbon, hydrochloric acid, hydrogenated palm oil, hypromelloses, lactose, lanolin, lecithin, light mineral oil, magnesium aluminum silicate, magnesium aluminum silicate hydrate, methylparaben, nitrogen, palm kernel oil, paraffin, petrolatum, white, polyethylene glycol 1000, polyethylene glycol 1540, polyethylene glycol 3350, polyethylene glycol 400, polyethylene glycol 4000, polyethylene glycol 6000, polyethylene glycol 8000, polysorbate 60, polysorbate 80, potassium acetate, potassium metabisulfite, propylene glycol, propylparaben, saccharin sodium, saccharin sodium anhydrous, silicon dioxide, colloidal, simethicone, sodium benzoate, sodium carbonate, sodium chloride, sodium citrate, sodium hydroxide, sodium metabisulfite, sorbitan monooleate, sorbitan sesquioleate, sorbitol, sorbitol solution, starch, steareth-10, steareth-40, sucrose, tagatose, d-, tartaric acid, dl-, trolamine, tromethamine, vegetable oil glyceride, hydrogenated, vegetable oil, hydrogenated, wax, emulsifying, white wax, xanthan gum and zinc oxide.

A pharmaceutical composition for vaginal administration may comprise at least one inactive ingredient. Any or none of the inactive ingredients used may have been approved by the US Food and Drug Administration (FDA). A non-exhaustive list of inactive ingredients for use in pharmaceutical compositions for vaginal administration includes adipic acid, alcohol, denatured, allantoin, anhydrous lactose, apricot kernel oil peg-6 esters, barium sulfate, beeswax, bentonite, benzoic acid, benzyl alcohol, butylated hydroxyanisole, butylated hydroxytoluene, calcium lactate, carbomer 934, carbomer 934p, cellulose, microcrystalline, ceteth-20, cetostearyl alcohol, cetyl alcohol, cetyl esters wax, cetyl palmitate, cholesterol, choleth, citric acid, citric acid monohydrate, coconut oil/palm kernel oil glycerides, hydrogenated, crospovidone, edetate disodium, ethylcelluloses, ethylene-vinyl acetate copolymer (28% vinyl acetate), ethylene-vinyl acetate copolymer (9% vinylacetate), fatty alcohols, fd&c yellow no. 5, gelatin, glutamic acid, dl-, glycerin, glyceryl isostearate, glyceryl monostearate, glyceryl stearate, guar gum, high density polyethylene, hydrogel polymer, hydrogenated palm oil, hypromellose 2208 (15000 mpa·s), hypromelloses, isopropyl myristate, lactic acid, lactic acid, dl-, lactose, lactose monohydrate, lactose, hydrous, lanolin, lanolin anhydrous, lecithin, lecithin, soybean, light mineral oil, magnesium aluminum silicate, magnesium aluminum silicate hydrate, magnesium stearate, methyl stearate, methylparaben, microcrystalline wax, mineral oil, nitric acid, octyldodecanol, peanut oil, peg 6-32 stearate/glycol stearate, peg-100 stearate, peg-120 glyceryl stearate, peg-2 stearate, peg-5 oleate, pegoxol 7 stearate, petrolatum, white, phenylmercuric acetate, phospholipon 90g, phosphoric acid, piperazine hexahydrate, poly(dimethylsiloxane/methylvinylsiloxane/methylhydrogensiloxane) dimethylvinyl or dimethylhydroxy or trimethyl endblocked, polycarbophil, polyester, polyethylene glycol 1000, polyethylene glycol 3350, polyethylene glycol 400, polyethylene glycol 4000, polyethylene glycol 6000, polyethylene glycol 8000, polyglyceryl-3 oleate, polyglyceryl-4 oleate, polyoxyl palmitate, polysorbate 20, polysorbate 60, polysorbate 80, polyurethane, potassium alum, potassium hydroxide, povidone k29/32, povidones, promulgen d, propylene glycol, propylene glycol monopalmitostearate, propylparaben, quaternium-15 cis-form, silicon dioxide, silicon dioxide, colloidal, silicone, sodium bicarbonate, sodium citrate, sodium hydroxide, sodium lauryl sulfate, sodium metabisulfite, sodium phosphate, dibasic, anhydrous, sodium phosphate, monobasic, anhydrous, sorbic acid, sorbitan monostearate, sorbitol, sorbitol solution, spermaceti, stannous 2-ethylhexanoate, starch, starch 1500, pregelatinized, starch, corn, stearamidoethyl diethylamine, stearic acid, stearyl alcohol, tartaric acid, dl-, tert-butylhydroquinone, tetrapropyl orthosilicate, trolamine, urea, vegetable oil, hydrogenated, wecobee fs, white ceresin wax and white wax.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents and/or excipients commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

Suspensions for oral dosage may contain the active materials in a mixture with excipients suitable for the manufacture of aqueous suspensions. Such excipients may be suspending agents, as a non-limiting example the suspending agents may be sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate; or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions for oral dosage can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

The oral dosage may also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents. The solid dosage forms may also dissolve once they come in contact with liquid such as, but not limited to, salvia and bile.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations.

Solid dosage forms may be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Dosage forms for oral delivery may also be chewable. The chewable dosages forms may be sustained release formulations such as, but not limited to, the sustained release compositions described in International Publication No WO2013082470 and US Publication No US20130142876, each of which is herein incorporated by reference in its entirety. The chewable dosage forms may comprise amphipathic lipids such as, but not limited to, those described in International Publication No WO2013082470 and US Publication No US20130142876, each of which is herein incorporated by reference in its entirety.

### Topical or Transdermal Administration

As described herein, compositions of the present disclosure may be formulated for administration transdermally. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of delivery. Two routes are commonly considered to deliver compositions to the skin: (ii) intradermal injection; and (iii) systemic delivery (e.g. for treatment of dermatologic diseases that affect both cutaneous and extracutaneous regions). Compositions can be delivered to the skin by several different approaches known in the art.

In some embodiments, the invention provides for the compositions or agents to be delivered in more than one injection.

In some embodiments, before transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In some embodiments, a device may be used to increase permeability of tissue before delivering formulations of compositions described herein. The permeability of skin may be measured by methods known in the art and/or described in U.S. Pat. No. 6,190,315, herein incorporated by reference in its entirety. As a non-limiting example, a formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In some embodiments, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In some embodiments, a device may be used to increase permeability of tissue before delivering formulations of compositions described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the compositions of the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

A pharmaceutical composition for transdermal administration may comprise at least one inactive ingredient. A non-exhaustive list of inactive ingredients for use in pharmaceutical compositions for transdermal administration includes acrylates copolymer, acrylic acid-isooctyl acrylate copolymer, acrylic adhesive 788, adcote 72a103, aerotex resin 3730, alcohol, alcohol, dehydrated, aluminum polyester, bentonite, butylated hydroxytoluene, butylene glycol, butyric acid, caprylic/capric triglyceride, carbomer 1342, carbomer 940, carbomer 980, carrageenan, cetylpyridinium chloride, citric acid, crospovidone, daubert 1-5 pestr (matte) 164z, diethylene glycol monoethyl ether, diethylhexyl phthalate, dimethicone copolyol, dimethicone mdx4-4210, dimethicone medical fluid 360, dimethylaminoethyl methacrylate-butyl methacrylate-methyl methacrylate copolymer, dipropylene glycol, duro-tak 280-2516, duro-tak 387-2516, duro-tak 80-1196, duro-tak 87-2070, duro-tak 87-2194, duro-tak 87-2287, duro-tak 87-2296, duro-tak 87-2888, duro-tak 87-2979, edetate disodium, ethyl acetate, ethyl oleate, ethylcelluloses, ethylene vinyl acetate copolymer, ethylene-propylene copolymer, fatty acid esters, gelva 737, glycerin, glyceryl laurate, glyceryl oleate, heptane, high density polyethylene, hydrochloric acid, hydrogenated polybutene 635-690, hydroxyethyl cellulose, hydroxypropyl cellulose, isopropyl myristate, isopropyl palmitate, lactose, lanolin anhydrous, lauryl lactate, lecithin, levulinic acid, light mineral oil, medical adhesive modified s-15, methyl alcohol, methyl laurate, mineral oil, nitrogen, octisalate, octyldodecanol, oleic acid, oleyl alcohol, oleyl oleate, pentadecalactone, petrolatum, white, polacrilin, polyacrylic acid (250000 mw), polybutene (1400 mw), polyester, polyester polyamine copolymer, polyester rayon, polyethylene terephthalates, polyisobutylene, polyisobutylene (1100000 mw), polyisobutylene (35000 mw), polyisobutylene 178-236, polyisobutylene 241-294, polyisobutylene 35-39, polyisobutylene low molecular weight, polyisobutylene medium molecular weight, polyisobutylene/polybutene adhesive, polypropylene, polyvinyl acetate, polyvinyl alcohol, polyvinyl chloride, polyvinyl chloride-polyvinyl acetate copolymer, polyvinylpyridine, povidone k29/32, povidones, propylene glycol, propylene glycol monolaurate, ra-2397, ra-3011, silicon, silicon dioxide, colloidal, silicone, silicone adhesive 4102, silicone adhesive 4502, silicone adhesive bio-psa q7-4201, silicone adhesive bio-psa q7-4301, silicone/polyester film strip, sodium chloride, sodium citrate, sodium hydroxide, sorbitan monooleate, stearalkonium hectorite/propylene carbonate, titanium dioxide, triacetin, trolamine, tromethamine, union 76 amsco-res 6038 and viscose/cotton.

A pharmaceutical composition for intradermal administration may comprise at least one inactive ingredient. A non-exhaustive list of inactive ingredients for use in pharmaceutical compositions for intradermal administration includes benzalkonium chloride, benzyl alcohol, carboxymethylcellulose sodium, creatinine, edetate disodium, glycerin, hydrochloric acid, metacresol, methylparaben, phenol, polysorbate 80, protamine sulfate, sodium acetate, sodium bisulfite, sodium chloride, sodium hydroxide, sodium phosphate, sodium phosphate, dibasic, sodium phosphate, dibasic, heptahydrate, sodium phosphate, monobasic, anhydrous and zinc chloride.

### Depot Administration

As described herein, in some embodiments, the composition is formulated in depots for extended release. Generally, a specific organ or tissue (a “target tissue”) is targeted for administration.

In some aspects, the compositions or agents are spatially retained within or proximal to a target tissue. Provided are method of providing a composition to a target tissue of a mammalian subject by contacting the target tissue (which contains one or more target cells) with the composition under conditions such that the composition is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue.

Aspects of the invention are directed to methods of providing a composition to a target tissue of a subject, by contacting the target tissue (comprising one or more target cells) with the composition under conditions such that the composition is substantially retained in the target tissue.

In some embodiments, the compositions may be retained near target tissue using a small disposable drug reservoir, patch pump or osmotic pump. Non-limiting examples of patch pumps include those manufactured and/or sold by BD® (Franklin Lakes, NJ), Insulet Corporation (Bedford, MA), SteadyMed Therapeutics (San Francisco, CA), Medtronic (Minneapolis, MN) (e.g., MiniMed), UniLife (York, PA), Valeritas (Bridgewater, NJ), and SpringLeaf Therapeutics (Boston, MA). A non-limiting example of an osmotic pump include those manufactured by DURECT® (Cupertino, CA) (e.g., DUROS® and ALZET®).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient). As a non-limiting example, the compositions described herein may be formulated for pulmonary delivery by the methods described in U.S. Pat. No. 8,257,685; herein incorporated by reference in its entirety.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

The compositions and formulations provided herein which may be used for pulmonary delivery may further comprise one or more surfactants. Suitable surfactants or surfactant components for enhancing the uptake of the compositions of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomelic and polymeric, poly(vinyl amine) with dextran and/or alkanoyl side chains, Brij 35, Triton X-100 and synthetic surfactants ALEC, Exosurf, Survan and Atovaquone, among others. These surfactants can be used either as single or part of a multiple component surfactant in a formulation, or as covalently bound to a component of a pharmaceutical composition herein.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2μιη to 500μιη. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods and may, for example, 0.10% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition for inhalation (respiratory) administration may comprise at least one inactive ingredient. A non-exhaustive list of inactive ingredients for use in pharmaceutical compositions for inhalation (respiratory) administration includes acetone sodium bisulfite, acetylcysteine, alcohol, alcohol, dehydrated, ammonia, apaflurane, ascorbic acid, benzalkonium chloride, calcium carbonate, carbon dioxide, cetylpyridinium chloride, chlorobutanol, citric acid, d&c yellow no. 10, dichlorodifluoromethane, dichlorotetrafluoroethane, edetate disodium, edetate sodium, fd&c yellow no. 6, fluorochlorohydrocarbons, gelatin, glycerin, glycine, hydrochloric acid, hydrochloric acid, diluted, lactose, lactose monohydrate, lecithin, lecithin, hydrogenated soy, lecithin, soybean, lysine monohydrate, mannitol, menthol, methylparaben, nitric acid, nitrogen, norflurane, oleic acid, polyethylene glycol 1000, povidone k25, propylene glycol, propylparaben, saccharin, saccharin sodium, silicon dioxide, colloidal, sodium bisulfate, sodium bisulfite, sodium chloride, sodium citrate, sodium hydroxide, sodium lauryl sulfate, sodium metabisulfite, sodium sulfate anhydrous, sodium sulfite, sorbitan trioleate, sulfuric acid, thymol, titanium dioxide, trichloromonofluoromethane, tromethamine and zinc oxide.

A pharmaceutical composition for nasal administration may comprise at least one inactive ingredient. Any or none of the inactive ingredients used may have been approved by the US Food and Drug Administration (FDA). A non-exhaustive list of inactive ingredients for use in pharmaceutical compositions for nasal administration includes acetic acid, alcohol, dehydrated, allyl .alpha.-ionone, anhydrous dextrose, anhydrous trisodium citrate, benzalkonium chloride, benzethonium chloride, benzyl alcohol, butylated hydroxyanisole, butylated hydroxytoluene, caffeine, carbon dioxide, carboxymethylcellulose sodium, cellulose, microcrystalline, chlorobutanol, citric acid, citric acid monohydrate, dextrose, dichlorodifluoromethane, dichlorotetrafluoroethane, edetate disodium, glycerin, glycerol ester of hydrogenated rosin, hydrochloric acid, hypromellose 2910 (15000 mpa·s), methylcelluloses, methylparaben, nitrogen, norflurane, oleic acid, petrolatum, white, phenylethyl alcohol, polyethylene glycol 3350, polyethylene glycol 400, polyoxyl 400 stearate, polysorbate 20, polysorbate 80, potassium phosphate, monobasic, potassium sorbate, propylene glycol, propylparaben, sodium acetate, sodium chloride, sodium citrate, sodium hydroxide, sodium phosphate, sodium phosphate, dibasic, sodium phosphate, dibasic, anhydrous, sodium phosphate, dibasic, dihydrate, sodium phosphate, dibasic, dodecahydrate, sodium phosphate, dibasic, heptahydrate, sodium phosphate, monobasic, anhydrous, sodium phosphate, monobasic, dihydrate, sorbitan trioleate, sorbitol, sorbitol solution, sucralose, sulfuric acid, trichloromonofluoromethane and trisodium citrate dihydrate.

### Ophthalmic and Auricular (Otic) Administration

A pharmaceutical composition may be prepared, packaged, and/or sold in a formulation suitable for delivery to and/or around the eye and/or delivery to the ear (e.g., auricular (otic) administration). Non-limiting examples of route of administration for delivery to and/or around the eye include retrobulbar, conjuctival, intracorneal, intraocular, intravitreal, ophthlamic and subconjuctiva. Such formulations may, for example, be in the form of eye drops or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention. A multilayer thin film device may be prepared to contain a pharmaceutical composition for delivery to the eye and/or surrounding tissue.

A pharmaceutical composition for ophthalmic administration may comprise at least one inactive ingredient. Any or none of the inactive ingredients used may have been approved by the US Food and Drug Administration (FDA). A non-exhaustive list of inactive ingredients for use in pharmaceutical compositions for ophthalmic administration includes acetic acid, alcohol, alcohol, dehydrated, alginic acid, amerchol-cab, ammonium hydroxide, anhydrous trisodium citrate, antipyrine, benzalkonium chloride, benzethonium chloride, benzododecinium bromide, boric acid, caffeine, calcium chloride, carbomer 1342, carbomer 934p, carbomer 940, carbomer homopolymer type b (allyl pentaerythritol crosslinked), carboxymethylcellulose sodium, castor oil, cetyl alcohol, chlorobutanol, chlorobutanol, anhydrous, cholesterol, citric acid, citric acid monohydrate, creatinine, diethanolamine, diethylhexyl phthalate, divinylbenzene styrene copolymer, edetate disodium, edetate disodium anhydrous, edetate sodium, ethylene vinyl acetate copolymer, gellan gum (low acyl), glycerin, glyceryl stearate, high density polyethylene, hydrocarbon gel, plasticized, hydrochloric acid, hydrochloric acid, diluted, hydroxyethyl cellulose, hydroxypropyl methylcellulose 2906, hypromellose 2910 (15000 mpa·s), hypromelloses, jelene, lanolin, lanolin alcohols, lanolin anhydrous, lanolin nonionic derivatives, lauralkonium chloride, lauroyl sarcosine, light mineral oil, magnesium chloride, mannitol, methylcellulose (4000 mpa·s), methylcelluloses, methylparaben, mineral oil, nitric acid, nitrogen, nonoxynol-9, octoxynol-40, octylphenol polymethylene, petrolatum, petrolatum, white, phenylethyl alcohol, phenylmercuric acetate, phenylmercuric nitrate, phosphoric acid, polidronium chloride, poloxamer 188, poloxamer 407, polycarbophil, polyethylene glycol 300, polyethylene glycol 400, polyethylene glycol 8000, polyoxyethylene-polyoxypropylene 1800, polyoxyl 35 castor oil, polyoxyl 40 hydrogenated castor oil, polyoxyl 40 stearate, polypropylene glycol, polysorbate 20, polysorbate 60, polysorbate 80, polyvinyl alcohol, potassium acetate, potassium chloride, potassium phosphate, monobasic, potassium sorbate, povidone k29/32, povidone k30, povidone k90, povidones, propylene glycol, propylparaben, soda ash, sodium acetate, sodium bisulfate, sodium bisulfite, sodium borate, sodium borate decahydrate, sodium carbonate, sodium carbonate monohydrate, sodium chloride, sodium citrate, sodium hydroxide, sodium metabisulfite, sodium nitrate, sodium phosphate, sodium phosphate dihydrate, sodium phosphate, dibasic, sodium phosphate, dibasic, anhydrous, sodium phosphate, dibasic, dihydrate, sodium phosphate, dibasic, heptahydrate, sodium phosphate, monobasic, sodium phosphate, monobasic, anhydrous, sodium phosphate, monobasic, dihydrate, sodium phosphate, monobasic, monohydrate, sodium sulfate, sodium sulfate anhydrous, sodium sulfate decahydrate, sodium sulfite, sodium thiosulfate, sorbic acid, sorbitan monolaurate, sorbitol, sorbitol solution, stabilized oxychloro complex, sulfuric acid, thimerosal, titanium dioxide, tocophersolan, trisodium citrate dihydrate, triton 720, tromethamine, tyloxapol and zinc chloride.

A pharmaceutical composition for retrobulbar administration may comprise at least one inactive ingredient. A non-exhaustive list of inactive ingredients for use in pharmaceutical compositions for retrobulbar administration includes hydrochloric acid and sodium hydroxide.

A pharmaceutical composition for intraocular administration may comprise at least one inactive ingredient. A non-exhaustive list of inactive ingredients for use in pharmaceutical compositions for intraocular administration includes benzalkonium chloride, calcium chloride, citric acid monohydrate, hydrochloric acid, magnesium chloride, polyvinyl alcohol, potassium chloride, sodium acetate, sodium chloride, sodium citrate and sodium hydroxide.

A pharmaceutical composition for intravitreal administration may comprise at least one inactive ingredient. A non-exhaustive list of inactive ingredients for use in pharmaceutical compositions for intravitreal administration includes calcium chloride, carboxymethylcellulose sodium, cellulose, microcrystalline, hyaluronate sodium, hydrochloric acid, magnesium chloride, magnesium stearate, polysorbate 80, polyvinyl alcohol, potassium chloride, sodium acetate, sodium bicarbonate, sodium carbonate, sodium chloride, sodium hydroxide, sodium phosphate dibasic heptahydrate, sodium phosphate monobasic monohydrate and trisodium citrate dehydrate.

A pharmaceutical composition for subconjunctival administration may comprise at least one inactive ingredient. A non-exhaustive list of inactive ingredients for use in pharmaceutical compositions for subconjunctival administration includes benzyl alcohol, hydrochloric acid and sodium hydroxide.

A pharmaceutical composition for auricular administration may comprise at least one inactive ingredient. A non-exhaustive list of inactive ingredients for use in pharmaceutical compositions for auricular administration includes acetic acid, aluminum acetate, aluminum sulfate anhydrous, benzalkonium chloride, benzethonium chloride, benzyl alcohol, boric acid, calcium carbonate, cetyl alcohol, chlorobutanol, chloroxylenol, citric acid, creatinine, cupric sulfate, cupric sulfate anhydrous, edetate disodium, edetic acid, glycerin, glyceryl stearate, hydrochloric acid, hydrocortisone, hydroxyethyl cellulose, isopropyl myristate, lactic acid, lecithin, hydrogenated, methylparaben, mineral oil, petrolatum, petrolatum, white, phenylethyl alcohol, polyoxyl 40 stearate, polyoxyl stearate, polysorbate 20, polysorbate 80, polyvinyl alcohol, potassium metabisulfite, potassium phosphate, monobasic, povidone k90f, povidones, propylene glycol, propylene glycol diacetate, propylparaben, sodium acetate, sodium bisulfite, sodium borate, sodium chloride, sodium citrate, sodium hydroxide, sodium phosphate, dibasic, anhydrous, sodium phosphate, dibasic, heptahydrate, sodium phosphate, monobasic, anhydrous, sodium sulfite, sulfuric acid and thimerosal.

### Dosing

Provided herein are methods comprising administering a vaccine composition to a subject. The specific dose level for any particular subject will depend upon a variety of factors including the species, the activity of the specific compound employed; the specific composition employed; the age, body weight, general health, sex, and diet of the subject; the time of administration, route of administration, and rate of excretion of the specific compound employed; drugs used in combination or coincidental with the specific compound employed; and like factors well known in the arts. Compositions in accordance with the present disclosure are typically formulated in dosage unit form for ease of administration and uniformity of dosage. It will be understood, however, that the total daily usage of the compositions of the present disclosure may be decided by the attending administrator within the scope of sound judgment.

In certain embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, from about 1 mg/kg to about 25 mg/kg, from about 0.1 mg/kg to about 250 mg/kg, or any range in between or any value in between, of subject body weight per day, one or more times a day, to obtain the desired therapeutic, diagnostic, prophylactic, or imaging effect.

In some embodiments, compositions in accordance with the present disclosure may be administered at dosage levels sufficient to deliver from about 0.001 mg to about 500 mg of polypeptide(s) and/or peptide(s), or any range in between or any value in between. In some embodiments, compositions may be administered at dosage levels sufficient to deliver from about 0.01 to about 50 mg, or any range in between or any value in between. For example, in some embodiments, the range may be between about 0.1 and about 5 mg, or any range in between or any value in between, e.g., between about 0.1 and about 2 mg, or any range in between or any value in between.

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, twenty, thirty, forty, or more administrations). When multiple administrations are employed, split dosing regimens such as those described herein may be used.

In some embodiments, an animal is administered with a prime (initial dose), followed by a boost (second dose) at least about 1, 1.5, 2, 2.5, 3, 3.5, or 4 weeks after the prime. In some embodiments, at least one additional dose is given after the boost, optionally after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the boost. In some embodiments, the at least one additional dose is repeated every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In preferred embodiments, an animal is administered with a prime, followed by a boost about 2 or 3 weeks after the prime. In yet other preferred embodiments, an animal is administered with (a) a prime, (b) a boost about 2 or 3 weeks after the prime, and (c) at least one additional dose every 3, 4, 5, or 6 months. Each dose in the dosing schedule may comprise the same or different amount of the vaccine composition.

According to the present disclosure, the compositions of the present disclosure may be administered in split-dose regimens. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In some embodiments, the compositions of the present disclosure are administered to a subject in split doses. The compositions may be formulated in buffer only or in a formulation described herein.

### Dosage Forms

Vaccines, antibodies, milk, animal feed, agents (e.g., an agent that reduces methane production in a subject, a probiotic bacterial strain, etc.), or other compositions of the present disclosure may be formulated into a dosage form described herein, such as an intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compositions then depends upon its rate of dissolution which, in turn, may depend upon crystal size and crystalline form. Alternatively, delayed absorption of a parenterally administered compositions may be accomplished by dissolving or suspending the compositions in an oil vehicle. Injectable depot forms are made by forming microencapsule matrices of the compositions in biodegradable polymers such as polylactide-polyglycolide. Depending upon the ratio of compositions to polymer and the nature of the particular polymer employed, the rate of a compound release can be controlled. Examples of other biodegradable polymers include, but are not limited to, poly(orthoesters) and poly(anhydrides). Depot injectable formulations may be prepared by entrapping the compositions in liposomes or microemulsions which are compatible with body tissues.

### Pulmonary Formulation

Formulations described herein as being useful for pulmonary delivery may also be used for intranasal delivery of a pharmaceutical composition. Another formulation suitable for intranasal administration may be a coarse powder comprising the active ingredient and having an average particle from about 0.2μιη to 500μιη. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some embodiments, vaccines, antibodies, milk, animal feed, agents (e.g., an agent that reduces methane production in a subject, a probiotic bacterial strain, a small molecule inhibitor, etc.), or other compositions of the present disclosure may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising same or different components. In some embodiments, the repeat dose may comprise substantially the same components provided at substantially the same mass and/or volume. In other embodiments, the repeat dose may comprise different components (e.g., different adjuvant for a vaccine composition).

### Adjuvant

Adjuvants or immune potentiators, may also be administered with or in combination with one or more vaccine composition of the present disclosure.

The term “adjuvant” refers to an agent that when administered in conjunction with or as part of a composition described herein augments, enhances, and/or boosts the immune response to a methanogen, but when the agent is administered alone does not generate an immune response. In some embodiments, the adjuvant generates an immune response to a methanogen and does not produce an allergy or other adverse reaction. Adjuvants can enhance an immune response by several mechanisms including, e.g., lymphocyte recruitment, stimulation of B and/or T cells, and stimulation of macrophages.

In some embodiments, an adjuvant acts as a co-signal to prime T-cells and/or B-cells and/or NK cells as to the existence of the cell surface protein of a methanogen in a vaccine composition of the present disclosure.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccines. These may be co-administered by any route, e.g., intramusculary, subcutaneous, IV or intradermal injections.

Adjuvants useful in the present invention may include, but are not limited to, natural or synthetic. They may be organic or inorganic.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (Glaxo SmithKline), AS04 (Glaxo SmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)).

Adjuvants may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compunds; (9) carbohydrates; or combinations thereof.

Other adjuvants which may be utilized in the vaccines of the present disclosure include any of those listed on the web-based vaccine adjuvant database, Vaxjo; World Wide Web at violinet.org/vaxjo/ and described in for example Sayers, et al., J. Biomedicine and Biotechnology, volume 2012 (2012), Article ID 831486, 13 pages, the content of which is incorporated herein by reference in its entirety.

Selection of appropriate adjuvants will be evident to one of ordinary skill in the art. Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's complete adjuvant, Freund's incomplete adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hlFN-gamma/Interferon-g, Interleukin-\ddot{ι}β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E1 12K of Cholera Toxin mCT-E1 12K, and/or Matrix-S.

In some embodiments, the at least one adjuvant comprises oil emulsions, e.g., comprising at least (a) mineral oil lipid and (b) aqueous phase (e.g., Freund's complete adjuvant, Freund's incomplete adjuvant, Montanide ISA series (e.g., ISA70, ISA61, ISA206, ISA50), squaline-based emulsion, (e.g., MF59 and/or AS03), saponins, (e.g., Quil-A, Spikoside, QS21, or ISCOMs, e.g., ISCOPREP 703), aluminum salts (e.g., aluminum hydroxide, aluminum phosphate, and potassium aluminum sulfate), also known to a skilled artisan as ‘alum’, (e.g., Imject Alum), dextran sulfate, chitosan thermogel, (e.g., monophosphoryl lipid A), Carbol, PLGA, MDP, CpG ODN, cytokine-based adjuvants such as IL-12 and/or GM-CSF, a lipid nanoparticle/cationic liposome adjuvant, an immune stimulating complex, or any combination of two or more thereof. In preferred embodiments, the at least one adjuvant comprises Freund's complete adjuvant and/or Freund's incomplete adjuvant. See Spickler and Roth (2003) J Vet Intern Med, 17:273-281, which is incorporated herein by reference.

In some embodiments, the at least one adjuvant comprises Emulsigen-D, Emulsigen, Emulsigen-P, and/or Polygen (MVP adjuvant, Omaha, NE). In some embodiments, the at least one adjuvant comprises ENABL 06 (HuvePharma, Peachtree City, GA). In some embodiments, the at least one adjuvant comprises Montainde ISA 201 and/or Montanide Gel 02 (Seppic Inc., New Jersey).

Other adjuvants which may be co-administered with the vaccine compositions of the invention include, but are not limited to interferons, TNF-alpha, TNF-beta, chemokines such as CCL21, eotaxin, HMGB1, SA100-8alpha, GCSF, GMCSF, granulysin, lactoferrin, ovalbumin, CD-40L, CD28 agonists, PD-1, soluble PD1, L1 or L2, or interleukins such as IL-1, IL-2, IL-4, IL-6, IL-7, IL-10, IL-12, IL-13, IL-21, IL-23, IL-15, IL-17, and IL-18.

In some embodiments, the adjuvant comprises Glucopyranosyl Lipid Adjuvant (GLA), CpG oligodeoxynucleotides (e.g., Class A or B), poly(LC), aluminum hydroxide, or Pam3CSK4.

In some embodiments, the adjuvant comprises: (a) (±)—N-(3-aminopropyl)-N,N-dimethyl-2,3-bis(syn-9-tetradeceneyloxy)-1-propanaminium bromide (GAP-DMORIE) and a neutral lipid; (b) a cytokine; (c) mono-phosphoryl lipid A and trehalosedicorynomycolateAF (MPL+TDM); (d) a solubilized mono-phosphoryl lipid A formulation; and/or (e) CRL1005/BAK.

In some embodiments, the neutral lipid in (a) comprises (a) 1,2-dioleoyl-sn-glycero-3-phosphoethanolamine (DOPE); (b) 1,2-diphytanoyl-sn-glycero-3-phosphoethanolamine (DPyPE); and/or (c) 1,2-dimyristoyl-glycer-3-phosphoethanolamine (DMPE).

In some embodiments, the adjuvant comprises saponin, Montanide ISA61, a chitosan thermogel, a lipid nanoparticle/cationic liposome adjuvant, or any combination thereof. In preferred embodiments, the adjuvant comprises Montanide ISA61.

### Antibody

Unless otherwise specified here within, the terms “antibody” and “antibodies” broadly encompass naturally-occurring forms of antibodies (e.g. IgG, IgA, IgM, IgE); and recombinant antibodies, such as single-chain antibodies, chimeric antibodies, and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., at least one cell surface protein or fragment thereof of at least one methanogen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osboum et al. 1998, Nature Biotechnology 16: 778). Such single chain antibodies are also intended to be encompassed within the term “antigen-binding portion” of an antibody. Any VH and VL sequences of specific scFv can be linked to immunoglobulin constant region cDNA or genomic sequences, in order to generate expression vectors encoding complete IgG polypeptides or other isotypes (e.g., IgGA). VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. chimeric, etc.). Antibodies may also be fully specific to the subject, e.g., the antibodies may be fully ruminant or fully human. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The present disclosure includes a monoclonal antibody that works particularly well in binding and neutralizing at least one methanogen. Upon immunizing a subject population (e.g., of milk-producing subject), the milk comprising the antibody can be screened for antibodies with superior activity (e.g., specific binding, neutralizing at least one methanogen, etc.). The amino acid sequence of such antibodies can be determined (e.g., mass spec-based sequencing, Next Gen Sequencing, or other methods known in the art), their expressing DNA vectors can be synthesized, and monoclonal antibodies can be produced. One or a combination of at least two or more antibodies can be added to the drinking water and/or animal feed, and be given to a subject population.

Alternatively, such monoclonal antibodies can be generated by immunizing a vehicle animal (e.g., mouse, rabbit, etc.), and hybridomas expressing the animals can be recovered. Standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)). Antibodies produced by hybridomas can be screened and utilized according to the methods described above and herein.

In some embodiments, the antibody is a polyclonal antibody. In some embodiments, the antibody is a monoclonal antibody. In some embodiments, the antibody is an IgG or IgA. In some embodiments, the antibody is IgA. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is lyophilized. In some embodiments, the antibody is in a pharmaceutical composition of the present disclosure or those known in the art. In some embodiments, a composition comprising an antibody further comprises at least one excipient and/or carrier. In some embodiments, the antibody is in the animal feed. In some embodiments, the antibody is in the solid animal feed. In other embodiments, the antibody is in the liquid animal feed. In some embodiments, the antibody is in the drinking water or milk. In preferred embodiments, the antibody is orally consumed by a subject such that the antibody comes in contact with at least one methanogen present in the gut of the subject. In preferred embodiments, oral administration of the antibody reduces the number and/or type of at least one methanogen.

### Methods for Detection of Antibody

In certain embodiments, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In some embodiments, the antibody titer is produced or reached by 10 days following vaccination, by 20 days following vaccination, by 30 days following vaccination, by 40 days following vaccination, or by 50 or more days following vaccination. In some embodiments, the titer is produced or reached following a single dose of vaccine administered to the subject. In other embodiments, the titer is produced or reached following multiple doses, e.g., following a first and a second dose (e.g., a repeat dose.).

In certain aspects, antigen-specific antibodies are measured in units of pg/ml or are measured in units of IU/L (International Units per liter) or mIU/ml (milli International Units per ml). In some embodiments of the invention, an efficacious vaccine produces >0.5 pg/ml, >0.1 pg/ml, >0.2 pg/ml, >0.35 pg/ml, >0.5 pg/ml, >1 pg/ml, >2 pg/ml, >5 pg/ml or >10 pg/ml. In some embodiments, an efficacious vaccine produces >10 mIU/ml, >20 mIU/ml, >50 mIU/ml, >100 mIU/ml, >200 mIU/ml, >500 mIU/ml or >1000 mIU/ml.

Methods of detecting the presence of antibodies are well known in the art.

In some embodiments, antibody level or concentration is determined or measured by neutralization assay, e.g., neutralization of at least one methanogen.

Other exemplary methods include, but are not limited to, immunodiffusion, immunoelectrophoresis, radioimmunoassay (RIA), enzyme-linked immunosorbent assays (ELISAs; including variants such as competitive ELISA, sandwich ELISA, etc.), immunofluorescent assays, Western blotting, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference).

For example, ELISA and RIA procedures may be conducted such that a desired protein standard (e.g., an extracellular domain of at least one cell surface protein or a fragment thereof of at least one methanogen) is labeled (with a radioisotope such as ¹²⁵I or ³⁵S, or an assayable enzyme, such as horseradish peroxidase or alkaline phosphatase), and is brought into contact with a sample comprising the antibody, whereon the amount of the labeled protein standard bound to the antibody is measured.

Enzymatic and radiolabeling of a protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzymes are non-specific (such as using formaldehyde), and will only yield a proportion of active enzyme.

It may be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

### Milk

In certain aspects, provided herein are milk and derivatives thereof. Milk produced by vaccinated female subjects (e.g., dairy cows) comprises antibodies that bind at least one cell surface protein or a fragment thereof of at least one methanogen. Such milk can be orally consumed by subjects such that the antibodies therein can come in contact with at least one methanogen present in the gut of the subjects. Upon contact, the antibodies in the milk can neutralize the at least one methanogen and contribute to reducing methane production by the subjects.

Thus, milk from vaccinated female subject can be used to treat breast-feeding animals, thereby reducing methane production and/or methanogen colonization in young animals. This can be especially important when vaccination of young animals occurs concurrently with weaning.

In some embodiments, the milk is pasteurized and/or homogenized. In some embodiments, the milk is lyophilized, filtered, concentrated, evaporated, or processed to form dry milk powder (e.g., boiling at low pressure at low temperature). In some embodiments, said processing may allow longer shelf life of the milk/milk product and the antibodies present therein. In some embodiments, the fat content is removed/reduced from the milk. Processing of milk and/or preparation of derivatives of milk are well known in the art.

Appropriate care is taken to preserve the structural and functional (e.g., binding a methanogen) aspects of the antibodies. For example, in some embodiments, high pressure (˜200 MPa) and low temperature (−4° C.) are used throughout the process as described at least by Kim et al. (2008) Journal of Dairy Science, 91:4176-4182. In other embodiments, milk may be pasteurized at low-temperature of 60° C. for 10 minutes at standard pressure. These conditions may pasteurize milk without significantly altering the antibody function.

Alternatively, milk can be filtered to remove microorganisms instead of pasteurizing. Microfiltration is a process that replaces pasteurization and produces milk with fewer microorganisms and longer shelf life without a change in the quality of the milk. In this process, cream is separated from the skimmed milk and the skimmed milk is forced through ceramic microfilters that trap 99.9% of microorganisms in the milk (as compared to 99.999% killing of microorganisms in standard high temperature short time pasteurization).

Ultrafiltration uses finer filters than microfiltration, which allow lactose and water to pass through while retaining fats, calcium and protein. As with microfiltration, the fat may be removed before filtration and added back in afterwards. Ultrafiltered milk is used widely in the industry in cheesemaking.

Colostrum may similarly be used in the milk embodiments disclosed herein.

### Animal Feed

Provided herein are animal feeds that are useful in reducing methane production by a subject. Animal feed encompasses any edible consumables that are suitable for consumption by the subject of the present disclosure. Accordingly, animal feed also includes drinking water or other food items that may be consumed by the subject including but not limited to humans, canines, felines, and ruminants.

Animal feed may be used in combination with or comprise any one of vaccines, antibodies, milk, agents (e.g., an agent that reduces methane production in a subject, a probiotic bacterial strain, a small molecule inhibitor, etc.), or other compositions of the present disclosure (e.g., those reducing methane production in a subject). Animal feed may comprise at least one agent, which reduces the methane production in a subject.

In some embodiments, the animal feed comprises an antibody that binds at least one cell surface antigen or a fragment thereof of at least one methanogen.

In some embodiments, the animal feed comprises a composition comprising an antibody that binds at least one cell surface antigen or a fragment thereof of at least one methanogen. For example, the animal feed may comprise milk or derivatives thereof comprising said antibody.

In some embodiments, the animal feed is liquid (e.g., drinking water, milk). An antibody that binds at least one cell surface antigen or a fragment thereof of at least one methanogen or a composition comprising same may be added to the liquid animal feed (e.g., drinking water) before being given to the subject. In some embodiments, said antibody or a composition comprising same may be added to the drinking water. In other embodiments, the milk or derivatives thereof comprising said antibody may be given directly to the subject, or added to other liquid animal feed (e.g., drinking water).

In other embodiments, the animal feed is solid. In some such embodiments, the animal feed may comprise hay, straw, silage, compressed and pelleted feeds, oils and mixed rations, and sprouted grains and legumes. An antibody that binds at least one cell surface antigen or a fragment thereof of at least one methanogen or a composition comprising same may be added to the solid animal feed before being given to the subject.

In some embodiments, an animal feed may comprise fats and fatty acids that further aid in reducing methane production in subjects. Based on a meta-analysis, fat supplementation reduced CH₄ by 3.77% in cattle and 4.30% in sheep per 1% dietary fats. Fat decreases CH₄ production (expressed as g/kg digestible dry matter (DM)) more from sheep than from cattle, which was attributed to the comparatively lower depression of DM digestion together with numerically larger depression of CH₄ production (g/kg DM) by fat in sheep. Among fatty acids, C12:0, C18:3 and other polyunsaturated fatty acids (PUFA) are more potent than saturated fatty acids. The CH₄-suppressing efficacy of fats generally persists, with persistent suppression being noted for 72 days and longer in cattle.

Fats supplemented up to 6% of the diet (DM) can also improve milk production while appreciably decreasing CH₄ emissions (15%) in cattle, but higher concentrations decreased production efficiency due to a reduction of feed digestion and fermentation. Medium-chain fatty acids (MCFA) and PUFA can lower abundance and metabolic activities of rumen methanogens and change their species composition. PUFA can also directly inhibit protozoa and serve as hydrogen sink through biohydrogenation. Both MCFA and PUFA appear to damage the cell membrane, thereby abolishing the selective permeability of cell membrane, which is required for survival and growth of methanogens and other microbes. The inhibitory effect of fat on methanogenesis is more pronounced in cattle fed concentrate-based diets than in cattle fed forage-based diets. Because C12; and C14:0 is more inhibitory to M. ruminantium at pH 5 than at pH 7, the concentrate level-dependent anti-methanogenic efficacy of MCFA and PUFA is probably attributed to the lower pH associated with high-concentrate diets.

In some embodiments, the animal feed comprises fat and/or fatty acid. In some embodiments, the animal feed comprises fat and/or fatty acid that is at least about 1%, 2%, 3%, 4%, 5%, or 6% of the diet (e.g., diet based on dry matter).

Numerous animal feed and feed additives are known in the art. Any agent that reduce methane production in a subject (e.g., small molecule inhibitors, e.g., Table 9, probiotic bacterial strain, etc.; see below) described herein or those known in the art may be used as a feed additive. Certain exemplary feed additives include: berberine, nitrate, eucalyptus oil, alliin, diallyl disulfide (DADS), flavanone glycoside (e.g., neohesperidin, isonaringin, poncirin, hesperidin), 3-nitrooxypropanol, rac-4-Phenylbutane-1,2-diyl dinitrate, 2-(hydroxymethyl)-2-(nitrooxymethyl)-1,3-propanediol, N-ethyl-3-nitro-oxy-propionic sulfonyl amide, 5-nitrooxy-pentanenitrile, 5-nitrooxy-pentane, 3-nitro-oxy-propyl propionate, 1,3-bis-nitrooxypropane, 1,4-bis-nitrooxybutane, 1,5-bis-nitrooxypentane, 3-nitro-oxy-propyl benzoate, 3-nitro-oxy-propyl hexanoate, 3-nitro-oxy-propyl 5-nitro-oxy-hexanoate, benzylnitrate, isosorbid-dinitrate, N-[2-(nitrooxy)ethyl]-3-pyridinecarboxamide, 3-nitrooxy propionic acid, methyl-3-nitrooxy propionate, ethyl-3-nitrooxy propionate, ethyl-4-nitrooxy butanoate, ethyl-3-nitrooxy butanoate, 5-nitrooxy pentanoic acid, ethyl-5-nitrooxy pentanoate, 6-nitrooxy hexanoic acid, ethyl-6-nitrooxy hexanoate, ethyl-4-nitrooxy-cyclohexylcarboxylate, 8-nitrooxy octanoic acid, ethyl-8-nitrooxy octanoate, 11-nitrooxy undecanoic acid, ethyl-11-nitrooxy undecanoate, 5-nitrooxy-pentanoic amide, 5-nitrooxy-N-methyl-pentanoic amide, lauric acid, and haloform (e.g., bromoform, chloroform, iodoform).

Agents that Reduce Methane and/or Hydrogen Production in Ruminants

### Combination Treatment

Vaccines, antibodies, milk, animal feed, and agents (e.g., an agent that reduces methane production in a subject, a probiotic bacterial strain, etc.) may be administered to a subject in any combination. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent (vaccines, antibodies, milk, animal feed, agents that reduce methane production in a subject) will be administered at a dose and/or on a time schedule determined for that agent. In some embodiments, the present disclosure encompasses the delivery of combinations that may improve immune response against at least one methanogen, and/or reduce methane production by a subject.

The combinations can conveniently be presented for use in the form of a pharmaceutical formulation and thus pharmaceutical compositions comprising a combination as defined above together with a pharmaceutically acceptable diluent or carrier represent a further aspect of the invention.

The individual compounds of such combinations can be administered either sequentially or simultaneously in separate or combined pharmaceutical formulations.

It will further be appreciated that (vaccines, antibodies, milk, animal feed, agents that reduce methane production in a subject) in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In some embodiments, the combinations, each or together may be administered according to the split dosing regimens described herein.

The term “conjoint” or “combination” administration, as used herein, refers to the administration of two or more agents that aid in reducing methane production in a subject. The different agents comprising the combination may be administered concomitant with, prior to, or following the administration of one or more agents.

In certain embodiments, combination administration can demonstrate synergisms between the two or more agents resulting in a greater methane reduction in subject than either agent alone.

Synergistic effects, also known as synergy, refer to the phenomenon where the combined effect of two or more factors or components is greater than the sum of their individual effects. In other words, when these factors or components interact, they create an amplified or enhanced effect that is greater than what would be expected based on their individual contributions.

In certain cases, the agents may have different modes of action or mechanisms by which they exert their effects, for example targeting different methanogens or different methanogen enzymes. When these agents are combined, their actions can complement each other, targeting different aspects of a problem or working on multiple pathways simultaneously. This complementary action enhances their overall effectiveness, resulting in a better outcome than either agent could achieve alone.

In certain cases, synergism can significantly enhance the efficacy of the agents involved. For example, the agents may interact in a way that enhances their absorption, distribution, or bioavailability, increasing their effectiveness in treating a particular condition.

In certain cases, one agent may enhance the effects of the other without contributing much individually. This is known as potentiation. The presence of one agent can increase the uptake, binding affinity, or sensitivity of the other, making it more potent and effective. The combined effect is greater than what would be achieved by either agent on its own.

In certain cases, one or more agents may have inherent weaknesses or face resistance from target organisms or systems. By combining them with another agent, the synergistic interaction can bypass or counteract these obstacles, leading to a more effective outcome. For example, antibodies generated via vaccination of a first vaccine may face resistance to ruminal proteases, thus, combining with protease inhibitors or one or more additional vaccine towards certain ruminal proteases may reduce the resistance of the first vaccine.

In certain cases, combining two agents can amplify the positive effects or benefits they provide individually. For example, combining a vaccine for a methanogen with a vaccine towards a separate microorganism that is syntrophic with the methanogen can amplify the positive effects or benefits of the vaccine by further reducing the fitness of the methanogen.

The level of synergism achieved when combining agents depends on numerous factors and is typically assessed through experimental studies or empirical observations specific to the agents and desired outcomes. Generally, combination of agents can lead to different degrees of synergisms ranging from No synergism to Supra-additive synergism.

No Synergism: In some cases, the combined effect of two or more agents may simply be additive or even less than additive. This means that the combined effect is equal to the sum of their individual effects or even lower. In such instances, no synergism is observed, and the agents may not interact in a way that amplifies their effects.

Mild to Moderate Synergism: A common outcome when combining agents is a mild to moderate level of synergism. This implies that the combined effect is greater than the sum of their individual effects, but not dramatically so. The degree of synergism may vary depending on the specific agents and the conditions of their interaction.

Strong Synergism: In some cases, the combination of agents can lead to a strong synergistic effect. This means that the combined effect is significantly greater than the sum of their individual effects. Strong synergism often results in an amplified and more potent effect, exceeding what would be expected based on the additive effects of the individual agents.

Supra-additive Synergism: In rare instances, the combined effect of two or more agents can be supra-additive, meaning it surpasses even strong synergism. Supra-additive synergism results in an exceptionally powerful effect that far exceeds the sum of the individual effects. Such cases are usually considered highly beneficial, as they can provide remarkable outcomes in terms of efficacy, efficiency, or other desired parameters.

Any suitable degree of synergism (%_{Synergism}) can be demonstrated with a combination therapy comprising any two or more compositions or agents of the present disclosure (e.g., a vaccine and another agent), such as an improvement of at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000%, for example 5-1000%, preferably 10-500%, more preferably 30-300%.

In certain embodiments, the improvement is measured in the amount of methane reduced when administered a combination therapy as compared to either agent alone, for example %_{Synergism}=Methane_{combo}*(Methane_{vaccine})⁻¹. In certain embodiments, the %_{Synergism} is measured in a herd of at least 10, 20, 30, 40, 50, 60, 70, 80, 90, 100, 500, or 1000 animals and/or not more than 20, 30, 40, 50, 60, 70, 80, 90, 100, 500, 1000, or 2000 animals and the statistical significance, e.g., coefficient of variation of %_{Synergism} within the herd is at least 50, 60, 70, 75, 80, 85, 90, 95, 99, 99.5, or 100%.

In certain embodiments, the synergism resulting from the combinatorial therapy results in a prolonged efficacy of the treatment as compared to either alone. In certain embodiments, the combinatorial therapy is effective for at least 1, 2, 3, 4, 5, 6, 7, 8, 9, 10, 11, 12, 16, or 20 months and/or not more than 2, 3, 4, 5, 6, 7, 8, 9, 10, 11, 12, 16, 20, or 24 months, for example 1-24 months. In certain embodiments, the length of efficacy of the treatment is at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000% greater than either agent alone, for example 5-1000%, preferably 10-500%, more preferably 30-300%.

An exemplary combinatorial therapy includes vaccination with a first vaccine encoding one or more methanogen surface proteins in combination with at least one additional vaccine encoding one or more of different methanogen surface proteins.

Another exemplary combinatorial therapy includes vaccination with a vaccine encoding one or more methanogen surface proteins in combination with administration of a small molecule inhibitor of methanogenesis. Without wishing to be bound to theory, it is hypothesized that the small molecule inhibitor of methanogenesis may remove the plurality of ruminal methanogens, and antibodies generated from the vaccination prevent new methanogens for colonizing the methanogen-deficient rumen.

Accordingly, in certain aspects, a vaccine of the present disclosure is administered to a subject conjointly or in a combination with at least one inhibitor of methane production described herein or those known in the art. In some embodiments, the at least one inhibitor is selected from Tables 9-13.

In some embodiments, the at least one inhibitor is administered to a subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some embodiments, the at least one inhibitor is administered to a subject daily, semiweekly, weekly, biweekly (every 2 weeks), monthly, semiannually, or annually. In some embodiments, the at least one inhibitor is administered to a subject every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the at least one inhibitor is administered to a subject for a duration of at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks. In some embodiments, the at least one inhibitor is administered to a subject for at least 1 week but no more than 1 month. In some embodiments, the at least one inhibitor is administered to a subject orally, intravenously, intramuscularly, or subcutaneously. In preferred embodiments, the at least one inhibitor is administered to a subject orally. In some embodiments, the at least one inhibitor is administered to a subject as a feed additive.

In some embodiments, the at least one inhibitor is administered to a subject concomitant with, prior to, or following the vaccination with a vaccine of the present disclosure. In some embodiments, the at least one inhibitor is administered to a subject on the same day as the subject is vaccinated. In some embodiments, a subject is administered with the at least one inhibitor one or more times to reduce the methane production by the subject, and said subject is vaccinated as a maintenance regimen.

In some embodiments, the at least one inhibitor comprises 3NOP. In some embodiments, a subject is administered at least about or no more than 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams (g) of 3NOP per dose. In some embodiments, 3NOP is administered to a subject every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

In some embodiments, at least about 0.5 g but no more than 25 g of 3NOP is administered to a subject in a given day. In preferred embodiments, about 2.5 g of 3NOP is administered to a subject in a given day. In some embodiments, about 2.5 g of 3NOP is administered to a subject per day for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

TABLE 9
Exemplary methanogenesis inhibitor

| Chemical | Reference |
|---|---|
| 1,3-bis-nitrooxypropane | US10154981B2 |
| 1,3-bis-nitrooxypropane | US9266814B2 |
| 1,4-bis-nitrooxybutane | US10806706B2 |
| 1,4-bis-nitrooxybutane | US10154981B2 |
| 1,4-bis-nitrooxybutane | US9266814B2 |
| 1,5-bis-nitrooxypentane | US10806706B2 |
| 1,5-bis-nitrooxypentane | US10154981B2 |
| 1,5-bis-nitrooxypentane | US9266814B2 |
| 11-nitrooxy undecanoic acid | US9365489B2 |
| 11-nitrooxy undecanoic acid | US10154981B2 |
| 2 mercaptoethanesulfonate | EP2747181A1 |
| 2-bromoethane sulfonic acid (2-BESA) | EP2747181A1 |
| 2-bromoethane sulphonate | US6251879B1 |
| 2-chloroethanesulfonate | EP2747181A1 |
| 2-nitropropanol | EP2747181A1 |
| 3-nitro-oxy-propyl 5-nitro-oxy-hexanoate | US10154981B2 |
| 3-nitro-oxy-propyl benzoate | US10154981B2 |
| 3-nitro-oxy-propyl hexanoate | US10154981B2 |
| 3-nitro-oxy-propyl propionate | US10154981B2 |
| 3-nitrooxy propionic acid | US9365489B2 |
| 3-nitrooxy propionic acid | US10154981B2 |
| 3-nitrooxy-propyl 5-nitrooxy-hexanoate | US9266814B2 |
| 3-nitrooxy-propyl benzoate | US9266814B2 |
| 3-nitrooxy-propyl hexanoate | US9266814B2 |
| 3-nitrooxy-propyl propionate | US9266814B2 |
| 3-nitrooxypropanol | US10806706B2 |
| 3-nitrooxypropanol | US10154981B2 |
| 3-nitrooxypropanol | US9266814B2 |
| 5-nitrooxy hexanoic acid | US10806706B2 |
| 5-nitrooxy pentanoic acid | US10806706B2 |
| 5-nitrooxy pentanoic acid | US9365489B2 |
| 5-nitrooxy pentanoic acid | US10154981B2 |
| 5-nitrooxy-N-methyl-pentanoic amide | US9365489B2 |
| 5-nitrooxy-N-methyl-pentanoic amide | US10154981B2 |
| 5-nitrooxy-N-pentanoic amide | US9365489B2 |
| 5-nitrooxy-pentane | US10154981B2 |
| 5-nitrooxy-pentane | US9266814B2 |
| 5-nitrooxy-pentanenitrile | US10154981B2 |
| 5-nitrooxy-pentanenitrile | US9266814B2 |
| 5-nitrooxy-pentanoic amide | US10154981B2 |
| 6-nitrooxy hexanoic acid | US9365489B2 |
| 6-nitrooxy hexanoic acid | US10154981B2 |
| 8-nitrooxy octanoic acid | US9365489B2 |
| 8-nitrooxy octanoic acid | US10154981B2 |
| 9-nitrooxynonanol | US10806706B2 |
| abietic acid | US10799544B2 |
| acetylene | EP2747181A1 |
| ajoene | US20220256890A1 |
| Alcaligenes faecalis | US20140072535A1 |
| allicin | US20220256890A1 |
| alliin | US20220256890A1 |
| allylpropyl disulfide | US20220256890A1 |
| Asparagopsis armata | US10881697B2 |
| Bacillus amyloliquefaciens | WO2021211548A1 |
| Bacillus strain 300 (NRRL No. B-50943) | US20210360945A1 |
| Bacillus strain 86 (NRRL No. B-50944) | US20210360945A1 |
| Bacillus, Lactobacillus, Strptocoecus, Candida and Pichia | WO2007072935A1 |
| bambermycin | AU2018229465A1 |
| benzylnitrate | US10154981B2 |
| berberine, nitrate, and eucalyptus oil | US10440975B2 |
| bis-(2-nitrooxyethyl) ether | US9266814B2 |
| bis(2-hydroxyethyl)amine dinitrate | US10806706B2 |
| bromochloromethane | US6251879B1 |
| Bromoforms | US20220175670A1 |
| chloroform | EP2747181A1 |
| Chloroforms | US20220175670A1 |
| Cysteine | US5843498A |
| dehydoabietic acid | US10799544B2 |
| diallyl disulfide | US20180093308A1 |
| diallyl disulphide | US20220256890A1 |
| diallyl trisulfide | US20180093308A1 |
| diallyl trisulfide | US20220256890A1 |
| egg powder from immunizing hens with methane-producing M. stadtmaniae | US7820171B2 |
| methanogens | |
| Eggs laid by hens innoculated with methanogen antigens | US20200121730A1 |
| essential oil | US20180001358A1 |
| ethyl 2 butynoate | EP2747181A1 |
| ethyl propionate | US20180093308A1 |
| ethyl-11-nitrooxy undecanoate | US9365489B2 |
| ethyl-11-nitrooxy undecanoate | US10154981B2 |
| ethyl-3-nitrooxy butanoate | US10154981B2 |
| ethyl-3-nitrooxy propionate | US9365489B2 |
| ethyl-3-nitrooxy propionate | US10154981B2 |
| ethyl-3-nitrooxy-butanoate | US9365489B2 |
| ethyl-4-nitrooxy butanoate | US10154981B2 |
| ethyl-4-nitrooxy-butanoate | US9365489B2 |
| ethyl-4-nitrooxy-cyclohexylcarboxylate | US9365489B2 |
| ethyl-4-nitrooxy-cyclohexylcarboxylate | US10154981B2 |
| ethyl-5-nitrooxy pentanoate | US9365489B2 |
| ethyl-5-nitrooxy pentanoate | US10154981B2 |
| ethyl-6-nitrooxy hexanoate | US9365489B2 |
| ethyl-6-nitrooxy hexanoate | US10154981B2 |
| ethyl-8-nitrooxy octanoate | US9365489B2 |
| ethyl-8-nitrooxy octanoate | US10154981B2 |
| ethylene | EP2747181A1 |
| eugenol | US7763273B2 |
| fluoroacetate | EP2747181A1 |
| fluvastatin | EP2747181A1 |
| halothane | US6251879B1 |
| hesperidin | EP2838376B1 |
| hop acid | US8197863B2 |
| hydrogen peroxide | WO2010071222A1 |
| hydroxycinnamic acid | WO2021038832A1 |
| Impatiens balsamina | NZ562783A |
| Iodoforms | US20220175670A1 |
| isonaringin | EP2838376B1 |
| isopimaric acid | US10799544B2 |
| isosorbid-dinitrate | US10154981B2 |
| isosorbid-dinitrate | US9266814B2 |
| laidlomycin | AU2018229465A1 |
| lasalocid | AU2018229465A1 |
| limonene | US7763273B2 |
| lovastatin | EP2747181A1 |
| lumazine | EP2747181A1 |
| medium and long chain fatty acids | EP2747181A1 |
| Megasphaera sp. & Coprococcus catus | US10961559B2 |
| methyl chloride | EP2747181A1 |
| methyl fluoride | EP2747181A1 |
| methyl-3-nitrooxy propionate | US9365489B2 |
| methyl-3-nitrooxy propionate | US10154981B2 |
| mevastatin | EP2747181A1 |
| Monascus sp. | US20130011384A1 |
| monensin | AU2018229465A1 |
| N-[2-(nitrooxy)ethyl]-3-pyridinecarboxamide | US10154981B2 |
| N-[2-(nitrooxy)ethyl]-3-pyridinecarboxamide | US9266814B2 |
| N-ethyl-3-nitro-oxy-propionic sulfonyl amide | US10154981B2 |
| neoabietic acid | US10799544B2 |
| neohesperidin | EP2838376B1 |
| nitrate | EP2747181A1 |
| nitrite | EP2747181A1 |
| nitroethane | EP2747181A1 |
| palustric acid | US10799544B2 |
| Peroxide source, peroxidase | US20210227850A1 |
| phosphate | EP2747181A1 |
| pimaric acid | US10799544B2 |
| poncirin | EP2838376B1 |
| Propionibacterium | US20120276058A1 |
| propynoic acid | EP2747181A1 |
| protease-resistant bacteriocin | EP1673983A1 |
| R(OC2H4)n(OH); R = C12-15; n = 7 | AU2013288441B2 |
| R1(CH2)nONO2; n = 1-15; R1 = H, C1-C6alkyl, phenyl, —OH, —NH2, —CN, | US10806706B2 |
| —COOH, —O(C═O)R8, —NHC(═O)R8, SO2NHR8, or —ONO2; R8 = C1-C6alkyl, | |
| phenyl, pyridyl; when n is >3 the hydrocarbon chain may be interrupted by —O— or | |
| —NH— | |
| rac-4-Phenylbutane-1,2-diyl dinitrate, 2-(hydroxymethyl)-2-(nitrooxymethyl)-1,3- | US10154981B2 |
| propanediol | |
| red yeast rice | US20140322798A1 |
| s-allyleysteine | US20220256890A1 |
| saponin | US20180001358A1 |
| Saponin & medium chain fatty acid | US10695393B2 |
| sodium bromoethenesulfonate (BES) | EP2747181A1 |
| vinyldithiines | US20220256890A1 |
| ω-nitrooxyC3-10alkane-1-ol | US20210392922A1 |
| 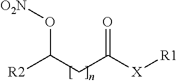 | US9365489B2 |
| 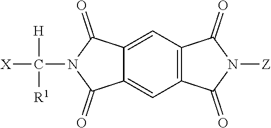 | WO2022103280A1 |
| 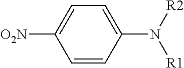 | US20160183564A1 |
| 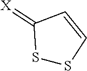 | EP3628169A1 |
| 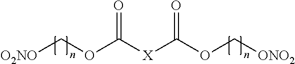 | US20210163397A1 |
| 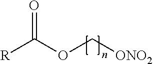 | WO2020221805A1 |
| 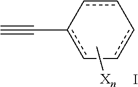 | AU2018383221A1 |
| 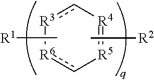 | US20030219467A1 |
| Asparagopsis taxiformis | USPP34607P3 |
| Lauric acid | |

*Each of the references in Table 9 is incorporated herein by reference.

Other exemplary inhibitors of methane production are discussed below.
Probiotics that Reduce Methane Production in Animals

Probiotics are a class of beneficial active microorganisms or their cultures. Probiotics are useful in reducing CH4 emissions in animals (Table 10). There are many types of probiotics, and different strains have different inhibitory effects on CH4 emissions. For example, the GA03 strain of Acetobacter is more effective at inhibiting CH4 production than other isolated strains. Most probiotics reduce CH4 production by influencing the activities of ruminal microorganisms, with no adverse effects on animals. In addition, probiotics enhance ruminal fermentation.

Lactic acid bacteria, which have been used as feed additives for a long time, not only reduce CH4 emissions per unit volatile fatty acid (VFA) output, but also improve the fermentation quality and fiber digestibility of silage. In addition, the denitrifying bacterium Bacillus 79R4 could prevent NO-2-N poisoning and microbial ecosystems from impairing fermentation efficiency. Furthermore, Bacillus licheniformis reduces CH4 production and increases feed energy and protein utilization.

TABLE 10
Exemplary probiotic bacteria that reduce methane production

| | | Addition amount; maximum | |
|---|---|---|---|
| Types of probiotics | Inhibitory effect | suppression methane amount | Inhibition mechanism |
| Propionic acid bacillus | [symbol] (Most propionic bacteria) | 100 μL of the propionic acid bacteria | Unknown |
| | [symbol] (P. jenscenii LMGT282 | culture (2 × 10 [symbol] to 4 × 10 [symbol] | |
| | and P. thoenii LMGT2827 or T [symbol] ) | colony forming units), Propioni- | |
| | | bacterium thoenii T159; 20% | |
| Lactic acid bacteria | [symbol] | [symbol] cfu/g fresh weight, Lacto- | Hydrogen consumption |
| | | bacillus plantarum, 8.8 ml/g(72 h) | |
| Acetic acid bacteria | [symbol] | 1% Proteiniphilum acetatigenes | Reduced the number of |
| | | GA [symbol] | methanogens |
| Enterococcus faccium | [symbol] | 0.1%; 2.08 mM/mL | Alters microbial flora |
| SROD | | | |
| Probiotic products | [symbol] | 2 g probiotic products in | Reduce the number of |
| of Ruminococcus | | powder 1.2 ml/g of dry matter | rumen protozoa |
| flavefaciens | | 10 ml probiotic products in liquid; | |
| | | 1.2 ml/g of dry matter | |
| Baccillus licheniformis | [symbol] | 2.5 × 10 [symbol] 2.7 [symbol] | Unknown |
| Saccharomyces | [symbol] ( [symbol] et al., 2019) | — | Affects rumen microbes |
| cerevistae | ( [symbol] et al., 2019) | | |

[symbol]

indicates data missing or illegible when filed

Prebiotics that Reduce Methane Production

Prebiotics are substances that are not easily digested or absorbed by the host. They selectively stimulate the growth and activity of one or several ruminal microorganisms with a positive effect on ruminal fermentation. Prebiotics suppress ruminal CH4 production in subjects. Prebiotics mainly reduce rumen CH4 production by altering the bacterial community structure, influencing the permeability of the cell walls of methanogenic archaea, and stimulating other bacteria to compete with methanogens for H2 (Table 11).

The prebiotic chitosan can influence bacterial community structures by altering microbial population compositions, for example, by replacing fibrinolytic enzyme-producing microbes (Firmicutes and Fibrobacteres) with amylolytic enzyme-producing microbes (Bacteroides and Proteus); in turn, reducing CH4 production. Chitosan could influence the ruminal fermentation process by altering VFA distributions and increasing propionic acid concentrations, which reduces CH4 production in turn. However, the reduction in CH4 is associated with the degree of chitosan deacetylation, which could alter the permeability of the methanogen cell wall. In addition, various yeast products could reduce CH4 emissions by stimulating acetic acid-producing bacteria to compete with methanogens or metabolize hydrogen.

TABLE 11
Exemplary prebiotic bacteria that reduce methane production

| | | Addition amount; maximum | |
|---|---|---|---|
| Types of prebiotics | Inhibitory effects | methane suppression amount | Inhibition mechanismi |
| Chitosan | — | 3000 (molecular weights) | (1) Alters microbial |
| | | dry matter; 22.9% | community structure |
| | | ml/day (Tong et al., 2020) | (Tong et al., 2020) |
| | | 2% Chitosan + 21% of crude | (2) Alters fermentation |
| | | glycerin; 53.679% ( [symbol] | pathway ( [symbol] |
| | | [symbol] 2020) | [symbol] |
| | | | [symbol] 2020) |
| | | | (3) Influences |
| | | | methanogenic bacteria |
| | | | cell wall permeability |
| | | | (Zanferari et al., 2018) |
| Yeast products | — | 4 mg/1 g dry matter; [symbol] | Indirect consumption of |
| | | | hydrogen |

[symbol]

indicates data missing or illegible when filed

Other Agents that Reduce Methane in Animals

Among the CH4 mitigation options, inhibiting the growth or the metabolic activity of methanogens is the most effective approach. Another strategy is to modulate rumen microbiome so that fermentation is shifted toward decreased H₂ production and increased production of reduced VFA (e.g., propionate). Provided herein are exemplary and non-exhaustive descriptions of anti-methanogenic compounds evaluated with a focus on their impact rumen methanogens.

### Coenzyme M Analogs

Methyl-CoM reductase (Mcr) mediates the final step of all the methanogenesis pathways and CoM (2-mercaptoethanesulfonic acid) is an essential cofactor serving as the methyl group carrier. Mcr reduces methyl-CoM to CH₄. CoM is found in all known methanogens but not in other archaea or bacteria. Several halogenated sulfonated compounds, including 2-bromoethanesulfonate (BES), 2-chloroethanesulfonate (CES), and 3-bromopropanesulfonate (BPS), are structural analogs of CoM, and they can competitively and specifically inhibit Mcr activity, lowering CH₄ production at relatively low concentrations. Different species of methanogens vary in sensitivity to these inhibitors. Of three species tested on BES, Mbb. Ruminantium was the most sensitive, while Methanosarcina mazei was the least sensitive, with Methanomicrobium mobile being intermediate. All three species appeared to be resistant to BPS up to 250 μmol/L in pure cultures. The different sensitivity to these CoM analogs has been attributed to varying ability to uptake these inhibitors into the cells. Methanogens able to synthesize their own CoM are less dependent on external CoM and are thus less sensitive. Mbb. Ruminantium is the only ruminal methanogen that requires CoM synthesized by other methanogens.

Halogenated Aliphatic C₁-C₂ Hydrocarbon

Halogenated aliphatic compounds with 1 or 2 carbons, such as chloroform, bromochloromethane (BCM), bromoform, bromodichloromethane, dibromochloromethane, carbon tetrachloride, trichloroacetamide, and trichloroethyladipate, can lower ruminal CH₄ production. These halogenated compounds block the function of corrinoid enzymes and inhibit cobamide-dependent methyl group transfer in methanogenesis. These halogenated compounds also competitively inhibit CH₄ production by serving as terminal electron (e) acceptors. Drenching chloroform to cattle inhibited methanogenesis substantially for up to 32 days without affecting feed digestion or basic rumen function. The addition of BCM depressed CH₄ production both in vitro and in vivo. In steers fed grain-based diets, BCM decreased CH₄ production by 50 to 60% with no signs of toxicity or residues in meat. It was also reported that the abundance of total bacteria and protozoa was not changed, but methanogenesis and growth of methanogens were drastically inhibited by BCM in both batch cultures and continuous fermenters. While the commercial use of chloroform, a recognized carcinogen, is not practical, it provides validation for the class of BCM compounds in reducing methane production.

Some marine plants such as red seaweed, and algae, lichen, and fungi can contain high concentrations of organobromine compounds such as bromomethane and bromoform. A recent in vitro study showed that red seaweed Asparagopsis taxiformis lowered CH₄ production by 99% at a dose of 2% of organic matter substrate. No adverse effect on feed digestion or fermentation was noted at ≤5% (of dry matter) inclusion. Thus, red seaweed, and probably other organobromine-rich plants, may offer a potentially practical natural approach to mitigate CH₄ emission.

### Nitrooxy Compounds

3-Nitrooxypropanol (3NOP) and ethyl-3NOP, two new synthetic compounds, have been shown to have specific anti-methanogenic properties. 3NOP appears to inactive Mcr by competitively binding to the Mcr active site and then oxidizing the Ni¹⁺ that is required for Mcr activity. Feeding of 3NOP at a dose rate of 2.5 g/day/cow mixed in diets decreased CH₄ emission by 60% per kg of DM intake. In a study using beef cattle, 3NOP fed at 2.0 g/day/cow decreased CH₄ yield by 59%, and the inhibition persisted for up to 112 days without much effect on feed intake, nutrient digestibility or total VFA concentrations. In one recent study, 3NOP fed at 40-80 mg/kg feed DM in dairy cows decreased CH₄ production by about 30% persistently for up to 84 days. Similarly, 3NOP fed at 2.5 g/day/cow decreased CH₄ yield by 37% in dairy cows. In sheep, 3NOP at 0.5 g/day also decreased CH₄ production by 29% without adverse effect on digestion or rumen fermentation. However, when 3NOP was directly added to the rumen through rumen cannula at a daily dose of 0.50 or 2.5 g per cow (equivalent to 25 to 125 mg/kg feed dry mailer), the degree of CH₄ suppression declined to 7-10%. The later study suggests that 3NOP needs to be fed together with the diet to achieve higher efficacy. Thus, 3NOP could be used to lower CH₄ emission from cows and sheep without adverse effects on nutrient utilization or animal performance. It has been demonstrated that 3NOP indeed decreased methanogen abundance while increasing the abundance of protozoa.

TABLE 12
Exemplary nitrogen-containing compounds that reduce methane production in animals

| | Inhibitory | Addition amount; maximum | |
|---|---|---|---|
| Types of nitrogenous compounds | effect | methane suppression amount | Inhibition mechanism |
| Nitrate | — | 20 mg/g dry matter; 21% | (1) Hydrogen consumption; |
| | | (Alvarez- [symbol] et al., 2019) | (2) Inhibits the proliferation |
| | | 5 mmol/L; 32.92% | of methanogen [symbol] reduces their |
| | | (Wa et al., 2019) | activity and abundance |
| | | 5 mM; 43.26% Lin rt al., 2017) | |
| Encapsulated nitrate (EN) | — | 70 g/100 kg of body | Reduces methane reducing |
| | | weight; 18.59% CH₄/kg of | bacteria |
| | | forage dry matter intake | |
| | | (Granja- [symbol] et al., 2019) | |
| | | 2.5%; 9.37 mM/d | |
| | | (Capelari et al., 2018) | |
| | | 2.5%, 2.8 g/kg Dry matter intake | |
| | | (Alema et al., 2019) | |
| Urea and nitrate mixture | — | 34 g/kg straw dry matter + 6 g/kg | Indirect consumption of hydrogen |
| | | dry matter of ammonium nitrate; | |
| | | 10.2% (Zhang et al., 2019) | |
| | | Urea + ammonium nitrate (34 + | |
| | | 6 g/kg of dry matter, respectively); | |
| | | 3.1 mL/g dry matter (Zhang et al., | |
| | | 2018) | |
| Nitroethane (NE), 2-Nitroethanol | — | 10 mmol/L; 96.7% (NE), 96.7% | (1) Inhibits the activity |
| (NEOH), 2-Nitro-1-Propanol | | (NEOH), 41.7% (NPOH) | of methanogens; |
| (NPOH) | | | (2) Inhibits methyl- |
| | | | coenzyme M gene expression; |
| | | | (3) Reduces the content of |
| | | | coenzyme F420 and F430 |
| 3-Nitrooxypropanol (NOP) | — | 0.08 mg/g dry matter; 44% | Inhibits methyl-Coenzyme M |
| | | (Alvarez- [symbol] et al., 2019) | activity |
| | | 2.5 g/animal/day; 38%/kg | |
| | | dry matter intake (Martinez- | |
| | | Fernandez et al., 2018) | |
| | | 60 mg/kg of feed dry matter; | |
| | | 26%/day (Melgar et al., 2020) | |
| | | 1.6 g; 28%(roughage), 23% | |
| | | (concentrate pellet) (Van | |
| | | [symbol] et al., 2019) | |

[symbol]

indicates data missing or illegible when filed

### Pterin Compounds

Pterin is a group of structural analogs of deazaflavin (F₄₂₀), which is a coenzyme involved in two steps of the hydrogenotrophic methanogenesis pathway. Therefore, pterin compounds can competitively inhibit CH₄ production. In one study, CH₄ production by Mbb. ruminantium, Ms. mazei, and Mm. mobile was significantly decreased by lumazin (2,4-pteridinedione) in a dose-dependent manner from 0.06 to 0.24 mmol/L. As expected, pterin is much less efficacious in mixed rumen cultures than in pure methanogen cultures. It was suggested that lumazine could be degraded or transformed by some microbes in mixed cultures or adsorbed to solid particles where it becomes unavailable to methanogens. Some N-substituted derivatives of p-aminobenzoic acid, which are inhibitors of methanopterin synthesis in methanogens, decreased methanogenesis in ruminal cultures without inhibiting VFA production.

### Hydroxymethylglutaryl-CoA (HMG-S-CoA) Reductase Inhibitors

All archaea contain long-chain isoprenoid alcohols as the major component of their cell membrane. Isoprenoid alcohols are unique to archaea. They are synthesized from mevalonate that is formed by reduction of 3-hydroxy-3-methylglutaryl coenzyme A (HMG-S-CoA) catalyzed by HMG-S-CoA reductase. This enzyme is also used for the synthesis of the same precursor ultimately used in cholesterol synthesis in humans. As an inhibitor of HMG-S-CoA reductase, statins (e.g., lovastatin and mevastatin) can inhibit the growth of methanogens by inhibiting the synthesis of mevalonate. Lovastatin and mevastatin may also act as a potential inhibitor of F₄₂₀-dependent NADP oxidoreductase as shown in the model structure of that enzyme. In the earliest reported study, mevastatin at 5.6 μmol/L inhibited the growth of all three strains of rumen Methanobrevibacter, but not rumen bacteria in vitro. In studies using a rumen simulation technique (Rusitec), lovastatin (150 mg/L) reduced CH₄ production by approximately 42% without altering bacterial counts or nutrient fermentation.

The high cost of pure statins promoted a search for natural sources of statins as agents to mitigate CH₄ production. Lovastatin is a secondary metabolite of idiophase of several fungal species (e.g., Penicillium spp., Aspergillus terreus, Monascus purpureus, and Pleurotus ostreatus), and it can reach a concentration up to 2.8% of the dry weight of P. ostreatus (oyster mushrooms) and 261 mg/kg fermented rice straw. Lovastatin extracted from fermented rice straw significantly reduced total CH₄ production by rumen methanogens by nearly 28% after 48 h in vitro incubation. Extract from A. terreus-fermented rice straw containing lovastatin (97 mg/g dry mass) also significantly reduced CH₄ production and abundance of methanogens, especially Methanobacteriales, and aerobic fungi, but increased several fiber-degrading bacteria. Lovastatin also altered the morphology of M. smithii significantly, resulting in abnormal membrane formation and asymmetric cell divisions and increased HMG-S-CoA reductase gene expression. Fermented rice straw extract also modulated expression of several genes associated with methanogenesis, increasing expression of mtr, mta, and mcr while decreasing expression of hmd and fno. Supernatant fractions containing statins produced by Mortierella wolfii also appeared promising to inhibit methanogenesis without reducing overall fermentation. In another study using sheep, fermented rice straw containing metabolites (possibly pravastatin and mevastatin) produced by Monascus spp. Decreased CH₄ emission (by 30%), the abundance of methanogens, and ruminal acetate:propionate ratio compared with the unfermented rice straw.

Diallyl disulfide, the main ingredient of garlic oil, is known to inhibit HMG-S-CoA reductase. Garlic oil (300 mg/L) was more effective than lovastatin as an inhibitor of CH₄ production (by up to 91% reduction); however, garlic oil also inhibited bacterial growth, which likely reduces the availability of methanogenesis substrates. Garlic oil lowered CH₄ production in vitro and growth of methanogens, altered community structure of methanogens after 24 h incubation. Moreover, interestingly, the anti-methanogenic efficacy increased over time up to 18 days of incubation.

### Plant Secondary Metabolites

Plants secondary metabolites (PSM), such as saponins, tannins, flavonoids, organosulphur compounds, and essential oils, have anti-microbial activities against several types of microorganisms. Numerous PSM extracts have been recognized as potential inhibitors of rumen methanogens and CH₄ production. Some forage plants rich in tannins and saponins have also shown promise at mitigating CH₄ emission from animals.

TABLE 13
Exemplary plant extracts that reduce methane production in animals

| | | Inhibitory | Addition amount; maximum | |
|---|---|---|---|---|
| Types of probiotics | | effects | suppression methane amount | Inhibition mechanisms |
| Plant extracts | Corymbia [symbol] leaf | — | 10 ml/calf/day; — | (1) Protozoa number |
| | | | | reduced (1.84 × 10 [symbol] /ml) |
| | | | | (2) Ratios of volatile fatty |
| | | | | acids altered |
| | Aloe vera, [symbol] | — | 25 mg/L and 50 mg/L Axadirachta | Unknown |
| | papaya, [symbol] | | indica, Carica papaya, Tithonia | |
| | indica, Moringa oleifern, | | diversifolia; 15% Jatropha | |
| | Tithania diversifolia, | | curcas and Moringa oleifera pods; | |
| | Jatropha curcas, and | | 30% ( [symbol] Hassen, 2018) | |
| | Moringa oleifera pod | | Eragrostis substrate, 4 ml plant | |
| | extracts | | extracts; 50% reduction in methane/ | |
| | | | Total gas production ( [symbol] | |
| | | | Hassen [symbol] 2020) | |
| | Pomegranate peel extract | — | 2% of dry matter intake, | Unknown |
| | and desert teak | | Punica granatum; 40% | |
| | extract | | T [symbol] mella undulata; 42% | |
| | Rhus [symbol] extract | — | 50 mg/L; lowest | Unknown |
| | [symbol] and Acacia | — | 2% dry matter basis, [symbol] | Unknown |
| | [symbol] extract | | Catechu; 21% Acacia [symbol] | |
| | | | 23% | |
| | [symbol] | — | 1.0%; 67.2% (Roque et al., 2019) | Unknown |
| | Garlic extract | — | [symbol] (Kim et al., 2018b) | Decreased abundance of |
| | | | 1 g the experimental mixture; 6.9 ± | methanogenic archaea |
| | | | 10.7 ml/d ( [symbol] et al., 2018) | |
| | Plant extract [symbol] | — | 25 mg, high-forage diets; 60% | Decreased abundance of |
| | | | high-concentrate diets; 41% | Methanobacter |
| | Plant extract [symbol] | — | 12 mM, [symbol] acid; 37 [symbol] % | Unknown |
| | acid and p-coumaric | | p-coumaric acid; 28 [symbol] % | |
| | acid | | | |

[symbol]

indicates data missing or illegible when filed

### 1. Tannins

Tannins decrease CH₄ production by directly inhibiting methanogens and indirectly decreasing H₂ production as a result of decreased fiber digestion and protozoal population in the rumen. The inhibitory activity of tannins extracted from Lotus pedunculatus was demonstrated on pure cultures of methanogens. The inhibition of methanogen populations was also shown by tannins in the rumen of goats fed diets containing tannins. Studies on structure-activity relationships have shown that types and molecular weights of tannins are important in determining their potency in lowering CH₄ production and abundance and diversity of rumen methanogens, with high molecular weight condensed tannins (CT) being more potent. Such structure-activity relationships have been demonstrated using members of Methanobacteriales including Methanobrevibacter.

### 2. Flavonoids

Flavonoids have not been extensively evaluated with respect to rumen methanogenesis. It was reported that inclusion of flavone, myricetin, naringin, rutin, quercetin, or kaempferol decreased in-vitro CH₄ production by 5 to 9 mL/g DM. Their potency ranked as follows: myricetin≥kaempferol≥flavone>quercetin≥naringin>rutin≥catechin. Catechin decreased CH₄ production both in vitro and in vivo. All the flavonoids, when fed at 0.2 g/kg DM, noticeably decreased relative abundances of hydrogenotrophic methanogens, and citrus (Citrus aurantium) extract rich in mixed flavonoids and its pure flavonoid components, neohesperidin and naringin, appeared to result in the greatest inhibition. Methanosarcina spp. Were also inhibited by poncirin, neohesperidin, naringin and their mixture. Flavonoids directly inhibit methanogens and also likely acts as H₂ sinks via cleavage of ring structures (e.g., catechin) and reductive dihydroxylation.

### 3. Saponins

The effects of saponins on rumen fermentation, rumen microbial populations, and ruminant productivity have been examined extensively. Quillaja saponin at 1.2 g/L, but not at 0.6 g/L, lowered CH₄ production in vitro and the abundance of methanogens (by 0.2-0.3 log) and altered their composition. Ivy fruit saponin decreased CH₄ production by 40%, modified the structure of the methanogen community, and decreased its diversity. Saponins from Saponaria officinalis decreased CH₄ and abundance of both methanogens and protozoa in vitro. It is hypothesized that saponins lower H2 production, thereby reduce CH4 production.

### 4. Essential Oils

The effects, mostly beneficial, of essential oils (EO) on rumen fermentation, microbial populations, and ruminant productivity have frequently been reviewed. Several EO compounds, either in pure form or in mixtures, are anti-methanogenic. The effects of EO on CH₄ production and methanogens are variable depending on dose, types, and diet. Five EO (clove, eucalyptus, peppermint, origanum, and garlic oil) that have different chemical structures in vitro at three different doses (0.25, 0.50 and 1.0 g/L) were tested for their effect on CH₄ production and archaeal abundance and diversity. Overall, all these EO suppressed CH₄ production and abundance of archaea and protozoa in a dose-dependent manner, but they differed in potency. Thyme oil or cinnamon oil fed to Holstein steers at 0.5 g/day decreased the relative abundance of total protozoa and methanogens. However, feeding beef cattle a blend of EO (CRINA®) did not affect CH₄ production, methanogen abundance or its diversity. Overall, methanogens may be directly inhibited or indirectly inhibited by Eos via inhibition of protozoa and H₂-producing bacteria in the rumen.

### Alternative Hydrogen Sinks

Compounds with a redox potential higher than CO₂ can thermodynamically outcompete CO₂ for reducing equivalents produced during rumen fermentation. These compounds, thus, can be used as alternative e⁻ acceptors to redirect e⁻ flux away from methanogenesis. The commonly evaluated alternative e⁻ acceptors are discussed below.

### 1. Nitrate and Sulfate

Nitrate (NO₃¹⁻) decreased CH₄ production both in vitro and in vivo. Mechanistically, nitrate decreases CH₄ production by outcompeting CO₂ as an e⁻ acceptor, and its reduction intermediates, nitrite (NO₂¹⁻) and nitrous oxide (N₂O), also directly inhibit methanogens as well as some H₂ producers. Sulfate also lowers CH₄ production, but much less effectively than nitrate. Archaeal abundance declined in goats receiving nitrate. While nitrate is not toxic to methanogens, it is toxic to protozoa, fungi and to a lesser extent to select bacterial species, suggesting a more general toxicity of nitrate. Nitrate can replace a portion of the dietary nitrogen as it is reduced to ammonia.

### 2. Nitrocompounds

A few organic nitrocompounds have been evaluated for their efficacy to decrease methanogens and CH₄ production. These compounds can serve as e⁻ acceptors by some bacteria competing with methanogens for reducing equivalents. This is demonstrated by nitroethane that can be used as a terminal e⁻ acceptor by Dentitrobacterium detoxificans, thereby indirectly decreasing CH₄ production. Nitrocompounds may also inhibit methanogenesis by directly inhibiting the activity of formate dehydrogenase/formate hydrogen lyase and hydrogenase, all of which are involved in the early step(s) of the hydrogenotrophic methanogenesis pathway, or inhibiting e⁻ transfer between ferredoxin and hydrogenase.

Nitrocompounds generally are quite effective in lowering CH₄ production, with 3-nitro-propionate, 2-nitropropanol, 2-nitroethanol and nitroethane being able to decrease CH₄ production by 57 to 98% in vitro. Using sheep, it was shown that nitroethane decreased CH₄ production by up to 45% and 69%, respectively, when orally administrated at 24 and 72 mg/kg body weight daily for 5 days. Although less effective than nitroethane, 2-nitropropanol also significantly lowered CH₄ production (by 37%) in steers.

### 3. Propionate and Butyrate Enhancers

Malate, acrylate, oxaloacetate, and fumarate are intermediates of carbohydrate fermentation. They can be converted to propionate or used in anabolism for the synthesis of amino acids or other molecules. They can accept reducing equivalents and thus stoichiometrically lower H₂ available for CH₄ production. When added at a concentration of 3.5 g/L, fumarate decreased CH₄ production by 38% in continuous fermenters with forages as a substrate. Types of forages and their combinations appeared to affect the anti-methanogenic efficacy of fumarate, ranging from 6 to 27% inhibition at 10 mmol/L. Acrylate also depresses CH₄ production in the rumen, but to a lesser extent than an equimolar level of fumarate. Malate was found to decrease CH₄ production by beef cattle in a dose-dependent manner, with a 16% decrease being noted when fed at 7.5% of DM intake, which corresponds to a 9% reduction per unit of DM intake. Different studies reported different anti-methanogenic potencies of this type of e⁻ acceptors. Fumarate fed to goats at 10 g/day/goat was found to decrease the abundance of methanogens and CH₄ production only by 11.9% while increasing concentrations of total VFA, acetate and propionate. Some of the intermediates of pyruvate conversion to butyrate can act as e⁻ acceptors, which could also decrease CH₄ production.

### 4. Unsaturated Organic Acids

Unsaturated fatty acids can act as hydrogen sinks during their biohydrogenation and thereby lower CH₄ production. Propynoic acid (an unsaturated analog of propionic acid), 3-butenoic acid and 2-butynoic acid (both unsaturated analogs of butyric acid), and ethyl 2-butynoate each at 6 to 18 mmol/L have been evaluated as alternative e⁻ sinks to lower methanogenesis in vitro. Only propynoic acid and ethyl 2-butynoate markedly lowered CH₄ production, by 65 to 76% and 24 to 79%, respectively. In another study, propynoic acid lowered CH₄ production by 67% and 78% at 6 and 12 mmol/L, respectively and decreased methanogen abundance. Propynoic acid and ethyl 2-butynoate are directly toxic to methanogens, and species of methanogens vary in their sensitivity to these two inhibitors, with Mbb. ruminantium being most sensitive, Ms. mazei least sensitive, and Mm. mobile intermediate.

### Inhibitors to Hydrogen-Producing Bacteria

### 1. Ionophores

Ionophores, such as monensin and lasalocid, are commonly used to improve rumen microbial metabolism. Being highly lipophilic ion carriers, they pass through the cell wall of Gram-positive bacteria and penetrate into the cell membrane. Therein, they serve as H⁺/Na⁻ and H⁺/K⁻ antiporters, dissipating ion gradients that are needed for ATP synthesis, nutrient transport, and other essential cellular activities and ultimately resulting in delayed cell division and even cell death. Ionophores preferentially inhibit Gram-positive bacteria, including members of class Clostridia, including Ruminococcus species that produce acetate and H₂. Ionophores can also inhibit some Gram-negative rumen bacteria, including bacteria that produce formate and H₂. Therefore, ionophores may lower CH₄ emission by decreasing H₂ production. For examples, monensin fed at 24-35 mg/kg diet lowered CH₄ production by up to 10% (g/kg DM intake), though no CH₄ suppression was observed at 10-15 ppm. In a recent in vivo study, however, monensin at 60 mg/day/cow did not lower CH₄ production by tropical cattle, though it decreased CH₄ production by about 30% when fed at 250 mg/day/cow. As repeatedly noted, at such high supplementation level, DM intake was lowered, which explains most of the observed decrease in CH₄ emission. Ionophores are not known to directly inhibit methanogens, but they can change the population dynamics of methanogen species. For example, monensin decreased the population of Methanomicrobium spp. While increasing that of Methanobrevibacter spp. Total methanogens were also decreased in cattle fed monensin. These can be explained by reduced availability of H₂ and differences in affinity for H₂ and growth kinetics among methanogen species.

### 2. Bacteriocins

Bacteriocins are proteins or peptides produced by bacteria and inhibit select microbial species in the rumen and other habitats. There are only a few studies investigating the effect of bacteriocins on CH₄ emission. Bovicin HC5, a bacteriocin produced by Streptococcus spp. From the rumen, was reported to suppress CH₄ by 50% in vitro. Nisin, a bacteriocin produced by Lactobacillus lactis subsp. Lactis, has also been shown to decrease CH₄ production in vitro by up to 40% depending upon its concentration. Similar to monensin, bacteriocins probably modulate rumen fermentation leading towards increased propionate, thereby decreasing CH₄ production.

Additional Agents that Reduce Methane in Animals

### Biochemical Pathways

In certain embodiments, the one or more deleterious atmospheric gases and/or precursors thereof are microbially derived through one or more biosynthetic pathway. The deleterious atmospheric gas can be any suitable deleterious atmosphere gas, such as carbon dioxide, methane, nitrous oxide, or a combination thereof. The deleterious atmospheric gas precursor can be any suitable precursor, such as acetate, hydrogen, carbon, methanol, monomethylamine, dimethylamine, trimethylamine, nitric oxide, or a combination thereof. In preferred embodiments, the deleterious atmosphere gas comprises carbon dioxide, hydrogen, or methane more preferably methane. In certain embodiments, wherein the resultant deleterious atmospheric gas comprises methane, the one or more biosynthetic pathways include the acetoclastic, hydrogenotrophic, and methylotrophic pathways, which differ based on the starting substrates, i.e., precursor, more preferably the acetoclastic or hydrogenotrophic pathways, even more preferably the acetoclastic pathway.

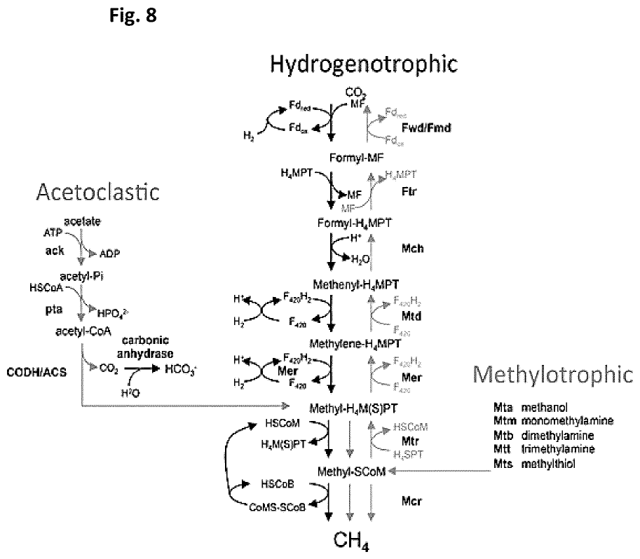

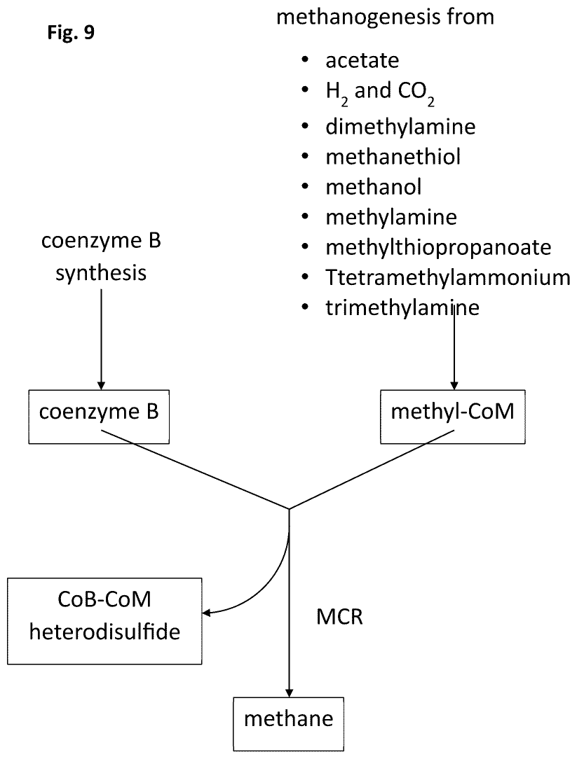

The acetoclastic pathway comprises a series of enzymes that convert the precursor acetate through a series of enzymatic conversions to methane. Starting from acetate, (1) acetate is converted to acetyl phosphate by acetate kinase (ack); (2) acetyl phosphate is converted to acetyl-CoA by phosphotransacetylase (pta); (3) the acetyl group from acetyla-CoA is transferred to a protein intermediate by acetyl-CoA decarbonylase; (4) the acetyl group is then transferred to tetrhydrosarcinapterin to form 5-methyl-tetrahydrosarcinapterin by methyltetrahydrosarcinapterin methyltransferase; (5) 5-methyl-tetrahydrosarcinapterin is converted to methyl-CoM by methyl-H₄SPT:CoM methyltransferase (Mtr); and (6) methyl-CoM is reduced to methane by methyl-CoM reductase (Mcr) (FIG. 8 and FIG. 9).

The hydrogenotrophic pathway comprises a series of enzymes that convert the precursors hydrogen and carbon dioxide to methane. Starting from carbon dioxide and hydrogen, (1) a formylmethanofuran dehydrogenase (Fwd/Fmd) produces a formylmethanofuran, (2) which is further converted into 5-formyl-tetrahydromethanopterin by a formylmethanofuran:H₄MPT formylatransfer (Ftr); (3) 5-formyl-tetrahydromethanopterin is further converted into 5,10-methenyltetrahydromethanopterin by methyl-H₄MPT cyclohydrolase (Mch); (4) 5,10-methenyltetrahydromethanopterin is converted to N₆-methyltetrahydromethanopterin by F₄₂₀-dependent methylene-H₄MPT reductase (Mer); (5) N₆-methyltetrahydromethanopterin is converted to methyl-CoM by methyl-H₄MPT:coenzyme M methyltransferase (Mtr); and (6) methyl-CoM is reduced to methane by methyl-CoM reductase (Mcr) (FIG. 8 and FIG. 9).

The methylotrophic pathway comprises a series of enzymes that convert one or more of dimethylamine, methanethiol, methanol, methylamine, methylthiopropanoate, tetramethylammonium, and/or trimethylamine into methyl-CoM, wherein methyl-CoM is reduced to methane by methyl-CoM reductase (Mcr) (FIG. 8 and FIG. 9).

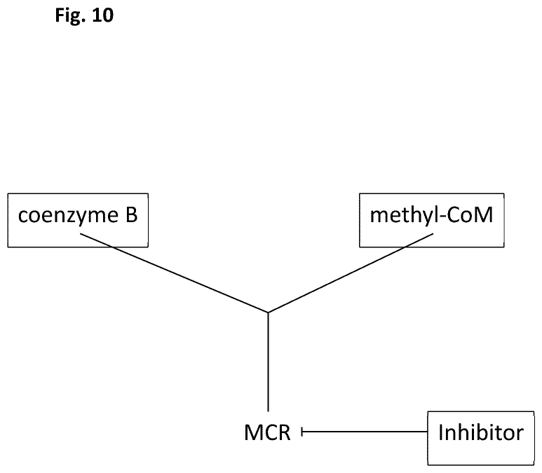

In certain embodiments, provided herein are compositions, methods, and/or kits comprising one or more small molecules that reduce the activity of one or more enzymes in one or more methane biosynthetic pathways. The enzyme can be any suitable enzyme, such as 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex β, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine-[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine-corrinoid protein Co-methyltransferase, or a combination thereof. In a preferred embodiment, the enzyme comprises methyl-CoM reductase (Mcr) (FIG. 10).

Compositions for Reducing Production of Deleterious Atmospheric Gases and/or Precursors Thereof In certain embodiments provided herein are compositions. In certain embodiments, provided herein are compositions comprising one or more small molecules. In preferred embodiments, provided herein are compositions comprising one or more small molecules that reduce the production of one or more deleterious atmospheric gases and/or precursors thereof.

The small molecule can be any suitable small molecule for reducing the production of one or more greenhouse gases and/or precursors thereof, for example a small molecule that interferes with the uptake and/or conversion of acetate, hydrogen, carbon dioxide, methanol, monomethylamine, dimethylamine, trimethylamine, nitric oxide, or a combination thereof, and/or a small molecule that interfere with the production of carbon dioxide, hydrogen nitrous oxide, or a combination thereof. In preferred embodiments, the small molecule interferes with the uptake and/or conversion of acetate, hydrogen and/or carbon dioxide and/or the production of carbon dioxide or methane, more preferably with the production of methane.

Small Molecules that Affect Production of Deleterious Atmospheric Gases and/or Precursors Thereof

In certain embodiments, provided herein is a composition for reducing emissions of deleterious atmospheric gasses and/or precursors thereof comprising: one or more small molecules that reduce the production of one or more deleterious atmospheric gasses and/or precursors thereof. The one or more small molecules that reduce the production of one or more deleterious atmospheric gasses and/or precursors can be any suitable molecule.

In certain embodiments, the one or more small molecules that reduce the production of one or more deleterious atmospheric gasses and/or precursors comprises a compound with the formula the formula R¹—[CH₂]ₙ—ONO₂
wherein
n is an integer from 1 to 15;
R¹ is selected from the group consisting of H, C₁-C₆alkyl, phenyl, —OH, —NH₂, —CN, —COOH, —O(C═O)R³, —NHC(═O)R³, SO₂NHR³, or —ONO₂, —SH and R³ is C₁-C₆alkyl, phenyl, pyridyl;
with the proviso that when n is >3 the hydrocarbon chain may be interrupted by —O— or —NH—.

In some embodiments, the one or more small molecules that reduce the production of one or more deleterious atmospheric gases and/or precursors comprises 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, or any combination thereof. Preferably, the one or more small molecules is 3-nitrooxypropanol (3NOP).

In some embodiments, the composition comprises about 1 to about 25% by weight of the small molecule, about 5 to about 20% by weight of the small molecule, or about 5 to about 15% by weight of the small molecule.

### Solid Carriers

In certain embodiments, the composition further comprises one or more solid carriers. As used herein, the term “solid carrier” includes additives commonly used in the preparation of powderous formulations such as thickeners, for example gums or cellulose derivatives such as xanthan gum, karaya gum and/or ethylcellulose. The one or more solid carriers can be any agriculturally suitable carrier, such as attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In certain embodiments, the one or more solid carriers comprises any carrier suitable for ingestion, such as a saccharide comprising cellulose, xantham gum, karaya gum, ethylcellulose, inositol, galactose, arabinose, lactose, lactulose, mannitol, mannose, sorbose, turanose, platinose, or a combination thereof.

In some embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In other embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chitosan, talc, calcium phosphate, arginine, lysine, calcium carbonate, carbon black, glutamine, betaine, bismuth phosphate, bismuth citrate, iron phosphate, or any combination thereof.

In some embodiments, the carrier comprises the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

In some embodiments, the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acetate phthalate, maltrodextran, dextran, inulin, corn starch, amylopectin, sodium starch glycolate, pentaerthritol, cyclodextrin, or a combination thereof.

In certain preferred embodiments, the solid carrier comprises silica and ethylcellulose, more particularly about 10% to about 50% by weight of the silica and about 50 to about 90% by weight of the ethylcellulose.

In other preferred embodiments, the solid carrier comprises silica and activated charcoal, particularly about 10% to about 90% by weight of the silica and about 10% to about 90% by weight of the activated charcoal.

In certain embodiments, the binder further comprises arginine, lysine, or both arginine and lysine. While not being bound by theory, it is believed that arginine and lysine are capable of forming hydrogen bonds with the small molecule, such as 3NOP, thereby altering the release rate.

In other preferred embodiments, the carrier comprises activated charcoal and ethylcellulose, particularly about 10% to about 50% by weight of the activated charcoal and about 40 to about 90% by weight of the ethylcellulose.

In some embodiments, the carrier further comprises about 1 to about 10% by weight of sodium lignosulfate. While not being bound by theory, it is believed that sodium lignosulfate improves coating adhesion to the tablet resulting in a reduction in release rate of the small molecule.

In other embodiments, the carrier comprises arginine and polycaprolactone, such as about 10 to about 60% by weight of the arginine and about 30 to about 90% by weight of the polycaprolactone.

In other preferred embodiments, the carrier comprises 25% silica, 66% polycaprolactone, such as about 10 to about 60% by weight of the silica and about 30 to about 90% by weight of the polycaprolactone.

In certain embodiments, the composition comprises a granular shape. The composition may comprise any suitable shape, such as a spherical-, square-, rectangular-, capsular-, cylindrical-, conical-, ovular-, triangular-, diamond-, disk-like shape, or a combination thereof. In certain embodiments, the shape of the particle affects the rate of dissolution of the particle.

The granular particle can comprise any suitable texture, for example hard or soft. In certain embodiments, the texture of the particle affects the rate of dissolution of the particle. In certain embodiments, the composition comprises a combination of differently textured pellets each of which release the small molecule at different rates.

In certain embodiments, the granular particles comprise a uniform size distribution, for example about ±20%, ±15%, ±10%, ±5%, ±2%, or ±1% size distribution in the median particle size. In certain embodiments, the granular particles comprise a non-uniform size distribution, for example greater than about ±20%. In certain embodiments, the granular particles comprise a plurality of differently sized populations of granular particles each of which comprise a uniform size distribution.

In certain embodiments, the one or more solid carrier dissolves and thereby releases the one or more small molecules that reduce the production of greenhouse gases and/or precursors thereof. In a preferred embodiment, the one or more solid carriers will dissolve in water.

### Extended and Delayed Release

It may be necessary to vary the rate of dissolution of the composition. For example, one may want to produce an extended-release formulation, wherein the composition releases the one or more small molecules over a period of time to maintain a suitable environmental concentration of the one or more small molecules. This can be beneficial to reduce the frequency of applications, for example to reduce labor costs and/or applications in rural and/or hard to reach environments. In certain embodiments, complete dissolution of the composition and full release of the one or more small molecules occurs over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, for example about 1 to about 147 days, preferably about 7 to about 63 days, more preferably about 7 to about 42 days even more preferably about 14 to about 42 days yet even more preferably about 14 to about 28 days.

In certain embodiments, the extended-release formula may comprise any suitable extended-release formula. In certain embodiments, the extended-release formula comprises one or more additives that reduce the rate of dissolution of the composition, one or more additives that reduce the rate of dissolution of the small molecule from the composition, one or more additives that comprise a membrane that dissolves over time, wherein the rate of dissolution of the membrane controls the rate of release of the one or more small molecules, a suitable alternative, or a combination thereof.

In certain embodiments, the population of granular particles comprises a plurality of populations of granular particles wherein each population comprises a different formulation and/or shape. In certain embodiments, the population of granular particles comprises a first population and a second population. In certain embodiments, the population of granular particles further comprises at least 1, 2, 3, 4, 5, 5, 6, 8, or 9 and/or no more than 4, 5, 6, 7, 8, 9, or 10 additional populations, for example a total of 3-10 additional populations, preferably 3-7 additional populations, more preferable 3-5 additional populations. In a preferred embodiment, each of the additional populations comprises a different formulation than the others.

In certain embodiments, the rate of dissolution of the granular particles is modulated by the size of the granular particle. In certain embodiments, smaller granular particles dissolve faster than larger granular particles, such that each successive larger population in the plurality of populations of differently size particles provides a delayed release compared to the smaller populations of particles. In certain embodiments, an increased proportion of larger to smaller granular particles in a population of granular particles results in slower rates of dissolution of the population of granular particles.

In certain embodiments, the first population of particles comprises an immediate release formulation. In certain embodiments, the second population comprises a delayed release formulation, wherein the second population dissolves and/or releases the one or more small molecules that reduce the production of one or more deleterious atmospheric gasses and/or precursors after the first population. In certain embodiments, each additional population comprises a delayed release formulation, wherein each population dissolves and/or releases the one or more small molecules that reduce the production of one or more deleterious atmospheric gasses and/or precursors at a different time than each of the other populations.

In some embodiments, an immediate release formulation releases the one or more small molecules that reduce the production of one or more deleterious atmospheric gases with about 1 day, 2 days, 3 days, 4 days, 5 days, or 6 days. In some embodiments, a delayed release formulation releases the one or small molecules after about 1 week or more, for example up to about 21 weeks.

In some embodiments, the first population of particles provides has a half-life for the one or more small molecules (such as about 1 to 12 hours), the second population has a longer half-life (such as about 24 or more hours), each additional population has a longer half-life than the previous population such that an effective amount of the one or more small molecules is maintained for weeks or months. Thus, the present compositions advantageously do not, in such embodiments, require repeated, frequent applications.

In certain embodiments, the extended release happens within the first 10% of the half-life and then the 1st delayed happens with 1-2 half lives, then the next with 1-2 of the delayed release.

In some embodiments, the particles have a size ranging from about 1 mm to about 20 mm, about 1 to about 15 mm, about 1 to about 10 mm, about 5 to about 20 mm, about 5 to about 15 mm, or about 5 to about 10 mm.

### Coatings

In certain embodiments, the composition may comprise a coating, for examples particles or tablet having a coating. The coating can comprise any suitable coating, such as a wax, a fat, or a synthetic polymer. In certain embodiments, the wax comprises organic compounds consisting of long alkyl chains, natural waxes (plant, animal) which are typically esters of fatty acids and long chain alcohols as well as synthetic waxes, which are long-chain hydrocarbons lacking functional groups. In certain embodiments, the fat comprises a wide group of compounds which are soluble in organic solvents and largely insoluble in water such as hydrogenated fats (or saturated fats) which are generally triesters of glycerol and fatty acids. Suitable fats can have natural or synthetic origin. In certain embodiment, the fat comprises glycerine monostearate, carnauba wax, candelilla wax, sugarcane wax, palmitic acid, stearic acid hydrogenated cottonseed oil, hydrogenated palm oil and hydrogenated rapeseed oil, or combinations thereof. Any suitable synthetic polymer can be used, such as poly-L-glutamic acid (PGA) and polylactic acid (PLA). In preferred embodiments, the synthetic polymer is at least partially water soluble.

The coating may be single layer or multiple layers, preferably two layers.

In some embodiments, the coating is selected from cellulose acetate phlalate, ethyl cellulose, hydroxypropyl cellulose, polycaprolactone, alginate, chitosan, polyethylene glycol, cellulose acetate, triacetin, propylene glycol, n-methyl-2-pyrollidone, and any combination thereof.

In certain preferred embodiments, the coating comprises two or more polyelectrolytes, such as polystyrene sulfonate, polyethyleneimine, sodium lignosulfate, polyglutamic acid and poly-L-lysine, poly-L-arginine, polyallylamine hydrochloride, polyacrylic acid, or any combination thereof.

In some preferred embodiments, the polyelectrolytes comprise polyallylamine hydrochloride and sodium lignosulfate.

In some preferred embodiments, the polyelectrolytes comprise polyallylamine hydrochloride and polystyrene sulfonate.

In other preferred embodiments, the polyelectrolytes comprise sodium lignosulfate and one of polyglutamic acid and poly-L-lysine, or poly-L-arginine, and sodium lignosulfate.

In still other preferred embodiments, the polyelectrolytes comprise polystyrene sulfonate and one of polyglutamic acid and poly-L-lysine, or poly-L-arginine.

The polyelectrolytes may, in certain embodiments, be chemically cross-linked with a cross-linking agent.

In certain embodiments, the composition comprises one or more coatings applied with minimal to no bubbles. Additionally or alternatively, the composition comprises one or more coatings that comprise a foam or a plurality of air bubbles. In certain cases, the foamed coating can temporarily alter the buoyancy of the composition. One such example includes a composition comprising a foamed coating that floats when initially applied, then, after a period of time, the air pockets in the foamed coating fill with water resulting in the composition sinking to the bottom.

Additives with a Density Greater than Water

In certain embodiments, the composition further comprises one or more additives with a density greater than water. For example, the additive may have a density greater than 1.1, preferably about 1.1 mg/mL to about 3 mg/mL, about 1.5 to about 3 mg/mL, about 1.5 to about 2.5 mg/mL, or about 1.5 to about 2 mg/mL. Suitable additives include silica, attapulgite, kaolinite, fuller's earth, calcium carbonate, perlite, diatomaceous earth, calcium silicate, fly ash, or a combination thereof. In certain embodiments, the one or more additives with a density greater than water result in the composition sinking below the surface. In certain embodiments, the one or more additives with a density greater than water result in the composition partially or completely sinking to the bottom. In preferred embodiments, the composition completely sinks to the bottom. In certain embodiments, the composition comprising the additive with a density greater than water has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

### Agriculturally Beneficial Additives

In certain embodiments, the composition further comprises one or more agriculturally beneficial additives. The agriculturally beneficial additive can be any suitable additive depending on the application, such a vitamin, a nutrient, an antibiotic, a fungicide, or a combination thereof.

In certain embodiments, the additive includes one or more suitable components that reduce methanogenesis by methanogens, such as, seaweed (e.g., Asparagopsis taxiformis), kelp, 3-nitrooxypropanol, anthraquinones, ionophores (e.g., monensin and/or lasalocid), polyphenols (e.g., saponins, tannins), organosulfurs (e.g., garlic extract), flavonoids (e.g., quercetin, rutin, kaempferol, naringin, and anthocyanidins; bioflavonoids from green citrus fruits, rose hips and black currants), carboxylic acid, terpenes (e.g., D-limonene, pinene and citrus extracts), or a combination thereof.

Methods for Reducing Production of Deleterious Atmospheric Gases and/or Precursors Thereof

In certain embodiments provided herein are methods. In certain embodiments, provided herein are methods for using one or more small molecules that reduce the production of one or more deleterious atmospheric gases and/or precursors thereof. In certain embodiments, provided herein are methods for applying one or more small molecules that reduce the production of one or more deleterious atmospheric gases and/or precursors thereof to any suitable environment. The suitable environment can comprise any suitable environment. In some embodiments, the suitable environment comprises an environment in which a ruminant (unvaccinated or vaccinated) lives or occupies, e.g., a habitat for a ruminant. In some embodiments, the suitable environment comprises the rumen.

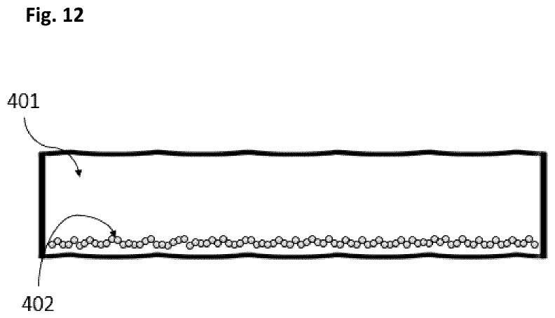

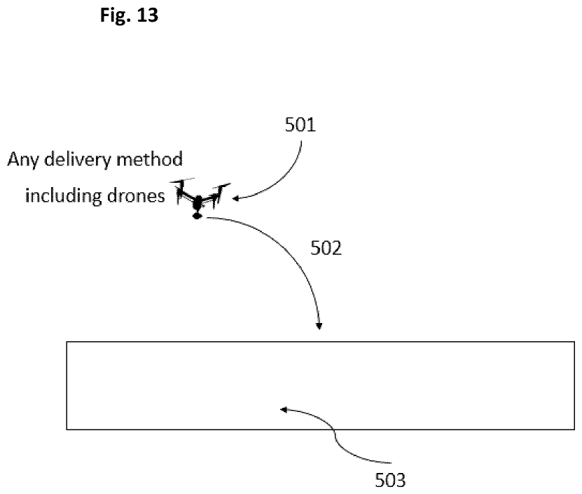

In certain embodiments, the method for reducing emissions of deleterious atmospheric gasses and/or precursors thereof comprises applying a composition comprising one or more small molecules that reduce the production of the deleterious atmospheric gasses and/or precursors thereof. The composition can comprise any suitable composition. In a preferred embodiment, the composition comprises any one of the compositions as described in the Compositions for reducing production of deleterious atmospheric gases and/or precursors thereof section above. In a more preferred embodiments, the composition comprises 3NOP. In certain embodiments, the composition is applied to a water source, such as a trough or a pond, from which a ruminant ingests the composition or a portion thereof from the water source into the rumen. For example as illustrated in FIGS. 12-13.

In certain cases, the composition needs to be reapplied periodically to maintain a suitable concentration of the one or more small molecules. In certain embodiments, the method further comprises reapplying after a period of time a composition comprising one or more small molecules that reduce the production of the deleterious atmospheric gasses and/or precursors thereof. In certain embodiments, the method further comprises, reapplying again after a period of time a composition comprising one or more small molecules that reduce the production of the deleterious atmospheric gasses and/or precursors thereof. Any suitable number of reapplications may be performed as needed to maintain a an effective amount of the one or more small molecules. In some embodiments, the composition is reapplied after about 7 to about 28 days, about 7 to 46 days, about 7 to 92 days or about 7 to 147 days.

In certain embodiments, the composition is delivered to one or more water sources.

In certain cases, the concentration of the one or more small molecules can be measured to ensure the presence of a suitable concentration of the one or more small molecules. Any suitable method may be used to measure the concentration, such as a strip test, liquid chromatography, or thin layer chromatography. The method can be performed with or without human intervention.

Kits for Reducing Production of Deleterious Atmospheric Gases and/or Precursors Thereof

In certain embodiments, provided herein are kits. In certain embodiments, the kit comprises any one of the compositions as described in the Compositions for reducing production of deleterious atmospheric gases and/or precursors thereof section. In certain embodiments, the kit further comprises a suitable container for shipping.

### Methods of Using Vaccines, Animal Feed, Antibodies, Milk

Provided herein are methods of using the vaccines, antibodies, milk, animal feed, agents (e.g., an agent that reduces methane production in a subject, a probiotic bacterial strain, etc.), or any combination thereof.

In certain aspects, provided herein are methods of inducing an immune response against at least one methanogen in a subject, the method comprising administering to the subject the vaccines or pharmaceutical compositions of the present disclosure.

In some embodiments, the immune response comprises a B cell response (e.g., to produce the antibodies). The antibodies produced in response to the vaccine are transferred to the saliva of the subjects, which are swallowed by the subjects to enter the rumen. Once in the rumen, the antibodies come in contact with at least one methanogen to bind/neutralize said methanogen.

As used herein, the term “neutralization of a methanogen” encompasses any reduction in one or more activities that are normally carried out by the methanogen in the absence of the antibodies that bind the methanogen.

In some embodiments, the activity of a methanogen includes but is not limited to, the activity that aids in producing methane gas. For example, binding of the antibodies to the methanogen may reduce the ability of the methanogen to carry out biochemical reactions that are necessary to produce methane, e.g., reduce the ability to convert hydrogen (H₂) and carbon dioxide (CO₂) or acetate into methane (CH₄) and ATP. In some embodiments, the reduced ability to produce methane may lower the fitness of methanogen in the rumen.

In some embodiments, the activity of a methanogen includes but is not limited to, the activity that aids in forming a granular colony with other bacteria. In some embodiments, such activity may be disrupted physically—e.g., antibodies binding to the methanogen would prevent physical association and/or film formation of the granular colony of bacteria. In some embodiments, a reduction in the activity of forming a granular colony may lead to the reduced ability of a methanogen to remain in the rumen. In some embodiments, such reduced ability may result in the reduction of the total number of methanogens inside the rumen.

In certain aspects, provided herein are methods of reducing the activity, number, and/or type of methanogens in the gut of a subject, the method comprising administering to the subject the vaccines or pharmaceutical compositions of the present disclosure.

In certain aspects, provided herein are methods of reducing the amount of methane (CH₄) and/or hydrogen (H₂) emitted by a subject, preferably eructated and/or exhaled, the method comprising administering to the subject a vaccine composition, antibody, milk, and/or animal feed of the present disclosure.

In some embodiments, the amount of methane (CH₄) and/or hydrogen (H₂) is reduced by about 5-100%, preferably by about 10-100%, compared to a control.

In some embodiments, the amount of methane (CH₄) and/or hydrogen (H₂) is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, the amount of methane (CH₄) and/or hydrogen (H₂) is reduced by about 20-100%, preferably by about 30-100%, compared to a control.

In some embodiments, the amount of methane (CH₄) is reduced by (a) about 1 kg-about 50 kg within 8 weeks from the time of first vaccination, or (b) about 5 kg-about 300 kg within a year from the time of first vaccination, compared to a control.

In some embodiments, the amount of methane (CH₄) normalized to an amount of CO₂ emitted by the subject (i.e., CH₄/CO₂) is reduced by about 5-100%, preferably by about 10-100%, compared to a control. In some embodiments, the amount of methane (CH₄) normalized to the amount of CO₂ is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, the amount of methane (CH₄) normalized to the amount of CO₂ is reduced by about 20-100%, preferably by about 30-100%, compared to a control.

In some embodiments, the amount of hydrogen (H₂) is reduced by (a) about 10 g-about 500 g within 8 weeks from the time of first vaccination, or (b) about 50 g-about 3 kg within a year from the time of first vaccination, compared to a control.

In certain aspects, provided herein are methods of increasing the amount of carbon dioxide (CO₂) emitted by a subject, preferably eructated and/or exhaled, the method comprising administering to the subject a vaccine composition, antibody, milk, and/or animal feed of the present disclosure.

In some embodiments, the amount of carbon dioxide (CO₂) is increased by about 1-100%, preferably by about 1-20%, compared to a control. In some embodiments, the amount of CO₂ is increased by at least about 1%, 5%, 10%, 15%, 20%, 25%, 30%, 35%, 40%, 45%, 50%, 55%, 60%, 65%, 70%, 75%, 80%, 85%, 90%, 95%, or 100% compared to a control.

In some embodiments, the amount of carbon dioxide (CO₂) is increased by about 3-10%, preferably by about 3-20%, compared to a control.

In some embodiments, the amount of carbon dioxide (CO₂) is increased by (a) about 29.8 kg-about 1,490 kg within 8 weeks from the time of first vaccination, or (b) about 149 kg-about 8,940 kg within a year from the time of first vaccination, compared to a control.

In some embodiments, the control is: (a) an accepted reference; (b) the amount of methane, CO₂-normalized methane, hydrogen, or carbon dioxide emitted by an unvaccinated subject; or (c) the amount of methane, CO₂-normalized methane, hydrogen, or carbon dioxide emitted by the vaccinated subject prior to vaccination.

In preferred embodiments, any one of the methods produces an antibody against at least one methanogen. In some embodiments, the antibody is an IgM, IgG, or an IgA. In preferred embodiments, the antibody is an IgA or IgM. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is produced in an amount sufficient to: (a) carry the antibody to the gut; (b) reduce the number and/or type of methanogens in the gut; and/or (c) reduce the amount of methane produced by the subject.

In some embodiments, the method reduces the methane production by the subject by at least about 1%, 5%, 10%, 20%, 30%, 40%, 50%, 60%, 70%, 80%, 90%, or 95% as compared with a control.

In some embodiments, the method reduces the H₂ emission from the subject from at least about 1%, 5%, 10%, 20%, 30%, 40%, 50%, 60%, 70%, 80%, 90%, or 95% as compared with a control.

In some embodiments, the method increases the feed conversion efficiency of the subject by about 0.50%, 10%, 1.5%, 2%, 2.5%, 3%, 3.5%, 4%, 4.50%, 50%, 5.50%, 6%, 6.5%, 7%, 7.5%, 80%, 8.5%, 9%, 9.5%, 10%, 10.5%, 11%, 11.5%, or 12% as compared with a control.

In some embodiments, the method increases the concentration of one or more volatile fatty acids (e.g., propionate, butyrate, acetate) in the rumen of the subject by about 0.5%, 1%, 1.50%, 2%, 2.5%, 3%, 3.5%, 4%, 4.5%, 5%, 5.50%, 6%, 6.5%, 7%, 7.5%, 80%, 8.5%, 9%, 9.5%, 10%, 10.5%, 11%, 11.5%, or 12% as compared with a control.

In some embodiments, the method increases the average daily gain (ADG) of the subject by about 0.50%, 10%, 1.50%, 2%, 2.5%, 3%, 3.5%, 4%, 4.5%, 5%, 5.50%, 6%, 6.5%, 7%, 7.5%, 80%, 8.5%, 9%, 9.5%, 10%, 10.5%, 11%, 11.5%, or 12% as compared with a control.

In some embodiments, the method increases the dry matter intake (DMI) of the subject by about 0.50%, 10%, 1.50%, 2%, 2.5%, 3%, 3.5%, 4%, 4.5%, 5%, 5.50%, 6%, 6.5%, 7%, 7.5%, 80%, 8.5%, 9%, 9.5%, 10%, 10.5%, 11%, 11.5%, or 12% as compared with a control. Additionally or alternatively, the method increases the milk production of the subject by about 0.5%, 1%, 1.5%, 2%, 2.50%, 3%, 3.5%, 4%, 4.5%, 5%, 5.5%, 6%, 6.5%, 7%, 7.5%, 8%, 8.50%, 9%, 9.5%, 10%, 10.5%, 11%, 11.5%, or 12% as compared with a control.

In some embodiments, the control is an accepted reference, or the amount of methane production in a subject that has not been vaccinated.

Notably, methane emission or production of methane by a subject can occur at any part of its intestinal track, which includes, e.g., a rumen and a lower bowel (lower intestinal track).

The rumen accounts for 90% of all methane production. The rumen has no adaptive immune response. Thus, to be effective in reducing the level of methane production, the rumen or a methanogen therein must be exposed to neutralizing antibodies that bind and inactivate the methanogen. By contrast, the lower bowel, which accounts for 10% of all methane production, has adaptive immune response such that any immune response due to a vaccine can be amplified in the lower bowel. Accordingly, in preferred embodiments, the result of immune response from a vaccine is exposed to the lower bowel of a subject, which then further amplifies the effect of the vaccine. In other words, in preferred embodiments, a vaccine of the present disclosure or the immune response it elicits is exposed to a methanogen in a lower bowel of the subject. Thus, the present disclosure encompasses a method of reducing (i) methane production and/or (ii) activity, number, and/or type of methanogens in the lower intestinal track of a subject, the method comprising administering to the subject a vaccine comprising at least one methanogen cell surface protein or a fragment thereof.

Accordingly, in preferred embodiments, the methods and compositions of the present disclosure elicit immune response that is exposed to a methanogen in a lower bowel of the subject.

In some embodiments, the methods and compositions of the present disclosure reduce the activity, number, and/or type of methanogens in the lower intestinal track (lower bowel) of a subject.

In some embodiments, the methods and compositions of the present disclosure reduce the amount of methane produced by a subject in the lower intestinal track of a subject.

In some embodiments, the methods and compositions of the present disclosure induce an immune response against at least one methanogen in the lower intestinal track of a subject.

In some embodiments, the method of the present disclosure results in at least about 1%, 2%, 3%, 4%, 5%, 6%, 7%, 80%, 9%, 10%, 110%, 12%, 13%, 14%, 15%, 16%, 17%, 18%, 19%, or 20% reduction in the level of methane produced by the subject.

In some embodiments, the reduction in the level of methane is compared to an untreated subject.

In some embodiments, the subject methane emissions are measured using a respiratory chamber.

As described herein, in some embodiments, the vaccine or the pharmaceutical composition is administered to the subject via a route selected from intramuscular administration, intradermal administration, subcutaneous administration, and nasal administration.

In some embodiments, the subject is administered with at least one dose of the vaccine or pharmaceutical composition.

In some embodiments, the subject is administered with at least one or two repeat doses of the vaccine or pharmaceutical composition (e.g., booster dose).

In some embodiments, the subject is administered with the repeat dose of the vaccine or pharmaceutical composition after at least about 2 weeks, 1 month, 6 months, or 12 months from the time the subject is administered with the preceding dose of the vaccine.

In some embodiments, the subject is administered with the repeat dose of the vaccine or pharmaceutical composition no more than about 3 months, 6 months, 12 months, or 24 months from the time the subject is administered with the preceding dose of the vaccine.

In some embodiments, the subject receives the repeat dose of the vaccine after at least about 2 weeks and no more than about 18 months from the time the subject is administered with the preceding dose of the vaccine.

In some embodiments, the subject receives a repeat dose of the vaccine after at least about 4 weeks and no more than about 12 months from the time the subject is administered with the preceding dose of the vaccine.

In certain embodiments, the methods of the present disclosure further comprises administering to the subject at least one agent (e.g., at least one additional agent) that reduces the level of methane produced by the subject.

The vaccine composition may be administered before, concurrently with, or after, any agent, milk, antibody, animal feed, or any composition of the present disclosure.

In some embodiments, the at least one agent is selected from 3-Nitrooxypropanol (3NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, and any combination thereof.

In preferred embodiments, the agent is 3NOP or ethyl-3NOP.

In certain aspects, provided herein are methods of reducing methane production in a subject, the method comprising orally administering to and/or feeding the subject the antibody, the milk and/or the derivatives thereof, and/or the animal feed of the present disclosure.

In certain aspects, the methods of the present disclosure relate to a subject. In some embodiments, the subject is selected from a cow, cattle, a bull, a bison, a yak, a buffalo, an antelope, a goat, a sheep, a deer, a giraffe, a caribou, a gazelle, a macropod, a llama, a camel, and an alpaca.

In some embodiments, the subject is an offspring (e.g., calf) of the vaccinated female subject that received the milk comprising an antibody that binds at least one methanogen.

In some embodiments, the subject is an adult subject. In other embodiments, the subject is a young subject (e.g., a calf). In some embodiments, a young subject includes a subject from birth to weaning. In some embodiments, a young subject includes a subject from birth up to two years of age, such from birth up to 3, 4, 5, 6, 7, 8, 9, 10, 11, or 12 months. Accordingly, in some embodiments, a young subject may be at least, about, or no more than 1, 2, 3, 4, 5, 6, 7, 8, 9, 10, 11, 12, 13, 14, 15, 16, 17, 18, 19, 20, 21, 22, 23, or 24 months in age. In preferred embodiments, a young subject is administered with a vaccine, antibodies, milk, animal feed, agent (e.g., an agent that reduces methane production in a subject, a probiotic bacterial strain, a small molecule inhibitor, etc.), or other composition of the present disclosure prior to establishment of methanogens. In some such embodiments, a lower dose of vaccine or other agents may be required.

In some embodiments, a subject is a subject born from a vaccinated parent(s). In some embodiments, a subject is a subject born from a vaccinated mother such that the subject received a high level of methanogen-neutralizing antibodies in the colostrum and milk fed to the subject at birth. Such a subject or a subject who received an early treatment may have low initial methanogen establishment, thereby enhancing a long term performance of the vaccines, antibodies, milk, animal feed, agents (e.g., an agent that reduces methane production in a subject, a probiotic bacterial strain, a small molecule inhibitor, etc.), or other compositions of the present disclosure.

In some embodiments, a subject is vaccinated with each change in hands and/or environment, such as from one owner to another, one ranch to another, or one geographical area to another. Typical ranch operations will vaccinate a newly arrived animal to manage disease, and vaccinating with a vaccine of the present disclosure (e.g., a vaccine that targets at least one methanogen cell surface protein) will fall into this standard agronomic practice.

In some embodiments, a subject is vaccinated with a vaccine of the present disclosure (e.g., a vaccine that targets at least one methanogen cell surface protein) at a time when the subject is subject to at least one other vaccination. For example, a subject (e.g., a domesticated ruminant, a dairy cow, a beef cow) is subject to vaccination against infectious bovine rhinotracheitis (IBR), bovine virus diarrhea (BVD), parainfluenza-3 (PI3), bovine respiratory syncytial virus (BRSV), clostridia, E. Coli mastitis, leptospirosis, Mannheimia hemolytica, Brucella, vibriosis, Campylobacter, trichomonas, trichomoniasis, rotavirus, foot and mouth disease virus (FMDV), coronavirus, and/or respiratory disease. An exemplary vaccination regimen for a ruminant throughout the life cycle is shown in Table 14.

Additionally or alternatively, CH₄ reducing vaccine compositions may be combined with one or more additional antigens configured to target infectious bovine rhinotracheitis (IBR), bovine virus diarrhea (BVD), parainfluenza-3 (PI3), bovine respiratory syncytial virus (BRSV), clostridia, E. Coli mastitis, leptospirosis, Mannheimia hemolytica, Brucella, vibriosis, Campylobacter, trichomonas, trichomoniasis, rotavirus, foot any mouth disease virus (FMDV) coronavirus, and/or respiratory disease to form a multivalent vaccine composition, which is then administered to the subject as disclosed herein.

TABLE 14
Exemplary administration schedule of USDA-approved vaccinations for a ruminant

| Animal | Life Stage | Exemplary Vaccinations |
|---|---|---|
| Dairy | Adult | 4-way viral-IBR/BVD/PI3/BRSV |
| | Dry-off (e.g., 45-60 days pre-calving) | Leptospirosis (5-way) |
| | | Clostridial (7-way) |
| | | J-5 (mastitis) |
| Dairy | Adult | 4-way viral-IBR/BVD/PI3/BRSV |
| | Pre-breeding (e.g., 30 days prior to breeding | |
| | season) | Leptospirosis (5-way) |
| Dairy | Calf | 4-way viral-IBR/BVD/PI3/BRSV |
| | Weaning (e.g., 6-12 weeks) | Leptospirosis (5-way) |
| | | Clostridial (7-way) |
| | | Mannheimia hemolytica |
| Dairy | Calf | 4-way viral-IBR/BVD/PI3/BRSV |
| | Yearling (i.e., 12-24 mo) | Leptospirosis (5-way) |
| | | Clostridial (7-way) |
| | | Mannheimia hemolytica |
| | | Brucella |
| Beef | Adult | 4-way viral-IBR/BVD/PI3/BRSV |
| | | Leptospirosis (5-way) |
| | Pre-breeding (e.g., 30 days prior to breeding | Vibriosis (Campylobacter)- |
| | season) | natural mating |
| | | Trichomonas-natural mating |
| Beef | Adult | 4-way viral-IBR/BVD/PI3/BRSV |
| | Pregnancy examination (e.g., 30-45 days | |
| | post-breeding season) | Leptospirosis (5-way) |
| | | Clostridial (7-way) |
| Animal | Life Stage | Exemplary Vaccinations |
| | | Scour vaccines-E. coli, possibly |
| | | combination with rota and |
| | | coronavirus |
| Beef | Calf | Clostridial vaccines |
| | Newborn (e.g., 0-3 mo) | Possibly respiratory vaccines, if |
| | | there is a history of disease in very |
| | | young calves |
| Beef | Calf | 4-way viral-IBR/BVD/PI3/BRSV |
| | | Leptospirosis (5-way) |
| | | Clostridial (7-way) |
| | Weaning (e.g., 6-8 mo) | Mannheimia hemolytica |
| | | Brucella |
| Beef | Adult | 4-way viral-IBR/BVD/PI3/BRSV |
| | Arrival at stocker operation (e.g., 6-8 mo) | Mannheimia hemolytica |
| Beef | Adult | 4-way viral-IBR/BVD/PI3/BRSV |
| | 14-21d post-arrival at stocker operation | Clostridial (7-way) |
| | | Mannheimia hemolytica |
| | | Leptospirosis (5-way) (optional) |
| Beef | Adult | 4-way viral-IBR/BVD/PI3/BRSV |
| | Arrival at feedlot operation (e.g., 9-11 mo) | Mannheimia hemolytica |
| Beef | Adult | 4-way viral-IBR/BVD/PI3/BRSV |
| | 14-21d post-arrival at feedlot operation | Clostridial (7-way) |
| | | Mannheimia hemolytica |
| | | Leptospirosis (5-way) (optional |

Vaccinating a large number of animals (e.g., in a ranch, e.g., herding ruminants) is a challenging process. Thus, in preferred embodiments, the administration of a vaccine of the present disclosure is incorporated into a pre-existing vaccination program that an animal is already subject to. Such a method reduces the cost, time, and efforts in administering a vaccine of the present disclosure to an animal.

### Methods for Detection of Methane Production

Various methods for detecting the level of methane produced by animals are known in the art and can be applied to the methods of the present disclosure.

In some embodiments, portable respiration hoods for tethered and non-tethered animals (Garnsworthy et al. (2012) J. Dairy Sci. 95:3166-3180; Garnsworthy et al. (2019) Animals 9:837; Zimmerman and Zimmerman WO2011130538; each of which is incorporated herein by reference) directly measure the gas concentration of incoming and exhaust air from individual animals.

In some embodiments, tracer-ratio gas releases from the animal (Johnson et al. (1994) Environ. Sci. Technol., 28, 359-362, which is incorporated herein by reference), such as Sulfur hexafluoride (SF6) (Grainger et al. (2007) J. Dairy Sci., 90:2755-2766; Vechi et al. (2022) Agriculture, Ecosystems and Environment 330:107885; each of which is incorporated herein by reference), assumes that the tracer gas and the emitted CH4 have similar transport paths, so that a tracer measurement can establish the CH4 emission rate.

In some embodiments, micrometeorological techniques are typically considered a herd-scale measurement, where the emission rate is calculated from the measurement of enhanced gas concentrations downwind of an animal herd (Harper et al. (2011) Anim. Feed Sci. Tech., 166-167, 227-239, which is incorporated herein by reference), and these include the mass balance technique (Laubach et al. (2008) Aust. J. Exp. Agr., 48:132-137; Lockyer and Jarvis (1995) Environ. Pollut. 90:383-390; each of which is incorporated by reference), eddy covariance (Dengel et al. (2011) Glob. Change Biol., 17:3524-3533; Felber et al. (2015) Biogeosciences, 12:3925-3940; each of which is incorporated herein by reference), and inverse dispersion techniques (Flesch et al., (2005) Atmos. Environ., 39:4863-4874; Todd et al. (2014) J. Environ. Qual., 43:1125-1130; Bai et al. (2021) Atmos. Meas. Tech., 14:3469-3479; each of which is incorporated herein by reference). The main advantage of micrometeorological techniques is that they do not interfere with the animals or the environment.

There are also devices that measure the level of methane (see e.g., Rey et al. (2019) Animals 9:563, Mapfumo et al. (2018) Pastoralism: Research, Policy and Practice 8:15; each of which is incorporated herein by reference). For example, the laser methane detector (LMD) is a hand held open path laser measuring device (e.g., LaserMethaneMini (Tokyo Gas Engineering Co., Ltd. Anritsu Devices Co., Ltd., Tokyo, Japan)). The principle of the LMD measuring technology is described (Chagunda et al. (2013) Animal, 7:394-400; Garnsworthy et al. (2012) J. Dairy Sci. 95:3166-3180; and Chagunda et al. (2009) Comput. Electron. Agric. 68:157-160; each of which is incorporated herein by reference). Briefly, this device is based on infrared absorption spectroscopy using a semiconductor laser for CH4 detection. The device must be pointed towards the nostrils of the cow from a fixed distance. Then, the LMD measures the density of the air column between the device and the animal's nostrils. The reflected laser beam is detected by the device, and its signal is processed and converted to the cumulative CH4 concentration along the laser path in ppm-m. The LMD is connected to a tablet (Samsung Galaxy Tab A6, New Jersey, USA) running GasViewer app (Tokyo Gas Engineering Solutions, Tokyo, Japan) via Bluetooth connection for exporting and storing the data in real time at 0.5 s intervals. The effect of atmospheric ambient CH4 concentration from the measurements is discounted using the offset function of the LMD.

The non-dispersive infrared analyzer CH4 analyzer (NDIR) (Guardian NG Edinburg Instruments Ltd., Livinstong, UK) is one of the so-called sniffer methods that measure CH4 concentration (ppm) in breath or exhaled air. These methods have been previously used (e.g., by Garnsworthy et al. (2012) J. Dairy Sci. 95:3166-3180) to assess the CH4 production of dairy cows at commercial farms. Briefly, a gas sampling tube from the front of a cow's head to a gas analyzer to continuously measure CH4 concentration in the cow's breath is used. Then, air is drawn through the instrument by an integral pump between the gas inlet port and analyzer. The device can have a range of 0 to 10,000 ppm, and air can be sampled continuously at a rate of 1 L/min through an 8 mm polyamide tube, using approximately 2 m of tube from the analyzer to cow's nostrils. Methane concentration can be recorded at 1 s intervals and stored in a datalogger (Data Recorder SRD-99; Simex Sp. Z o.o, Gdansk, Poland). Baseline or ambient CH4 concentration can be calculated as mean CH4 concentration before starting the measurements and subtracted from the measured data. Each day before starting measurements, the NDIR analyzer should be verified using standard mixtures of CH4 in N₂ (0.0%, 0.25%, 0.50%, 0.75% and 1.0%; MESA International Technologies INC, Santa Ana, CA, USA).

Certain methods and devices are described further below and in Table 15.

TABLE 15
Features of exemplary methods for measuring methane output by individual animals

| | Purchase | Running | | | Behaviour | |
|---|---|---|---|---|---|---|
| Method | Cost ² | Costs ² | Labour ² | Repeatability | Alteration ³ | Throughput |
| Respiration chamber | High | High | High | High | High | Low |
| SF₆ technique | Medium | High | High | Medium | Medium | Medium |
| Breath sampling during | Low ⁴ | Low | Low | Medium | None | High |
| milking and feeding | | | | | | |
| GreenFeed | Medium | Medium | Low | Medium | Low | Medium |
| Laser methane detector | Low | Low | High | Low | Low-Medium | Medium |

As indicated above and shown in Table 15, exemplary methods include respiration chambers, the sulfur hexafluoride (SF6) tracer technique, breath sampling during milking or feeding, the GreenFeed system, and the laser methane detector. Each method measures different components of methane output. Only respiration chambers measure total emissions from the animal via the oral, nasal and anal routes; all other methods ignore emissions via the anus and only measure methane emitted in breath. Breath measurements are justified because 99% of methane is emitted from the mouth and nostrils, and only 1% via the anus. The SF6 technique samples breath over 24 h, whereas other techniques use spot samples of breath over periods of minutes throughout the day, so diurnal variation has to be considered. The majority of methane (87%) is released by eructation, which provides a clear signal for sample processing.

### Respiration Chamber

Respiration chambers for open- or closed-circuit indirect calorimetry are considered the ‘Gold Standard’, and were used extensively in nutrition studies when establishing the Metabolisable Energy system. A single animal (or occasionally more) is confined in a chamber for between 2 and 7 days. Concentration of methane (and other gases if required) is measured at the air inlet and outlet vents of the chamber. The difference between outlet and inlet concentrations is multiplied by airflow to indicate methane emissions rate. In most installations, a single gas analyser is used to measure both inlet and outlet concentrations, often for two or more chambers. This involves switching the analyser between sampling points at set intervals, so concentrations are actually measured for only a fraction of the day.

Respiration chambers vary in construction materials, size of chamber, gas analysis equipment and airflow rate, all of which can influence results. Validation of 22 chambers at six UK research sites revealed an uncertainty of 25.7% between facilities, which was reduced to 2.1% when correction factors were applied to trace each facility to the international standard for methane. The main sources of uncertainty were stability and measurement of airflow, which are crucial for measuring methane emission rate. It was concluded, however, that chambers were accurate for comparing animals measured at the same site. It is an added challenge, when benchmarking alternative methods against respiration chambers, that the respiration chambers themselves have not been benchmarked against respiration chambers at other facilities.

For large-scale evaluation of methane emissions by individual animals, respiration chambers are challenging, with only a single study in growing Angus steers and heifers exceeding 1000 animals, which found methane production to be moderately heritable h²=0.27 0.07. Installation costs and running costs are high, and only one animal can be measured at a time. If the monitoring time is three days per animal, and chambers are run continuously, then maximum throughput would be approximately 100 animals per chamber per year. In practice, throughput is likely to be 30 to 50 animals per year. Cows are social animals, and confinement in a chamber may ultimately influence their feeding behaviour, resulting in less feed being consumed and in a different meal pattern compared with farm conditions. Altered feeding patterns or levels is not a problem for metabolic studies evaluating feeds, but can be a problem when evaluating individual animals. Furthermore, the representativeness of respiration chambers to grazing systems has been called into question. However, promising developments have led to more animal friendly respiration chambers constructed from cheaper, transparent materials. These lower the cost and reduce the stress of confinement with minimal disruptions to accuracy, precision and no drop in feed intake of the cows (Hellwing et al. (2012) J. Dairy Sci. 95:6077-6085, which is incorporated herein by reference).

### The SF₆ Method

The SF₆ tracer gas technique was developed in an attempt to measure methane emissions by animals without confinement in respiration chambers. Air is sampled near the animal's nostrils through a tube attached to a halter and connected to an evacuated canister worn around the animal's neck or on its back. A capillary tube or orifice plate is used to restrict airflow through the tube so that the canister is between 50 and 70% full after approximately 24 h. A permeation tube containing SF₆ is placed into the rumen of each animal. The pre-determined release rate of SF₆ is multiplied by the ratio of methane to SF₆ concentrations in the canister to calculate methane emission rate.

Many research centres have used the SF₆ technique with variations in design of sampling and collection equipment, permeation tubes, and gas analysis. Reliable results depend on following standard protocols, with greatest variation coming from accuracy of determining SF6 release rate from permeation tubes and control of sampling rate. With capillary tubes, sampling rate decreases as pressure in the canister increases, whereas an orifice plate gives a steadier sampling rate over 24 h. A source of error that has not been evaluated is that animals might interact and share methane emissions when the sampling tube of one animal is near the head of another animal. There is good agreement between methane emissions measured by the SF6 technique and respiration chambers, although results from the SF₆ technique are more variable.

For large-scale evaluation of methane emissions by individual animals, the SF₆ technique is more useful than respiration chambers. Animal behaviour and intake might be affected by wearing the apparatus, and by daily handling to exchange canisters, but the technique is considerably less intrusive than respiration chambers, because cows remain in the herd. Labour and monetary costs for changing canisters each day and for lab analysis are high. Throughput is limited by the number of sets of apparatus available, handling facilities, labour, and the capacity of the lab for gas analysis. Animals need to be measured for 5 to 7 days, and it is recommended that group size should be less than 15 animals, so maximum throughput would be about 750 animals per year. Heritability has been estimated for methane production in grazing Holstein cows at h²=0.33±0.15.

### Breath Sampling During Milking and Feeding

Several research groups have developed methods to measure methane concentration in breath of cows during milking and/or feeding. These are often referred to as ‘sniffer methods’ because they use devices originally designed to detect dangerous gas leaks. Air is sampled near the animal's nostrils through a tube fixed in a feed bin and connected directly to a gas analyser. The feed bin might be in an automatic milking station or in a concentrate feeding station. Different research centres use different gas analysers (Nondispersive Infrared (NDIR), Fourier-transform infrared (FTIR) or photoacoustic infrared (PAIR)) and different sampling intervals (1, 5, 20 or 90-120 s). Methane concentration during a sampling visit of typically between 3 and 10 min may be specified as the overall mean, or the mean of eructation peaks. Some centres use CO₂ as a tracer gas and calculate daily methane output according to ratio of methane to CO₂ and daily CO₂ output predicted from performance of the cow. Repeatability and rank correlations were higher for eructation peaks than for mean concentrations, and were higher for eructation peaks than for methane to CO₂ ratio. However, all methods show good repeatability.

For large-scale evaluation of methane emissions by individual animals, breath-sampling methods have significant advantages compared with other methods. Breath-sampling methods are non-invasive because, once installed, animals are unaware of the equipment and are in their normal environment. Animals follow their normal routine, which includes milking and feeding, so no training of animals, handling, or change of diet is required. Equipment is relatively cheap, although more expensive gas analysers are available, and running costs are negligible.

The compromise for non-invasiveness of breath-sampling is that concentrations of gasses in the sampled air are influenced by cow head position relative to the sampling tube. The use of head position sensors and data filtering algorithms can remove the effects when the cow's head is completely out of the feed bin, but not within the feed bin. Consequently, sniffer measurements are more variable than flux methods, with factors like variable air flow in the barn increasing measurement error (imprecision), and head position, a highly repeatable characteristic, inflating between-cow variability.

Using CO₂ as a tracer gas partly addresses the issue but, because CO₂ arises from metabolism as well as rumen fermentation, variability of CO₂ emissions has to be considered. A further consideration is diurnal variation in breath concentrations of methane and CO₂ because animals are spot-sampled at different times of day and night. Diurnal variation can be accounted for either by fitting a model derived from the whole group of animals, or by including time of measurement in the statistical model.

The number of observations per analyser is limited only by number of cows assigned to one automatic milking station or concentrate feeding station and length of time equipment is installed. Typically, each analyser will record 40 to 70 animals 2 to 7 times per day for 7 to 10 days, although the number of sampling stations per analyser can be increased by using an automatic switching system. Throughput per analyser is likely to be 2000 to 3000 animals per year. Estimates of heritability for methane production measured using this method range from h²=0.12 to 0.45 over multiple studies.

### GreenFeed

GreenFeed (C-Lock Inc., Rapid City, SD, USA) is a sophisticated sniffer system where breath samples are provided when animals visit a bait station. As with other sniffer systems, GreenFeed samples breath from individual animals several times per day for short periods (3 to 7 min). GreenFeed is a portable standalone system used in bam and pasture applications, and incorporates an extractor fan to ensure active airflow and head position sensing for representative breath sampling. Measurements are pre-processed by the manufacturer, and data are available in real time through a web-based data management system. As GreenFeed captures a high proportion of emitted air and measures airflow, which can be calibrated using a tracer gas, methane emission is estimated as a flux at each visit. Providing visits occur throughout the 24 h, methane emission can be estimated directly as g/day.

A limitation of the GreenFeed system Is that animals require training to use the system, although animals which have been trained to use the system will readily use it again. However, some animals will not use the system or will use it infrequently, and frequency of visits is affected by diet. This can be a challenge when screening commercial herds for methane emission under genetic evaluation.

The manufacturer recommends 15 to 25 animals per GreenFeed unit, and recordings are made typically for 7 days. If all animals visit the unit adequately, throughput per unit is likely to be 750 to 1250 animals per year.

### Laser Methane Detector (LMD)

The laser methane detector (LMD) is a highly responsive, hand-held device that is pointed at an animal's nostrils and measures methane column density along the length of the laser beam (ppm·m). In the first implementation of LMD on a farm, measurements for each cow were taken over periods of 15 to 25 s between eructation events, and could detect methane emitted each time the animal breathed out. In a later study with sheep and beef cattle, monitoring periods of 2 to 4 min allowed authors to separate breathing cycles from eructation events. Typically, animals are restrained either manually or in head yokes at a feed fence for the required length of time. The operator has to stand at the same distance (1 to 3 m) from each animal every time and must be careful to keep the laser pointed at the animal's nostrils throughout the measurement period.

The LMD can be used in the animal's normal environment, although for consistency restraint is required during measurement. Because the LMD measures methane in the plume originating from the animal's nostrils, results can be a_ected by factors such as: distance from the animal; pointing angle; animal's head orientation and head movement; air movement and temperature in the barn; adjacent animals; and operator variation. Operator variation is likely to be one of the biggest factors, because the operator controls distance and pointing angle, and is responsible for ensuring that the laser remains on target. The structure of the barn and the resulting ventilation conditions and wind speed at the location of the measurement are also considerable sources of variation in recorded methane.

Assuming operator fatigue does not limit measurements, each LMD could record up to 10 animals per hour. If each animal is recorded 3 times (on 3 consecutive days, for example), throughput is likely to be up to 1000 animals per year.

Methods for Producing of Recombinant Protein and/or Antibodies

The terms“expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, gPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

Antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-ruminant chimeras, or one ruminant-another ruminant (e.g., goat-cow) chimeras, or ruminant-human chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. Chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/0226; Akira et al. European Patent Application 184,18; Taniguchi, M. European Patent Application 171,49; Morrison et al. European Patent Application 173,49; Neuberger et al. PCT Application WO 86/0153; Cabilly et al. U.S. Pat. No. 4,816,56; Cabilly et al. European Patent Application 125,02; Better et al. (1988) Science 24:1041-104; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques:21; Winter U.S. Pat. 5,225,53; Jones et al. (1986) Nature 32:552-52; Verhoeyan et al. (1988) Science 239:1534; and Beidler et al. (1988)J Immunol. 141:4053-4060.

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present invention can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present invention can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv.

### Methods for Treating Diseases or Conditions

The vaccine compositions and methods of the present disclosure are also useful for treating disease(s) and/or condition(s). It is well documented that methanogens are associated with various diseases, including inflammatory bowel disease (IBD), gingivitis, and bloat. The vaccines of the present disclosure are also useful in treating diseases in subjects (e.g., animals, mammals, ruminants, humans) that are associated with elevated, increased, or severe lactic acidosis. Thus, the vaccine compositions methods of the present disclosure have utility in treating these diseases or conditions in subjects (e.g., humans, felines, canines, ruminants).

### Kits

The present invention also encompasses kits. For example, the kit can comprise a vaccine of the present disclosure, any one of pharmaceutical compositions described herein, at least one additional agent that reduces methane production in a subject described herein, or any combination thereof, packaged in a suitable container and can further comprise instructions for using such reagents. The kit may also contain other components, such as administration tools packaged in the same or separate container.

### Production of Low Carbon Animal Products

In some aspects, the vaccine compositions and methods disclosed herein may be used to produce low carbon animal products. The production of low carbon animal products may involve administering a vaccine composition to an animal, determining greenhouse gas emissions from the vaccinated animal, harvesting an animal product, and determining the carbon intensity of the harvested product compared to a product from an unvaccinated animal.

In some embodiments, a method of producing a low carbon animal product may comprise:
(a) Administering to an animal a vaccine composition comprising cells and/or cell parts of at least one methanogen, as described in any of the embodiments herein. The animal may be a mammal, such as a ruminant.
(b) Determining an amount of emissions of at least one greenhouse gas from the vaccinated animal following administration until animal product harvesting. The greenhouse gas may include, but is not limited to, CO₂, CH₄, N₂O, and/or H₂. In some cases, the emissions may be measured using a GreenFeed system or other suitable emissions measurement technique.
(c) Harvesting an animal product from the vaccinated animal. The animal product may include, but is not limited to, meat, milk, wool, or other products derived from the animal.
(d) Determining an amount of the harvested animal product.
(e) Determining a first carbon intensity as a ratio of the amount of emissions from the vaccinated animal and the amount of harvested animal product.
(f) Determining a second carbon intensity as a ratio of an amount of carbon emissions from an untreated animal and the same amount of harvested animal product.
(g) Determining the difference between the first carbon intensity and the second carbon intensity.

In some embodiments, the method may further comprise administering to the animal at least one agent that reduces methane production, in addition to the vaccine composition. This may provide a synergistic effect in reducing greenhouse gas emissions.

In some aspects, the method may include certifying the animal product as a low carbon intensity product based on the determined difference between the first carbon intensity and the second carbon intensity. This certification may provide added value to the animal product in markets where low carbon products are desired.

The methods described herein may allow for the production of animal products with a reduced carbon footprint compared to conventional production methods. This may be beneficial for meeting sustainability goals, reducing environmental impact, and potentially commanding premium prices for low carbon animal products in certain markets.

### EXEMPLARY EMBODIMENTS

1. A vaccine composition comprising at least one polypeptide and/or at least one peptide of at least one cell surface protein or a fragment thereof of at least one methanogen.

2. The vaccine composition of embodiment 1, wherein
(a) the at least one methanogen is of a family Methanobacteriaceae;
(b) the at least one methanogen is of a genus selected from: Methanobrevibacter, Methanosphaera, Methanomicrobium, Methanobacterium, Methanocorpusulum, Methanosaeta, Methanoculleus, Methanosarcina, and Thermoplasmatales, optionally Methanobrevibacter, Methanomicrobium, and Methanosarcina; and/or
(c) the at least one methanogen comprises Methanobacterium formicicum, Methanobacterium bryantii, Methanobrevibacter ruminantium, Methanobrevibacter millerae, Methanobrevibacter olleyae, Methanomicrobium mobile, Methanoculleus olentangyi, Methanosarcina barkeri, Methanobrevibacter boviskoreani, Methanobacterium beijingense, Methanoculleus marisnigri, Methanoculleus bourgensis, Methanosarcina mazei, Thermoplasmatales archaeon BRNA1, Methanobrevibacter gottschalkii, Methanobrevibacter thaueri, Methanobrevibacter smithii, Methanosphaera stadtmanae, Methanococcoides burtonii, Methanolobus psychrophilus R15, Methanobacterium paludism, Methanohalobium evestigatum, Methanomethylovorans hollandica, Methanothrix soehngenii, Methanocaldococcus vulcanius, Methanosalsum zhilinae, Methanocorpusculum labreanum, Methanoregula formicica, Methanoculleus marisnigri, Methanocella arvoryzae, Methanoculleus bourgensis, Methanolacinia petrolearia, Methanospirillum hungatei, Methanoplanus limicola, Methanohalophilus mahii, Methanococcus aeolicus, Methanosphaerula palustris, Methanocaldococcus fervens, Methanocaldococcus jannaschii, Methanocaldococcus sp. FS406-22, Methanoregula boonei, Methanobrevibacter sp. AbM4, Methanobrevibacter ruminantium, Methanosphaera, Methanobacterium formicicum, Methanocaldococcus villosus, Methanosarcina barkeri, Methanobacterium lacus, Methanotorris igneus, Methanotorris formicicus, Methanocaldococcus infernus, Methanofollis liminatans, Methanothermococcus okinawensis, Methanobrevibacter smithii, Methanobrevibacter, Methanocella conradii, Methanothermococcus thermolithotrophicus, Methanococcus maripaludis, Methanococcus maripaludis, Methanococcus vannielii, Methanothermus fervidus, Methanosarcina acetivorans, Methanosarcina mazei, Methanosaeta harundinacea 6Ac, Methanococcus maripaludis, Methanococcus voltae, Methanolinea tarda, Methanolobus psychrophilus, Methanosaeta harundinacea, or any combination thereof.

3. The vaccine composition of embodiment 1 or 2, wherein the at least one methanogen comprises Methanobrevibacter gottschalkii and/or Methanobrevibacter ruminantium.

4. The vaccine composition of any one of embodiments 1-3, wherein the vaccine composition comprises at least 2, 3, 4, 5, 6, 7, 8, 9, or 10 polypeptides and/or peptides.

5. The vaccine composition of embodiment 4, wherein the at least two polypeptides and/or peptides are of the same cell surface protein or of different cell surface proteins.

6. The vaccine composition of any one of embodiments 1-5, wherein the at least one cell surface protein or a fragment thereof comprises at least 90%, 91%, 92%, 93%, 94%, 95%, 96%, 97% 98%, 99%, or 100% sequence identity to an amino acid sequence of the present disclosure, optionally wherein the at least one cell surface protein or a fragment thereof comprises at least 90%, 91%, 92%, 93%, 94%, 95%, 96%, 97% 98%, 99%, or 100% sequence identity to an amino acid sequence set forth in any one of Tables C-F, 1A, 1B, 2A, 2B, 3A, 3B, 4A, 4B, 5A, 5B, and 6A-6G, or a fragment thereof.

7. The vaccine composition of any one of embodiments 1-6, wherein the at least one cell surface protein or a fragment thereof comprises at least 90%, 91%, 92%, 93%, 94%, 95%, 96%, 97% 98%, 99%, or 100% sequence identity to an amino acid sequence encoded by at least one nucleic acid of the present disclosure, optionally wherein the at least one nucleic acid comprises the nucleotide sequence set forth in any one of Tables C-F, 1A, 1B, 2A, 2B, 3A, 3B, 4A, 4B, 5A, 5B, and 6A-6G, or a fragment thereof.

8. The vaccine composition of any one of embodiments 1-7, wherein the vaccine composition comprises at least one full-length protein of at least one cell surface protein.

9. The vaccine composition of any one of embodiments 1-7, wherein the vaccine composition comprises at least one fragment of at least one cell surface protein, optionally wherein the fragment is a polypeptide or a peptide.

10. The vaccine composition of embodiment 9, wherein the at least one fragment:
(a) comprises an extracellular domain or a portion thereof;
(b) lacks a signal peptide, optionally a native signal peptide; and/or
(c) lacks a transmembrane domain, optionally a native transmembrane domain.

11. The vaccine composition of embodiment 9 or 10, wherein the at least one fragment lacks at least, about, or no more than 1, 5, 10, 20, 25, 30, 35, 40, 50, 60, 70, 80, 90, or 100 amino acids, optionally from the N-terminus and/or the C-terminus.

12. The vaccine composition of any one of embodiments 1-11, wherein the at least one cell surface protein or a fragment thereof comprises an adhesin-like protein; adhesin-like protein with cysteine protease domain; tetrahydromethanopterin S-methyltransferase subunit; ATP-processing protein; cell wall biosynthesis protein; cofactor biosynthesis protein; CRISPR protein; energy metabolism protein; enzyme; fatty acid synthesis protein; general metabolism protein; membrane protein; metal-binding protein; methanogenesis protein; Mtr protein; MtrE protein; phage-related protein; proteolysis protein; transcription regulation protein; ribosomal protein; substrate binding protein; transcription protein; transport protein; protein whose expression changes in response to lauric acid stress; a fragment thereof; and/or any combination thereof (optionally those listed in e.g., Table 6G).

13. The vaccine composition of any one of embodiments 1-12, wherein the at least one polypeptide and/or at least one peptide is formulated in lipid or saline.

14. The vaccine composition of any one of embodiments 1-13, wherein in the vaccine composition is a pharmaceutical composition, optionally comprising at least one carrier and/or at least one excipient.

15. The vaccine composition of any one of embodiments 1-14, wherein the vaccine composition comprises about 0.01 mg to about 50 mg of protein per mL, optionally about 0.1 mg to about 5 mg of protein per mL.

16. The vaccine composition of any one of embodiments 1-15, wherein the vaccine composition comprises about 0.01 mg to about 50 mg of protein, optionally about 0.1 mg to about 5 mg of protein.

17. The vaccine composition of any one of embodiments 1-16, wherein the at least one polypeptide and/or at least one peptide is lyophilized.

18. The vaccine composition of any one of embodiments 1-16, wherein the at least one polypeptide and/or at least one peptide is in a liquid composition.

19. The vaccine composition of any one of embodiments 1-18, wherein the vaccine composition comprises at least one adjuvant.

20. The vaccine composition of embodiment 19, wherein the at least one adjuvant comprises:
(a) inorganic salts, preferably aluminum and calcium salts;
(b) an oil emulsion;
(c) saponins;
(d) lipid or liposomes
(e) an immune-stimulating complex;
(f) a carrier protein (e.g., KLH, optionally conjugated to the at least one polypeptide and/or at least one peptide);
(g) a bacterial product or derivatives thereof (e.g., bacterial toxins, lipopolysaccharide, etc.);
(h) a cytokine; or
(i) any combination of two or more selected from (a)-(h).

21. The vaccine composition of embodiment 19 or 20, wherein the at least one adjuvant comprises Complete Freund's adjuvant, Incomplete Freund's adjuvant, Montanide ISA70, Montanide ISA61, Saponin, chitosan thermogel, lipid (e.g., monophosphoryl lipid A), a lipid nanoparticle/cationic liposome adjuvant, Emulsigen-D, Emulsigen, Emulsigen-P, Polygen, ENABL 06, Montainde ISA 201, Montanide Gel 02, or any combination of two or more thereof.

22. The vaccine composition of any one of embodiments 1-21, wherein the vaccine composition induces immune response against at least one cell surface protein or a fragment thereof of the at least one methanogen.

23. A method of treating a disease in a subject, the method comprising administering to the subject a vaccine composition of any one of embodiments 1-22.

24. The method of embodiment 23, wherein the disease is a periodontal disease, Inflammatory Bowel Disease (IBD), irritable bowel syndrome (ISB), ISB-C, small intestinal bacterial overgrowth (SIBO), colorectal cancer, obesity, metabolic syndrome, diverticulosis and diverticulitis, liver abscess, gingivitis, and/or bloat.

25. The method of embodiment 23 or 24, wherein the disease is associated with elevated, increased, or severe lactic acidosis.

26. A method of inducing an immune response against at least one methanogen in a subject, the method comprising administering to the subject the vaccine of any one of embodiments 1-22.

27. The method of embodiment 26, wherein the immune response comprises a B cell response and/or a T cell response, preferably a B cell response.

28. A method of reducing rumen lactate, increasing pH, or combination thereof in a subject, the method comprising administering to the subject the vaccine of any one of embodiments 1-22.

29. A method of reducing the activity, number, and/or type of methanogens in a digestive tract of a subject, the method comprising administering to the subject the vaccine composition of any one of embodiments 1-22.

30. The method of embodiment 29, wherein the digestive track comprises rumen, reticulum, omasum, abomasum, small intestine, and/or large intestine, preferably rumen.

31. A method of reducing the amount of methane (CH₄) and/or hydrogen (H₂) emitted by a subject, preferably eructated and/or exhaled, the method comprising administering to the subject the vaccine composition of any one of embodiments 1-22.

32. The method of embodiment 31, wherein the amount of methane (CH₄) and/or hydrogen (H₂) is reduced by about 5-100%, preferably by about 10-100%, compared to a control, optionally wherein the amount of methane (CH₄) and/or hydrogen (H₂) is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control.

33. The method of embodiment 31 or 32, wherein the amount of methane (CH₄) and/or hydrogen (H₂) is reduced by about 20-100%, preferably by about 30-100%, compared to a control.

34. The method of any one of embodiments 31-33, wherein the amount of methane (CH₄) is reduced by (a) about 1 kg-about 50 kg within 8 weeks from the time of first vaccination, or (b) about 5 kg-about 300 kg within a year from the time of first vaccination, compared to a control.

35. The method of any one of embodiments 31-34, wherein the amount of methane (CH₄) normalized to an amount of CO₂ emitted by the subject (i.e., CH₄/CO₂) is reduced by about 5-100%, preferably by about 10-100%, compared to a control, optionally wherein the amount of methane (CH₄) normalized to the amount of CO₂ is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control.

36. The method of embodiment 35, wherein the amount of methane (CH₄) normalized to the amount of CO₂ is reduced by about 20-100%, preferably by about 30-100%, compared to a control.

37. The method of any one of embodiments 31-36, wherein the amount of hydrogen (H₂) is reduced by (a) about 10 g-about 500 g within 8 weeks from the time of first vaccination, or (b) about 50 g-about 3 kg within a year from the time of first vaccination, compared to a control.

38. A method of increasing the amount of carbon dioxide (CO₂) emitted by a subject, preferably eructated and/or exhaled, the method comprising administering to the subject the vaccine composition of any one of embodiments 1-22.

39. The method of embodiment 38, wherein the amount of carbon dioxide (CO₂) is increased by about 1-100%, preferably by about 1-20%, compared to a control, optionally wherein the amount of CO₂ is increased by at least about 1%, 5%, 10%, 15%, 20%, 25%, 30%, 35%, 40%, 45%, 50%, 55%, 60%, 65%, 70%, 75%, 80%, 85%, 90%, 95%, or 100% compared to a control.

40. The method of embodiment 38 or 39, wherein the amount of carbon dioxide (CO₂) is increased by about 3-10%, preferably by about 3-20%, compared to a control.

41. The method of any one of embodiments 38-40, wherein the amount of carbon dioxide (CO₂) is increased by (a) about 29.8 kg-about 1,490 kg within 8 weeks from the time of first vaccination, or (b) about 149 kg-about 8,940 kg within a year from the time of first vaccination, compared to a control.

42. The method of any one of embodiments 32-41, wherein the control is:
(a) an accepted reference;
(b) the amount of methane, CO₂-normalized methane, hydrogen, or carbon dioxide emitted by an unvaccinated subject; or
(c) the amount of methane, CO₂-normalized methane, hydrogen, or carbon dioxide emitted by the vaccinated subject prior to vaccination.

43. The method of any one of embodiments 23-42, wherein the subject produces an antibody against at least one methanogen.

44. The method of embodiment 43, wherein the antibody is an IgG, IgM, or an IgA, preferably an IgA or IgM.

45. The method of embodiment 43 or 44, wherein the antibody is produced in an amount sufficient to:
(a) be detectable in the digestive tract;
(b) reduce the number and/or alter the relative abundances of methanogens in the digestive tract;
(c) reduce the amount of methane, the CO₂-normalized methane, and/or hydrogen emitted by the subject; and/or
(d) increase the amount of CO₂ emitted by the subject.

46. The method of any one of embodiments 23-45, wherein the vaccine composition is administered to the subject via a route selected from intramuscular administration, intradermal administration, subcutaneous administration, and nasal administration.

47. The method of any one of embodiments 23-46, wherein the vaccine composition is administered to the subject via intramuscular administration or subcutaneous administration, preferably subcutaneous administration.

48. The method of any one of embodiments 23-47, wherein the subject is administered with at least one repeat dose of the vaccine composition of any one of embodiments 1-22.

49. The method of embodiment 48, wherein the subject is administered with at least two repeat doses of the vaccine composition.

50. The method of embodiment 48 or 49, wherein the subject is administered with the vaccine composition at least 3 times per year.

51. The method of any one of embodiments 48-50, wherein the at least one repeat dose comprises the same dose or a different dose compared to the preceding dose of the vaccine composition.

52. The method of any one of embodiments 48-51, wherein the at least one repeat dose comprises the same adjuvant or a different adjuvant compared to the preceding dose of the vaccine composition.

53. The method of any one of embodiments 48-52, wherein the subject is administered with the repeat dose of the vaccine composition after at least about 2 weeks, about 3 weeks, about 1 month, about 6 months, or about 12 months from the time the subject is administered with the preceding dose of the vaccine composition.

54. The method of any one of embodiments 48-53, wherein the subject is administered with the repeat dose of the vaccine composition no later than about 1 month, about 2 months, about 3 months, 6 months, 12 months, 18 months, or 24 months from the time the subject is administered with the preceding dose of the vaccine composition.

55. The method of any one of embodiments 48-54, wherein the subject receives the repeat dose of the vaccine after at least about 2 weeks and no more than about 18 months from the time the subject is administered with the preceding dose of the vaccine.

56. The method of any one of embodiments 48-55, wherein the subject is administered with at least two repeat doses of the vaccine composition, and the subject receives:
(a) the first repeat dose (“boost 1”) after about 2 weeks to about 4 weeks from the time the subject is administered with the initial vaccine dose (“prime”); and/or
(b) the second repeat dose (“boost 2”) after about 3 months to about 6 months from the time the subject is administered with the initial vaccine dose (“prime”).

57. The method of any one of embodiments 23-56, wherein the subject is administered with a dosage of between about 0.1 mg of protein per kg of animal body weight and about 250 mg of protein per kg of animal body weight of the vaccine composition each time of vaccination.

58. The method of any one of embodiments 23-57, further comprising administering to the subject (a) at least one agent that reduces the level of methane and/or hydrogen produced by the subject; and/or (b) at least one agent that increases production efficiency.

59. The method of embodiment 58, wherein the at least one agent is administered to a subject concomitant with, prior to, or after the vaccination.

60. The method of embodiment 58 or 59, wherein the at least one agent is administered to a subject after the vaccination.

61. The method of any one of embodiments 58-60, wherein the at least one agent is administered to a subject daily, semiweekly, weekly, biweekly (every two weeks), or monthly.

62. The method of any one of embodiments 58-61, wherein the at least one agent is administered to a subject for a duration of at least 1 week but no more than 1 month.

63. The method of any one of embodiments 58-62, wherein the at least one agent comprises:
(a) an agent selected from the agents listed in Tables 9-13;
(b) 3-Nitrooxypropanol (3NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, or any combination thereof; and/or
(c) Monensin (Rumensin®), Optigrid® 45 (ractopamine hydrochloride), Amprolium (Corid), Bacitracin (Albac, BMD), Bambermycin (GainPro), Decoquinate (Deccox), Fenbendazole (Safe-Guard), Laidlomycin (Cattlyst), Lasalocid (Bovetec), Melengestrol Acetate (MGA), Methoprene (Altosid), Morantel (Rumatel), Poloxalene (Bloat Guard), Ractopamine (Optaflexx, Actogain), Tetraclovinphos (Rabon), or any combination thereof.

64. The method of any one of embodiments 58-63, wherein the at least one agent is 3NOP or ethyl-3NOP.

65. The method of embodiment 64, wherein the subject is administered with
(a) at least about 0.5 g but no more than 25 g of 3NOP per day;
(b) at least about 1 g but no more than 5 g of 3NOP per day; or
(c) about 2.5 g of 3NOP per day.

66. The method of embodiment 64 or 65, wherein the subject is administered with 3NOP for a duration of at least 1 week but no more than 1 month.

67. The method of any one of embodiments 55-66, wherein the at least one agent is formulated in animal feed.

68. The method of embodiment 58, wherein the at least one agent is a composition comprising one or more small molecules that reduce the production of one or more deleterious atmospheric gases and/or precursors thereof and one or more agriculturally suitable carriers.

69. The method of embodiment 68, wherein the one or more agriculturally suitable carriers comprises a solid carrier.

70. The method of embodiment 69, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

71. The method of embodiment 69, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chotsan, talc, calcium phosphate, arginine, lysine, calcium carbonate, carbon black, glutamine, betaine, bismuth phosphate, bismuth citrate, iron phosphate, or any combination thereof.

72. The method of any one of embodiments 69-71, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

73. The method of any one of embodiments 69-71, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acetate phthalate, maltrodextran, dextran, inulin, corn starch, amylopectin, sodium starch glycolate, pentaerthritol, cyclodextrin, or a combination thereof.

74. The method of any one of embodiments 69-73, wherein the solid carrier comprises silica and ethylcellulose.

75. The method of embodiment 74, wherein the carrier comprises about 10% to about 50% by weight of the silica and about 50 to about 90% by weight of the ethylcellulose.

76. The method of any one of embodiments 69-73, wherein the carrier comprises silica and activated charcoal.

77. The method of any one of embodiments 69-73, wherein the carrier comprises about 10% to about 90% by weight of the silica and about 10% to about 90% by weight of the activated charcoal.

78. The method of any one of embodiments 77, wherein the carrier further comprises arginine, lysine, or both arginine and lysine.

79. The method of any one of embodiments 69-73, wherein the carrier comprises activated charcoal and ethylcellulose.

80. The method of embodiment 79, wherein the carrier comprises about 10% to about 50% by weight of the activated charcoal and about 40% to about 90% by weight of the ethylcellulose.

81. The method of embodiment 79 or 80, wherein the carrier further comprises about 1 to about 10% by weight of sodium lignosulfate or about 1 to about 10% by weight of hydroxyethyl cellulose.

82. The method of any one of embodiments 69-73, wherein the carrier comprises arginine and poly caprolactone.

83. The method of embodiment 82, wherein the carrier comprises about 10 to about 60% by weight of the arginine and about 30 to about 90% by weight of the polycaprolactone.

84. The method of any one of embodiments 69-73, wherein the carrier comprises silica and polycaprolactone.

85. The method of embodiment 84, wherein the carrier comprises about 10 to about 60% by weight of the silica and about 30 to about 90% by weight of the polycaprolactone.

86. The method of any one of embodiments 69-85, wherein the one or more solid carriers is inert.

87. The method of any one of embodiments 69-86, wherein the one or more solid carriers is water soluble.

88. The method of any one of embodiments 69-87, further comprising one or more additives with a density greater than water and/or one or more additives that reduces the rate of dissolution of the composition in water.

89. The method of embodiment 88, wherein the composition has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

90. The method of embodiment 88 or 89, wherein the one or more additives with a density greater than water comprises silica, attapulgite, kaolinite, fuller's earth, calcium carbonate, perlite, diatomaceous earth, calcium silicate, fly ash, or any combination thereof.

91. The method of any one of embodiments 88-90, wherein the one or more additives that reduces the rate of dissolution of the composition further reduces a rate of release of the one or more small molecules into water.

92. The method of embodiment 91, wherein the composition dissolves over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, about 1 to about 147 days, more preferably 7-63 days, more preferably about 7-42 days, even more preferably 14-42 days yet even more preferably 14-28 days.

93. The method of any one of embodiments 69-92, wherein about 40 to about 80% of the small molecules that reduce the production of one or more deleterious atmospheric gases and/or precursors is thereof is released in water after 15 days.

94. The method of any one of embodiments 69-93, wherein the composition comprises particles having a uniform size distribution.

95. The method of any one of embodiments 69-94, wherein the composition comprises particles having a non-uniform size distribution.

96. The method of embodiment 94 or 95, wherein the particles comprise a spherical-, square-, rectangular-, capsular-, cylindrical-, conical-, ovular-, triangular-, diamond-, or disk-like shape.

97. The method of any one of embodiments 69-96, wherein the particles have a size ranging from about 1 mm to about 20 mm, about 1 to about 15 mm, about 1 to about 10 mm, about 5 to about 20 mm, about 5 to about 15 mm, or about 5 to about 10 mm.

98. The method of any one of embodiments 69-97, wherein the particles further comprises a coating.

99. The method of embodiment 98, wherein the coating comprises at least two layers.

100. The method of embodiment 98 or 99, wherein the coating is selected from cellulose acetate phlalate, ethyl cellulose, hydroxypropyl cellulose, polycaprolactone, alginate, chitosan, polyethylene glycol, cellulose acetate, triacetin, propylene glycol, n-methyl-2-pyrollidone, and any combination thereof.

101. The method of embodiment 98 or 99, wherein the coating comprises two or more polyelectrolytes.

102. The method of embodiment 101, wherein the polyelectrolytes comprise polystyrene sulfonate, polyethyleneimine, sodium lignosulfate, polyglutamic acid and poly-L-lysine, poly-L-arginine, polyallylamine hydrochloride, polyacrylic acid, or any combination thereof.

103. The method of embodiment 102, wherein the polyelectrolytes comprise polyallylamine hydrochloride and sodium lignosulfate.

104. The method of embodiment 102, wherein the polyelectrolytes comprise polyallylamine hydrochloride and polystyrene sulfonate.

105. The method of embodiment 102, wherein the polyelectrolytes comprise sodium lignosulfate and one of polyglutamic acid and poly-L-lysine, or poly-L-arginine, and sodium lignosulfate.

106. The method of embodiment 102, wherein the polyelectrolytes comprise polystyrene sulfonate and one of polyglutamic acid and poly-L-lysine, or poly-L-arginine.

107. The method of any one of embodiments 101-106, wherein the two or more polyelectrolytes are crosslinked.

108. The method of any one of embodiments 69-107, wherein the one or more small molecules comprise a molecule that interferes with the uptake and/or conversion of acetate, H₂, CO², methanol, monomethylamine, dimethylamine, trimethylamine, nitric oxide, or a combination thereof.

109. The method of any one of embodiments 69-108, wherein the one or more small molecules that reduce the production of one or more deleterious atmospheric gases and/or precursors interact with an enzyme selected from the group consisting of 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex β, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine-[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine-corrinoid protein Co-methyltransferase, and any combination thereof.

110. The method of embodiment 109, wherein the one or more small molecules that reduce the production of one or more deleterious atmospheric gases and/or precursors interact with methyl-coenzyme M reductase (MCR).

111. The method of any one of embodiments 69-110, wherein the one or more small molecules that reduce the production of one or more deleterious atmospheric gases and/or precursors comprise a compound having the formula R¹—[CH₂]ₙ—ONO₂ wherein
n is an integer from 1 to 15;
R¹ is selected from the group consisting of H, C₁-C₆alkyl, phenyl, —OH, —NH₂, —CN, —COOH, —O(C═O)R³, —NHC(═O)R³, SO₂NHR³, or —ONO₂, —SH and R³ is C₁-C₆alkyl, phenyl, pyridyl;
with the proviso that when n is >3 the hydrocarbon chain may be interrupted by —O— or —NH—.

112. The method of embodiment 111, wherein the one or more small molecules that reduce the production of one or more deleterious atmospheric gases and/or precursors is selected from 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, and any combination thereof.

113. The method of embodiment 111, wherein the one or more small molecules that reduce the production of one or more deleterious atmospheric gases and/or precursors comprises 3-nitrooxypropanol (3NOP).

114. The method of any one of embodiments 69-113, wherein the composition comprises about 1 to about 25% by weight of the small molecule, about 5 to about 20% by weight of the small molecule, or about 5 to about 15% by weight of the small molecule.

115. The method of any one of embodiments 69-114, wherein the composition comprises a plurality of populations of particles, wherein each population or particles comprises a different formulation, a different shape, and/or a different size distribution.

116. The method of embodiment 115, wherein the plurality of populations of particles comprises a first population and a second population.

117. The method of embodiment 116, wherein the population of granular particles further comprises at least 1, 2, 3, 4, 5, 5, 6, 8, or 9 and/or no more than 4, 5, 6, 7, 8, 9, or 10 additional populations, for example 3-10 additional populations, preferably 3-7 additional populations, more preferably 3-5 additional populations.

118. The method of embodiment 115 or 116, wherein the first population comprises an immediate release formulation.

119. The method of any one of embodiments 116-118, wherein the second population comprises a delayed release formulation.

120. The method of any one of embodiments 117-119, wherein each additional population comprises a delayed release formulation, wherein each population dissolves and/or releases the one or more small molecules that reduce the production of one or more deleterious atmospheric gases and/or precursors at a different time than each of the other populations.

121. An antibody produced by the method of any one of embodiments 23-120, or an antigen-binding fragment thereof.

122. The antibody of embodiment 121, wherein the antibody is a monoclonal antibody.

123. The antibody of embodiment 121 or 122, wherein the antibody is an IgM, an IgG or an IgA, preferably an IgA or an IgM.

124. The antibody of any one of embodiments 121-123, wherein the antibody is lyophilized.

125. The antibody of any one of embodiments 121-124, wherein the antibody is in a pharmaceutical composition, optionally comprising at least one excipient and/or carrier.

126. Milk and/or a derivative thereof produced by the subject of any one of embodiments 23-120.

127. The milk and/or a derivative thereof of embodiment 126, wherein the milk and/or derivatives thereof comprises an antibody that binds at least one methanogen.

128. The milk and/or a derivative thereof of embodiment 126 or 127, wherein the milk and/or derivatives thereof is pasteurized and/or homogenized.

129. The milk and/or a derivative thereof of any one of embodiments 127-128, wherein the milk and/or derivatives thereof is lyophilized or evaporated to form dry milk powder (e.g., boiling at low pressure at low temperature).

130. The milk and/or a derivative thereof of any one of embodiments 126-129, further comprising at least one agent that reduces methane and/or hydrogen production in a subject, optionally wherein the at least one agent is selected from the agents in Table 9.

131. An animal feed comprising:
(a) the antibody of any one of embodiments 121-125;
(b) at least one agent that reduces methane and/or hydrogen production in a subject, optionally wherein the at least one agent is selected from the agents in Tables 9-13;
(c) the milk of any one of embodiments 126-130; or
(d) any combination of two or more of (a)-(c).

132. The animal feed of embodiment 131, wherein the animal feed is liquid (e.g., drinking water, milk) or solid (e.g., fodder).

133. The animal feed of embodiment 131 or 132, wherein the animal feed comprises fat and/or fatty acid, optionally wherein the animal feed comprises fat and/or fatty acid that is at least about 1%, 2%, 3%, 4%, 5%, or 6% of the diet.

134. A method of reducing methane and/or hydrogen production or increasing CO₂ production in a subject, the method comprising orally administering to and/or feeding the subject the antibody of any one of embodiments 121-125, the milk and/or a derivative thereof of any one of embodiments 126-130, the animal feed of any one of embodiments 131-133, or any combination of two or more thereof.

135. The method of embodiment 134, further comprising administering at least one agent that reduces methane and/or hydrogen production in a subject, optionally wherein the at least one agent is selected from the agents in Tables 9-13.

136. The method of embodiment 134 or 135, further comprising administering the subject with the at least one vaccine composition of any one of embodiments 1-22.

137. The method of any one of embodiments 23-120 and 134-136, wherein the subject is a mammal, a human, a canine, a feline, or a ruminant.

138. The method of embodiment 137, wherein the ruminant is selected from a cow, cattle, a bull, a bison, a yak, a buffalo, an antelope, a goat, a sheep, a deer, a giraffe, a caribou, a gazelle, a macropod, a llama, a camel, and an alpaca.

139. The method of any one of embodiments 23-120 and 134-138, wherein the subject is cattle.

140. The method of embodiment 139, wherein the cattle is selected from a pregnant cow, heifer, bull, and steer.

141. The method of any one of embodiments 23-120 and 134-140, wherein the subject is an adult.

142. The method of embodiment 141, wherein the subject is adult cattle selected from:
(a) a cow that is about 6-8 months to about 2-7 years old;
(a) a beef adult that is about 1.5 years to about 2.5 years old; and
(b) a dairy adult that is about 4 years to 7 years old.

143. The method of any one of embodiments 23-120 and 134-140, wherein the subject is a young subject (e.g., before weaning or below 2 years of age).

144. The method of embodiment 143, wherein the subject is young cattle selected from:
(a) a newborn calf and a pre-weaned calf that is about 0 month to about 3 months old; and
(b) a weaned cow that is about 3 months to 8 months old.

145. The method of any one of embodiments 23-120 and 134-140, wherein the subject is a pregnant female subject.

146. The method of any one of embodiments 23-120 and 134-140, wherein the subject is an offspring (e.g., calf) of the vaccinated female subject that received the milk comprising an antibody that binds at least one methanogen.

147. The method of any one of embodiments 23-120 and 134-146, wherein the vaccine is administered to a subject as a part of a pre-existing vaccination program to which the subject is subject (e.g., Table 14).

148. The method of any one of embodiments 23-120 and 134-147, wherein the vaccine is administered to a subject when the subject is subject to or receives at least one other vaccine, wherein the at least one other vaccine is against infectious bovine rhinotracheitis (IBR), bovine virus diarrhea (BVD), parainfluenza-3 (PI3), bovine respiratory syncytial virus (BRSV), clostridia, E. Coli mastitis, leptospirosis, Mannheimia hemolytica, Brucella, vibriosis, Campylobacter, trichomonas, trichomoniasis, rotavirus, coronavirus, and/or respiratory disease.

149. The method of any one of embodiments 23-120 and 134-148, wherein the vaccine is administered to a subject when the subject changes in hands and/or a changes in environment.

150. The method of any one of embodiments 23-120 and 134-149, wherein the vaccine reduces methane and/or hydrogen production in the lower intestinal track (lower bowel) or the rumen of the subject.

151. The method of embodiment 150, wherein the method results in at least about 1%, 2%, 3%, 4%, 5%, 6%, 7%, 8%, 9%, 10, 1%, 12%, 13%, 14%, 15%, 16%, 17%, 18%, 19%, or 20% reduction in the level of methane and/or hydrogen produced by the subject, optionally wherein the reduction in the level of methane and/or hydrogen is compared to an untreated subject.

152. A kit comprising the vaccine composition of any one of embodiments 1-22.

153. The kit of embodiment 152, wherein the vaccine composition comprises no more than one polypeptide or peptide.

154. The kit of embodiment 152, wherein the vaccine composition comprises:
(a) at least two polypeptides;
(b) at least two peptides; or
(c) at least one polypeptide and at least one peptide.

155. The kit of embodiment 152, wherein the kit comprises at least two vaccine compositions, each comprising different polypeptide(s) and/or peptide(s).

156. The kit of embodiment 155, wherein the at least two vaccine compositions are in separate containers.

157. The kit of any one of embodiments 152-156, wherein the kit comprises about or at least about 1, 2, 3, 4, 5, 10, 20, 50, 100, 250, 500, 1000, 1500, 2000, 2500, or 3000 doses of the vaccine composition.

158. The kit of embodiment 157, wherein
(a) all doses are in a single container;
(b) each dose is in an individual container; or
(c) about or at least about 2, 3, 4, 5, 10, 25, 50, or 100 doses are pooled in a single container.

159. The kit of any one of embodiments 152-158, further comprising at least one adjuvant.

160. The kit of embodiment 159, wherein the vaccine composition and the at least one adjuvant are in separate containers.

161. The kit of embodiment 159 or 160, wherein the kit comprises at least about 1, 2, 3, 4, 5, 10, 20, 50, 100, 250, 500, 1000, 1500, 2000, 2500, or 3000 doses; and
(a) all doses are in a single container, and the at least one adjuvant for all doses are in a separate single container;
(b) each dose is in an individual container, and the at least one adjuvant for all doses are in a separate single container;
(c) each dose is in an individual container, and the at least one adjuvant for each dose is in a separate individual container; or
(d) about or at least about 2, 3, 4, 5, 10, 25, 50, or 100 doses are pooled in a single container, and the pooled amount of at least one adjuvant for the pooled doses are in a separate single container.

162. The kit of any one of embodiments 159-161, wherein the kit comprises at least two adjuvants that are different.

163. A method of treating a subject afflicted with a disease, the method comprising to the subject:
(a) the vaccine composition of any one of embodiments 1-22;
(b) the antibody of any one of embodiments 121-125;
(c) the milk and/or derivative thereof of any one of embodiments 126-130;
(d) the animal feed of any one of embodiments 131-133;
(e) at least one agent that reduces methane and/or hydrogen production in a subject, optionally wherein the at least one agent is selected from the agents in Tables 9-13; or
(f) any combination of two or more thereof.

164. The method of embodiment 163, wherein the subject is selected from a mammal, ruminant, a canine, a feline, and a human, optionally wherein the ruminant is selected from a cow, cattle, a bull, a bison, a yak, a buffalo, an antelope, a goat, a sheep, a deer, a giraffe, a caribou, a gazelle, a macropod, a llama, a camel, and an alpaca.

165. A method of reducing CH₄ emissions in a ruminant comprising administering to the ruminant a vaccine composition comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the CH₄ emissions are reduced by at least about 10%, 11%, 12%, 13%, 14%, 15%, 16%, 17%, 18%, 19%, or 20% as compared to an untreated control ruminant.

166. A method of reducing H₂ emissions in a ruminant comprising administering to the ruminant a vaccine composition comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the H₂ emissions are reduced by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% as compared to an untreated control ruminant.

167. A method of increasing the productivity of a ruminant comprising administering to the ruminant a vaccine composition comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the productivity is increased by at least about 0.50%, 1%, 1.5%, 2%, 2.5%, 3%, 3.5%, 4%, 4.5%, 5%, 5.50%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% as compared to an untreated control ruminant.

168. The method of any one of embodiments 165-167, wherein the vaccine composition comprises the vaccine composition of any one of embodiments 1-22.

169. The method of any one of embodiments 165-168, wherein the ruminant is cattle.

170. An animal injected subcutaneously with the vaccine composition of any one of embodiments 1-22, wherein the vaccine composition comprises about a dosage of between 0.1 mg/kg and 250 mg/kg, or any range in between or any value in between.

171. A method of producing a low carbon animal product, the method comprising:
(a) administering to an animal (e.g., a mammal, a ruminant) a vaccine composition of any one of embodiments 1-22;
(b) determining an amount of emissions of at least one greenhouse gas (e.g., CO₂, CH₄, N₂O, and/or H₂) from the animal of (a) following administration until animal product harvesting;
(c) determining a first carbon intensity as a ratio of a first amount of emissions from the vaccinated animal in (b) and the amount of harvested animal product;
(d) determining a second carbon intensity as a ratio of a second amount of carbon emissions from an untreated animal and the same amount of harvested animal product; and
(e) determining the difference between the first carbon intensity and the second carbon intensity.

172. The method of embodiment 171, wherein determining the amount of emissions comprises measuring the emissions using a GreenFeed system.

173. The method of embodiment 171, wherein the animal product is selected from the group consisting of meat, milk, and wool.

174. The method of embodiment 171, further comprising administering to the animal at least one agent that reduces methane and/or hydrogen production.

175. The method of embodiment 171, further comprising certifying the animal product as a low carbon intensity product based on the determined difference between the first carbon intensity and the second carbon intensity.

### EXAMPLES

### Example 1: Computational Selection of M. gottschalkii Antigens

### Dataset Generation

The complete list of 1,800 predicted proteins from the Methanobrevibacter gottschalkii DSM 11977 genome was obtained from NCBI and included both the protein identifiers, amino acid sequence, and native or genomic nucleic acid (DNA) sequence.

### Protein Metadata Generation

### Localization Predictions

A successful protein antigen would need to be located on the exterior of the methanogen cell in order for it to be accessible to antibodies for binding. Therefore, we were interested in understanding the subcellular localization of all proteins in the M. gottschalkii genome. Several computational tools were used to predict both the overall localization of the entire protein and the residue-specific localization.

Subcellular localization for whole proteins was performed using the pSORTb (World Wide Web at hub.docker.com/r/brinkmanlab/psortb_commandline/) command line interface (CLI) tool. PSORTb is a bioinformatics tool for predicting subcellular localization for a given set of protein sequences. We report the localization with the highest score per protein.

Localization per-residue was predicted using TMbed (World Wide Web at github.com/BernhoferM/TMbed). This tool predicts for every residue whether it is predicted to be inside the cell, outside the cell, or embedded in the plasma membrane. We used the predictions from this tool to select extracellular fragments from proteins that may be only partially exterior to the cell.

### Specificity and Conservation

A successful antigen in a methanogen vaccine should be both specific to methanogens and conserved among them; that is, widespread among methanogen species but not among non-methanogen microbial species. To identify proteins that are both specific to and conserved among methanogens, we analyzed the prevalence of two types of annotations within ruminal microbes (both methanogens and non-methanogens). We analyzed Gene Ontology (GO terms), which describe the predicted function and localization of a protein, and Pfam domain annotations, which describe conserved protein sequence features shared among members of a protein family. Both analyses were performed using the same methods.

To perform a comparative genomics analysis of methanogen genomes, we used data from Stewart et al., 2019 (World Wide Web at doi.org/10.1038/s41587-019-0202-3). In this paper, rumen samples from 283 beef cattle were DNA sequenced and 4941 genomes were assembled, of which 126 were archaea and 111 were from the Methanobrevibacter genus.

Stewart et al. annotated genes using DIAMOND to search against several public databases including UniRef; 9 million UniRef100 genes were annotated across the 4,941 genomes. Stewart et al. classified each genome's taxonomy. We defined “methanogens” as archaeal genomes containing methanogenesis genes and identified ˜235,000 genes from those genomes for further analysis.

The UniRef100 genes' Gene Ontology terms were obtained and GO term enrichment analysis was performed on each genome. A hyper-geometric test was used to quantify the degree of enrichment of each GO term in every genome (p-values adjusted with the Benjamini-Hochberg method). Then, for each GO term we used a two-sided t-test to measure the significance of the difference between the means of the adjusted p-values from the methanogen and non-methanogen genomes. GO terms that were enriched (hypergeometric test; adjusted p-value <0.05) in at least 50% of the methanogen genomes (i.e., at least 63 genomes) and were significantly different between methanogens and non-methanogens (two-sided t-test; adjusted p-value <0.05) were considered “preferred GO terms”. See Table A below for a list of preferred GO terms.

TABLE A

| go_id | count | go_name | ttest_pval_adj |
|---|---|---|---|
| GO: 0016491 | 126 | oxidoreductase activity | 1.112e−164 |
| GO: 0044272 | 122 | sulfur compound biosynthetic process | 0 |
| GO: 0015948 | 117 | methanogenesis | 0 |
| GO: 0000178 | 117 | exosome (RNase complex) | 0 |
| GO: 1901576 | 116 | organic substance biosynthetic process | 0 |
| GO: 0016020 | 114 | membrane | 8.154e−47 |
| GO: 0019386 | 112 | methanogenesis, from carbon dioxide | 0 |
| GO: 0032259 | 112 | methylation | 0 |
| GO: 0016151 | 111 | nickel cation binding | 0 |
| GO: 0000287 | 109 | magnesium ion binding | 1.933e−142 |
| GO: 0006082 | 108 | organic acid metabolic process | 0 |
| GO: 0009236 | 106 | cobalamin biosynthetic process | 2.213e−183 |
| GO: 0006730 | 101 | one-carbon metabolic process | 0 |
| GO: 0006777 | 100 | Mo-molybdopterin cofactor biosynthetic process | 5.948e−273 |
| GO: 0003899 | 96 | DNA-directed 5′-3′ RNA polymerase activity | 0 |
| GO: 0030677 | 93 | ribonuclease P complex | 0 |
| GO: 0000428 | 93 | DNA-directed RNA polymerase complex | 0 |
| GO: 0046982 | 91 | protein heterodimerization activity | 0 |
| GO: 0050454 | 84 | coenzyme F420 hydrogenase activity | 0 |
| GO: 0015252 | 83 | proton channel activity | 0 |
| GO: 0008270 | 81 | zinc ion binding | 1.057e−173 |
| GO: 0005267 | 77 | potassium channel activity | 0 |
| GO: 0030269 | 74 | tetrahydromethanopterin S-methyltransferase activity | 0 |
| GO: 0003743 | 72 | translation initiation factor activity | 0 |
| GO: 0050524 | 71 | coenzyme-B sulfoethylthiotransferase activity | 0 |
| GO: 0008234 | 71 | cysteine-type peptidase activity | 5.831e−42 |
| GO: 0003735 | 71 | structural constituent of ribosome | 3.140e−14 |
| GO: 0006412 | 70 | translation | 4.23le−13 |
| GO: 0008168 | 66 | methyltransferase activity | 0 |
| GO: 0052645 | 66 | F420-0 metabolic process | 0 |
| GO: 0006824 | 65 | cobalt ion transport | 0 |
| GO: 0046872 | 63 | metal ion binding | 2.974e−17 |

Pfam enrichment analysis was performed similarly; however as Stewart et al. did not perform Pfam annotation, we annotated these protein sequences using eggNOG-mapper (World Wide Web eggnog-mapper.embl.de/), a tool that first determines the orthology of a sequence based on pre-computed phylogenies prior to annotation. This approach can be more accurate than homology-based approaches (e.g. BLAST) since orthologs, or genes in a different species that evolved from a common ancestor, tend to retain their function as they evolve. EggNOG-mapper provides annotations from multiple public databases, one of which is the Pfam database composed of protein families, domains, motifs, and repeats. Pfam IDs were obtained for each genome's set of protein sequences, and similar to the GO term enrichment, all combinations of Pfam IDs and genomes were analyzed for enrichment. Pfams passing the same statistical tests were considered “preferred Pfams”. See Table B for a list of preferred Pfams.

TABLE B
Pfams enriched in at least 63 methanogen genomes
and with an adjusted p-value of <0.05

| pfam_id | count | ttest_pval_adj |
|---|---|---|
| Fer4 | 126 | 2.66E−11 |
| DNA_pol_B | 124 | 0 |
| Rad51 | 118 | 0 |
| RLI | 116 | 0 |
| TGT_C2 | 116 | 0 |
| eRF1_2 | 115 | 0 |
| DUF1743 | 115 | 0 |
| eRF1_3 | 115 | 0 |
| eRF1_1 | 115 | 0 |
| TF_Zn_Ribbon | 114 | 0 |
| TFIIB | 114 | 0 |
| ECR1_N | 113 | 0 |
| Big_3_5 | 113 | 0 |
| CBFD_NFYB_HMF | 113 | 0 |
| FMN_red | 113 | 6.00E−40 |
| PMBR | 112 | 0 |
| Beta_helix | 110 | 1.38E−78 |
| DUF4013 | 109 | 0 |
| Fer4_4 | 109 | 4.74E−60 |
| DUF2115 | 107 | 0 |
| FTR | 107 | 0 |
| Class_IIIsignal | 107 | 0 |
| FTR_C | 107 | 0 |
| HTH_5 | 107 | 3.34E−58 |
| Fer4_17 | 106 | 1.46E−24 |
| CBS | 106 | 1.19E−07 |
| DUF3100 | 103 | 0 |
| HEAT_2 | 103 | 4.49E−166 |
| NosD | 102 | 0 |
| UDG | 101 | 3.42E−08 |
| AAA_17 | 99 | 0 |
| TFIIS_C | 99 | 0 |
| RNA_POL_M_15KD | 99 | 0 |
| DUF1724 | 99 | 0 |
| LSM | 99 | 0 |
| DUF447 | 98 | 0 |
| DUF11 | 97 | 1.30E−280 |
| zinc_ribbon_2 | 97 | 6.25E−74 |
| Aldo_ket_red | 97 | 9.41E−09 |
| DUF1211 | 95 | 0 |
| PIN_6 | 94 | 0 |
| DUF2112 | 94 | 0 |
| Transglut_core | 94 | 2.68E−36 |
| HycI | 93 | 0 |
| NiFeSe_Hases | 93 | 8.00E−101 |
| S-AdoMet_synt_C | 93 | 0.00127743 |
| S-AdoMet_synt_M | 93 | 0.00131966 |
| S-AdoMet_synt_N | 93 | 0.0018158 |
| FrhB_FdhB_N | 92 | 2.40E−239 |
| Rubredoxin | 92 | 2.13E−29 |
| HIGH_NTasel_ass | 91 | 0 |
| FrhB_FdhB_C | 91 | 9.65E−178 |
| GGGtGRT | 91 | 9.47E−16 |
| HTH_12 | 87 | 0 |
| DUF2097 | 86 | 0 |
| MMR_HSR1_C | 86 | 0 |
| FlpD | 86 | 0 |
| Peptidase_C1 | 85 | 1.60E−31 |
| Regulator_TrmB | 84 | 0 |
| DUF2149 | 83 | 0 |
| CbiM | 83 | 5.61E−180 |
| zf-ribbon_3 | 82 | 1.32E−247 |
| FdhD-NarQ | 81 | 0 |
| Oxidored_q6 | 81 | 1.55E−107 |
| Flavodoxin_4 | 81 | 8.47E−29 |
| HTH_45 | 79 | 0 |
| Chlam_PMP | 79 | 0 |
| TOBE | 79 | 1.84E−25 |
| Ribosomal_S28e | 78 | 0 |
| CbiZ | 78 | 0 |
| DUF4012 | 78 | 0 |
| Ion_trans_2 | 78 | 1.34E−166 |
| S_layer_C | 77 | 0 |
| O_anti_polymase | 75 | 0 |
| Glyphos_transf | 75 | 8.67E−76 |
| Abi | 75 | 7.14E−55 |
| MTD | 73 | 0 |
| MtrA | 71 | 0 |
| DUF2085 | 71 | 0 |
| MFS_1 | 71 | 0.00344293 |
| PKD | 70 | 2.11E−67 |
| Radical_SAM | 70 | 3.62E−10 |
| Fer4_3 | 69 | 0 |
| HTH_33 | 69 | 2.44E−296 |
| ThiC_Rad_SAM | 69 | 2.30E−92 |
| NAD_binding_4 | 69 | 2.16E−71 |
| MCM_OB | 68 | 0 |
| MCM | 68 | 0 |
| OrfB_Zn_ribbon | 68 | 2.49E−53 |
| UDPG_MGDP_dh_C | 68 | 9.87E−06 |
| UDPG_MGDP_dh_N | 68 | 1.03E−05 |
| HxIR | 67 | 2.36E−47 |
| MarR | 67 | 3.57E−13 |
| UDPG_MGDP_dh | 67 | 1.65E−05 |
| CARDB | 66 | 5.57E−172 |
| MCM_N | 65 | 0 |
| DEAD_2 | 65 | 1.09E−74 |
| Pilin_N | 64 | 0 |
| DDE_Tnp_IS240 | 64 | 7.40E−266 |
| Condensation | 64 | 2.44E−39 |
| PadR | 64 | 3.96E−22 |
| DDE_Tnp_1 | 64 | 3.59E−10 |
| DUF3344 | 63 | 0 |
| TelA | 63 | 1.69E−36 |

### Gene Expression—Metatranscriptomics and Transcriptomics

### Methods

Growth of M. gottschalkii Monocultures

Triplicate M. gottschalkii DSM 11977 cultures were grown anaerobically in 100 mL of BY medium in 500 mL bottles at 38° C. The headspace was regularly exchanged and pressurized to ˜10 psi with 80% H₂/20% CO₂. Growth of the cultures was monitored via methane measurements. While the cultures were in exponential phase, 50 mL samples were anaerobically collected at 4° C. after centrifugation at 5000×g for 6 min. Cell pellets were immediately frozen on dry ice and stored at −80° C. until sent on dry ice to Texas A&M University (TAMU).

### Rumen Sampling Via Ruminal Cannula

Rumen samples were provided by the Wickersham Lab at Texas A&M University (TAMU). Samples were previously collected via the ruminal cannula, strained through three layers of cheesecloth, and separated into liquid and solid phases. Individual 50 mL samples of both liquid and solid phases were retained in conical tubes. Sub-samples of the solid phase were immediately flash frozen using liquid nitrogen and transferred to −20° C. storage until further processing and analysis.

### Total RNA Extraction, Library Preparation, and Sequencing

Previously frozen biological samples were thawed on wet ice and further processed by the Dass Lab at TAMU. Total RNA extraction was completed using the protocol defined in Wang et al. 2011, and RNA cleanup using the RNeasy mini kit (Qiagen) was performed. All total RNA samples were treated with DNAase to remove any DNA contamination. Total RNA was delivered to Texas A&M AgriLife Research's TxGen for sequencing library preparation and subsequent RNA-sequencing. Total RNA samples were stored at −80° C. before and after transport and were transported to TxGen on dry ice.

Samples were QC'ed according to TxGen's SOP prior to library preparation. The bulk of the ribosomal RNA was removed using the Illumina Ribo-Zero Plus rRNA Microbiome depletion kit. The remaining RNA was processed using the PerkinElmer NextFlex Rapid Directional RNA-Seq kit 2.0 to obtain the final libraries. All libraries were stored at or below −20° C. prior to sequencing. RNA-Seq libraries were sequenced using the Illumina NovaSeq 6000 platform and 2×150 bp paired-end reads.

M. gottschalkii Transcriptomic Analysis

The M. gottschalkii transcriptomic data was processed using the latest release of the standardized RNA-Seq Nextflow pipeline (nf-core/maseq: World Wide Web at nf-co.re/rnaseq/3.13.2). Using various tools, the pipeline trims and filters low quality reads (TrimGalore!: World Wide Web at bioinformatics.babraham.ac.uk/projects/trim_galore/), filters out ribosomal RNA (SortMeRNA: World Wide Web at github.com/sortmema/sortmema), builds a transcriptome assembly (StringTie: World Wide Web at ccb.jhu.edu/software/stringtie/), performs multiple alignment (STAR: World Wide Web at github.com/alexdobin/STAR) and quantification (Salmon: World Wide Web at combine-lab.github.io/salmon/) of reads against the transcriptome and reference genome, and compiles a quality assessment report (MultiQC: World Wide Web at multiqc.info/). No major changes were made to the pipeline. The M. gottschalkii DSM 11977 genome in FASTA and GTF file format was used as a reference (NCBI RefSeq Assembly GCF_003814835.1).

### Bovine Rumen Meta-Transcriptomic Analysis: Metatdenovo Nextflow Pipeline Development

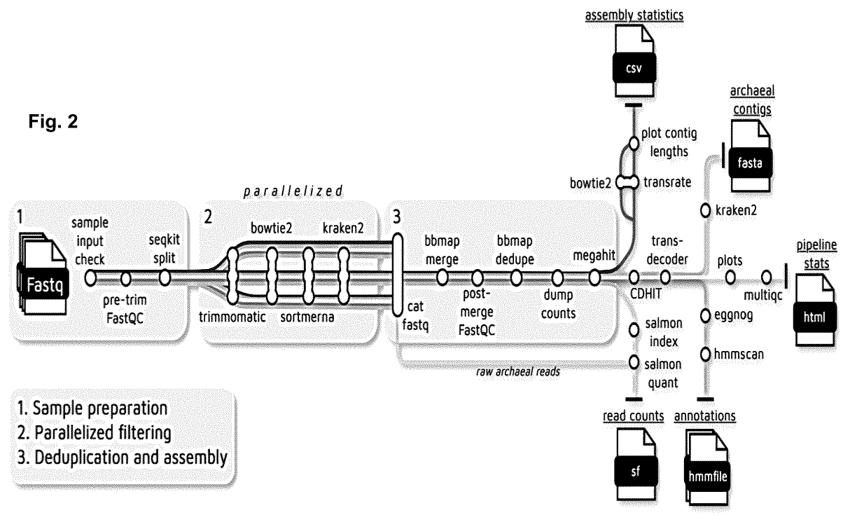

In order to analyze the meta-transcriptomic data we utilized the development version of the metatdenovo Nextflow pipeline, published by nf-core (metatdenovo: World Wide Web at nf-co.re/metatdenovo/dev). The metatdenovo pipeline is still under development, so we added a number of improvements to adapt the pipeline to our dataset (FIG. 2). First, we added filtering steps to filter unnecessary input reads: adapter sequences and low-quality reads (Trimmomatic: World Wide Web at usadellab.org/cms/?page=trimmomatic), contamination from bovine sequences (bowtie2: World Wide Web at github.com/BenLangmead/bowtie2), ribosomal RNA (SortMeRNA: World Wide Web at github.com/sortmema/sortmema), and unnecessary bacterial and non-archaeal reads (Kraken2: World Wide Web at ccb.jhu.edu/software/kraken2/). We also developed a novel parallelization strategy in Nextflow to split input reads into smaller file chunks to increase analysis throughput by leveraging cloud resources to run multiple filtering steps simultaneously.

We then combine the filtered reads into concatenated FastQ files, which are merged and deduplicated (BBMap: World Wide Web at jgi.doe.gov/data-and-tools/software-tools/bbtools/bb-tools-user-guide/bbmap-guide/). The deduplicated reads are then assembled (Megahit: World Wide Web at github.com/voutcn/megahit), as in the original nf-core/metatdenovo pipeline. We then use this assembly to: quantify reads (Salmon: World Wide Web at combine-lab.github.io/salmon/about/), annotate contig information (eggNOG-mapper: World Wide Web at github.com/eggnogdb/eggnog-mapper and HMMER: World Wide Web at hmmer.org/), identify archaeal contigs (Kraken2), and report some pipeline and assembly statistics (MultiQC, Transrate: World Wide Web at hibberdlab.com/transrate/, Kraken2).

### Generation of Heatmaps

Heatmaps of expressed transcripts were generated in R with Pretty Heatmaps (pheatmap: Pretty Heatmaps: World Wide Web at cran.r-project.org/package=pheatmap). Counts matrices containing read mapping values for both the M. gottschalkii transcriptome data and the rumen meta-transcriptome data were first normalized for library size and variance-scaled transformed. The normalized and transformed counts were used as inputs for the heatmaps.

### Nextflow Tower & AWS Batch

While our Nextflow pipeline can be run on standard personal computer machines, the scale of our experiment makes that impractical for the billions of reads sequenced in a meta-transcriptomic experiment. We designed our pipeline to be compatible with Seqera's Nextflow Tower (now known as Segera Platform) to connect our cloud-stored data with computational resources. Seqera Platform utilizes a Fusion file system to access input and reference data without copying data, saving on wall time.

Our pipeline is run on AWS Batch, a batch computing cloud platform that allows for scalable computational jobs. This synergizes with our parallelized pipeline to allow for thousands of pipeline steps to be run at the same time, reducing 30 years worth of compute time to only 24 hours of wall time. We reduced the amount of unnecessary bacterial reads present in the meta-transcriptome, and our meta-assembly was able to complete in less than 2 hours because of the parallelized effort.

### Data Storage on AWS S3

After sequencing was completed on the rumen and cultured methanogen samples, raw FastQ files were uploaded to our AWS S3 bucket. Reference databases and files are also stored in S3, which allows us to scale up storage capacity nearly infinitely.

### Metatranscriptomic and Transcriptomic Results

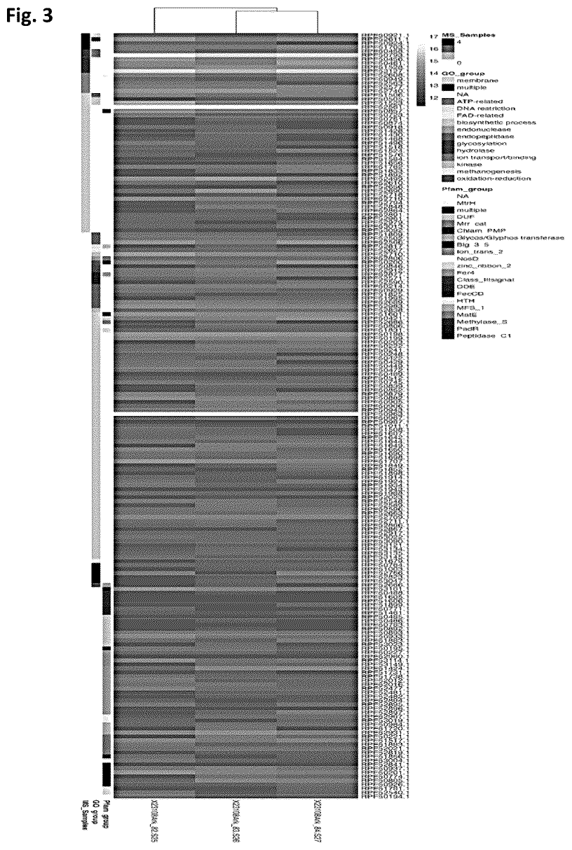

From the M. gottschalkii transcriptomic data, 192 genes were selected for comparison of their gene expression values. These genes were selected based on their potential for use as protein antigens in future vaccine products. Three lines of evidence were used to assess their potential: whether they contain a Gene Ontology (GO) term enriched in methanogens, whether they contain a Pfam enriched in methanogens, and their presence in four mass spectrometry samples of M. gottschalkii proteins exposed and reactive to ArkeaBio's Trial 2 sera. Details of the GO and Pfam enrichment analyses are described above. In total 192 genes met at least 1 of these lines of evidence and 32 genes met at least 2. Of the 48 genes whose corresponding protein was found in at least 1 mass spectrometry sample, 15 were present in at least two samples. In addition, several genes of interest are expressed by M. gottschalkii well above baseline, indicating their active use during normal growing conditions (FIG. 3). Common GO term groups include those related to membrane localization, enzymatic activity (hydrolase, endopeptidase, etc.), ion transport and binding, methanogenesis, and energy use or production (ATP-related or FAD-related). Common Pfam domains include those involved in metal ion binding or transport (zinc_ribbon_2, Fer4, Ion_trans 2), glycosyl- or glycerophospho-transferases, methanogenesis (MtrH), and several domains of unknown function (DUF).

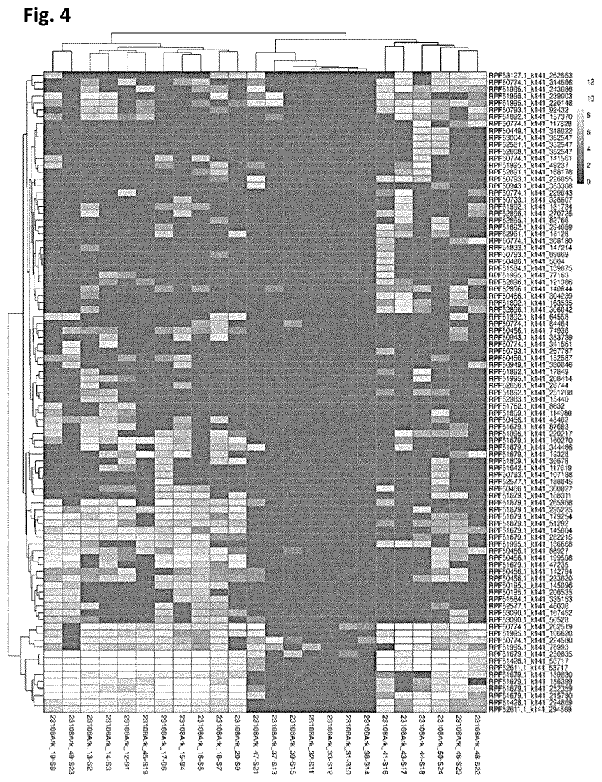

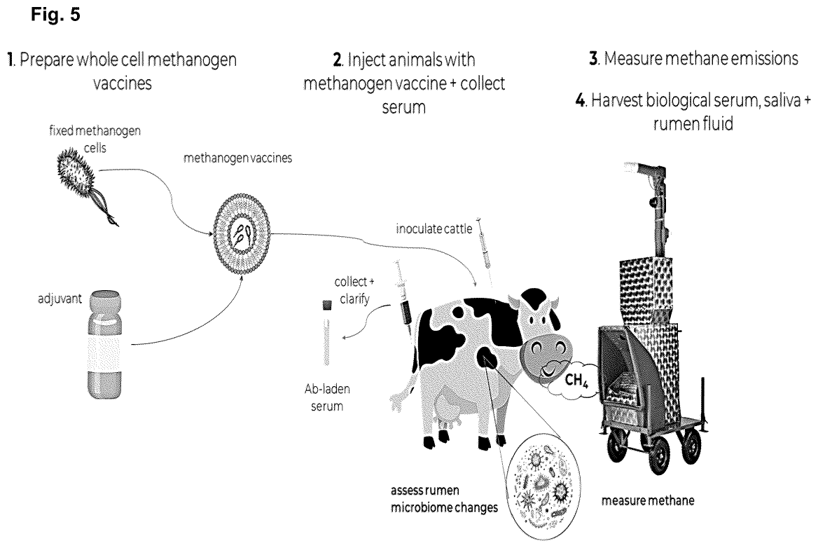

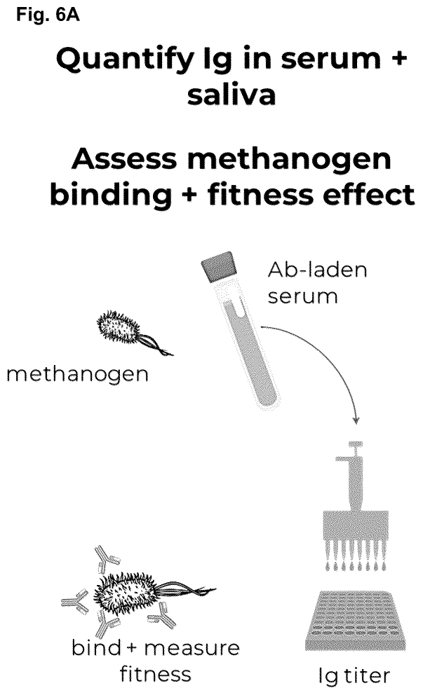

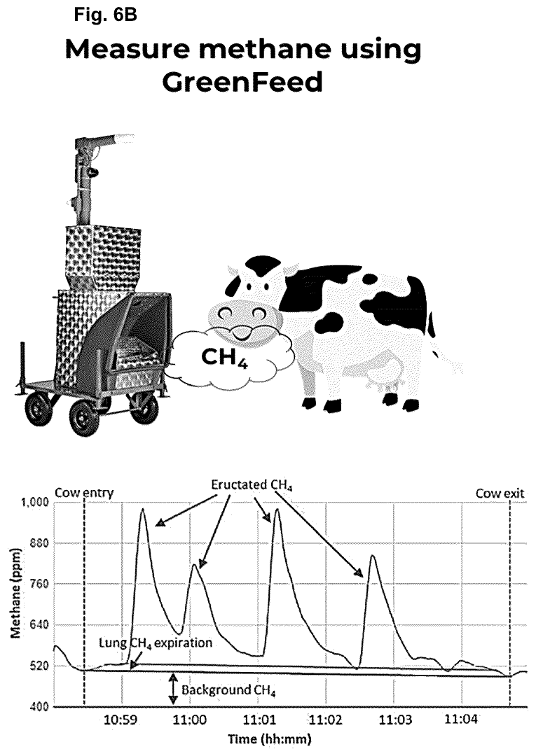

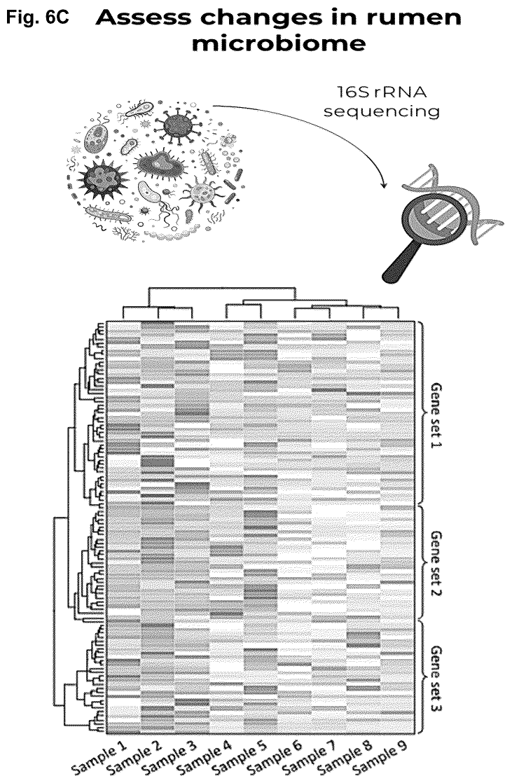

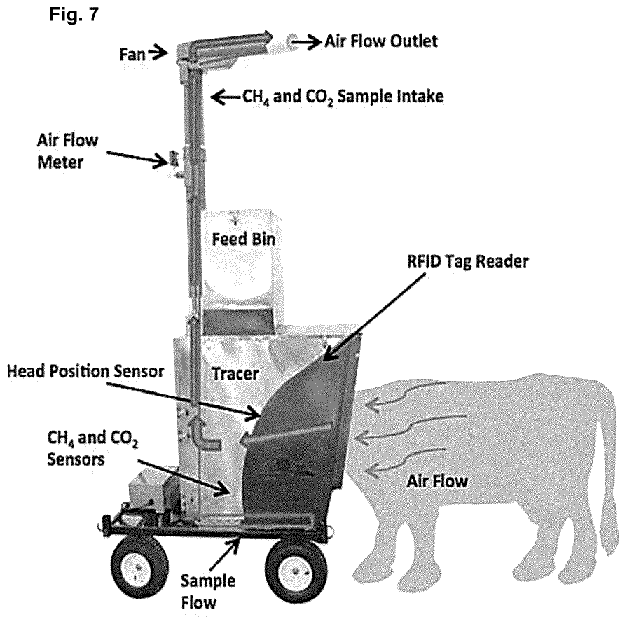

The same 192 M. gottschalkii genes were compared using DIAMOND BLASTp against the metatranscriptomic de novo assembly to determine if they were expressed in the bovine rumen. Of the 192 genes, 127 matched to at least one assembly contig, with 1700 different hits overall. To visualize this, a stringent filter (bit score >80 and percent sequence similarity >50%) was used to obtain the best hits, yielding 97 different gene and contig pairings. When combined with the metatranscriptome expression data from the 24 rumen samples, all but 3 contigs had read mapping support for expression in the rumen after low abundance filtering (>1 read in at least one sample; FIG. 4). This indicates that the 192 M. gottschalkii genes of interest not only match sequences sourced from the rumen in terms of homology but are also being actively expressed within it. Both gene rows and sample columns are also clustered by expression values, indicating similarity of expression patterns across different conditions. Table C lists the metatranscriptomic de novo assembly contigs with the highest average expression values across all rumen samples, the unique M. gottschalkii proteins that mapped to them, and a short description of their functional annotation.

Mean expression Z-scores were calculated as follows: the metatranscriptomic and transcriptomic counts per sample were transformed into per-sample Z-scores, then the mean Z-score was calculated per protein.

The Z-score is a statistical measurement that describes a value's relationship to the mean of a group of values. Z-score denotes the number of standard deviations a value is away from the mean. If a Z-score is 0, it indicates that the data point's score is identical to the mean score.

The difference in expression between cultured and rumen samples was calculated by subtracting the cultured Z-score from the rumen Z-score. A large positive value indicates that the gene has higher relative expression in the rumen than in culture, while a large negative value indicates the opposite.

TABLE C
The 10 highest expressed metaT de novo assembly contigs on average across all rumen samples,
the M. gottschalkii proteins that mapped to them, and their functional annotations.

| | | metaT de novo | Mean | |
|---|---|---|---|---|
| | M. gottschalkii | assembly contig | normalized | |
| SEQ ID NO | protein ID | ID | expression | functional annotation |
| 71358 | RPF51679.1 | k141_250835 | 7.991 | MCR beta subunit |
| 71521 | RPF52611.1 | k141_53717 | 7.186 | MtrA |
| 71178 | RPF50774.1 | k141_202519 | 5.732 | hypothetical protein |
| 71282 | RPF51428.1 | k141_294869 | 4.664 | MtrA |
| 71469 | RPF51995.1 | k141_106620 | 4.612 | cation diffusion facilitator family |
| | | | | transporter |
| 71124 | RPF50456.1 | k141_88927 | 2.903 | V/A-type H+-transporting ATPase |
| | | | | subunit A |
| 71694 | RPF53127.1 | k141_262553 | 1.998 | ferrous iron transport protein B |
| 71189 | RPF50793.1 | k141_92432 | 1.657 | P-type E1-E2/heavy metal translocating |
| | | | | ATPase |
| 71432 | RPF51892.1 | k141_157370 | 1.527 | multimeric flavodoxin WrbA |
| 71679 | RPF53090.1 | k141_167452 | 1.352 | FlaA1/EpsC-like NDP-sugar epimerase |

### Proteomic Analysis of Trial 2 Active Sera

Briefly, sera identified as having binding antibodies in the whole cell methanogen ELISA assay were used as the source of primary antibodies in Western blots to identify bands of interest for proteomic analysis. The rationale is that identified proteins bind to antibodies from vaccinated animals and therefore might represent antigens of interest. A protein is more preferred if it was identified in more than one proteomic sample (maximum of four).

### Preferred Antigens

Preferred antigens were selected based on the following criteria.
Full-length proteins—Set 1
   Antigens include full-length proteins from the M. gottschalkii genome with a localization of “Cellwall”, “Cytoplasmic Membrane”, “Extracellular”, or “Unknown” by pSORTb predictions, or at least one amino acid residue predicted to be outside the cell by TMbed.
Full-length proteins—Set 2
   Antigens include any full-length protein from Set 1 that also was annotated with at least one preferred GO term or at least one preferred Pfam.
Full-length proteins—Set 3
   Antigens include any full-length protein from Set 2 with either above average expression in the rumen transcriptome (Z-score >0), or a rumen-cultured difference >2, or was detected in the Western blot proteomics analysis.
Peptide Fragments—Set 4
   Antigens include any portion or a fragment of a protein from the M. gottschalkii genome where the amino acid residues are predicted to be outside the cell by TMbed. The peptide fragment contains at least 10 amino acids.
Peptide Fragments—Set 5
   Antigens include any peptide fragments from Set 4 that originated from a protein that was annotated with at least one preferred GO term or at least one preferred Pfam.
Peptide Fragments—Set 6
   Antigens include any peptide fragments from Set 5 that originated from a protein with either above average expression in the rumen transcriptome (Z-score >0), or a rumen-culture difference >2, or was detected in the Western blot proteomics analysis.

### Example 2: Informatic Analysis of Antigen Targets and Vaccination

This example demonstrates informatic selection of 40 methanogen cell surface proteins and generation of nucleic acid vaccines encoding those cell surface proteins

### Cell Surface Protein Selection

Briefly, Methanobrevibacter ruminantium and Methanobrevibacter gottschalkii were identified as the two most prevalent ruminal methanogens in the abundance analysis. Each open reading frame (ORF) from their published genomes were curated first database comprising ˜4500 ORFS. Subcellular localization for each ORF was predicted using pSORTdb (Lau (2020) Nucleic Acids Research) and the predicted localization was appended to the first database. The 741 predicted non-intracellular proteins were then ranked based on published gene expression changes in response to lauric acid stress (Zhou (2018) BMC Research Notes) resulting in a second database comprising 45 proteins. The DNA sequences of the proteins were collected from NCBI and were used to search a subset of rumen transcriptomic studies in the Sequence Read Archive (SRA). Proteins were given additional emphasis for inclusion in the final set of candidates based on if there was evidence that either the exact nucleotide sequence or a highly similar sequence was found in transcriptomic data. From examination of the combination of all of these datasets, as well as information based on the predicted functions of the proteins (adhesin or metal binding for example), a final list of sequences was output for additional studies comprising 40 sequences. The protein IDs were used to download the protein sequences from NCBI. As an additional step prior to ordering constructs, the proteins were analyzed using a signal peptide predictor and signal peptides were removed from the final designs. In addition, some of the proteins were too long for synthesis of the template for mRNA production. In this case, the proteins were examined and truncated versions of the protein were chosen based on predicted domain boundaries. This final list of sequences (Table D, ARK016-055) encompasses a list of proteins from two methanogens commonly found in the rumen of cattle. These proteins are predicted to be outside of the cells and there is evidence indicating that they are expressed.

### Table D. Selected Methanogen Cell-Surface Proteins

See SEQ ID NO: 16689 to SEQ ID NO: 16728 for the native nucleic acid sequences of selected methanogen cell-surface proteins (ARK016-ARK055, respectively). See SEQ ID NO: 16729 to SEQ ID NO: 16768 for the Bos Taurus codon-optimized and uridine-depleted nucleic acid sequences of selected methanogen cell-surface proteins (ARK016-ARK055, respectively). See SEQ ID NO: 16769 to SEQ ID NO: 16808 for the amino acid sequences of selected methanogen cell-surface proteins (ARK016-ARK055, respectively).

### Example 3: Ruminal Metagenomic Mining of Methanogen Cell Surface Proteins

This example demonstrates informatic selection of candidate methanogen cell surface proteins, generation of protein and/or peptide vaccines encoding those cell surface proteins or a fragment thereof.

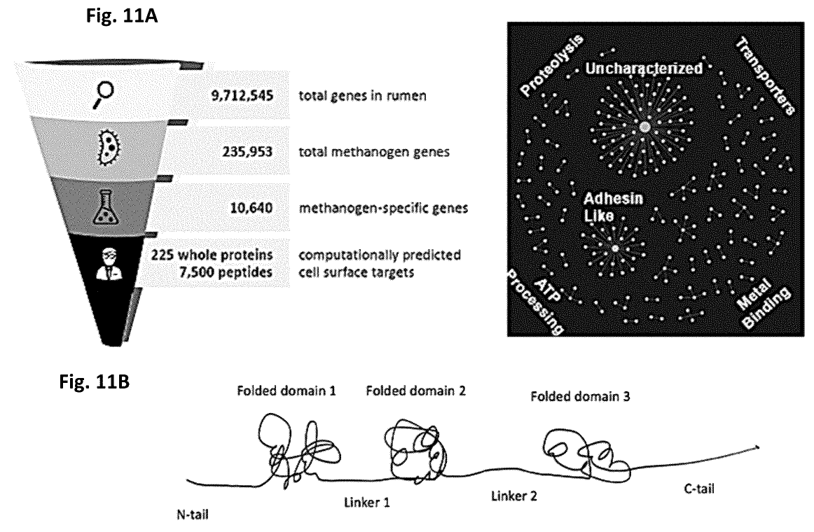

To identify candidate methanogen cell surface proteins, a comparative genomics analysis of ruminal methanogen genomes from rumen samples from 283 beef cattle was used (Stewart (2019) Nature Biotechnology, which is incorporated herein by reference). In total, the analysis comprised 4941 genomes were assembled, of which 126 were archaea and 111 were Methanobrevibacter. Genes were annotated in the 4941 genomes using DIAMOND, resulting in first database comprising 9,712,545 total open reading frames. Methanogen genes were identified by narrowing the total 4941 genomes to those that were archaeal genomes comprising one or more methanogenesis metabolic pathway genes. From those, a second database comprising 235,935 methanogen open reading frames was generated. UniRef100 gene's Gene Onotology terms were obtained and GO term enrichment analysis was performed on each of the ORFs in the second database. The p-values for the enrichment analysis were used as input to a machine learning classification model to classify genomes as methanogen or non-methanogen. This resulted in a third database comprising 10,640 methanogen-specific genes. GO terms most specific to methanogens were used to select candidate genes for localization prediction. pSORTb and TMbed was used to predict subcellular localization and selected whole genes and domains predicted to be accessible to antibodies (e.g., localized to the cell membrane or extracellularly). Based on the subcellular localization, 225 whole proteins and ˜7500 peptide fragments were selected for vaccine production. The bioinformatic selection is shown in FIG. 11A (left). FIG. 11A (right) further shows a network map of the enriched cell surface protein functions, with key methanogen enriched cell surface proteins having functions related to adhesin-like proteins, metal binding, ATP processing, proteolysis, and transport. Many of the adhesin-like proteins comprise cysteine protease domains that are important for cell health, survival, and membrane remodeling.

### Table E. Select Non-Methanobrevibacter Genes

See SEQ ID NO: 16809 to SEQ ID NO: 16832 for the native nucleic acid sequences of select non-Methanobrevibacter genes. See SEQ ID NO: 16833 to SEQ ID NO: 16856 for the bovine codon-optimized nucleic acid sequences of select non-Methanobrevibacter genes. See SEQ ID NO: 16857 to SEQ ID NO: 16880 for the amino acid sequences of select non-Methanobrevibacter genes.

### Table F. Select Protein Fragment Sequences

These fragments are isolated functional domains based on predicted computation folding. For example, a hypothetical protein in FIG. 11B comprising 3 folded domains would yield 3 fragments, each of which is a stable domain on its own and can be used as an antigen for vaccine.

See SEQ ID NO: 16999 to SEQ ID NO: 35021 for the native nucleic acid sequences encoding the select protein fragments. See SEQ ID NO: 35022 to SEQ ID NO: 53044, and SEQ ID NO: 71068 for the bovine codon-optimized and uracil-depleted nucleic acid sequences encoding the select protein fragments. See SEQ ID NO: 53045 to SEQ ID NO: 71067 for the amino acid sequences of the select protein fragments.

### Example 4: Vaccination of Animals with a Protein Vaccine Composition

This example demonstrates a method of preparing and administering a vaccine composition to an animal (e.g., cattle). The vaccine composition used in this study contains a full-length protein of Methanobrevibacter gottschalkii, which upon vaccination to animals resulted in reduction in the animal emitted methane, hydrogen, and/or carbon dioxide normalize methane; and/or increase emitted carbon dioxide.

### Preparation of a Vaccine Composition

A full-length protein of Methanobrevibacter gottschalkii is expressed in bacteria (e.g., E. Coli) or yeast (e.g., S. cerevisiae or P. pastoris). The purified protein is combined with an equal volume of Freund's complete adjuvant (ThermoFisher, Waltham, MA) for primary injections. The purified protein is combined with an equal volume of Freund's incomplete adjuvant (ThermoFisher, Waltham, MA) for booster injections.

### Vaccination and Sample Collection

Healthy, weaned, Angus cross steers are weaned for a minimum of sixty days and received pre-weaned vaccinations a minimum of sixty days prior to initiation of the study. Animals are transferred to the study site fourteen days prior to vaccination to allow for acclimation to the study diet and environment. Each vaccine formulation is administered by intramuscular (IM) injection to three animals. In total, 120 animals are vaccinated with the protein vaccine comprising the full-length protein of Methanobrevibacter gottschalkii and 3 animals are vaccinated with adjuvant alone. Intramuscular injections are delivered into the cow's right neck muscle by veterinary-trained staff using an 18-gauge 1-1.5″ needle. Animals are boosted with a second vaccine administration on day 21. Total blood samples (˜100 mL) are collected from the jugular vein by veterinary-trained staff and processed by ultracentrifugation within 24 hours of sample collection. Isolated serum samples are aliquoted, labeled and stored in cryogenic tubes at −20° C. until use.

### Example 5: Vaccination of Animals with a Peptide-Fragment Vaccine Composition

This example demonstrates a method of preparing and administering a vaccine composition to an animal (e.g., cattle). The vaccine composition used in this study contains a peptide fragment of Methanobrevibacter gottschalkii, which upon vaccination to animals resulted in reduction in the animal emitted methane, hydrogen, and/or carbon dioxide normalize methane; and/or increase emitted carbon dioxide.

### Preparation of a Vaccine Composition

A peptide fragment of Methanobrevibacter gottschalkii is synthesized in vitro using methods known in the art via a vendor (GenScript, Piscataway, NJ). The purified peptide fragment is combined with an equal volume of Freund's complete adjuvant (ThermoFisher, Waltham, MA) for primary injections. The purified protein is combined with an equal volume of Freund's incomplete adjuvant (ThermoFisher, Waltham, MA) for booster injections.

### Vaccination and Sample Collection

Healthy, weaned, Angus cross steers are weaned for a minimum of sixty days and received pre-weaned vaccinations a minimum of sixty days prior to initiation of the study. Animals are transferred to the study site fourteen days prior to vaccination to allow for acclimation to the study diet and environment. Each vaccine formulation is administered by intramuscular (IM) injection to three animals. In total, 120 animals are vaccinated with the protein vaccine comprising the peptide fragment of Methanobrevibacter gottschalkii and 3 animals are vaccinated with adjuvant alone. Intramuscular injections are delivered into the cow's right neck muscle by veterinary-trained staff using an 18-gauge 1-1.5″ needle. Animals are boosted with a second vaccine administration on day 21. Total blood samples (˜100 mL) are collected from the jugular vein by veterinary-trained staff and processed by ultracentrifugation within 24 hours of sample collection. Isolated serum samples are aliquoted, labeled and stored in cryogenic tubes at −20° C. until use.

### Example 6: Ruminal Microbiome Measurement

˜50 mL of fresh rumen samples are collected by esophageal tubing and retained in conical vials. Samples are strained through three layers of cheesecloth, with the liquid phase aliquoted into 50 mL conical vials. All samples re snap-frozen in liquid N₂ and stored at ˜20° C. for subsequent processing. Samples are processed within twenty-four hours of collection. Sample aliquots are labeled with animal number, collection method, collection date and time.

Isolation of DNA from the rumen follows the methods defined in Henderson (Henderson, G. et al. Effect of DNA Extraction Methods and Sampling Techniques on the Apparent Structure of Cow and Sheep Rumen Microbial Communities. PLOS One 8, e74787 (2013), which is incorporated herein by reference), with preference given to methods involving both phenol-chloroform and mechanical lysis steps (PCQI, PCBB, PCSA). PCR amplification of the hypervariable V6-V8 regions of the 16S rRNA gene is performed using the archaea-specific Ar915aF/Ar1386R primer set with Illumina adapters as defined in Table 1 of Kittelmann 2015 (Kittelmann et al. Buccal swabbing as a noninvasive method to determine bacterial, archaeal, and eukaryotic microbial community structures in the rumen. Appl Environ Microbiol 81:7470-7483 (2015), which is incorporated herein by reference). and PCR cycle conditions as defined in Kittelmann 2013 (Kittelmann et al. Simultaneous Amplicon Sequencing to Explore CoOccurrence Patterns of Bacterial, Archaeal and Eukaryotic Microorganisms in Rumen Microbial Communities. PLOS One 8(2): e47879 (2013), which is incorporated herein by reference). PCR products are stored at the appropriate conditions until subsequent use. The PCR products are then purified, quality checked, prepared into sequencing libraries, and analyzed for 16S rRNA sequencing within three weeks of the final rumen sample collection (Day 90). Libraries are generated using the PerkinElmer NextFlex DNA-Seq kit. The 16S rRNA amplicon libraries are sequenced on the Illumina MiSeq v3 600-cycle (2×300 bp) platform. Following the completion of 16S rRNA sequencing, the resulting data is analyzed for methanogen abundance. Methanogen abundance is reduced by ˜10-80% after treatment with vaccines encoding methanogen cell surface proteins as compared to pre-vaccination.

### Example 7: Methane Production Measurements

Cow enteric methane production is monitored using GreenFeed Systems (C-Lock, Inc., Rapid City, SD). Briefly, feed intake is recorded daily throughout the study period using GrowSafe Systems. Animals are fed once daily in the morning and have access to a water source at all times. Body weight is recorded periodically, at the time of total blood draws. Enteric methane, hydrogen, and carbon dioxide emissions are measured daily throughout the study period using GreenFeed Systems (C-Lock, Inc., Rapid City, SD). Animals have free access to the GreenFeed System throughout the study period; animals are coerced to use the system. Methane yield and intensity is calculated using dry matter intake for each measurement period separately. Methane production is reduced by ˜10-80% when treated with nucleic acid vaccines encoding methanogen cell surface proteins as compared to pre-vaccination.

This example demonstrates informatic selection of methanogen cell surface proteins for formulation into protein vaccines, vaccination into subjects, and subsequent reductions in methane in vivo.

### Example 8: Analysis of Antibody Binding

### Sera Collection and Confirmation of Sera Antibody Binding to Methanogens

Total blood samples are collected under the supervision of veterinary-trained staff. Samples are collected via jugular vein needle puncture into blood tubes. Collected blood samples are then clarified via ultracentrifugation within 24 hours of collection in order to harvest serum. Clarified serum samples are aliquoted, labeled and stored at ≤−20° C.

Specifically, jugular blood draws are accomplished by first restraining the animal in a squeeze chute with a head gate. The neck area was then cleaned by wiping the area with isopropyl alcohol, e.g., rubbing alcohol, and gauze to remove any superficial dirt and debris. The jugular vein is occluded by applying pressure at the base of the jugular groove in order to visualize the raised vein. After a sufficient volume of blood is collected, the needle is removed and disposed of in an approved sharps container.

### Confirmation of Sera Antibody Binding to Methanogens Using a Methanogen ELISA.

In order to determine a humoral immune response to the vaccine in the immunized animals, a methanogen ELISA is developed and utilized to assess sera antibody binding to M. gottschalkii.

Specifically, each well of a 96-well high binding ELISA plate, e.g., plate, is coated with 50 μL of 0.001% polylysine. The plate is then stored at 4° C. for at least 10 hours, and up to 2 weeks. Polypeptide and/or peptide samples are diluted in DPBS to an empirically determined dilution for each plate preparation (typically 1:10). Next, the volume of 0.001% polylysine is removed from the plate, and 50 μL of diluted, the polypeptide and/or peptide sample is added to each well. The plate is then centrifuged for 5 minutes at 900×g to facilitate binding of the polypeptide and/or peptide sample. Next, 50 μL of 0.1% glutaraldehyde is added to each well, mixed well, and incubated for 20 minutes at room temperature. The liquid from each well is removed, and 200 μL of 5% goat serum in PBST (blocking solution) is added to each well. The plate is then incubated for 1 hour at room temperature to prevent non-specific binding of the detection antibodies in subsequent steps, also referred to as ‘blocking’.

The clarified sera collected from the animals enrolled in this study as described above are diluted in 5% goat serum in PBST, e.g., blocking solution. Next, the blocked plates are washed twice by adding 200 μL of PBST to each well, and then removing the liquid. 50 μL of PBST-diluted sera is added to each well and incubated for 1 hour at room temperature. The plate is then washed three times with 200 μL of PBST per well. 50 μL of 1:1,000 rabbit anti-bovine-HRP conjugate is added to each well and incubated for 1 hour at room temperature. The plate is then washed three times with 200 μL of PBST per well. Next, 50 μL of TMB solution is added to each well, incubated for 20 minutes at room temperature, to develop. Finally, 50 μL of 2 M H2SO4 is added to each well to stop the reaction. OD450 is measured for each well using a Synergy plate reader (BioTek, Winooski, VT).

### Confirmation of Sera Antibody Binding to Methanogens Using FACS.

In order to confirm the development of a humoral immune response to the vaccine in animals immunized with a vaccine composition comprising a protein or a fragment thereof of M. gottschalkii, fluorescence-activated cell sorting (FACS) is utilized to observe sera antibody binding to M. gottschalkii.

Specifically, 5 μL of fixed M. gottschalkii cells are added to 50 μL of PBS-T in an Eppendorf tube. Then, where applicable, 1 μL of clarified sera collected from the animals enrolled in this study as described above, is added to the relevant Eppendorf tube and mixed well. A detection solution is prepared by adding 150 μL of PBST to 1.5 μL of rabbit anti-bovine IgG biotin and 1.5 μL of SA-PECy7 and mixing well. Once prepared, 50 μL of detection solution is added to sample in Eppendorf tube(s) and mixed well. Sample in Eppendorf tubes, including detection solution, is incubated for 30 minutes at room temperature. Finally, 5 μL of each previously incubated sample is added to 1 mL of DPBS immediately before running on SONY SH800 (FSC gain: 5; threshold: 0.04%; all other settings: default).

### Confirmation of Sera Antibonding Binding to Methanogens Using Western Blot.

In order to confirm the development of a humoral immune response to the vaccine in animals immunized with the vaccine composition, a methanogen Western blot is utilized to observe the sera antibody binding to the polypeptide and/or peptide to which the animals are vaccinated. The Western blot analysis detects the interaction of (a) the sera antibodies previously collected from the animals enrolled in this study as described above, and (b) methanogen protein antigens resolved on an SDS-PAGE gel.

Specifically, a polypeptide and/or peptide sample is mixed with 100 to 200 μL of 1×SDS sample buffer. Next, the polypeptide and/or peptide sample is boiled for 5 minutes at 100° C.

Between 5 and 10 μL of the boiled sample is loaded into individual wells of a 4-15% SDS-PAGE gel. Next, the SDS-PAGE gel is run for 10 minutes at 100V, and then 30 minutes at 200V. The proteins are then transferred using wet transfer technique for 1 hour at 100V onto a 0.22 μm PVDF membrane. Next, the 0.22 μm PVDF membrane is incubated in SuperBlock SuperBlock T20 TBS buffer solution (blocking solution) for 1 hour at room temperature to prevent non-specific binding of the detection antibodies in subsequent steps. The blocked 0.22 μm PVDF membrane is then exposed to various dilutions of clarified sera samples, previously collected from the animals enrolled in this study as described above, in blocking solution for 1 hour at room temperature. Next, the 0.22 μm PVDF membrane is washed three times (5 minutes for each wash) in TBS-T20 to remove any unbound sera and/or antibodies. The 0.22 μm PVDF membrane is then exposed to HRP-conjugated anti-bovine IgG secondary antibody in SuperBlock T20 TBS buffer solution for 1 hour at room temperature to detect any bound bovine sera antibodies. Next, the 0.22 μm PVDF membrane is washed three times (10 minutes for each wash) in TBS-T20 to remove any unbound HRP-conjugated anti-bovine IgG antibodies. Finally, the 0.22 μm PVDF membrane is developed using chromogenic TMB blotting solution, or an appropriate chemiluminescence substrate.

### Example 9: Sample Collection and Additional Analyses

### Saliva Collection

Saliva samples are collected using a suction tube in the oral cavity, into a conical flask or directly into a sterile Eppendorf tube. Equipment contacting saliva, e.g., collection flask or plastic tubing, is changed out after each animal's collection; either single-use or autoclavable materials are used. Sample are immediately put on wet ice, and then transported to the lab for storage at ≤−80° C.

### Rumen Fluid Collection

First, the ruminant, e.g., steer, is restrained in a squeeze chute with a head gate and nose tucked down toward the chest. Then, a long flexible plastic tube is inserted into the animal's nostril and down into the stomach. A hand-operated rumen fluid pump, attached to the flexible plastic tube, is then used to harvest 20-50 mL of rumen fluid per collection. Harvested rumen fluid samples are stored in conical vials and transferred to the lab. Finally, within 24 hours of collection, rumen fluid samples are strained through three layers of cheesecloth, and the liquid phase is aliquoted into 50 mL conical vials and snap-frozen in liquid nitrogen for cryostorage at ≤−20° C.

### Recording and Analysis of Dry Matter Intake

This example describes a method to record and analyze daily dry matter intake of vaccinated animals in order to determine whether the administration of a vaccine has an adverse effect on individual daily feed intake. Furthermore, such a record may be used to normalize inter-animal, e.g., ruminant, for example, cattle, comparisons of daily emitted methane.

Animals are fed once daily in the morning and had access to a water source at all times for the duration of the study. Feed intake is recorded continuously using GrowSafe systems (GrowSafe Systems, Calgary, AB, CA). Briefly, an individual animal's electronic identification tag, e.g., EID, which is typically affixed to the ear, is read by the GrowSafe feed bunk when the animal's head is in sufficiently close proximity to the bunk, e.g., when the animal is feeding from the bunk. During recorded feed events, the animal's EID is associated with the amount of feed that animal consumes from the bunk, which is automatically measured using scales equipped within the bunk. A comparable method for tracking individual dry matter intake may be used where GrowSafe systems are unavailable.

### Recording and Analysis of Animal Body Weight

This example demonstrates a method of recording individual animal body weight (lb) in order to confirm that administration of a vaccine composition does not have an adverse effect on the rate of individual animal weight gain.

Animal body weight is recorded periodically, at the time of total blood draws, throughout the study. Body weight is recorded using a livestock scale. Specifically, animals to be weighed are walked through a pipe alley from their housing area to the holding pens. After being weighed, they are released to return to their holding pen with other animals from the same pen. At the end of the weighing, animals are then walked back to their assigned pen.

### Measurement of Animal Emitted Methane, Hydrogen and Carbon Dioxide

The amount of animal-emitted methane, hydrogen, and carbon dioxide is measured using GreenFeed systems (C-Lock, Inc., Rapid City, SD). Briefly, the GreenFeed system uses a pelletized feed to incentivize animals to visit multiple times per day. When an animal visits a GreenFeed system to consume the dispensed feed pellets, its individual animal's electronic identification tag, e.g., EID, which is typically affixed to the ear, is read by GreenFeed and a measurement period associated with that animal is recorded. During the measurement period, GreenFeed records the amount of animal-emitted methane, hydrogen, and carbon dioxide.

### Example 10: Pregnant and Postpartum Animal Vaccination

A vaccine is used to vaccinate a pregnant animal (e.g., ruminant). The unborn fetus is exposed to the antibodies against methanogen generated by the mother. After birth, the offspring receives milk and/or colostrum laden with the antibodies against the at least one methanogen surface antigen. The offspring is vaccinated with at least one vaccine of the present disclosure prior to weaning. The offspring may be vaccinated with a polypeptide and/or peptide vaccine that is the same or different from the vaccine used to vaccinate the mother.

### Example 11: Combinatorial Therapies

A subject is treated with a combinatory therapy, which comprises any two or more selected from a vaccine, antibodies, milk and/or derivative thereof, animal feed, an agent (e.g., an agent that reduces methane production in a subject, a probiotic bacterial strain, a small molecule inhibitor, etc.), and other composition of the present disclosure (e.g., those reducing methane production in a subject). Any one of the combinatory therapy may be given in any order, i.e., before, concurrently with, or after any other combinatory therapy. Here, a reduction in methane production that is greater than the use of a single therapy alone is achieved.

### Example 12: Synthesis of 3NOP

Adapted from “Structure-Based Design, Synthesis, and Biological Evaluation of Indomethacin Derivatives as Cyclooxygenase-2 Inhibiting Nitric Oxide Donors” (J. Med. Chem. 2007, 50, 6367-6382).

All reagents purchased from Sigma-Aldrich and Fisher Scientific and used as received.

3-Bromopropanol (78.0 g, 0.56 mol) in acetonitrile (300 mL) was added dropwise within minutes to a solution of silver nitrate (145.9 g, 0.86 mol) in acetonitrile (600 mL) and stirred at room temperature for 24 hr. The solution was protected from light by being covered with aluminum foil. After 24 hr, 5:1 excess of brine was added to the reaction mixture and stirred for 1 hr. Silver halide was filtered through Celite and filtrate was extracted with diethyl ether (300 mL×3). The organic layer was washed with brine (300 mL×3). Dried over sodium sulfate, and concentrated (59 g, 86% yield). Product confirmed via NMR (>97% purity based on HPLC).

3NOP pH and Temperature Stability: Confirmed via HPLC over a 1-month time period at temperature between 4-30 C and pH 4-10.

3NOP Volatility: 500 mg of 3NOP in a 5 dram vial was left open to air at ˜18-24 C at 15-30% RH over the course of 6 days. Average mass loss is ˜0.25% per day.

### Compaction Study

The viability of different binders for densification were examined. 0.5 g of powder was tableted under 20 kN in a 13 mm pellet die. Potential binders are summarized in Table 16.

TABLE 16
Potential binders ranked based on densification.

| | | 3: powdery | 2: compacts, |
|---|---|---|---|
| | | break, | no strength |
| | 4: clean | retains | 1: compacts, |
| 5: Unbreakable | braking, | moderate | disintegrates |
| tablet by hand | no dust | strength | 0: no compaction |
| Xanthan gum | Lignin | Pea protein | Chitosan |
| k-carrageenan | Inulin | Soy protein | Hydroxyethyl |
| | | | cellulose |
| Sodium alginate | Wood flour | Gelatin | Talc |
| Cellulose acetate | Corn Starch | Casein | Sodium starch |
| | | | glycolate |
| Ethyl cellulose | Amylopectin | | Pentaerythritol |
| Hydroxypropyl | Hemp protein | | Calcium |
| cellulose | | | phosphate |
| Cellulose acetate | | | Diatomaceous |
| phthalate | | | earth (Celite) |
| Maltodextrin | | | Biochar |
| Dextran | | | |

### Tablets for Encapsulation of 3NOP

A multilayer encapsulation system was employed for extended-release formulations consisting of 3 main parts:
1) Adsorbent—inert, non-toxic, solid-support for 3NOP (ie. silica, biochar, activated charcoal, talc, arginine, lysine, calcium carbonate, carbon black, cyclodextrin, calcium phosphate, celite (diatomaceous earth), glutamine, betaine, bismuth phosphate, bismuth citrate, iron phosphate, etc)
2) Binder/matrix—allow for granulation of adsorbent to form solid core (ie, starches, flours, sugars, proteins, gums, gelatin, cellulose and derivatives, etc)
3) Coating—single to multilayer coatings to decrease water penetration and modify release kinetics

All materials purchased from Sigma-Aldrich and Fisher Scientific and used as received unless otherwise specified.

### Tablet Preparation

3NOP was adsorbed onto the corresponding adsorbent at a given weight percent (noted in formulations) by dropwise addition to adsorbent stirring on a hot plate. The mixture was left to stir for more than 30 minutes until a free-flowing powder was obtained. The resulting mixture was speed mixed with the corresponding binder for 1 minute followed by compaction of in a carver press using an 8-13 mm pellet die under various forces of 10-60 kN.

### Coated Tablet Preparation

Selected coating materials were dissolved in various solvents at 5-20 wt %. Tablets were then dip coated multiple times allowing for drying in between to give the desired coating weight.

### 3NOP Release Quantification and Corresponding Tablet Formulations

13 mm, 0.5 g tablets were placed in a sealed 5 dram vial with 10 mL of distilled water and incubated at 70 F until full dissolution of 3NOP. 1 mL aliquots were taken every few days and refreshed with 1 mL of distilled water. Aliquots were filtered through a 0.22 um PVDF filter before being quantified via HPLC.

### Uncoated Tablets

All formulation percentages are based on weight. 2 replicates of 13 mm disc-shaped, 0.5 g tablets were compacted under 20 kN. The uncoated formulations had the following compositions:
AH-01-7.5% 3NOP, 12.5% propylene glycol, 60% Cargill hemp protein
AH-02-8.57% 3NOP, 22.86% Celite, 68.57% hemp protein
AH-03-8.57% 3NOP, 22.86% Wakefield Biochar, 68.57% Cargill hemp protein
AH-04-8.57% 3NOP, 22.86% K10 montmorillonite, 68.57% Cargill hemp protein
AH-05-8.57% 3NOP, 22.86% activated charcoal, 68.57% Cargill hemp protein

The 3NOP release of the uncoated tablets
after 12 hours is shown in Table 17.

| | Total Average | |
|---|---|---|
| | Concentration of 3NOP | Standard deviation |
| Formula | (mM) | (mM) |
| AH-01 | 24.56 | 2.09 |
| AH-02 | 24.50 | 1.76 |
| AH-03 | 19.61 | 0.17 |
| AH-04 | 33.10 | 0.10 |
| AH-05 | 18.58 | 0.07 |

### Coated Tablets

Tablets for coating experiments consist of 10% 3NOP, 20% Wakefield biochar, and 70% cellulose acetate (Mn: 50,000). Tablets were then coated with ˜0.2 wt % of the corresponding coating material, as shown in the table in Table 18. The release profile over periods of 4, 6 and 11 days for each formula is also shown.

TABLE 18
3NOP release profile of coated tablets over 4 days.

| | | Total Average | Standard |
|---|---|---|---|
| | Time | Concentration | deviation |
| | (days) | (mM) | (mM) |
| Cellulose Acetate Phthalate | 4 | 34.21 | 2.23 |
| (CAP) | | | |
| CAP | 6 | 30.65 | 0.04 |
| CAP | 11 | 32.38 | 0.24 |
| Ethyl cellulose (EC) | 4 | 29.92 | 0.06 |
| EC | 6 | 31.07 | 0.01 |
| EC | 11 | 31.50 | 0.26 |
| Hydroxy propyl cellulose (HPC) | 4 | 31.70 | 0.05 |
| HPC | 6 | 30.35 | 0.01 |
| HPC | 11 | 32.53 | 0.4 |
| Polycaprolactone (CAPA 6800) | 4 | 32.08 | 0.29 |
| CAPA 6800 | 6 | 31.00 | 0.14 |
| CAPA 6800 | 11 | 31.99 | 0.23 |
| Alignate (layer 1) Chitosan | 4 | 30.30 | 0.17 |
| (layer 2) | | | |
| Alignate (layer 1) Chitosan | 6 | 30.22 | 0.17 |
| (layer 2) | | | |
| Alignate (layer 1) Chitosan | 11 | 31.90 | 0.10 |
| (layer 2) | | | |
| Polycaprolactone Mn: 2000 | 4 | 33.68 | 0.01 |
| Polycaprolactone Mn: 2000 | 6 | 32.25 | 0.18 |
| Polycaprolactone Mn: 2000 | 11 | 33.23 | 0.09 |
| PEG Mn: 1500 | 4 | 32.74 | 0.22 |
| PEG Mn: 1500 | 6 | 31.88 | 1.30 |
| PEG Mn: 1500 | 11 | 33.48 | 0.29 |

Further formulations were prepared and tested. 3 replicates of 13 mm disc-shaped, 0.5 g tablets were compacted under 6 kN. All formulas consist of 23.8% 3NOP, 28.6% silica, and 47.6% ethyl cellulose. Tablets were then coated with 60-80 wt % of the corresponding coating material. Control tablet (CTRL) is uncoated. “R” denotes replicate number. CAP coating solution was 18 wt % in 70% ethanol/water. Cellulose acetate (CA) coating was 6 wt % in 70% ethanol/ethyl acetate with an additional 5 wt % triethyl citrate, 5 wt % propylene glycol, and 5 wt % N-methyl-2-pyrollidone. The release profiles were analyzed at 1, 4, 8, 11, and 15 days, as summarized in Table 19.

TABLE 19
3NOP release profile of coated tablets over 15 days.
Total concentration of 3NOP is listed in mM.

| | | | | | |
|---|---|---|---|---|---|
| CAP R1 | 25.74 | 47.79 | 59.39 | 61.05 | 59.68 |
| CAP R2 | 18.72 | 43.71 | 52.56 | 53.74 | 53.37 |
| CAP R3 | 23.12 | 50.66 | 57.23 | 59.23 | 58.75 |
| AVG | 22.53 | 47.39 | 56.39 | 58.01 | 57.26 |
| STD | 3.54 | 3.49 | 3.49 | 3.81 | 3.41 |
| CA R1 | 50.90 | 75.98 | 73.80 | 66.97 | 61.48 |
| CA R2 | 71.29 | 78.41 | 73.11 | 66.66 | 60.18 |
| CA R3 | 63.86 | 69.38 | 65.56 | 61.28 | 57.00 |
| AVG | 62.01 | 74.59 | 70.82 | 64.97 | 59.55 |
| STD | 10.32 | 4.67 | 4.57 | 3.20 | 2.31 |
| CTRL R1 | 45.80 | 80.64 | 80.33 | 73.64 | |
| CTRL R2 | 45.80 | 84.41 | 83.39 | 76.22 | 69.25 |
| CTRL R3 | 45.80 | 77.15 | 75.92 | 70.11 | 62.74 |
| AVG | 45.80 | 80.73 | 79.88 | 73.32 | 66.00 |
| STD | 0.00 | 3.63 | 3.76 | 3.07 | 4.60 |

### Coated Tablets

All formulation percentages are based on weight. 3 replicates of 13 mm disc-shaped, 0.5 g tablets were compacted under 60 kN. All formulas consist of 23% 3NOP, 30% silica, and 47% ethyl cellulose. Tablets were then coated with 60-80 wt % of the corresponding coating material. Control tablet (CTRL) is uncoated. V1 coating solution was 20 wt % cellulose acetate Mn: 50000 in 70% acetone/ethyl acetate with BYK coating additives. V2 coating solution was 20 wt % in 70% acetone/ethyl acetate cellulose acetate Mn: 50000 with BYK coating additives. Ethyl cellulose coating solution was 20 wt % in 70% ethanol/water with 15 wt % triacetin. CAP coating solution was 18 wt % in 70% ethanol/water with 10 wt % propylene glycol. The 3NOP release profile of the coated tablets over 18 days is shown in Table 20. PGP-49;

TABLE 20
3NOP release profile of coated tablets over 18 days.
Total concentration of 3NOP is listed in mM.

| Time | 1 | 5 | 8 | 12 | 18 |
|---|---|---|---|---|---|
| V1 R1 | 33.04 | 70.32 | 70.57 | 66.04 | 60.88 |
| V1 R2 | 57.54 | 77.54 | 77.42 | 72.65 | 67.44 |
| V1 R3 | 61.42 | 71.21 | 70.08 | 65.71 | 61.19 |
| AVG | 50.67 | 73.02 | 72.69 | 68.13 | 63.17 |
| STD | 15.39 | 3.93 | 4.10 | 3.91 | 3.70 |
| V2 R1 | 31.15 | 66.47 | 65.62 | 61.03 | 56.78 |
| V2 R2 | 32.55 | 66.81 | 67.61 | 63.67 | 59.55 |
| V2 R3 | 53.59 | 69.37 | 71.28 | 67.53 | 63.01 |
| AVG | 39.10 | 67.55 | 68.17 | 64.08 | 59.78 |
| STD | 12.57 | 1.59 | 2.87 | 3.27 | 3.12 |
| EC R1 | 17.99 | 39.67 | 45.39 | 47.12 | 50.00 |
| EC R2 | 15.84 | 38.15 | 43.53 | 45.83 | 49.27 |
| EC R3 | 21.21 | 52.28 | 58.64 | 60.42 | 63.56 |
| AVG | 18.35 | 43.37 | 49.19 | 51.13 | 54.28 |
| STD | 2.70 | 7.75 | 8.24 | 8.08 | 8.05 |
| CAP R1 | 24.07 | 57.62 | 63.10 | 69.28 | 68.61 |
| CAP R2 | 23.24 | 56.30 | 61.85 | 60.67 | 59.42 |
| CAP R3 | 37.58 | 63.57 | 71.80 | 69.89 | 70.48 |
| AVG | 28.30 | 59.17 | 65.58 | 66.61 | 66.17 |
| STD | 8.05 | 3.87 | 5.42 | 5.15 | 5.92 |
| CTRL R1 | 61.62 | 97.29 | 89.52 | 81.26 | 74.20 |
| CTRL R2 | 45.40 | 82.69 | 79.42 | 73.16 | 67.07 |
| CTRL R3 | 53.75 | 83.30 | 76.77 | 70.18 | 64.12 |
| AVG | 53.59 | 87.76 | 81.90 | 74.87 | 68.47 |
| STD | 8.11 | 8.26 | 6.73 | 5.74 | 5.18 |

### Bilayer Coated Tablets

All formulation percentages are based on weight. 3 replicates of 13 mm disc-shaped, 0.5 g tablets were compacted under 60 kN. All formulas consist of 15% 3NOP, 20% silica, and 65% ethyl cellulose. Bilayer coatings were applied with ˜10 wt % of the first coating material and ˜20 wt % of the second coating material. CAP/EC denoted first and second coating material. The 3NOP release profile of bilayer coated tablets over 27 days is shown in Table 21.

TABLE 21
3NOP release profile of bilayer coated tablets over
27 days. Total concentration of 3NOP is listed in mM.

| | Time (days) | | | | | | | | |
|---|---|---|---|---|---|---|---|---|---|
| | 1.25 | 2.5 | 5.5 | 8 | 10 | 14 | 17 | 21 | 27 |
| CAP/EC | 6.27 | 8.80 | 15.97 | 18.49 | 20.41 | 24.15 | 24.59 | 24.46 | 26.21 |
| R1 | | | | | | | | | |
| CAP/EC | 4.39 | 7.77 | 15.50 | 17.78 | 19.54 | 22.90 | 23.44 | 23.74 | 25.94 |
| R2 | | | | | | | | | |
| CAP/EC | 5.63 | 8.43 | 15.44 | 17.84 | 19.29 | 20.36 | 20.94 | 21.18 | 22.65 |
| R3 | | | | | | | | | |
| AVG | 5.43 | 8.33 | 15.64 | 18.04 | 19.75 | 22.47 | 22.99 | 23.13 | 24.93 |
| STD | 0.96 | 0.52 | 0.29 | 0.39 | 0.59 | 1.93 | 1.87 | 1.73 | 1.98 |
| EC/CAP | 9.75 | 14.58 | 22.35 | 23.09 | 23.76 | 26.40 | 26.04 | 26.12 | 26.82 |
| R1 | | | | | | | | | |
| EC/CAP | 10.72 | 17.13 | 24.89 | 26.10 | 26.78 | 29.51 | 28.89 | 28.52 | 29.31 |
| R2 | | | | | | | | | |
| EC/CAP | 9.84 | 15.34 | 22.33 | 24.08 | 25.21 | 28.03 | 27.91 | 27.56 | 28.17 |
| R3 | | | | | | | | | |
| AVG | 10.10 | 15.68 | 23.19 | 24.42 | 25.25 | 27.98 | 27.61 | 27.40 | 28.10 |
| STD | 0.54 | 1.31 | 1.47 | 1.54 | 1.51 | 1.56 | 1.45 | 1.21 | 1.25 |
| CTRL | 26.11 | 28.76 | 36.26 | 37.49 | 36.75 | 35.77 | 33.69 | 31.26 | 32.66 |
| R1 | | | | | | | | | |
| CTRL | 26.12 | 28.43 | 38.45 | 39.57 | 38.58 | 37.45 | 34.91 | 32.16 | 33.30 |
| R2 | | | | | | | | | |
| CTRL | 25.23 | 29.94 | 40.65 | 41.23 | 45.02 | 42.91 | 39.10 | 27.92 | −2.04 |
| R3 | | | | | | | | | |
| AVG | 25.82 | 29.05 | 38.45 | 39.43 | 40.12 | 38.71 | 35.90 | 30.45 | 21.31 |
| STD | 0.51 | 0.79 | 2.19 | 1.87 | 4.34 | 3.73 | 2.84 | 2.24 | 20.22 |

### Adsorbent Study

Various adsorbents were assessed for potential binding affinity and adsorptive capacity of 3NOP. Activated carbon/charcoal and silica were identified as the most suitable adsorbents based on their high binding affinity and adsorptive capacity.

Briefly, 0.2 g of adsorbent was added to 4 mL of 2.5% 3NOP/H2O stock solution or 10 mL of 1% 3NOP/H2O stock solution. The solution was then placed in an ultrasonic bath for ˜4 hr and then allowed to equilibrate over 24 h. All materials were purchased from Sigma-Aldrich and used as received. After 24 h, adsorbents were tested for their binding affinity and adsorptive capacity by looking at the reduction of 3NOP concentration via HPLC at 230 nm.

The following adsorbents were tested and integrated areas were converted to mM concentrations of 3NOP based on calibration standards of known 3NOP concentration in distilled water, as seen in Table 22. FIG. 14 is graph showing the concentration of 3NOP (mM) v. adsorbent (20 mM stock solution).

TABLE 22
Change in concentration v. control of 0.2
g of adsorbent in 4 mL of 2.5% 3NOP/H2O.

| Adsorbent | 3NOP concentration (mM) |
|---|---|
| Control | 20.649 |
| Calcium Carbonate | 20.649 |
| Talc | 20.463 |
| Silica Gel (40-60 um) | 20.043 |
| Silica (5-20 nm) | 19.920 |
| Arginine | 19.782 |
| Lysine | 18.583 |
| Carbon Black | 17.422 |
| Activated Charcoal | 11.063 |

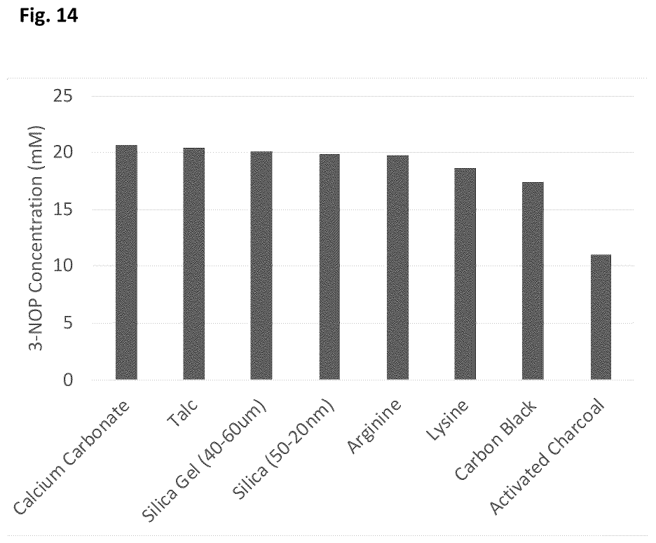

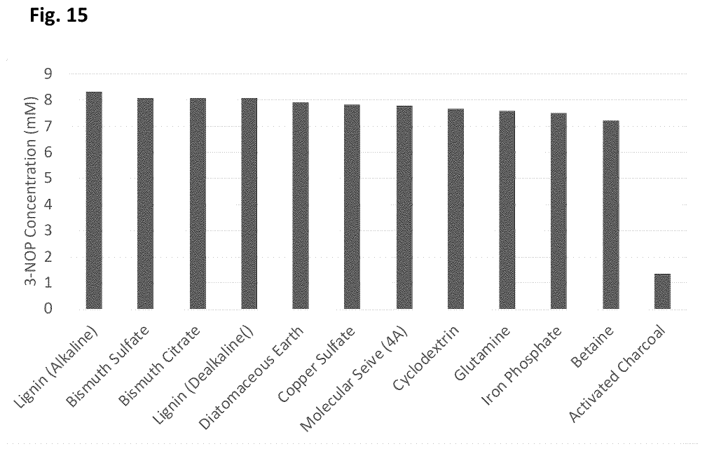

Table 23 shows the change in concentration v. control of 0.2 g of adsorbent in 10 mL of 1% 3NOP/H2O, and FIG. 15 shows 3NOP concentration (mM) v. adsorbent (˜8 mM stock solution).

TABLE 23
Change in concentration v. control of 0.2
g of adsorbent in 10 mL of 1% 3NOP/H2O.

| Adsorbent | 3NOP concentration (mM) |
|---|---|
| Control | 8.258 |
| Lignin (Alkaline) | 8.326 |
| Bismuth Sulfate | 8.079 |
| Bismuth Citrate | 8.072 |
| Lignin (Dealkaline) | 8.056 |
| Diatomaceous Earth | 7.895 |
| Copper Sulfate | 7.814 |
| Molecular Sieve (4 Å) | 7.771 |
| Cyclodextrin | 7.676 |
| Glutamine | 7.588 |
| Iron Phosphate | 7.503 |
| Betaine | 7.202 |
| Activated Charcoal | 1.358 |

Activated carbon shows strong binding affinity for 3NOP filtering ˜45 wt % in concentrated (20 mM) conditions with ˜80 wt % in more dilute concentrations (8 mM). It appears that the adsorbent pore size should be greater than 4 Å for adsorption of 3NOP. Mesoporous adsorbents with high surface area such as silica and activated carbon show high adsorptive capacity with no visible welting seen until ˜75 wt %. Layer-by-Layer

### Polyelectrolytes Coatings

Formulated tablets containing 3NOP, an adsorbent, and a binder can be coated with sequential layers of oppositely charged poly electrolytes. While not being bound by theory, it is believed that ionic crosslinking between the layers can result in a controlled release of 3NOP out of the tablets. Examples of polyelectrolyte pairs that can be used include polyglutamic acid and polylysine, polyallylamine hydrochloride and polyacrylic acid, and polyallylamine hydrochloride and polystyrene sulfonate. An exemplary formulated tablet includes an adsorbent of silica, arginine, lysine, and activated charcoal (30-45%) and a binder of ethyl cellulose and hydroxypropyl cellulose (40-55%).

TABLE 24
Formulation examples of 3NOP tablets.
Tablet weight varied from 0.2-0.5 g.
Tablet Formulation

| | Part per | | Part per |
|---|---|---|---|
| | formulation | | formulation |
| Adsorbent | (wt %) | Binder | (wt %) |
| Silica, Arginine, | 30-45 | Ethyl Cellulose, | 40-55 |
| Lysine, Activated | | Hydroxypropyl | |
| Charcoal | | Cellulose | |

The release data of 3NOP tablets coated with multilayers of some polyelectrolytes is shown in Table 25.

TABLE 25
Release (%) of 3NOP out of tablets formulated with
15% 3NOP, 30% silica, and 55% ethyl cellulose.

| | Release Compared to Control Tablets (%) | | | | | | | |
|---|---|---|---|---|---|---|---|---|
| PE Pair | Day 1 | Day 3 | Day 6 | Day 8 | Day 14 | Day 16 | Day 21 | Day 30 |
| PLK20 + | 58.1 | 56.6 | 61.3 | 62.6 | 64.7 | 63.6 | 63.8 | 63.2 |
| PRE20 | | | | | | | | |
| PAH + | 61.5 | 62.5 | 67.1 | 70.2 | 70.4 | 71.4 | 71.7 | 71.6 |
| PAA | | | | | | | | |
| PAH + | 61 | 66.5 | 74.7 | 76 | 77.5 | 76.6 | 76.7 | 74.8 |
| PSS | | | | | | | | |

Weight of tablets ~0.5 g. Calculations are based on a comparison to control tablets with no coating.

### 3NOP to Adsorbent Ratio Study

Optimal loading of 3NOP onto adsorbents was determined by varying ratios of 3NOP to adsorbent. 3NOP and adsorbent were speed mixed at 2000 rpm for 1 min at the following ratios by weight: 1:1.5, 1:2, 1:2.5. The adsorbed 3NOP powder mixture was then placed in distilled water at 1% 3NOP loading. The solution was allowed to equilibrate for 24 hr and then an aliquot was taken for HPLC to determine the reduction in 3NOP concentration. Table 26 shows the average integrates are of 3NOP in solution at various adsorbent ratios.

TABLE 26
Average integrated area of 3NOP in solution at varying 3NOP:adsorbent ratios

| | 3NOP:Adsorbent | | | | | |
|---|---|---|---|---|---|---|
| | 1:1.5 | | 1:2 | | 1:2.5 | |
| | Average | | Average | | Average | |
| | Integrated | Standard | Integrated | Standard | Integrated | Standard |
| | Area | Deviation | Area | Deviation | Area | Deviation |
| 3NOP | 2566.4 | 51.3 | 2566.4 | 51.3 | 2566.4 | 51.3 |
| Silica | 2499.4 | 293.2 | 2321.9 | 99.3 | 2340.9 | 30.3 |
| Activated | 1751.2 | 29.6 | 1581.9 | 8.5 | 1403.7 | 14.5 |
| charcoal | | | | | | |
| Lysine | 2336.2 | 61.7 | 2308.9 | 23.9 | 2266.6 | 56.1 |
| Arginine | 2406.0 | 113.2 | 2372.8 | 61.2 | 2435.2 | 230.2 |
| Glutamic | 2445.9 | 149.9 | 2452.5 | 78.9 | 2446.3 | 85.0 |
| acid | | | | | | |

Higher ratios of activated carbon to 3NOP result in higher adsorptive capacity. At ratio of 1:2.5 an adsorption capacity of ˜45% is observed and at a ratio of 1:1.5 an adsorption capacity of ˜30% is observed.

### Activated Carbon Tablets

Based on the 3NOP:adsorbent ratio study, the same ratios were tested for tablet integrity, friability, porosity, etc to determine the optimal 3NOP loading. 500 mg, 13 mm tablets were compacted under 60 kN.

High ratios of activated carbon (1:2.5 3NOP:adsorbent) resulted in tablets that absorb more water and coating solutions. This water/solvent absorption of non-wetted charcoal causes difficulty in producing uniform, defect free coatings. High loadings of activated charcoal >25% in tablets result in poor tablet integrity characterized by loss in hardness, increased porosity, tablet spilling, and poor shape retention. The preferred loading for tablets with poor disintegration was 1:1.5 3NOP:adsorbent.

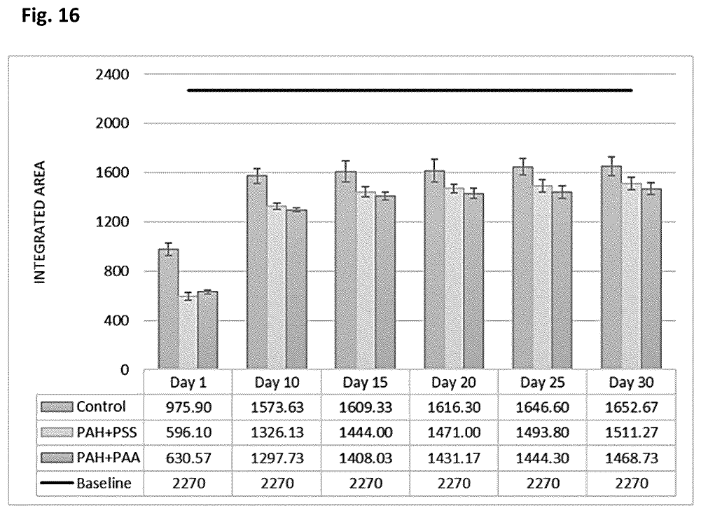

FIG. 16 shows a multilayer tablet formulation above consisting of 10 wt % 3NOP, 15 wt % activated carbon, and 75 wt % ethyl cellulose. The first day release of coated tablets is ˜40% less than uncoated tablets and after 30 days, ˜30% 3NOP remains adsorbed on activated carbon.

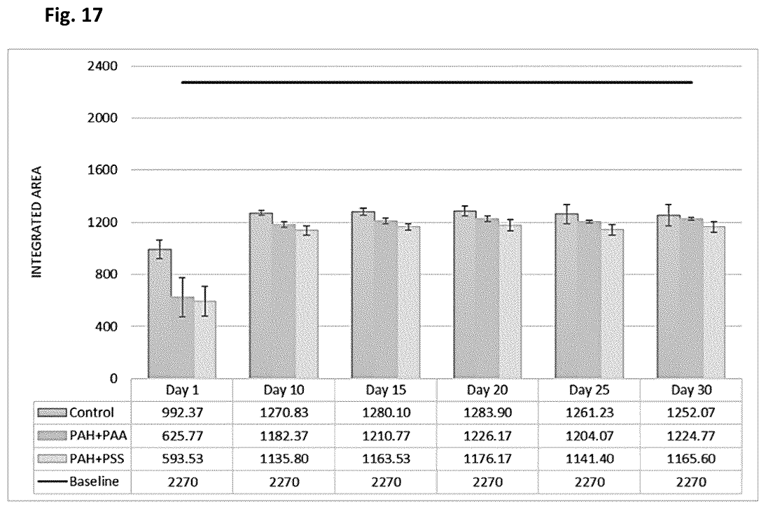

FIG. 17 shows a multilayer tablet formulation consisting of 10 wt % 3NOP, 25 wt % activated carbon, and 75 wt % ethyl cellulose. After 30 days, ˜50% 3NOP remains adsorbed on activated carbon. These adsorptive capacities are in-line with what was observed during the 3NOP:adsorbent ratio study.

FIG. 18 shows a multilayer formulation consisting of 10 wt % 3NOP, 15 wt % activated carbon, 5 wt % sodium lignosulfonate, and 70 wt % ethyl cellulose. The addition of 5 wt % sodium lignosulfonate improves coating adhesion to the tablet resulting in ˜30% reduction in the first day 3NOP release. Over time, the release rate remains similar to previous formulations with a residual 30% 3NOP remaining adsorbed to activated carbon.

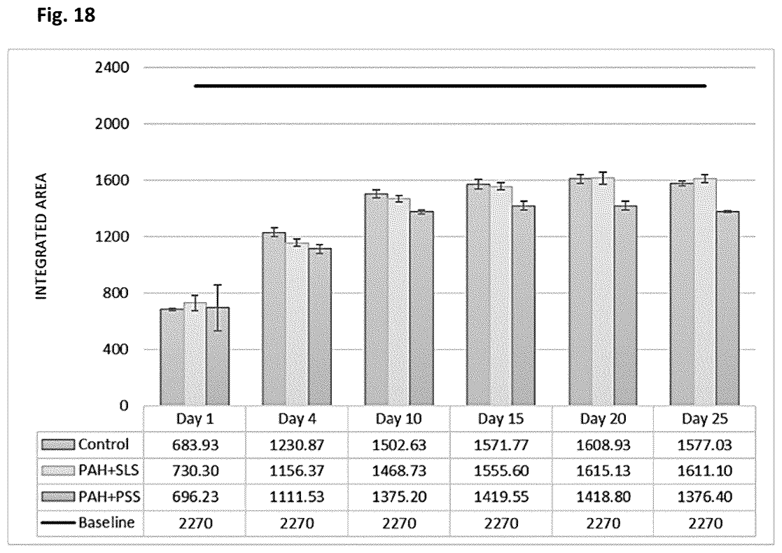

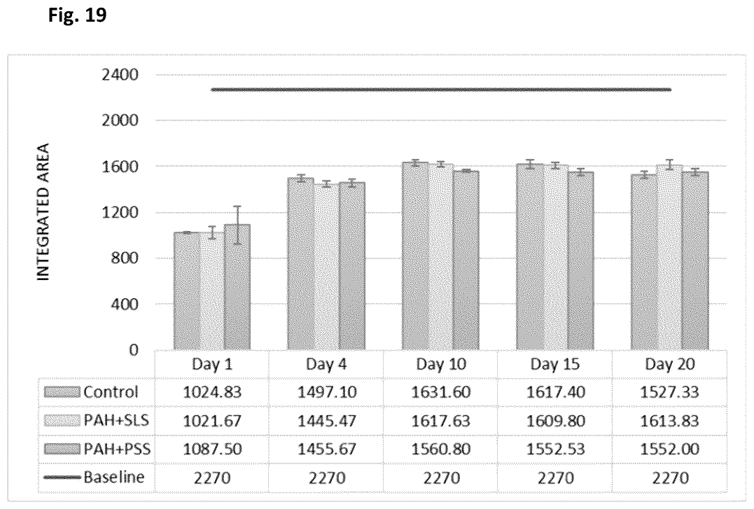

FIG. 19 shows a multilayer formulation consisting of 10 wt % 3NOP, 15 wt % activated carbon, 5 wt % hydroxypropyl cellulose, and 70 wt % ethyl cellulose. The addition of 5 wt % hydroxypropyl cellulose, a hydrophilic water-soluble binder, results in increased release between day 4 and day 10. After 30 days, ˜30% 3NOP remains adsorbed to activated carbon.

Silica v. Activated Carbon Controls to Assess Binding Affinity of 3NOP

Various control experiments were conducted to confirm the adsorbed 3NOP to activated carbon after 30 days. Activated carbon was compared to silica as preferred adsorbents. 500 mg of 1:1.5 3NOP:adsorbent were speed mixed at 2000 rpm for 1 min. The resulting powder mixture was placed in 10 mL of distilled water and 10 mL of acetonitrile (ACN) separately. The solutions were placed in a sonic bath for ˜5 hr, removed, and allowed to equilibrate for 24 hr.

Aliquots were filtered through a 0.22 um PTFE filter, and taken for HPLC to measure the 3NOP concentration in solution.

The same trend was observed in the adsorbent study. However, 3NOP is recoverable by washing the activated carbon with acetonitrile or a similar organic solvent with high 3NOP solubility (ie. ethanol, acetone, ethyl acetate, tetrahydrofuran, chloroform).

TABLE 27
Silica powder release v. activated carbon release
in distilled water and acetonitrile.

| | 24 hr in H2O | 24 hr in ACN |
|---|---|---|
| | Integrated Area | Integrated Area |
| Si-1 | 8415.7 | 8847 |
| Si-2 | 8507.3 | 8822.2 |
| Average | 8461.5 | 8834.6 |
| AC-1 | 4940.8 | 8103.9 |
| AC-2 | 5577.5 | 8082.8 |
| Average | 5259.2 | 8093.4 |

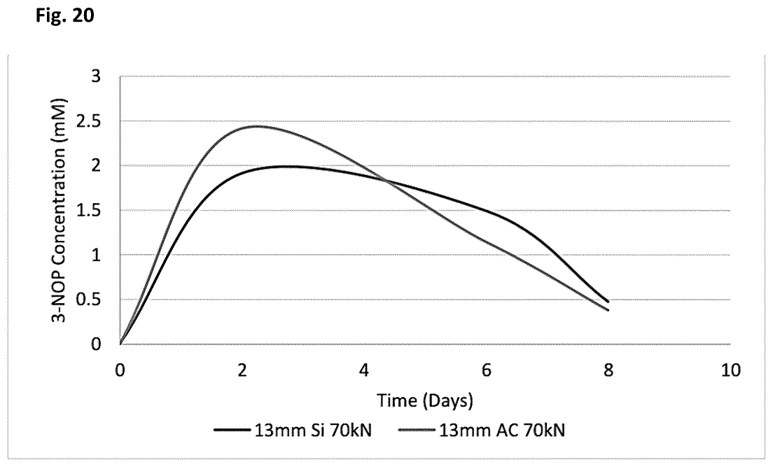

After 24 hr in water, less than 5 wt % 3NOP remained adsorbed to silica compared to the powder mix placed in acetonitrile. After 24 hr in water, ˜35 wt % 3NOP remained adsorbed to activated carbon compared to the powder mix placed in acetonitrile. By washing the activated carbon in acetonitrile, the adsorbed 3NOP can be recovered in solution proving that the remaining 30-50% 3NOP that is seen in FIG. 11-FIG. 14 remains adsorbed. Further studies need to be conducted to determine the driving forces for 3NOP desorption from activated carbon under different environmental conditions to utilize adsorptive capacity of activated carbon. This study also confirms that from previous tablet formulations, diffusion is the primary driver for 3NOP release of unbound 3NOP. This can be seen by very similar release profiles between silica-based adsorbent tablets and activated carbon-based adsorbent tablets. FIG. 20 compares a silica-based tablet and activated carbon-based tablet over 8 days. Small differences in release profiles could be due to differences in porosity between tablets using silica or activated carbon.

### Compounding of 3NOP

3NOP was compounded with a variety of matrix materials using a DSM Xplore MC15 conical twin-screw extruder. The extrudate was pelletized and used as is or further compression molded into tablets as a suitable 3NOP extended-release form.

3NOP was adsorbed onto the corresponding adsorbent and speed mixed with binder prior to compounding is a DSM Xplore MC15 conical twin-screw extruder at 100 C and 50 rpm. The strand was then pelletized, heated, and molded into a 13 mm disc-shaped tablet using a carver press under 40 kN of compaction force. Formula consists of 8.3% 3NOP, 25.0% arginine, 66.7% CAPA 6800.

TABLE 28
3NOP release profile of compounded tablets over 24 days.

| Time | 1 | 3 | 7 | 10 | 14 | 20 | 24 |
|---|---|---|---|---|---|---|---|
| Arg R1 | 8.97 | 16.37 | 22.55 | 23.42 | 24.11 | 23.07 | 24.56 |
| Arg R2 | 12.49 | 15.85 | 20.43 | 21.45 | 21.60 | 19.33 | 19.97 |
| Arg R3 | 9.90 | 17.78 | 24.52 | 24.50 | 24.36 | 22.60 | 22.33 |
| AVG | 10.45 | 16.67 | 22.50 | 23.13 | 23.36 | 21.67 | 22.29 |
| STD | 1.82 | 1.00 | 2.04 | 1.55 | 1.52 | 2.04 | 2.29 |

Total concentration of 3NOP is listed in mM.

Formulas consists of 11.1% 3NOP, 13.9% silica, 75.0% CAPA 6800 denoted “11PCL” and 19.4% 3NOP, 24.2% silica, 56.5% CAPA 6800 denoted “19PCL.”

TABLE 29
3NOP release profile of compounded tablets over 24
days. Total concentration of 3NOP is listed in mM.

| Time | 1 | 3 | 7 | 13 | 17 |
|---|---|---|---|---|---|
| 11PCL R1 | 9.79 | 18.77 | 24.31 | 29.15 | 27.93 |
| 11PCL R2 | 10.34 | 19.81 | 24.99 | 30.02 | 29.49 |
| 11PCL R3 | | 19.05 | 22.51 | 27.14 | 26.95 |
| AVG | 10.06 | 19.21 | 23.94 | 28.77 | 28.12 |
| STD | 0.39 | 0.54 | 1.28 | 1.48 | 1.28 |
| 19PCL R1 | 23.55 | 40.56 | 49.99 | 55.00 | 3.48 |
| 19PCL R2 | 24.19 | 40.59 | 51.03 | 53.46 | 51.64 |
| 19PCL R3 | 25.05 | 42.76 | 52.21 | 57.93 | 55.77 |
| AVG | 24.26 | 41.30 | 51.08 | 55.47 | 36.96 |
| STD | 0.75 | 1.26 | 1.11 | 2.27 | 29.07 |

### Compounded Polyelectrolyte Complexes for Extended Release of 3NOP

Solid polyelectrolyte complexes can be processible when plasticized with aqueous electrolytes to act as a matrix for 3NOP molecules. An example of a polyelectrolyte pair for compounding is polydiallyldimethylammonium chloride (PDADMAC), as a cationic polyelectrolyte, and polystyrene sulfonate (PSS), as an anionic polyelectrolyte. At no salt conditions, the complex is brittle and not ideal for processing and the addition of salt, such as sodium chloride, can help with the plasticization and further processing. A trial formulation used for compounding includes mixing of equal charge stoichiometry of PDADMAC and PSS solutions with a concentration of 100 mM, with respect to each polymer, and a total volume of 200 mL (as per preparation method described in ACS Appl. Mater. Interfaces 2015, 7, 895-901). The polyelectrolyte complex can be separated, removed from the supernatant phase, and soaked in salt water with a concentration of 1M for 24 h. The polyelectrolyte complex should be decanted and ready for compounding. A typical formulation used for compounding is summarized in Table 30.

TABLE 30
Compounding formulation with polyelectrolyte complexes for extended
release of 3NOP. Total materials used 20 g. 3NOP was mixed with
arginine (as in solid powder) overnight at 30 C.
PEC Compounding Formulation

| | Part per | | Part per |
|---|---|---|---|
| | formulation | | formulation |
| 3NOP Complex | (wt %) | PEC Complex | (wt %) |
| 3NOP: Arginine | 25 | PDADMAC: PSS | 75 |
| (ratio 1:3, wt %) | | (Equimolar with | |
| | | respect to the | |
| | | monomer) | |

Table 31 shows release (%) of 3NOP out of compounded PEC formulated as per Table 21. The compounded PECs were cut into 5-10 mm pellets and soaked in water (10 mL) for the release study.

TABLE 31
Release percentages
Release (%)

| Day 1 | Day 4 | Day 6 | Day 12 |
|---|---|---|---|
| 22.4 | 34.4 | 35 | 35.7 |

### Compounded Pellets

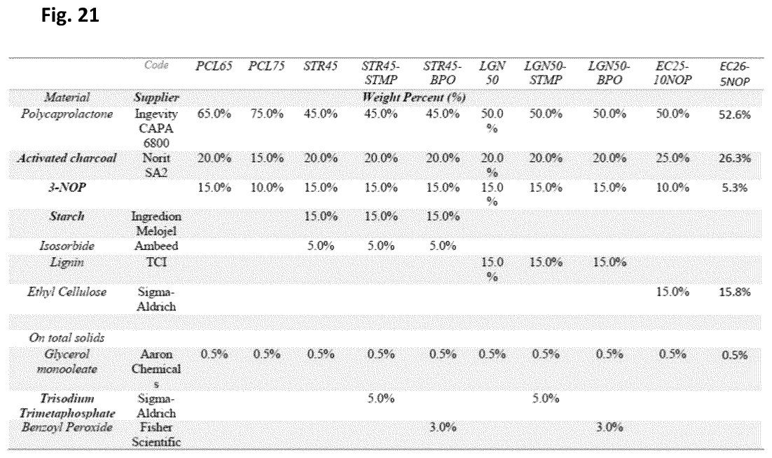

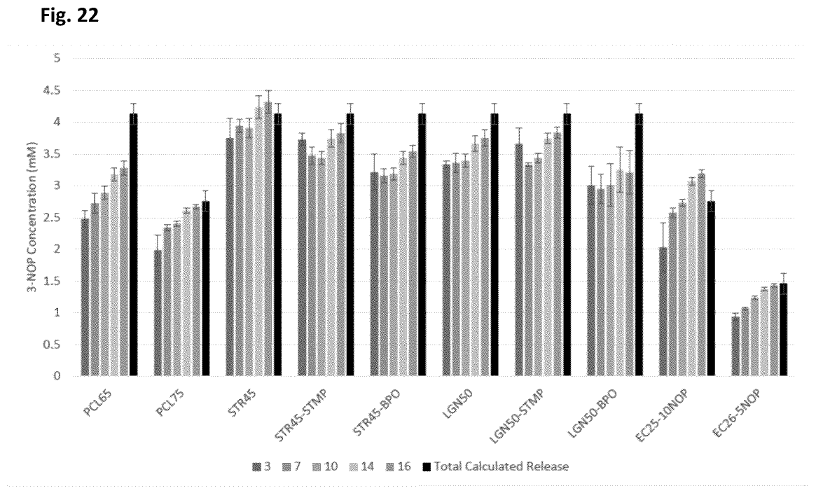

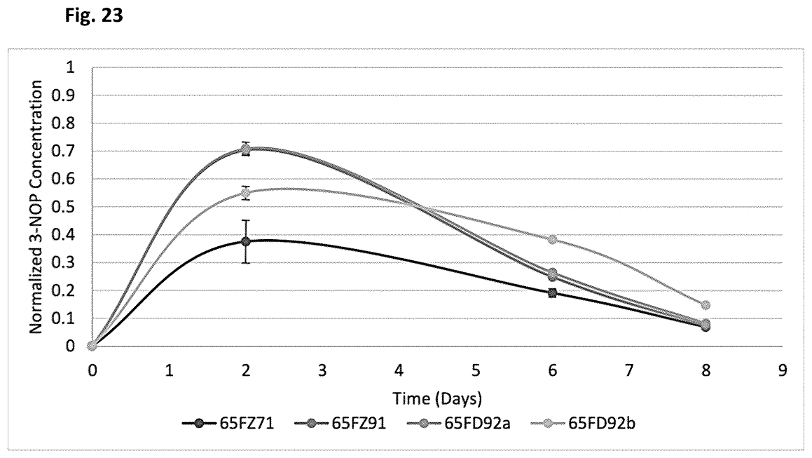

3NOP was adsorbed onto the corresponding adsorbent and speed mixed with binder prior to compounding is a DSM Xplore MC15 conical twin-screw extruder at temperatures of 110-130 C and 100 rpm. The extrudate was then pelletized forming ˜2 mm pellets. Material suppliers are listed in Table 30. Polybutylene succinate grades FZ71, FZ91, FD92 were supplied by Mitsubishi Chemical. Polycaprolactone-based formulations are summarized in the Table in FIG. 21. FIG. 22 shows 3NOP release in mM for polycaprolactone-based formulations.

TABLE 32
Polybutylene succinate-based formulations

| | | | | |
|---|---|---|---|---|
| Code | 65FZ71 | 65FZ91 | 65FD92a | 65FD92b |

Material

| | | | | |
|---|---|---|---|---|
| Polybutylene succinate | 65.0% | 65.0% | 65.0% | 65.0% |
| Activated charcoal | 20.0% | 20.0% | 20.0% | 20.0% |
| 3NOP | 15.0% | 15.0% | 15.0% | 15.0% |

On total solids

| | | | | |
|---|---|---|---|---|
| Glycerol monooleate | 0.5% | 0.5% | 0.5% | 0.0% |
| Compounding Temperature | 120 C. | 120 C. | 130 C. | 110 C. |

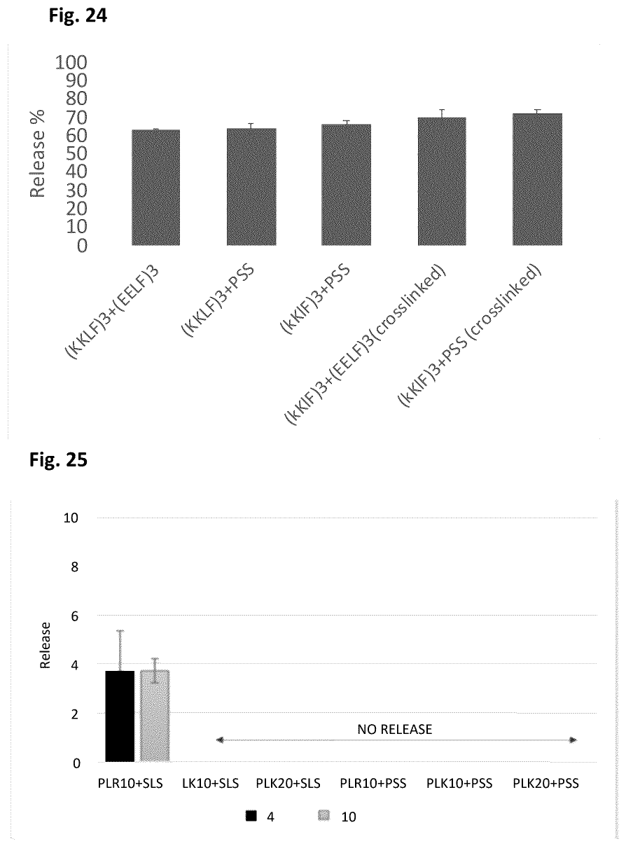

FIG. 24 is chart showing the normalized 3NOP concentration v. Release time in days of polybutylene succinate-based formulations. Lower 3NOP loadings (<10%) have a less pronounced burst release over the first 7 days of monitored release. Glycerol monooleate was identified at a potent processing aid at loadings <1%. High levels <25 wt % activated carbon can easily be processed unlike silica which leads to high viscosity and screw torque. Cross-linked systems show slower release in comparison to un-crosslinked systems. High shear conditions of compounding appear to destroy activated carbon structure and adsorption capacity is lost.

### Microcapsules for the Encapsulation of 3NOP

Polyelectrolyte complexes (PECs) are used to form microcapsules and encapsulate 3NOP. PECs are formed through attractive forces such as ionic interactions, hydrogen bonding, hydrophobic, and pi-interactions between the polyelectrolytes. Polyelectrolytes are polymers with ionic groups bonded to counter ions and can dissociate in a solution to make positively or negatively charged polymers. Herein, PEC microcapsules refer to interconnecting networks of polyelectrolytes formed upon the interaction between oppositely charged polymers.

Examples of polyelectrolytes used in this invention include synthetic polyelectrolytes such as polystyrene sulfonate (PSS), polyacrylic acid (PAA), and polyallylamine hydrochloride (PAH). Further examples include naturally occurring polyelectrolytes such as chitosan and alginate or ionic biopolymers such as proteins, enzymes, and charged polypeptides.

Examples of Polyelectrolytes (charged polymers with positively or negatively charged repeating units) Used:

### Synthetic Polyelectrolytes

Polystyrene sulfonate (PSS), molecular weight ˜200,000 Da, Sigma Aldrich
Sodium Lignosulfonate (SLS), molecular weight ˜52000 Da, Sigma Aldrich
Polyethyleneimine (PEI), molecular weight ˜2000 Da, Polysciences
Polyallylamine hydrochloride (PAH), molecular weight ˜17,500 Da, Sigma Aldrich

### Commercially Available Peptide Sequences

Poly (L-lysine) (PLK10), degree of polymerization: 10, molecular weight: 1600 Da, Alamanda Polymers
Poly (L-lysine) (PLK20), degree of polymerization: 20, molecular weight: 3300 Da, Alamanda Polymers
Poly (L-arginine) (PLR10), degree of polymerization: 10, molecular weight: 1900 Da, Alamanda Polymers
Examples of Crosslinker Used:
Glutaraldehyde, 25% solution in water, Sigma Aldrich
EDC (1-ethyl-3-(3-dimethylaminopropyl) carbodiimide hydrochloride), Thermo Fisher.

### PEC Microcapsules Preparation

Equal stoichiometry of oppositely charged polyelectrolytes is sequentially added to water or water-3NOP solutions followed by vortexing for 10 s, after the addition of each component, to form PEC microcapsules. Some samples are chemically crosslinked (glutaraldehyde is an example of the crosslinker used). The PEC microcapsules may also be formed with non-stoichiometric ratios of polyelectrolytes. While stoichiometric ratios of polyelectrolytes provide almost a neutral microcapsule, microcapsules prepared with non-stoichiometric ratios are positively or negatively charged. All polyelectrolyte solutions are prepared in water and pH adjusted. The concentration of solutions is based on the monomer charge. The stock solution of 3NOP is prepared in water with a concentration of 100 mM and pH adjusted to 8.

High-performance liquid chromatography (HPLC) is used to determine the concentration of 3NOP in PEC microcapsules and calculate the release of 3NOP from the microcapsules. Samples are centrifuged to separate the supernatant phase from the complex phase. The supernatant phase is then removed carefully by using a micropipette and transferred to a 2 mL glass vial for further analysis. The 3NOP release of some PEC microcapsules prepared at neutral pH conditions and after 96 h of preparation is summarized in Table 31.

TABLE 33
3NOP release (%) from the PEC microcapsules. Samples
are prepared with a 3NOP concentration of 100 μM. Final
pH solution ~7. K, L, F, and E refer to lysine, leucine,
phenylalanine, and glutamic acid, respectively. PSS
refers to polystyrene sulfonate. Table 33 discloses
(KKLF)3 as SEQ ID NO: 79312, (EELF)3 as SEQ ID NO:
79313 and (kKlF)3 as SEQ ID NO: 79314.

| | | 3NOP Concentration |
|---|---|---|
| | Release (%) at | in Microcapsules |
| Polyelectrolyte Pair | 96 h timepoint | (μM) |
| (KKLF)3 + (EELF)3 | 63 | 37 |
| (KKLF)3 + PSS | 64 | 36 |
| (kKlF)3 + PSS | 66 | 34 |
| (kKlF)3 + (EELF)3(crosslinked) | 70 | 30 |
| (kKlF)3 + PSS (crosslinked) | 72 | 28 |

FIG. 24 is a graph showing 3NOP release (%) from the PEC microcapsules. Samples are prepared with a 3NOP concentration of 100 μM. Final pH solution ˜7. K, L, F, and E refer to lysine, leucine, phenylalanine, and glutamic acid, respectively. PSS refers to polystyrene sulfonate.

Examples of release profiles of some microcapsules made of positively charged polypeptides with either polystyrene sulfonate (PSS) or sodium lignosulfonate (SLS) are shown in FIG. 25 (3NOP release (%) from the PEC microcapsules. Samples are prepared with a 3NOP concentration of 100 μM. Final pH solution ˜7. PLR refers to poly (L-arginine), and PLK refers to poly (L-lysine). SLS and PSS refer to sodium lignosulfonate and polystyrene sulfonate).

The encapsulation efficiency of some PEC microcapsules prepared at neutral pH conditions is summarized in Table 34. Table 35 shows the encapsulation efficiency of samples prepared at pH-8.

TABLE 34
Encapsulation efficiency of the PEC microcapsules. Samples are prepared
with a 3NOP concentration of 100 μM. Final pH solution ~7.
Upper and lower cases in (kKlF)3 sequences represent L and D-chirality,
respectively. Table 34 discloses (KKLF)3 as SEQ ID NO: 79312, (EELF)3
as SEQ ID NO: 79313 and (kKlF)3 as SEQ ID NO: 79314.

| | Total Concentration | Encapsulation | 3NOP Concentration |
|---|---|---|---|
| | (monomer charge basis) | Efficiency | in Microcapsules |
| Polyelectrolyte Pair | (mM) | (%) | (μM) |
| PLK20 + PRE20 | 5 | 7.4 | 7.4 |
| PLR10 + PRE20 | 5 | 0 | 0 |
| (KKLF)3 + (EELF)3 | 5 | 100 | 100 |
| (kKlF)3 + (EELF)3 | 5 | 22.2 | 22.2 |
| (KKLF)3 + PSS | 5 | 37 | 37 |
| (kKlF)3 + PSS | 5 | 100 | 100 |
| PAH + PSS | 5 | 30.9 | 30.9 |

TABLE 35
Encapsulation efficiency of the PEC microcapsules. Samples are prepared with
a 3NOP concentration of 2.5 mM. Final pH solution ~8. Upper and lower cases
in (kKlF)3 (SEQ ID NO: 79314) sequences represent L and D-chirality of the amino
acid residue, respectively. Table 35 discloses (kKlF)3 as SEQ ID NO: 79314.

| | Total Concentration | Encapsulation | 3NOP |
|---|---|---|---|
| | (monomer charge | Efficiency | Concentration in |
| Polyelectrolyte Pair | basis) (mM) | (%) | Microcapsules (mM) |
| PLR10 + PRE20 | 5 | 50 | 1.25 |
| PLK20 + PRE20 | 5 | 53.8 | 1.34 |
| (kKlF)3 + PRE20 | 5 | 50.2 | 1.26 |

Physical Cross/Inking of 3NOP with Small Molecules

3NOP molecules can form hydrogen bonds with small molecules such as amino acids to make a larger size conjugate resulting in enhanced encapsulation efficiency. For example, an equal stoichiometry of 3NOP and two selective amino acids, arginine and lysine, were reacted in aqueous solution for 24 h, and the solution was used for encapsulation studies. The results of such encapsulation with some PEC microcapsules are shown in Table 36.

TABLE 36
Encapsulation efficiency of the PEC microcapsules. Samples are prepared
with a 3NOP concentration of 125 μM. Final pH solution ~7. Table
36 discloses (KKLF)3 as SEQ ID NO: 79312 and (EELF)3 as SEQ ID NO: 79313.

| | Encapsulation | | 3NOP Concentration in | |
|---|---|---|---|---|
| Polyelectrolyte | Efficiency (%) | | Microcapsules (μM) | |
| Pair | Arginine-3NOP | Lysine-3NOP | Arginine-3NOP | Lysine-3NOP |
| PLR10 + PRE20 | 4.6 | 19.1 | 5.75 | 23.9 |
| PLK20 + PRE20 | 34.3 | 28.4 | 42.9 | 35.5 |
| (KKLF)3 + (EELF)3 | 27.4 | 34.8 | 34.25 | 43.5 |
| PAH + PAA | 30.3 | 47.1 | 37.9 | 58.9 |
| PAH + PSS | 67.4 | 67.6 | 84.3 | 84.5 |

### Example 13: Vaccination of Cattle Population

It is demonstrated herein that vaccination of exemplary vaccines of the present disclosure resulted in generation of antibodies which were effective in neutralizing ruminal methanogens.

### Treatment Groups

TABLE 37
Treatment descriptions

| Treatment ID | Count | Treatment Description |
|---|---|---|
| 9A | (n = 20) | Untreated |
| 9G | (n = 5) | 1 mL of RPF51698.1 (SEQ ID NO: 11032) + |
| | | ADC46800.1 (SEQ ID NO: 5382) |
| | | 1 mL of Enable C1 |
| 9H | (n = 4) | 0.5 mL of RPF51698.1 (SEQ ID NO: 11032) |
| | | 0.5 mL of Enable C1 |
| 9I | (n = 2) | 0.5 mL of ADC46800.1 (SEQ ID NO: 5382) |
| | | 0.5 mL of Enable C1 |

### 1. Conclusions

### 1.1 Vaccine Formulations

Treatment with protein antigen formulations (9G+9H+91) did not adversely affect daily feed intake nor average daily gain. Any trends in enteric emissions changes reported herein are present when CH₄ production (g/d) is normalized for DMI (i.e., CH₄ yield) or ADG (i.e., CH₄ intensity). Furthermore, no adverse events were reported for animals treated with protein antigen formulations.

Antigen-specific ELISA assay of sera from animals vaccinated with treatments 9G and 9H demonstrated elevated IgG's against RPF51698.1 protein. IgG response peaked in the d42 sample. Antigen-specific ELISA assay of sera from animas vaccinated with treatments 9G, 9H, and 9I demonstrated elevated IgG's reactive to ADC46800.1 protein. This further demonstrates that vaccines comprising RPF51698.1 and/or ADC46800.1 protein antigen induce an immune response generating antibodies capable of binding ADC46800.1. This observation suggests one or more epitopes on ADC46800.1 raise cross reactive IgG response to both protein homologs.

Treatment 9G and 91 formulations appeared to be efficacious following primary vaccination (d0), while treatment 9H formulations appeared to be efficacious following secondary vaccination. Efficacy for protein antigen formulations was determined using a covariate analysis (i.e., AA) using the pre-prime period (p0) as the baseline for emissions.

When compared to the untreated control animals, treatment with protein antigen formulations resulted in mitigation of ˜46 kg of CO₂ₑ over 10 weeks (p1, p2+p3). Specifically, treatment resulted in a reduction in CH₄ intensity of ˜10%, ˜15 and ˜17% in p1, p2 and p3, respectively, as compared to p0.

### 2. Protein Antigen Vaccine Formulation

### 2.1. Preparation of Recombinant Protein

Recombinant proteins were expressed using an E. coli T7 based bacterial expression system. Briefly, genes encoding the proteins of interest were amplified from genomic DNA using PCR primers designed to facilitate Gibson assembly into a linearized pRSET expression plasmid. Sanger or nanopore sequencing was used to confirm the sequence of the target gene and its intended subcloning into the expression vector. Subcloning included the addition of an N-terminal histidine affinity tag to facilitate purification and confirmation of expression. Expression plasmids were used to transform a lon-expression strain of E. coli such as BL21(DE3) pLysS, BL21(DE3), or clear coli (a DE3 lysogen strain engineered for reduced levels of endotoxin). Shake flask expression conditions were determined empirically and used either LB medium with IPTG induction or commercial autoinduction medium (Magic Medium). Cells were lysed with BugBuster reagent.

Purifications were conducted using HiTrap HP Nickel columns using either an FPLC to apply a linear imidazole elution gradient or manually driven syringes to create a step gradient. Fractions containing target protein were pooled and concentrated using Centricon concentrators. Concentrated purified protein was dialyzed against DPBS using slide-alyzer cassettes. If needed, endotoxin levels of preparations were reduced using polymixin-B resin followed by another round of concentration and dialysis. To reduce the likelihood of cross contamination of the proteins, purification media was used for a single target protein only. Preparations were sterilized by passing through 0.22 um syringe filters. Endotoxin levels were monitored either out-of-house by Charles River Laboratory or in-house using a Charles River NextGen-PTS instrument. Protein concentrations were routinely monitored by use of OD280 and final concentrations were determined relative to BSA standards using Bradford Assay.

Purified proteins were diluted to a concentration of 200 μg per 500 uL in DPBS for single protein treatment group doses or 200 ug each protein/1 mL for the dual protein treatment group. Purity of final material was confirmed by SDS-PAGE with coomassie brilliant blue staining. Purity of at least 85% was confirmed using densiometry of gel images with ImageJ. Endotoxin levels were confirmed to be less than 2000 EU per dose. Table 38 describes the final protein preparations.

TABLE 38
Protein preparations summary

| Protein | % Purity | EU per dose | Group |
|---|---|---|---|
| RPF51698.1 | 87 | 780 | 9H |
| ADC46800.1 | 85 | 1,100 | 9I |
| RPF51698.1 + ADC46800.1 | 86 (avg) | 1,900 | 9G |

### 3. Vaccine Preparation+Administration

### 3.1. Treatment Assignments

Prior to the study's initiation, steers were transferred to the study site, McGregor Research Center (McGregor, TX 76657) for a GreenFeed training period lasting approximately 8 weeks to learn behaviors associated with GreenFeed systems, as well as to allow for adaptation to a grower's diet (i.e., 40% rolled corn, 25% dried distillers grains, 2.5% mineral premix, 7.5% molasses and 25% sudan hay).

Only healthy, weaned Black Angus cross steers were utilized for this Project. All animals were weaned and received pre-weaned vaccinations a minimum of sixty days (60 d) prior to initiation of the study. In addition, animals demonstrated no clinical signs of health concerns (e.g., dull or sunken eye, depression, signs of scours, listlessness, weakness, or raspy breathing) at the time of enrollment. All animals selected were within approximately one hundred (100) lbs of each other at the time of enrollment.

### 3.2. Vaccine Preparation

Vaccines were prepared in their final formulation, i.e., antigen mixed with adjuvant, onsite on the following dates: Jun. 10, 2024 and Jul. 1, 2024.

Briefly, vaccines were prepared by mixing antigen prepared as described above, and the appropriate adjuvant at a 1:1 volumetric ratio via inversion less than 24 hours prior to injection. Exact mixing volumes are defined in Table 37.

Specifically, frozen protein antigens were shipped on dry ice overnight to the Research Center, with storage at −80° C. before and after shipment. Protein antigens were packaged as single aliquots, e.g., 1 mL each. Prior to injection, antigen aliquots were thawed on wet ice and mixed with equal volume of veterinary adjuvant (1:1 mix ratio) using gentle inversion to prepare a final vaccine volume of 0.4-2 mL. Vaccines were kept at 4° C. and/or on wet ice until injection, less than 24 hours.

### 3.3. Adjuvant Selection

ENABL C1 (Huvepharma, cat. #7010201) was chosen due to its known performance profile and USDA-approved 21 d withdrawal period.

### 3.4. Vaccine Administration

Animals were assigned to one of nine treatment groups, detailed below in Appendix A. Vaccines were administered subcutaneously by veterinary-trained staff. Just prior to injection, animals were restrained using a cattle silencer, and injections were administered into the animal's neck region using an 18-gauge 0.5-0.75″ needle 4. Dry matter intake, body weight, and daily gain 4.1. Dry matter intake (DMI) To ensure that vaccination did not adversely affect intake, dry matter intake was recorded daily using GrowSafe feed bunks. DMI for Individual animals was recorded by GrowSafe using known RFIDs.

Throughout the entirety of the study period, animals were fed once daily in the morning and had access to a water source at all times.

Individual daily feed intake (kg/d) was used to normalize enteric methane emissions, as well as to confirm that vaccination did not have a deleterious effect on intake. Intake was recorded from May 21, 2024 through Aug. 13, 2024 using GrowSafe feed bunks. Animals also consumed alfalfa pellets from the GreenFeed systems.

Total daily feed intake was determined by summing the feed intake recorded using the GrowSafe feed bunks and the estimated pellet consumption from the GreenFeeds, using the reported good visit duration. Daily dry matter intake was then calculated by correcting the total feed intake for the dry matter content of the feed type; average dry matter content of the feed consumed from the GrowSafe bunks and the pellets consumed from the GreenFeed System.

Dry matter content of the growing diet and GreenFeed pellets were determined.

### 5. Results

### 5.1. Body Weight

To ensure that vaccination did not adversely affect animal weight gain, body weight (kg) was recorded weekly throughout the study period. Body weight was recorded using a livestock scale. Specifically, animals to be weighed were walked through a pipe alley from their housing area to the holding pens. After being weighed, they were released to return to their holding pen with other animals from the same pen. At the end of the weighing, animals were walked back to their assigned pen in the barn.

Bodyweight measurements were used to determine average daily gain (ADG) throughout the study period (Jun. 4, 2024-Aug. 13, 2024). ADG was determined by taking the linear regression of weekly body weight measurements collected over 11 weeks.

ADG=SLOPE(body weight_{period X-Y},measurement date_{period X-Y})

Except for Group 9I, ADG over the entire study period was not significantly different from the untreated control (Group 9 A+9B). Group 9I was significantly greater (P=0.0004) than the untreated control, suggesting that there was no deleterious effect following any of the treatments; the observed difference between Group 9I and the control (Group 9 A+9B) may be attributable to the number of biological replicates in Group 9I (n=2), as compared to other treatment groups. When all protein antigen vaccines were considered together (Group 9G+9H+9I), there was no significant difference in ADG as compared to the untreated animals (Group 9 A+9B).

TABLE 39
ADG (kg per day) averaged per treatment group

| Treatment Group | ADG (kg/d) | P value |
|---|---|---|
| Untreated control | 1.12 | N/A |
| 9 A + 9B | | |
| All gen2 | 1.20 (↑) | 0.187 |
| 9G + 9H + 9I | | |
| 9G | 1.16 (↑) | 0.368 |
| 9H | 1.17 (↑) | 0.373 |
| 9I | 1.33 (↑) | 0.0004 |

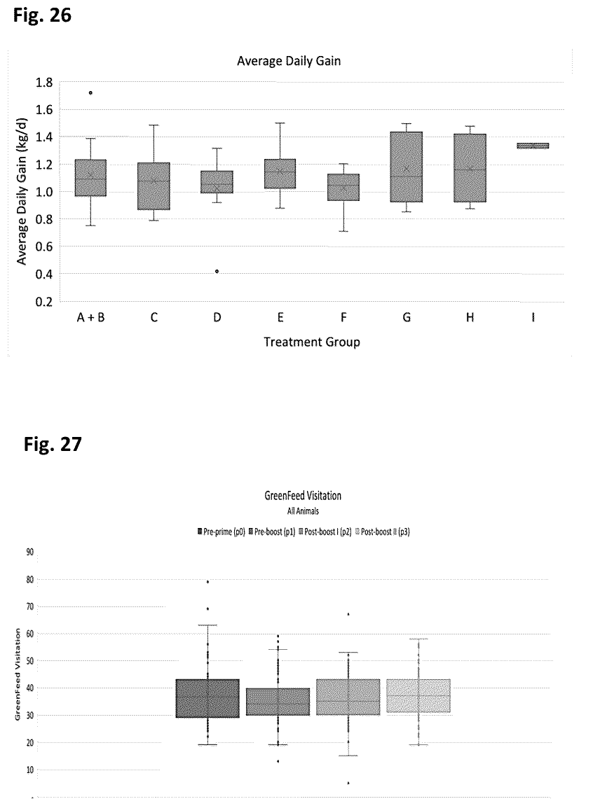

ADG (kg/per day) for individual animals is shown in FIG. 26 and Appendix B.

### 6.1. Enteric Emissions Measurement

Enteric methane, hydrogen and carbon dioxide emissions were measured daily throughout the study period using GreenFeed skid systems and GrowSafe feed bunks. Animals had free access to the GreenFeed systems throughout the study period; animals were not physically coerced to use the system. Methane yield and intensity was normalized for dry matter intake for each measurement period separately. Measurement periods were binned into 21-day intervals to align with the prime-boost vaccination schedule, as well as allow for sufficient GreenFeed Spot visits; ≥20 spot visits per measurement period are recommended for analysis.

TABLE 40
GreenFeed measurement periods

| | | | Avg. GreenFeed |
|---|---|---|---|
| Period | Start Date | End Date | Spot Visits |
| 0 | May 21, 2024 | Jun. 10, 2024 | 35 |
| 1 | Jun. 11, 2024 | Jul. 1, 2024 | 38 |
| 2 | Jul. 2, 2024 | Jul. 22, 2024 | 37 |
| 3 | Jul. 23, 2024 | Aug. 19, 2024* | 37 |

*_{#}Extended + 7 d beyond the planned period end date (28 d total). Drop in wk 7 GreenFeed visitation would have resulted in 15 animals failing GreenFeed visitation criteria (>=20 visits) if p3 were not extended.

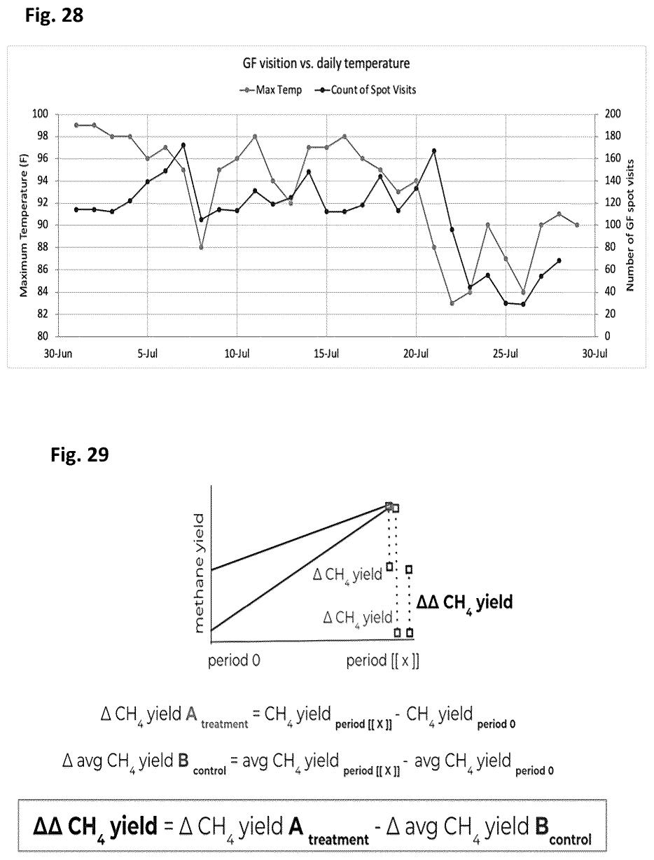

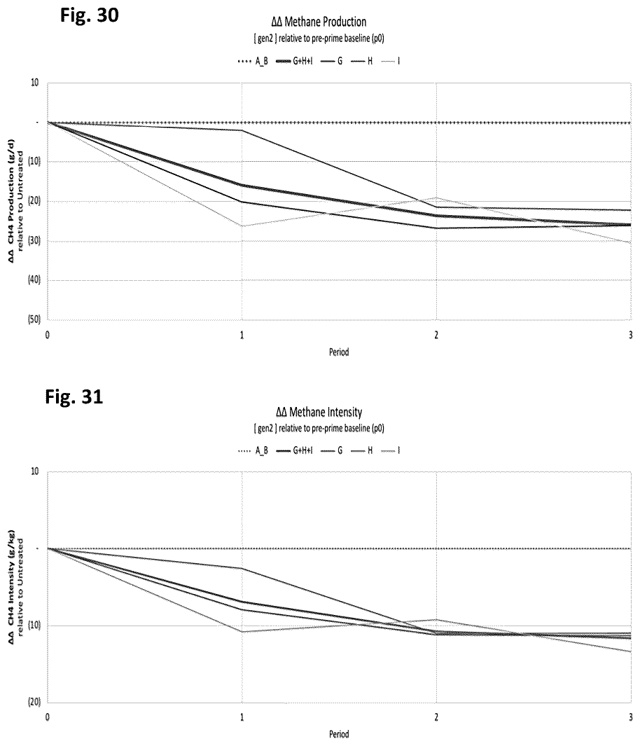

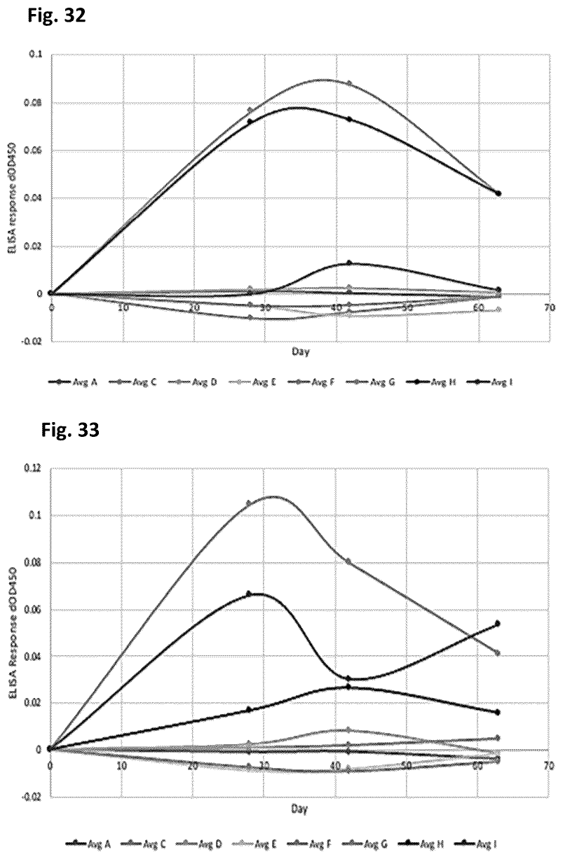

Period 3 (p3) was extended +7 d to from Aug. 12, 2024 to Aug. 19, 2024 in order to meet the minimum visitation threshold (>=20 spot visits) for the majority of animals. GreenFeed visitation greatly decreased in the first week of p3 (i.e., wk 7). If p3 had not been extended, 15 of 71 animals would have been excluded from the final emissions analysis for poor visitation; between Jul. 23, 2024 and Aug. 12, 2024, the average GreenFeed visitation was 27. The decrease in GreenFeed visits during wk 7 may be attributable to a drop in ambient air temperature, as no equipment malfunctions or loss of data was reported. The GreenFeed units are located beneath a shade structure, and, with the corresponding drop in temperature, it's possible that animals spent more time in the sun, away from the GreenFeed unit and therefore did not visit as often (FIG. 28).

### 7.1. Methane Emissions

A covariate analysis was used to interpret average daily methane production, yield and intensity. The rate of change between measurement periods in treatment groups was compared against the rate of change between measurement periods in the untreated control (Group 9A+9B) in a AA analysis (FIG. 29). The following equations were used to perform this analysis Definitions:

$$C {H}_{4} production = grams of CH4 per day$$

$${CH}_{4} yield = \frac{grams of CH4 per day}{kg of DMI per day}$$

$${CH}_{4} intensity = \frac{grams of CH4 per day}{kg of bodyweight gained per day ( ADG )}$$

### Equations:

$$A vg {CH}_{4} production = AVERAGE ( {CH4 massflow values}_{period X} )$$

$$Avg DMI = [ [ DEFINE ] ]$$

$${CH}_{4} yield = [ [ DEFINE ] ]$$

$${CH}_{4} intensity = \frac{{( Avg CH4 production )}_{period X}}{ADG}$$

$$Δ C {H}_{4} production = {( Avg CH4 production )}_{period X} - {( Avg CH4 production )}_{period Y}$$

$$ΔΔ C {H}_{4} production = Δ {CH}_{4} {production}_{group X ; period X - Y} - Δ Avg CH4 {production}_{control ; period X - Y}$$

Efficacy for protein antigen formulations was determined using a covariate analysis (i.e., AA) using the pre-prime period (p0) as the baseline for emissions.

Treatment with protein antigen formulations (Group 9G+9H+91) resulted in an average mitigation of ˜46.5 kg of CO₂ₑ per animal over 10 weeks (p1, p²+p3).

TABLE 41
ΔΔ CH₄ production (g/d) for protein antigen formulations

| | | Avg ΔΔ CH₄ Production | |
|---|---|---|---|
| Treatment Groups | Period | (g/d), per animal* | P value |
| 9G + 9H + 9I | p1 - p0 | −16.01 (↓) | 0.006 |
| (n = 10) | p2 - p0 | −23.71 (↓) | 0.002 |
| | p3 - p0 | −25.89 (↓) | 0.001 |
| 9G + 9I | p1 - p0 | −21.97 (↓) | 0.0003 |
| (n = 7) | p2 - p0 | −24.64 (↓) | 0.00001 |
| | p3 - p0 | −27.43 (↓) | 0.001 |

*_{#}Δ CH₄ production relative to untreated animals (Groups A + B) over the same periods of time.

CH₄ intensity was significantly reduced following treatment protein antigen formulations, as compared to changes in CH₄ intensity in the control animals over the same measurement periods (Table 42). Treatment with protein antigen formulations resulted in an average reduction in CH₄ intensity of ˜0.10%, ˜15% and ˜17% per animal in p1, p2 and p3, respectively, as compared to p0.

TABLE 42
ΔΔ CH₄ intensity (g/kg ADG) for protein antigen formulations

| | | Avg ΔΔ CH₄ Intensity | |
|---|---|---|---|
| Treatment Groups | Period | (g/kg ADG), per animal* | P value |
| 9G + 9H + 9I | p1 - p0 | −15.27 (↓) | 0.003 |
| (n = 10) | p2 - p0 | −23.73 (↓) | 0.0002 |
| | p3 - p0 | −25.67 (↓) | 0.0002 |
| 9G + 9I | p1 - p0 | −19.36 (↓) | 0.001 |
| (n = 7) | p2 - p0 | −23.44 (↓) | 0.00001 |
| | p3 - p0 | −26.26 (↓) | 0.0004 |

*_{#}Δ CH₄ intensity relative to untreated animals (Groups A + B) over the same periods of time.

8.1. Sample collection+analysis

Total blood samples were collected periodically throughout the duration of the immune monitoring period for subsequent processing. For a detailed sample collection, see Table 43 below.

TABLE 43
Sample collection schedule

| | | |
|---|---|---|
| Date | Vaccination | Total blood |

| | | | |
|---|---|---|---|
| Jun. 11, 2024 | d 0 | X | X |
| Jul. 2, 2024 | d 21 | X | |
| Jul. 9, 2024 | d 28 | | X |
| Jul. 23, 2024 | d 42 | | X |
| Aug. 13, 2024 | d 63 | | X |

No adverse events were observed.

### 8.1.1. Sera

### 8.1.1.1. Sample Collection

Generally, total blood samples were collected under the supervision of veterinary-trained staff. Samples were collected via jugular vein needle puncture into blood tubes. Collected blood samples were then clarified, via ultracentrifugation, to collect serum within twenty-four hours of collection. Clarified serum samples were aliquoted, labeled and stored at or under −20° C.

### 8.1.1.2. Sera Sample Receipt+Processing

Frozen sera samples returned from the field were thawed at 4° C. overnight or rapidly in an ambient temperature water bath. Thawed samples were mixed by inversion at least 3 times, aliquoted to 4×1 mL in 1.5 mL Eppendorf tubes. Additionally, a 5 mL pooled sera sample was made for each treatment group, which was also aliquoted. A working aliquot of each sample was held at 4° C., and the remaining volumes refrozen and held at −80° C.

### 8.1.1.3. Determination of IgG Response Using WC or Protein ELISA

Protein ELISA plates were prepared from recombinant methanogen protein preparations. Briefly, ˜0.2 micrograms of protein in carbonate buffer was applied to wells of a high binding plate and allowed to coat overnight at 4° C. Coated plates were washed with PBS-T and then blocked with SuperBlock T20 (ThermoFisher) for 3 hrs at 4° C. After blocking, plates were emptied, dried at room temperature overnight, vacuum sealed with a desiccant pack, and held at 4° C. until use.

Sera samples diluted to 1:25,000 were applied to ELISA plates and binding IgG's were detected after washing using a commercially available anti-bovine IgG H & L chain secondary antibody. The WC ELISA data shown below represents the average of technical triplicates (i.e., the same sera dilution run in multiple ELISA plates).

APPENDIX A
Treatment assignments

| Visual ID | RFID | Treatment ID |
|---|---|---|
| 112L | 000000000840003242071747 | 9A |
| 1165L | 000000000840003234349454 | 9A |
| 674L | 000000000840003242071873 | 9A |
| 459L | 000000000840003234349414 | 9A |
| 687L | 000000000840003242071828 | 9A |
| 598L | 000000000840003234349578 | 9A |
| 587L | 000000000840003242071497 | 9A |
| 1025L | 000000000840003234349264 | 9A |
| 135L | 000000000840003242071667 | 9A |
| 078L | 000000000840003242071732 | 9A |
| 1186L | 000000000840003234349477 | 9A |
| 294L | 000000000840003242071736 | 9A |
| 695L | 000000000840003242071637 | 9A |
| 637L | 000000000840003242071841 | 9A |
| 299L | 000000000840003234349545 | 9A |
| 649L | 000000000840003242071881 | 9A |
| 629L | 000000000840003242071463 | 9A |
| 334L | 000000000840003242071454 | 9A |
| 443L | 000000000840003242071471 | 9A |
| 163L | 000000000840003242071646 | 9A |
| 121L | 000000000840003242071647 | 9G |
| 336L | 000000000840003242071449 | 9G |
| 335L | 000000000840003242071444 | 9G |
| 349L | 000000000840003242071455 | 9G |
| 1100L | 000000000840003234349456 | 9G |
| 705L | 000000000840003242071763 | 9H |
| 1168L | 000000000840003234349470 | 9H |
| 155L | 000000000840003242071658 | 9H |
| 617L | 000000000840003242071483 | 9H |
| 673L | 000000000840003234349556 | 9I |
| 1130L | 000000000840003234349458 | 9I |

APPENDIX B
Average daily gain (kg/d), measured weekly

| Treatment Group | Visual ID | ADG (kg/d) |
|---|---|---|
| 9A | 078L | 1.33 |
| 9A | 1025L | 1.22 |
| 9A | 112L | 1.23 |
| 9A | 1165L | 1.04 |
| 9A | 135L | 1.15 |
| 9A | 459L | 0.93 |
| 9A | 587L | 1.07 |
| 9A | 598L | 0.96 |
| 9A | 674L | 1.00 |
| 9A | 687L | 0.85 |
| 9A | 1186L | 1.10 |
| 9A | 163L | 1.72 |
| 9A | 294L | 0.89 |
| 9A | 299L | 1.39 |
| 9A | 334L | 1.33 |
| 9A | 443L | 1.23 |
| 9A | 629L | 0.98 |
| 9A | 637L | 0.75 |
| 9A | 649L | 1.09 |
| 9A | 695L | 1.09 |
| 9F | 511L | 0.93 |
| 9F | 577L | 1.00 |
| 9G | 1100L | 1.49 |
| 9G | 121L | 1.37 |
| 9G | 335L | 0.85 |
| 9G | 336L | 1.11 |
| 9G | 349L | 0.99 |
| 9H | 1168L | 1.07 |
| 9H | 155L | 1.25 |
| 9H | 617L | 1.48 |
| 9H | 705L | 0.87 |
| 9I | 1130L | 1.35 |
| 9I | 673L | 1.31 |

### Abbreviations and Acronyms

| | |
|---|---|
| ADG | Average daily gain |
| BSA | Bovine serum albumin |
| CH₄ | Methane |
| CO₂ | Carbon dioxide |
| COA | Certificate of Analysis |
| CVB | Center for Veterinary Biology |
| DMI | Dry matter intake |
| DMSO | Dimethyl sulfoxide |
| DNA | Deoxyribonucleic acid |
| DPBS | Dulbecco's PBS |
| ELISA | Enzyme-Linked Immunosorbent Assay |
| FPLC | Fast protein liquid chromatography |
| H₂ | Hydrogen |
| GHG | Greenhouse gas |
| Ig | Immunoglobulin |
| IgA | Immunoglobulin A |
| IgG | Immunoglobulin G |
| O₂ | Oxygen |
| OD | Optical density |
| PCR | Polymerase chain reaction |
| PBS | Phosphate buffered saline |
| PBST | PBS Tween 20 |
| USDA | US Department of Agriculture |
| WC | Whole cell |
| WCVL | Whole cell vaccine lot |

### Ranges

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A vaccine composition comprising at least one polypeptide and/or at least one peptide of at least one cell surface protein or a fragment thereof of at least one methanogen.

2. (canceled)

3. The vaccine composition of claim 1, wherein the at least one methanogen comprises Methanobrevibacter gottschalkii and/or Methanobrevibacter ruminantium.

4-22. (canceled)

23. A method of treating a disease in a subject, the method comprising administering to the subject a vaccine composition of claim 1.

24-25. (canceled)

26. A method of inducing an immune response against at least one methanogen in a subject, the method comprising administering to the subject the vaccine of claim 1.

27. (canceled)

28. A method of reducing rumen lactate, increasing pH, or combination thereof in a subject, the method comprising administering to the subject the vaccine of claim 1.

29. A method of reducing the activity, number, and/or type of methanogens in a digestive tract of a subject, the method comprising administering to the subject the vaccine composition of claim 1.

30. (canceled)

31. A method of reducing the amount of methane (CH₄) and/or hydrogen (H₂) emitted by a subject, preferably eructated and/or exhaled, the method comprising administering to the subject the vaccine composition of claim 1, optionally wherein the amount of methane (CH₄) is normalized to an amount of CO₂ emitted by the subject (i.e., CH₄/CO₂).

32-37. (canceled)

38. A method of increasing the amount of carbon dioxide (CO₂) emitted by a subject, preferably eructated and/or exhaled, the method comprising administering to the subject the vaccine composition of claim 1.

39-57. (canceled)

58. The method of claim 26, further comprising administering to the subject (a) at least one agent that reduces the level of methane and/or hydrogen produced by the subject; and/or (b) at least one agent that increases production efficiency.

59-62. (canceled)

63. The method of claim 58, wherein the at least one agent comprises:
(a) an agent selected from the agents listed in Tables 9-13;
(b) 3-Nitrooxypropanol (3NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, or any combination thereof, and/or
(c) Monensin (Rumensin®), Optigrid® 45 (ractopamine hydrochloride), Amprolium (Corid), Bacitracin (Albac, BMD), Bambermycin (GainPro), Decoquinate (Deccox), Fenbendazole (Safe-Guard), Laidlomycin (Cattlyst), Lasalocid (Bovetec), Melengestrol Acetate (MGA), Methoprene (Altosid), Morantel (Rumatel), Poloxalene (Bloat Guard), Ractopamine (Optaflexx, Actogain), Tetraclovinphos (Rabon), or any combination thereof.

64-120. (canceled)

121. An antibody produced by the method of claim 26, or an antigen-binding fragment thereof.

122-125. (canceled)

126. Milk and/or a derivative thereof produced by the subject of claim 26.

127-130. (canceled)

131. An animal feed comprising:
(a) the antibody produced according to the method of claim 26;
(b) at least one agent that reduces methane and/or hydrogen production in a subject, optionally wherein the at least one agent is selected from the agents in Tables 9-13;
(c) the milk and/or a derivative thereof produced by the subject of claim 26; or
(d) any combination of two or more of (a)-(c).

132-133. (canceled)

134. A method of reducing methane and/or hydrogen production or increasing CO₂ production in a subject, the method comprising orally administering to and/or feeding the subject the antibody produced according to the method of claim 26, the milk and/or a derivative thereof produced by the subject of claim 26, the animal feed comprising the antibody and/or milk and/or a derivative thereof, at least one agent that reduces methane and/or hydrogen production in a subject, or any combination of two or more thereof.

135-151. (canceled)

152. A kit comprising the vaccine composition of claim 1.

153-162. (canceled)

163. A method of treating a subject afflicted with a disease, the method comprising to the subject:
(a) the vaccine composition of claim 1;
(b) the antibody produced by a subject administered with the vaccine composition of claim 1;
(c) the milk and/or derivative thereof produced by a subject administered with the vaccine composition of claim 1;
(d) the animal feed comprising the antibody of (b), and/or the milk and/or derivative thereof of (c);
(e) at least one agent that reduces methane and/or hydrogen production in a subject, optionally wherein the at least one agent is selected from the agents in Tables 9-13; or
(f) any combination of two or more thereof.

164. (canceled)

165. A method of reducing CH₄ emissions in a ruminant comprising administering to the ruminant a vaccine composition of claim 1 comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the CH₄ emissions are reduced by at least about 10%, 11%, 12%, 13%, 14%, 15%, 16%, 17%, 18%, 19%, or 20% as compared to an untreated control ruminant.

166. A method of reducing H₂ emissions in a ruminant comprising administering to the ruminant a vaccine composition of claim 1 comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the H₂ emissions are reduced by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% as compared to an untreated control ruminant.

167. A method of increasing the productivity of a ruminant comprising administering to the ruminant a vaccine composition comprising at least one polypeptide and/or at least one peptide of at least one cell surface protein or a fragment thereof (e.g., an antigenic fragment, e.g., a fragment comprising an epitope, e.g., a fragment comprising an extracellular domain or a portion thereof) of at least one methanogen, wherein the productivity is increased by at least about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% as compared to an untreated control ruminant.

168-169. (canceled)

170. An animal injected subcutaneously with the vaccine composition of claim 1, wherein the vaccine composition comprises about a dosage of between 0.1 mg/kg and 250 mg/kg, or any range in between or any value in between.

171. A method of producing a low carbon animal product, the method comprising:
(a) administering to an animal (e.g., a mammal, a ruminant) a vaccine composition of claim 1;
(b) determining an amount of emissions of at least one greenhouse gas (e.g., CO₂, CH₄, N₂O, and/or H₂) from the animal of (a) following administration until animal product harvesting;
(c) determining a first carbon intensity as a ratio of a first amount of emissions from the vaccinated animal in (b) and the amount of harvested animal product;
(d) determining a second carbon intensity as a ratio of a second amount of carbon emissions from an untreated animal and the same amount of harvested animal product; and
(e) determining the difference between the first carbon intensity and the second carbon intensity.

172-175. (canceled)
